(19) United States
(12) Patent Application Publication
(10) Pub. No.: US 2020/0361864 A1
(43) Pub. Date: Nov. 19, 2020

(54) Peptide Conjugates, Conjugation Process, and Uses Thereof

(71) Applicant: Auckland UniServices Limited, Auckland (NZ)

(72) Inventors: Margaret Anne Brimble, Auckland (NZ); Peter Roderick Dunbar, Auckland (NZ); Daniel Verdon, Auckland (NZ); Geoffrey Martyn Williams, Auckland (NZ)

(21) Appl. No.: 16/638,185

(22) PCT Filed: Aug. 30, 2018
(86) PCT No.: PCT/IB2018/056611

§ 371 Date: Feb. 11, 2020

(30) Foreign Application Priority Data
Aug. 30, 2017 (NZ) 735008

(51) Int. Cl. C07C 323/58; C07K 1/36; C07K 14/47; A61K 47/54

(52) U.S. Cl. CPC C07C 323/58; A61K 47/542; C07K 14/4748; C07K 1/36

(57) ABSTRACT

The invention relates to peptide conjugates, methods for making peptide conjugates, conjugates produced by the methods, and pharmaceutical compositions comprising the conjugates. Methods of eliciting immune responses in a subject and methods of vaccinating a subject, uses of the conjugates for the same, and uses of the conjugates in the manufacture of medicaments for the same are also contemplated.

### TECHNICAL FIELD

The present invention relates to peptide conjugates, methods for making peptide conjugates, conjugates produced by the methods, pharmaceutical compositions comprising the conjugates, methods of eliciting immune responses in a subject and methods of vaccinating a subject, uses of the conjugates for the same, and uses of the conjugates in the manufacture of medicaments for the same. The present invention also relates to methods of making compounds useful in the synthesis of peptide conjugates of the invention and to such compounds.

### BACKGROUND ART

Synthetic peptide vaccines generally comprise a synthetic copy of an immunogenic part of protein antigens. This approach to vaccine development has a number of advantages, including ease of synthesis, avoidance of potentially toxic biological by-products and straightforward characterisation.

A key issue in the development of peptide vaccines is the lack of immunogenicity displayed by peptides as sole vaccine components. It is usually necessary to include in the vaccine an adjuvant, designed to activate components of the innate immune system (e.g. Freund's adjuvant).

An alternative strategy in peptide vaccine design is to create self-adjuvanting vaccines in which the peptide epitope of interest is covalently linked to an appropriate adjuvant. Such self-adjuvanting vaccines may have enhanced antigen uptake, presentation and dendritic cell maturation compared to simple co-formulation of the antigen with an external adjuvant.

Several self-adjuvanting vaccines have been developed, but preparation of the vaccines can be complicated.

There is an ongoing need for new self-adjuvanting vaccines and new methods of making self-adjuvanting vaccines. It is an object of the present invention to go some way towards meeting these needs; and/or to at least provide the public with a useful choice.

Other objects of the invention may become apparent from the following description which is given by way of example only.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention broadly consists in a peptide conjugate compound of the formula (I):

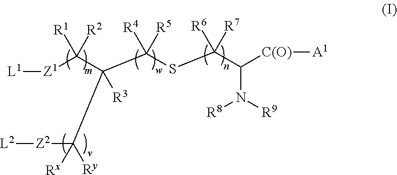

wherein
m and w are each independently an integer from 0 to 7 and v is an integer from 0 to 5,
provided that:
   the sum of m, v, and w is at least 3; and
   the sum of m and w is from 0 to 7;
n is 1 or 2;
Z1 and Z2 are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
R1, R2, Rx, Ry, R4, R5, R6, and R7 at each instance of m, v, w, and n are each independently hydrogen or C1-6aliphatic;
R, R3, and R8 are each independently hydrogen or C1-6aliphatic;
R9 is hydrogen, C1-6aliphatic, an amino protecting group, L3-C(O)—, or A2;
L1 and L2 are each independently selected from is C5-21aliphatic or C4-20heteroaliphatic;
L3 is C1-21aliphatic or C2-20heteroaliphatic;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any aliphatic or heteroaliphatic present in any of R, R1, R2, R3, R4, R5, R6, R7, R8, R9, Rx, Ry, L1, L2, and L3 is optionally substituted;
or a pharmaceutically acceptable salt or solvate thereof;
with the proviso that:
(1) at least one of R9 and A1 is a peptide comprising, consisting essentially of, or consisting of an amino acid sequence selected from the group consisting of:
(a) 8 or more contiguous amino acid residues from the sequence Xaa₁Xaa₂Xaa₃Xaa₄LQQLSLLMWITQXaa₂₂FLPVFLAQPPSGQRR [SEQ ID NO:1], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids,
(b) 8 or more contiguous amino acid residues from the sequence Xaa₁Xaa₂Xaa₃LQQLSLLMWITQXaa₂₂FLPVFLAQPPSGQRR [SEQ ID NO:2], wherein Xaa₁ is absent or is S, Xaa₂ is absent or is a hydrophilic amino acid, and Xaa₃ is absent or is from one to ten hydrophilic amino acids,
(c) 8 or more contiguous amino acid residues from the sequence Xaa₁Xaa₂LQQLSLLMWITQXaa₂₂FLPVFLAQPPSGQRR [SEQ ID NO:3], wherein Xaa₁ is absent or is S, and Xaa₂ is absent or is from one to four hydrophilic amino acids,
(d) 8 or more contiguous amino acid residues from the sequence

[SEQ ID NO: 4]
SKKKKLQQLSLLMWITQXaa₂₂FLPVFLAQPPSGQRR,

(e) the sequence of any one of SEQ ID NOs: 1 to 4,
(f) 8 or more contiguous amino acid residues from the sequence

[SEQ ID NO: 5]
LQQLSLLMWITQXaa₂₂FLPVFLAQPPSGQRR,

(g) the sequence of SEQ ID NO: 5,
(h) 8 or more contiguous amino acid residues from the sequence SLLMWITQXaa₂₂FLPVF [SEQ ID NO:6],
(i) the sequence of SEQ ID NO: 6,
(j) 8 or more contiguous amino acid residues from the sequence SKKKKSLLMWITQXaa₂₂ [SEQ ID NO:7],
(k) the sequence of SEQ ID NO: 7,
(l) 8 or more contiguous amino acid residues from the sequence SLLMWITQXaa₂₂ [SEQ ID NO:8],
(m) the sequence of SEQ ID NO: 8,
(n) or any combination of two or more of (a) to (m) above,
   wherein Xaa₂₂ in each sequence is independently any naturally occurring amino acid except C (for example V, I, or L), and any sequence of 8 or more contiguous amino acid residues from any of the above sequences comprises Xaa₂₂; or
   (2) m is an integer from 3 to 7, and at least one of R9 and A1 is an amino acid or a peptide.

In another aspect, the present invention broadly consists in a peptide conjugate compound of the formula (I):

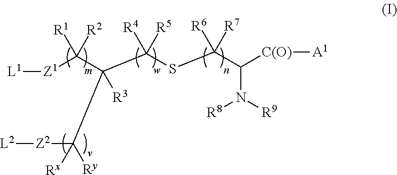

wherein
m and w are each independently an integer from 0 to 7 and v is an integer from 0 to 5,
provided that:
   the sum of m, v, and w is at least 3; and
   the sum of m and w is from 0 to 7;
n is 1 or 2;
Z1 and Z2 are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
R1, R2, Rx, Ry, R4, R5, R6, and R7 at each instance of m, v, w, and n are each independently hydrogen or C1-6aliphatic;
R, R3, and R8 are each independently hydrogen or C1-6aliphatic;
R9 is hydrogen, C1-6aliphatic, an amino protecting group, L3-C(O)—, or A2;
L1 and L2 are each independently selected from is C5-21aliphatic or C4-20heteroaliphatic;
L3 is C1-21aliphatic or C2-20heteroaliphatic;
A1 is an amino acid, a peptide, OH, OP1, NH₂, or NHP2, wherein P1 is a carboxyl protecting group, and wherein P2 is a carboxamide protecting group;
A2 is an amino acid or a peptide;
wherein any aliphatic or heteroaliphatic present in any of R, R1, R2, R3, R4, R5, R6, R7, R8, R9, Rx, Ry, L1, L2, and L3 is optionally substituted;
or a pharmaceutically acceptable salt or solvate thereof;
with the proviso that:
(1) at least one of R9 and A1 is a peptide comprising, consisting essentially of, or consisting of an amino acid sequence selected from the group consisting of:
(a) 8 or more contiguous amino acid residues from the sequence Xaa₁Xaa₂Xaa₃Xaa₄LQQLSLLMWITQXaa₂₂FLPVFLAQPPSGQRR [SEQ ID NO:1], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids
(b) 8 or more contiguous amino acid residues from the sequence Xaa₁Xaa₂Xaa₃LQQLSLLMWITQXaa₂₂FLPVFLAQPPSGQRR [SEQ ID NO:2], wherein Xaa₁ is absent or is S, Xaa₂ is absent or is a hydrophilic amino acid, and Xaa₃ is absent or is from one to ten hydrophilic amino acids,
(c) 8 or more contiguous amino acid residues from the sequence Xaa₁Xaa₂LQQLSLLMWITQXaa₂₂FLPVFLAQPPSGQRR [SEQ ID NO:3], wherein Xaa₁ is absent or is S, and Xaa₂ is absent or is from one to four hydrophilic amino acids,
(d) 8 or more contiguous amino acid residues from the sequence

[SEQ ID NO: 4
SKKKKLQQLSLLMWITQXaa₂₂FLPVFLAQPPSGQRR,

(e) the sequence of any one of SEQ ID NOs: 1 to 4,
(f) 8 or more contiguous amino acid residues from the sequence

[SEQ ID NO: 5]
LQQLSLLMWITQXaa₂₂FLPVFLAQPPSGQRR,

(g) the sequence of SEQ ID NO: 5,
(h) 8 or more contiguous amino acid residues from the sequence SLLMWITQXaa₂₂FLPVF [SEQ ID NO:6],
(i) the sequence of SEQ ID NO: 6,
(j) 8 or more contiguous amino acid residues from the sequence SKKKKSLLMWITQXaa₂₂ [SEQ ID NO:7],
(k) the sequence of SEQ ID NO: 7,
(l) 8 or more contiguous amino acid residues from the sequence SLLMWITQXaa₂₂ [SEQ ID NO:8],
(m) the sequence of SEQ ID NO: 8,
(n) or any combination of two or more of (a) to (m) above,
   wherein Xaa₂₂ in each sequence is independently any naturally occurring amino acid except C (for example V, I, or L), and any sequence of 8 or more contiguous amino acid residues from any of the above sequences comprises Xaa₂₂.

In another aspect, the present invention broadly consists in a peptide conjugate compound of the formula (I):

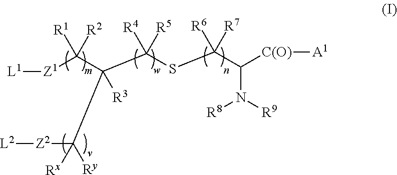

wherein
m and w are each independently an integer from 0 to 7 and v is an integer from 0 to 5,
provided that:
   the sum of m, v, and w is at least 3; and
   the sum of m and w is from 0 to 7;
n is 1 or 2;
Z1 and Z2 are each independently selected from the group consisting of —O—, —NR—, —S—, S(O)—, —SO₂—, —C(O)O—, —OC(O)—, —C(O)NR—, —NRC(O)—, —C(O)S—, SC(O)—, —OC(O)O—, —NRC(O)O—, —OC(O)NR—, and —NRC(O)NR—;
R1, R2, Rx, Ry, R4, R5, R6, and R7 at each instance of m, v, w, and n are each independently hydrogen or C1-6aliphatic;
R, R3, and R8 are each independently hydrogen or C1-6aliphatic;
R9 is hydrogen, C1-6aliphatic, an amino protecting group, L3-C(O)—, or A2;
L1 and L2 are each independently selected from is C5-21aliphatic or C4-20heteroaliphatic;
L3 is C1-21aliphatic or C2-20heteroaliphatic;
A1 is an amino acid, a peptide, OH, OP1, NH₂, or NHP2, wherein P1 is a carboxyl protecting group, and wherein P2 is a carboxamide protecting group;
A2 is an amino acid or a peptide;
wherein any aliphatic or heteroaliphatic present in any of R, R1, R2, R3, R4, R5, R6, R7, R8, R9, Rx, Ry, L1, L2, and L3 is optionally substituted;
or a pharmaceutically acceptable salt or solvate thereof;
with the proviso that:
(2) m is an integer from 3 to 7, and at least one of R9 and A1 is an amino acid or a peptide.

Any of the embodiments or preferences described herein may relate to any of the aspects herein alone or in combination with any one or more embodiments or preferences described herein, unless stated or indicated otherwise.

In various embodiments,
R1, R2, Rx, Ry, R4, R5, R6, and R7 at each instance of m, v, w, and n are each independently hydrogen, C1-6alkyl, C2-6alkenyl, C2-6alkynyl, or C3-6cycloalkyl;
R, R3, and R8 are each independently hydrogen, C1-6alkyl, C2-6alkenyl, C2-6alkynyl, or C3-6cycloalkyl;
R9 is hydrogen, C1-6alkyl, C2-6alkenyl, C2-6alkynyl, C3-6cycloalkyl, an amino protecting group, L3-C(O), or A2;
L1 and L2 are each independently selected from C5-21alkyl, C5-21alkenyl, C5-21alkynyl, or C4-20heteroalkyl;
L3 is C1-21alkyl, C5-21alkenyl, C5-21alkynyl, C3-6cycloalkyl, or C2-20heteroalkyl;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any alkyl, alkenyl, alkynyl, cycloalkyl or heteroalkyl present in any of R, R1, R2, R3, R4, R5, R6, R7, R8, R9, Rx, Ry, L1, L2, and L3 is optionally substituted.

In various embodiments,
R1, R2, Rx, Ry, R4, R5, R6, and R7 at each instance of m, v, w, and n are each independently hydrogen, C1-6alkyl, C2-6alkenyl, or C3-6cycloalkyl; R, R3, and R8 are each independently hydrogen, C1-6alkyl, C2-6alkenyl, or C3-6cycloalkyl;
R9 is hydrogen, C1-6alkyl, C2-6alkenyl, C3-6cycloalkyl, an amino protecting group, L3-C(O), or A2;
L1 and L2 are each independently selected from C5-21alkyl, C5-21alkenyl, or C4-20heteroalkyl;
L3 is C1-21alkyl, C5-21alkenyl, C3-6cycloalkyl, or C2-20heteroalkyl;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any alkyl, alkenyl, cycloalkyl or heteroalkyl present in any of R, R1, R2, R3, R4, R5, R6, R7, R8, R9, Rx, Ry, L1, L2, and L3 is optionally substituted.

In various embodiments,
R1, R2, Rx, Ry, R4, R5, R6, and R7 at each instance of m, v, w, and n are each independently hydrogen, C1-6alkyl, or C3-6cycloalkyl;
R, R3, and R8 are each independently hydrogen, C1-6alkyl, or C3-6cycloalkyl;
R9 is hydrogen, C1-6alkyl, C3-6cycloalkyl, an amino protecting group, L3-C(O), or A2;
L1 and L2 are each independently selected from C5-21alkyl, C5-21alkenyl, or C4-20heteroalkyl;
L3 is C1-21alkyl, C2-21alkenyl, C3-6cycloalkyl, or C2-20heteroalkyl;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any alkyl, alkenyl, cycloalkyl or heteroalkyl present in any of R, R1, R2, R3, R4, R5, R6, R7, R8, R9, Rx, Ry, L1, L2, and L3 is optionally substituted.

In various embodiments,
R1, R2, Rx, Ry, R4, R5, R6, and R7 at each instance of m, v, w, and n are each independently hydrogen, C1-6alkyl, or C3-6cycloalkyl;
R, R3, and R8 are each independently hydrogen, C1-6alkyl, or C3-6cycloalkyl; R9 is hydrogen, C1-6alkyl, C3-6cycloalkyl, an amino protecting group, L3-C(O), or A2;
L1 and L2 are each independently selected from is C5-21alkyl or C4-20heteroalkyl;
L3 is C1-21alkyl, C3-6cycloalkyl, or C2-20heteroalkyl;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any alkyl, cycloalkyl or heteroalkyl present in any of R, R1, R2, R3, R4, R5, R6, R7, R8, R9, Rx, Ry, L1, L2, and L3 is optionally substituted.

In various embodiments, Z1 and Z2 are each independently selected from the group consisting of —C(O)O—, —C(O)NR—, and —C(O)S—.

In various embodiments, the compound of the formula (I) is a compound of the formula (IA):

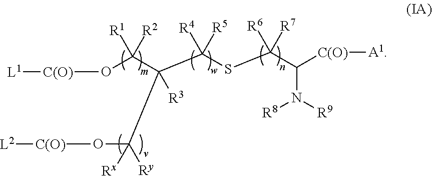

In various embodiments, v is from 0 to 4, 0 to 3, or 0 to 2, or v is 0 or 1, for example 0.

In certain embodiments, v is from 0 to 3. In exemplary embodiments, v is 0.

In various embodiments, m and w are each independently from 0 to 6, 0 to 5, 0 to 4, 0 to 3, 0 to 2, 1 to 7, 1 to 6, 1 to 5, 1 to 4, 1 to 3, or 1 to 2.

In various embodiments, m and w are each independently from 0 to 5.

In certain embodiments, m and w are each independently from 1 to 4.

In various embodiments, m is from 1 to 6, for example from 2 to 6, 1 to 5, or 2 to 5. In various embodiments, m is from 1 to 5. In various embodiments, m is from 1 to 3. In exemplary embodiments, m is 2.

In various embodiments, m is from 3 to 6. In certain embodiments, m is from 3 to 5.

In various embodiments, w is 1 or 2. In exemplary embodiments, w is 1.

In various embodiments, the sum of m and w is from 0 to 6, 0 to 5, 0 to 4, 0 to 3, 1 to 7, 1 to 6, 1 to 5, 1 to 4, 1 to 3, 2 to 7, 2 to 6, 2 to 5, 2 to 4, or 2 to 3.

In various embodiments, the sum of m and w is from 2 to 7.

In certain embodiments, the sum of m and w is from 2 to 5.

In exemplary embodiments, the sum of m and w is 3.

In various embodiments, v is from 0 to 3; m and w are each independently from 0 to 5;

and the sum of m and w is from 2 to 7.

In various embodiments, v is from 1 or 0; m and w are each independently from 0 to 5;

and the sum of m and w is from 2 to 7.

In various embodiments, v is 1 or 0; m and w are each independently from 1 to 4; and

the sum of m and w is from 2 to 7.

In various embodiments, v is 1 or 0; m and w are each independently from 1 to 4; and

the sum of m and w is from 2 to 5.

In certain embodiments, v is 1 or 0; m is from 1 to 6; and w is 1 or 2. In certain embodiments, v is 1 or 0; m is from 1 to 5; and w is 1 or 2.

In certain embodiments, v is 0; m is from 1 to 6; and w is 1.

In certain embodiments v is 0 or 1; m is from 1 to 3; and w is 1 or 2.

In exemplary embodiments, v is 0; m is from 2 to 5; and w is 1.

In exemplary embodiments, v is 0; m is 2; and w is 1.

In certain embodiments, v is 0; m is from 3 to 5; and w is 1.

In exemplary embodiments, n is 1.

In certain embodiments, L1 and L2 are each independently C5-21aliphatic, for example C9-21aliphatic, C11-21aliphatic, or C11-, C13-, C15-, C17-, or C19-aliphatic.

In certain embodiments, L1 and L2 are each independently C5-21alkyl.

In various embodiments, L1 and L2 are each independently C9-21alkyl. In yet another embodiment, L1 and L2 are each independently C11-21alkyl.

In various exemplary embodiments, L1 and L2 are each independently C11, C13, C15, C17, or C19alkyl, preferably n-alkyl.

In various specifically contemplated embodiments, L1 and L2 are each independently C15alkyl.

In various embodiments, L1 and L2 each independently comprise a linear chain of 9-21 carbon atoms.

In exemplary embodiments, L1 and L2 are each independently linear C15alkyl.

In some embodiments, L3 is C1-21alkyl.

In various embodiments, L3 is methyl or linear C15alkyl.

In exemplary embodiments, L3 is methyl (that is, R9 is acetyl).

In some embodiments, the amino protecting group is Boc, Fmoc, Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

In various embodiments, the amino protecting group is Boc or Fmoc.

In some embodiments, the amino protecting group is Fmoc.

In some embodiments, the carboxyl protecting group is tert-butyl, benzyl, or allyl.

In various embodiments, the carboxamide protecting group is Dmcp or Trityl.

In various embodiments, R1 and R2 at each instance of m are each independently C1-6alkyl or hydrogen. In various specifically contemplated embodiments, R1 and R2 at each instance of m are each hydrogen.

In various embodiments, R3 is C1-6alkyl or hydrogen. In various specifically contemplated embodiments, R3 is hydrogen.

In various embodiments, R4 and R5 at each instance of w are each independently C1-6alkyl or hydrogen, preferably hydrogen. In various specifically contemplated embodiments, R4 and R5 at each instance of w are each hydrogen.

In various embodiments, Rx and Ry at each instance of v are each independently C1-6alkyl or hydrogen. In various specifically contemplated embodiments, Rx and Ry at each instance of v are each hydrogen.

In various embodiments, R6 and R7 at each instance of n are each independently C1-6alkyl or hydrogen. In various specifically contemplated embodiments, R6 and R7 are each hydrogen.

In various embodiments, R8 is independently C1-6alkyl or hydrogen. In exemplary embodiments, R8 is hydrogen.

In various embodiments, R9 is C1-6alkyl, hydrogen, an amino protecting group, L3-C(O), or A2. In exemplary embodiments, R9 is hydrogen, an amino protecting group,

L3-C(O), or A2.

In various embodiments, R8 is hydrogen and R9 is hydrogen, an amino protecting group, L3-C(O), or A2.

In various embodiments, R8 and R9 are each hydrogen; or R9 is L3-C(O) or A2.

In various exemplary embodiments, R8 is hydrogen and R9 is L3-C(O). In various specifically contemplated embodiments, R9 is L3-C(O), wherein L3 is methyl.

In various embodiments, the compound of formula (I) is a compound of the formula (IF):

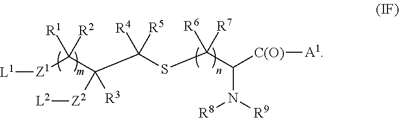

In various embodiments, the compound of formula (IF) is a compound of the formula (IF-1):

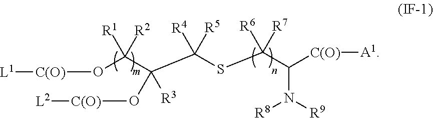

In various embodiments, the compound of formula (I) is a compound of the formula (IB):

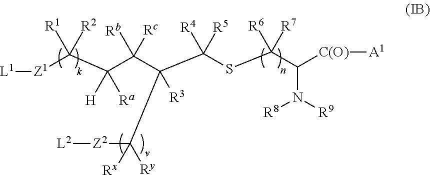

wherein
k is an integer from 0 to 4 (i.e. k is from 0 to 4 when proviso (1) of the first aspect applies and from 1 to 4 when proviso (2) of the first aspect applies); and
Ra, Rb, and Rc are each independently hydrogen or C1-6aliphatic.

In various embodiments, the compound of formula (IB) is a compound of the formula (IC):

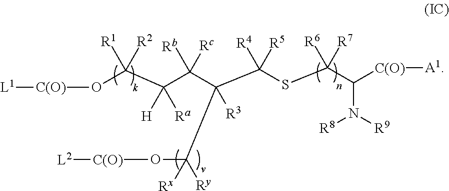

In various embodiments, k is from 0 to 3, 0 to 2, 0 to 1, 1 to 4, 1 to 3, or 1 to 2, or k is 0 or 1.

In certain embodiments, k is 0 to 3.

In certain embodiments, k is 0 or 1.

In exemplary embodiments, k is 0.

In certain embodiments k is equal to v.

In certain embodiments k is from 1 to 3.

In various, embodiments, Ra, Rb, and Rc are each independently hydrogen, C1-6alkyl, C2-6alkenyl, C2-6alkynyl, or C3-6cycloalkyl.

In various, embodiments, Ra, Rb, and Rc are each independently hydrogen, C1-6alkyl, C2-6alkenyl, or C3-6cycloalkyl.

In various, embodiments, Ra, Rb, and Rc are each independently hydrogen, C1-6alkyl, or C3-6cycloalkyl.

In various embodiments, Ra, Rb, and Rc are each independently selected from hydrogen or C1-6alkyl, preferably hydrogen. In exemplary embodiments, Ra, Rb, and Rc are each hydrogen.

In various embodiments, the compound of formula (I) is a compound of the formula (ID-1):

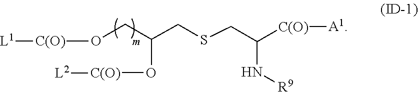

In various embodiments, m in the compound of formula (ID-1) is from 3 to 5.

In various embodiments, the compound of the formula (I) is a compound of the formula (ID):

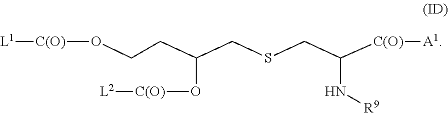

In certain embodiments, the compound is a compound of the formula (ID) wherein L1 and L2 are each linear C15alkyl.

In various embodiments, L1 and L2 are each independently C11-21alkyl; m is 2-5; v is 0; w is 1; R1 and R2 at each instance are each hydrogen; R3 is hydrogen; and R4 and R5 are each hydrogen.

In various embodiments, L1 and L2 are each independently C11-21alkyl; m is 3-5; v is 0; w is 1; R1 and R2 at each instance are each hydrogen; R3 is hydrogen; and R4 and R5 are each hydrogen.

In various embodiments, L1 and L2 are each independently C11-21alkyl; m is 2; v is 0; w is 1; R1 and R2 at each instance are each hydrogen; R3 is hydrogen; and R4 and R5 are each hydrogen.

In various embodiments, n is 1; R6, R7, and R8 are each hydrogen; and R9 is hydrogen, an amino protecting group, L3-C(O), or A2.

In various embodiments, n is 1; R6, R7, and R8 are each hydrogen; and R9 is hydrogen, an amino protecting group, or L3-C(O), wherein L3 is linear C15alkyl or methyl.

In various embodiments, L1 and L2 are each independently C11-21alkyl; m is 2-5; v is 0; w is 1; R1 and R2 at each instance are each hydrogen; R3 is hydrogen; R4 and R5 are each hydrogen; n is 1; R6, R7, and R8 are each hydrogen; and R9 is hydrogen, an amino protecting group, or L3-C(O), wherein L3 is linear C15alkyl or methyl.

In various embodiments, L1 and L2 are each independently C11-21alkyl; m is 3-5; v is 0; w is 1; R1 and R2 at each instance are each hydrogen; R3 is hydrogen; R4 and R5 are each hydrogen; n is 1; R6, R7, and R8 are each hydrogen; and R9 is hydrogen, an amino protecting group, or L3-C(O), wherein L3 is linear C15alkyl or methyl.

In various embodiments, L1 and L2 are each independently C11-21alkyl; m is 2; v is 0; w is 1; R1 and R2 at each instance are each hydrogen; R3 is hydrogen; R4 and R5 are each hydrogen; n is 1; R6, R7, and R8 are each hydrogen; and R9 is hydrogen, an amino protecting group, or L3-C(O), wherein L3 is linear C15alkyl or methyl.

In various embodiments, the compound of formula (I) has the formula (IE):

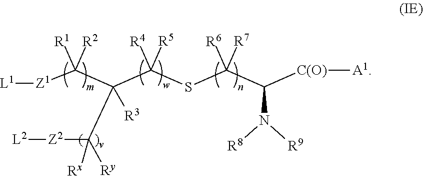

In various embodiments, the compound of formula (I) has the formula (IEE):

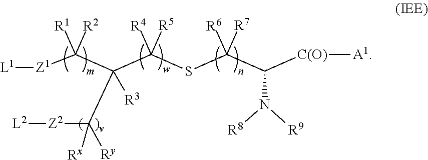

In various embodiments, the compound of formula (I) has the formula (IE-1):

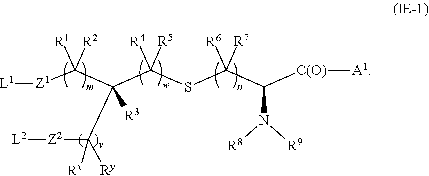

In various embodiments, the compound of formula (I) has the formula (IEE-1):

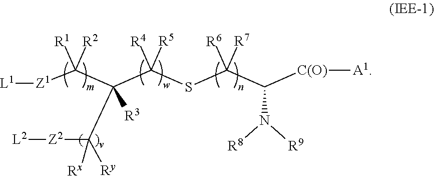

In various embodiments, the compound of formula (I) has the formula (IE-2):

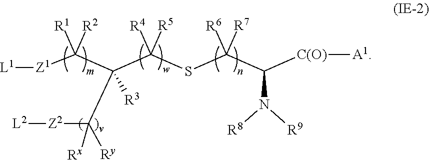

In various embodiments, the compound of formula (I) has the formula (IEE-2):

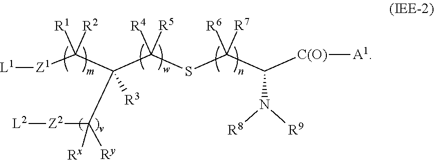

In various embodiments, the compound of formula (I) has the formula (IEE-3):

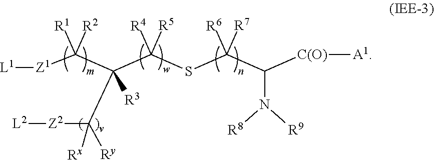

In various embodiments, the compound of formula (I) has the formula (IEE-4):

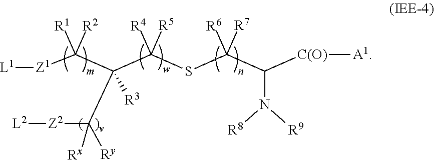

In various embodiments, the amino acid of the amino acid- or peptide conjugate to which the lipid moieties are conjugated is a cysteine residue.

Those skilled in the art will appreciate that, in certain embodiments, the moieties L1-Z1- and L2-Z2- may be fatty acid groups, for example fatty acid esters. In various embodiments, the moieties may be saturated or unsaturated fatty acid esters. In some embodiments, the fatty acid is saturated.

In various embodiments, the fatty acid is a C4-22 fatty acid. In some embodiments, the fatty acid is a C6-22 fatty acid.

In certain embodiments, the fatty acid is a C10-22 fatty acid. In certain specifically contemplated embodiments, the fatty acid is a C12-22 fatty acid. In various exemplary embodiments, the fatty acid is a C12, C14, C16, C18, or C20 fatty acid.

In some embodiments, the fatty acid is lauric acid, myristic acid, palmitic acid, stearic acid, arachic acid, palmitoleic acid, oleic acid, elaidic acid, linoleic acid, α-linolenic acid, and arachidonic acid.

In various embodiments, the fatty acid is lauric acid, myristic acid, palmitic acid, or stearic acid.

In certain exemplary embodiments, the fatty acid is palmitic acid (and the moieties L1-Z1- and L2-Z2- are each palmitoyl groups).

In some embodiments, A1 is OH, OP1, NH2, or NHP2 or R9 is hydrogen, C1-6alkyl, C3-6cycloalkyl, an amino protecting group, or L3-C(O).

In some embodiments, A1 is OP1 or OH or R9 is hydrogen, an amino protecting group or

L3-C(O).

In various embodiments, R9 is hydrogen, an amino protecting group or L3-C(O). In some embodiments, R9 is hydrogen or L3-C(O).

In various embodiments, the compound of formula (I) is a peptide conjugate.

In various embodiments, at least one of A1 and R9 is a peptide comprising, consisting essentially of, or consisting of an amino acid sequence selected from the group consisting of those defined in proviso (1) of the first aspect.

In various embodiments, the peptide comprises, consists of, or consists essentially of an amino acid sequence selected from the group consisting of:
(a) 8 or more contiguous amino acid residues from the sequence SLLMWITQXaa₂₂FLPVF [SEQ ID NO:6],
(b) the sequence of SEQ ID NO: 6,
(c) 8 or more contiguous amino acid residues from the sequence SKKKKSLLMWITQXaa₂₂ [SEQ ID NO:7],
(d) the sequence of SEQ ID NO: 7,
(e) 8 or more contiguous amino acid residues from the sequence SLLMWITQXaa₂₂ [SEQ ID NO:8],
(f) the sequence of SEQ ID NO: 8,
(g) or any combination of two or more of (a) to (f) above.

In various embodiments, the peptide comprises, consists of, or consists essentially of an amino acid sequence selected from the group consisting of:
(a) 8 or more contiguous amino acid residues from the sequence SKKKKSLLMWITQXaa₂₂ [SEQ ID NO:7],
(b) the sequence of SEQ ID NO: 7,
(c) 8 or more contiguous amino acid residues from the sequence SLLMWITQXaa₂₂ [SEQ ID NO:8],
(d) the sequence of SEQ ID NO: 8,
   or any combination of two or more of (a) to (d) above.

In various embodiments, the peptide comprises, consists of, or consists essentially of an amino acid sequence selected from the group consisting of:
(a) 8 or more contiguous amino acid residues from the sequence SKKKKSLLMWITQXaa₂₂ [SEQ ID NO:7],
(b) the sequence of SEQ ID NO: 7.

In various embodiments, the peptide comprises, consists of, or consists essentially of an amino acid sequence selected from the group consisting of:
(a) 8 or more contiguous amino acid residues from the sequence SLLMWITQXaa₂₂ [SEQ ID NO:8],
(b) the sequence of SEQ ID NO: 8.

In various embodiments, Xaa₂₂ in each sequence is independently V, I, or L (that is, each Xaa₂₂ is independently V, I, or L).

In exemplary embodiments, Xaa₂₂ in each sequence is V (that is, each Xaa₂₂ is V).

In various embodiments, m is from 3 to 7 and at least one of A1 and R9 is an amino acid or peptide as defined in proviso (2) of the first aspect. In some embodiments, at least one of A1 and R9 is a peptide. In various embodiments, A1 and/or A2 is an amino acid or a peptide. That is, in various embodiments, A1 is an amino acid or a peptide and/or R9 is an amino acid or a peptide.

In some embodiments, A1 and/or A2 is a peptide. That is, in various embodiments, A1 is a peptide and/or R9 is a peptide.

In one embodiment A1 and/or A2 is a peptide comprising an epitope.

In some embodiments, A1 and/or A2 is a peptide comprising a peptide epitope.

In another embodiment, A1 and/or A2 is a peptide, wherein the peptide comprises a peptide epitope.

In some embodiments, A1 and/or A2 is a peptide substituted with an epitope.

In some embodiments, the epitope is bound to the peptide via a linker group.

In certain embodiments, A1 is a peptide.

In certain exemplary embodiments, A1 is a peptide and R9 is not A2 (that is, R9 is not an amino acid or a peptide).

In certain exemplary embodiments, A1 is a peptide and R9 is hydrogen or L3-C(O), for example Me-C(O).

In various embodiments, the peptide comprises an epitope.

In various embodiments, the epitope is a peptide epitope.

In certain embodiments, the epitope is coupled or bound via a linker group.

In various embodiments, the amino acid of the peptide conjugate to which the lipid moieties are conjugated is an N-terminal amino acid residue.

In various embodiments, A1 is serine or a peptide comprising serine as the first N-terminal amino acid residue.

In some embodiments, A1 is a peptide comprising serine as the first N-terminal amino acid residue.

In various embodiments, the peptide conjugate comprises one or more solubilising groups.

In some embodiments, the solubilising group comprises an amino acid sequence comprising two or more hydrophilic amino acid residues in the peptide chain.

In various embodiments, the solubilising group is an amino acid sequence comprising a sequence of two or more consecutive hydrophilic amino acid residues in the peptide chain.

In various embodiments, the two or more hydrophilic amino acid residues are adjacent to the serine residue.

In some embodiments, A1 and/or A2 is a peptide comprising a solubilising group.

In various embodiments, A1 and/or A2 is a peptide comprising a solubilising group comprising an amino acid sequence comprising two or more hydrophilic amino acid residues in the peptide chain.

In certain embodiments, A1 is a peptide comprising a solubilising group comprising an amino acid sequence comprising two or more hydrophilic amino acid residues in the peptide chain.

In some embodiments, A1 is a peptide comprising serine as the first N-terminal amino acid residue and a solubilising group comprising an amino acid sequence comprising two or more hydrophilic amino acid residues in the peptide chain adjacent to the serine.

In some embodiments, the compound comprises a linker or one or more amino acids thereof. In some embodiments, the peptide comprises a linker or one or more amino acids thereof.

In some embodiments, the peptide comprises a peptide epitope bound via a linker to the amino acid to which the lipid moieties are bound.

In some embodiments, the peptide comprises two or more epitopes.

In some embodiments, the peptide comprises a peptide antigen.

In some embodiments, the linker is an amino acid sequence from about 2 to 20, 2 to 18, 2 to 16, 2 to 14, 2 to 12, 2 to 10, or 2 to 8 amino acids in length.

In some embodiments, the compound of formula (I) comprises 3 or more, 4 or more, or 5 or more contiguous amino acids.

In various embodiments, the peptide conjugate is a lipopeptide.

In some embodiments, the compound of formula (I) is a self adjuvanting peptide.

In some embodiments, A1 and/or A2 are each independently a peptide comprising from about 8 to 220, 8 to 200, 8 to 175, 8 to 150, 8 to 125, 8 to 100, 8 to 90, 8 to 80, 8 to 70, 8 to 60, 8 to 50, 8 to 40, 8 to 30, 8 to 25, 8 to 20, or 8 to 15 amino acids. In one exemplary embodiment, A1 and A2 are each independently a peptide comprising from about 8 to 60 amino acids.

In other embodiments, A1 and/or A2 are each independently a peptide comprising from about 8 to 220, 8 to 200, 8 to 175, 8 to 150, 8 to 125, 8 to 100, 8 to 90, 8 to 80, 8 to 70, 8 to 60, 8 to 50, 8 to 40, 8 to 30, 8 to 25, 8 to 20, or 8 to 15 amino acids.

In other embodiments, A1 and/or A2 are each independently a peptide comprising from about 5 to 150, 5 to 125, 5 to 100, 5 to 75, 5 to 60, 5 to 50, 5 to 40, 5 to 30, 5 to 25, 5 to 20, 8 to 150, 8 to 125, 8 to 100, 8 to 75, 8 to 60, 8 to 50, 8 to 40, 8 to 30, 8 to 25, or 8 to 20 amino acids.

In some embodiments, A1 and/or A2 are each independently a peptide, wherein the peptide comprises 8 to 60 amino acids.

In some embodiments, A1 and/or A2 are each independently a peptide comprising or substituted with a peptide epitope, wherein the peptide epitope comprises from 8 to 60 amino acids.

Suitable peptide epitopes include without limitation those described in WO 2016/103192 filed 22 Dec. 2015, the entirety of which is incorporated herein by reference.

In various embodiments, the peptide comprises, consists essentially of, or consists of one or more EBV LMP2 epitopes. In various embodiments, the one or more EBV LMP2 epitopes are MHCI epitopes. In various embodiments, the peptide comprises one or more EBV LMP2 epitopes selected from the group consisting of any one of SEQ ID NOs 84-109. In various embodiments, the peptide comprises a peptide comprising or consisting of 8 or more contiguous amino acids from the amino acid sequence of any one of SEQ ID NOs 9-83. In various embodiments, the peptide comprises a peptide comprising or consisting of 12 or more contiguous amino acids from the amino acid sequence of any one of SEQ ID NOs 9-83. In various embodiments, the peptide comprises a peptide comprising or consisting of 15 or more contiguous amino acids from the amino acid sequence of any one of SEQ ID NOs 9-83, or comprising or consisting of 20 or more contiguous amino acids from the amino acid sequence of any one of SEQ ID NOs 9-83.

In various embodiments, the peptide comprises a recombinant peptide comprising or consisting of 12 or more contiguous amino acids from the amino acid sequence of any one of SEQ ID NOs 9-83. In various embodiments, the recombinant peptide comprises or consists of 15 or more contiguous amino acids from the amino acid sequence of any one of SEQ ID NOs 9-83, or comprises or consists of 20 or more contiguous amino acids from the amino acid sequence of any one of SEQ ID NOs 9-83.

In various embodiments, the peptide comprises, consists of, or consists essentially of an amino acid sequence selected from the group consisting of any one of SEQ ID NOs 9-83.

In various embodiments, the peptide comprises, consists of, or consists essentially of an amino acid sequence selected from the group consisting of
(a) 8 or more contiguous amino acid residues from the sequence Xaa₁Xaa₂Xaa₃Xaa₄DRHSDYQPLGTQDQSLYLGLQHDGNDGL [SEQ ID NO:9], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(b) 8 or more contiguous amino acid residues from the sequence Xaa₁Xaa₂Xaa₃DRHSDYQPLGTQDQSLYLGLQHDGNDGL [SEQ ID NO:10], wherein Xaa₁ is absent or is S or a hydrophilic amino acid, Xaa₂ is absent or is a hydrophilic amino acid, and Xaa₃ is absent or is from one to ten hydrophilic amino acids,
(c) 8 or more contiguous amino acid residues from the sequence Xaa₁Xaa₂DRHSDYQPLGTQDQSLYLGLQHDGNDGL [SEQ ID NO:11], wherein Xaa₁ is absent or is S or a hydrophilic amino acid, and Xaa₂ is absent or is from one to four hydrophilic amino acids,
(d) 8 or more contiguous amino acid residues from the sequence

[SEQ ID NO: 12]
SKKKKDRHSDYQPLGTQDQSLYLGLQHDGNDGL,

(e) 8 or more contiguous amino acid residues from the sequence

[SEQ ID NO: 13]
DRHSDYQPLGTQDQSLYLGLQHDGNDGL,

(f) 8 or more contiguous amino acid residues from the sequence Xaa₁Xaa₂Xaa₃Xaa₄SLYLGLQHDGNDGLPPPPYSPRDDSSQHIYEEA [SEQ ID NO:14], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(g) 8 or more contiguous amino acid residues from the sequence Xaa₁Xaa₂Xaa₃SLYLGLQHDGNDGLPPPPYSPRDDSSQHIYEEA [SEQ ID NO:15], wherein Xaa₁ is absent or is S or a hydrophilic amino acid, Xaa₂ is absent or is a hydrophilic amino acid, and Xaa₃ is absent or is from one to ten hydrophilic amino acids,
(h) 8 or more contiguous amino acid residues from the sequence Xaa₁Xaa₂SLYLGLQHDGNDGLPPPPYSPRDDSSQHIYEEA [SEQ ID NO:16], wherein Xaa₁ is absent or is S or a hydrophilic amino acid, and Xaa₂ is absent or is from one to four hydrophilic amino acids,)
(i) 8 or more contiguous amino acid residues from the sequence

[SEQ ID NO: 17]
SKKKKSLYLGLQHDGNDGLPPPPYSPRDDSSQHIYEEA,

(j) 8 or more contiguous amino acid residues from the sequence

[SEQ ID NO: 18]
SLYLGLQHDGNDGLPPPPYSPRDDSSQHIYEEA,

(k) 8 or more contiguous amino acid residues from the sequence Xaa₁Xaa₂Xaa₃Xaa₄SDYQPLGTQDQSLYLGLQHDGNDGL [SEQ ID NO:19], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(l) 8 or more contiguous amino acid residues from the sequence Xaa₁Xaa₂Xaa₃SDYQPLGTQDQSLYLGLQHDGNDGL [SEQ ID NO:20], wherein Xaa₁ is absent or is S or a hydrophilic amino acid, Xaa₂ is absent or is a hydrophilic amino acid, and Xaa₃ is absent or is from one to ten hydrophilic amino acids,
(m) 8 or more contiguous amino acid residues from the sequence Xaa₁Xaa₂SDYQPLGTQDQSLYLGLQHDGNDGL [SEQ ID NO:21], wherein Xaa₁ is absent or is S or a hydrophilic amino acid, and Xaa₂ is absent or is from one to four hydrophilic amino acids,
(n) 8 or more contiguous amino acid residues from the sequence

[SEQ ID NO: 22]
SKKKKSDYQPLGTQDQSLYLGLQHDGNDGL,

(o) 8 or more contiguous amino acid residues from the sequence

[SEQ ID NO: 23]
SDYQPLGTQDQSLYLGLQHDGNDGL,

(p) 8 or more contiguous amino acid residues from the sequence Xaa₁Xaa₂Xaa₃Xaa₄DRHSDYQPLGTQDQSLYLGLQHDGNDGLPPPPYSPRDDSSQHIYEEA [SEQ ID NO:24], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(q) 8 or more contiguous amino acid residues from the sequence Xaa₁Xaa₂Xaa₃DRHSDYQPLGTQDQSLYLGLQHDGNDGLPPPPYSPRDDSSQHIYEEA [SEQ ID NO:25], wherein Xaa₁ is absent or is S or a hydrophilic amino acid, Xaa₂ is absent or is a hydrophilic amino acid, and Xaa₃ is absent or is from one to ten hydrophilic amino acids,
(r) 8 or more contiguous amino acid residues from the sequence Xaa₁Xaa₂DRHSDYQPLGTQDQSLYLGLQHDGNDGLPPPPYSPRDDSSQHIYEEA [SEQ ID NO:26], wherein Xaa₁ is absent or is S or a hydrophilic amino acid, and Xaa₂ is absent or is from one to four hydrophilic amino acids,
(s) 8 or more contiguous amino acid residues from the sequence

[SEQ ID NO: 27]
SKKKKDRHSDYQPLGTQDQSLYLGLQHDGNDGLPPPPYSPRDDSSQHIY
EEA,

(t) 8 or more contiguous amino acid residues from the sequence

[SEQ ID NO: 28]
DRHSDYQPLGTQDQSLYLGLQHDGNDGLPPPPYSPRDDSSQHIYEEA,

(u) 8 or more contiguous amino acid residues from the sequence Xaa₁Xaa₂Xaa₃Xaa₄LLWTLVVLLICSSCSSCPLSKILLARLFLYALALLL [SEQ ID NO:29], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(v) 8 or more contiguous amino acid residues from the sequence Xaa₁Xaa₂Xaa₃LLWTLVVLLICSSCSSCPLSKILLARLFLYALALLL [SEQ ID NO:30], wherein Xaa₁ is absent or is S or a hydrophilic amino acid, Xaa₂ is absent or is a hydrophilic amino acid, and Xaa₃ is absent or is from one to ten hydrophilic amino acids,
(w) 8 or more contiguous amino acid residues from the sequence Xaa₁Xaa₂LLWTLVVLLICSSCSSCPLSKILLARLFLYALALLL [SEQ ID NO:31], wherein Xaa₁ is absent or is S or a hydrophilic amino acid, and Xaa₂ is absent or is from one to four hydrophilic amino acids,
(x) 8 or more contiguous amino acid residues from the sequence

[SEQ ID NO: 32]
SKKKKLLWTLVVLLICSSCSSCPLSKILLARLFLYALALLL,

(y) 8 or more contiguous amino acid residues from the sequence

[SEQ ID NO: 33]
LLWTLVVLLICSSCSSCPLSKILLARLFLYALALLL,

(z) 8 or more contiguous amino acid residues from the sequence

Xaa₁Xaa₂Xaa₃Xaa₄LMLLWTLVVLLICSSCSSCPLSKILLARLFLYALALLLLA [SEQ ID NO:34], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(aa) 8 or more contiguous amino acid residues from the sequence Xaa₁Xaa₂Xaa₃LMLLWTLVVLLICSSCSSCPLSKILLARLFLYALALLLLA [SEQ ID NO:35], wherein Xaa₁ is absent or is S or a hydrophilic amino acid, Xaa₂ is absent or is a hydrophilic amino acid, and Xaa₃ is absent or is from one to ten hydrophilic amino acids,
(bb) 8 or more contiguous amino acid residues from the sequence Xaa₁Xaa₂LMLLWTLVVLLICSSCSSCPLSKILLARLFLYALALLLLA [SEQ ID NO:36], wherein Xaa₁ is absent or is S or a hydrophilic amino acid, and Xaa₂ is absent or is from one to four hydrophilic amino acids,
(cc) 8 or more contiguous amino acid residues from the sequence

[SEQ ID NO: 37]
SKKKKLMLLWTLVVLLICSSCSSCPLSKILLARLFLYALALLLLA,

(dd) 8 or more contiguous amino acid residues from the sequence

[SEQ ID NO: 38]
LMLLWTLVVLLICSSCSSCPLSKILLARLFLYALALLLLA,

(ee) 8 or more contiguous amino acid residues from the sequence Xaa₁Xaa₂Xaa₃Xaa₄LMLLWTLVVLLICSSCSSCPLSKILL [SEQ ID NO:39], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(ff) 8 or more contiguous amino acid residues from the sequence Xaa₁Xaa₂Xaa₃LMLLWTLVVLLICSSCSSCPLSKILL [SEQ ID NO:40], wherein Xaa₁ is absent or is S or a hydrophilic amino acid, Xaa₂ is absent or is a hydrophilic amino acid, and Xaa₃ is absent or is from one to ten hydrophilic amino acids,
(gg) 8 or more contiguous amino acid residues from the sequence Xaa₁Xaa₂LMLLWTLVVLLICSSCSSCPLSKILL [SEQ ID NO:41], wherein Xaa₁ is absent or is S or a hydrophilic amino acid, and Xaa₂ is absent or is from one to four hydrophilic amino acids,
(hh) 8 or more contiguous amino acid residues from the sequence

[SEQ ID NO: 42]
SKKKKLMLLWTLVVLLICSSCSSCPLSKILL,

(ii) 8 or more contiguous amino acid residues from the sequence

[SEQ ID NO: 43]
LMLLWTLVVLLICSSCSSCPLSKILL,

(jj) 8 or more contiguous amino acid residues from the sequence Xaa₁Xaa₂Xaa₃Xaa₄LLICSSCSSCPLSKILLARLFLYALALLLLA [SEQ ID NO:44], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(kk) 8 or more contiguous amino acid residues from the sequence Xaa₁Xaa₂Xaa₃LLICSSCSSCPLSKILLARLFLYALALLLLA [SEQ ID NO:45], wherein Xaa₁ is absent or is S or a hydrophilic amino acid, Xaa₂ is absent or is a hydrophilic amino acid, and Xaa₃ is absent or is from one to ten hydrophilic amino acids,
(ll) 8 or more contiguous amino acid residues from the sequence Xaa₁Xaa₂LLICSSCSSCPLSKILLARLFLYALALLLLA [SEQ ID NO:46], wherein Xaa₁ is absent or is S or a hydrophilic amino acid, and Xaa₂ is absent or is from one to four hydrophilic amino acids,
(mm) 8 or more contiguous amino acid residues from the sequence

[SEQ ID NO : 47]
SKKKKLLICSSCSSCPLSKILLARLFLYALALLLLA,

(nn) 8 or more contiguous amino acid residues from the sequence

[SEQ ID NO: 48]
LLICSSCSSCPLSKILLARLFLYALALLLLA,

(oo) 8 or more contiguous amino acid residues from the sequence Xaa₁Xaa₂Xaa₃Xaa₄LNLTTMFLLMLLWTLVVLLICSSCSSCPLSKILLARLFLYALALLLLASALIA GGSI [SEQ ID NO:49], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(pp) 8 or more contiguous amino acid residues from the sequence Xaa₁Xaa₂Xaa₃LNLTTMFLLMLLWTLVVLLICSSCSSCPLSKILLARLFLYALALLLLASALIAGGS I [SEQ ID NO:50], wherein Xaa₁ is absent or is S or a hydrophilic amino acid, Xaa₂ is absent or is a hydrophilic amino acid, and Xaa₃ is absent or is from one to ten hydrophilic amino acids,
(qq) 8 or more contiguous amino acid residues from the sequence Xaa₁Xaa₂LNLTTMFLLMLLWTLVVLLICSSCSSCPLSKILLARLFLYALALLLLASALIAGGSI [SEQ ID NO:51], wherein Xaa₁ is absent or is S or a hydrophilic amino acid, and Xaa₂ is absent or is from one to four hydrophilic amino acids,
(rr) 8 or more contiguous amino acid residues from the sequence

[SEQ ID NO: 52]
SKKKKLNLTTMFLLMLLWTLVVLLICSSCSSCPLSKILLARLFLYALALL
LLASALIAGGSI,

(ss) 8 or more contiguous amino acid residues from the sequence

[SEQ ID NO: 53]
LNLTTMFLLMLLWTLVVLLICSSCSSCPLSKILLARLFLYALALLLLASA
LIAGGSI,

(tt) 8 or more contiguous amino acid residues from the sequence Xaa₁Xaa₂Xaa₃Xaa₄FLLMLLWTLVVLLICSSCSSCPLSKILLARLFLYALALLLLASA [SEQ ID NO:54], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(uu) 8 or more contiguous amino acid residues from the sequence Xaa₁Xaa₂Xaa₃FLLMLLWTLVVLLICSSCSSCPLSKILLARLFLYALALLLLASA [SEQ ID NO:55], wherein Xaa₁ is absent or is S or a hydrophilic amino acid, Xaa₂ is absent or is a hydrophilic amino acid, and Xaa₃ is absent or is from one to ten hydrophilic amino acids,
(vv) 8 or more contiguous amino acid residues from the sequence Xaa₁Xaa₂FLLMLLWTLVVLLICSSCSSCPLSKILLARLFLYALALLLLASA [SEQ ID NO:56], wherein Xaa₁ is absent or is S or a hydrophilic amino acid, and Xaa₂ is absent or is from one to four hydrophilic amino acids,
(ww) 8 or more contiguous amino acid residues from the sequence

[SEQ ID NO: 57]
SKKKKFLLMLLWTLVVLLICSSCSSCPLSKILLARLFLYALALLLLASA,

(xx) 8 or more contiguous amino acid residues from the sequence

[SEQ ID NO: 58]
FLLMLLWTLVVLLICSSCSSCPLSKILLARLFLYALALLLLASA,

(yy) 8 or more contiguous amino acid residues from the sequence Xaa₁Xaa₂Xaa₃Xaa₄LQGIYVLVMLVLLILAYRRRWRRLTVCGGIMFLACVLVLIVDAVLQLSPLL [SEQ ID NO:59], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(zz) 8 or more contiguous amino acid residues from the sequence Xaa₁Xaa₂Xaa₃LQGIYVLVMLVLLILAYRRRWRRLTVCGGIMFLACVLVLIVDAVLQLSPLL [SEQ ID NO:60], wherein Xaa₁ is absent or is S or a hydrophilic amino acid, Xaa₂ is absent or is a hydrophilic amino acid, and Xaa₃ is absent or is from one to ten hydrophilic amino acids,
(aaa) 8 or more contiguous amino acid residues from the sequence Xaa₁Xaa₂LQGIYVLVMLVLLILAYRRRWRRLTVCGGIMFLACVLVLIVDAVLQLSPLL [SEQ ID NO:61], wherein Xaa₁ is absent or is S or a hydrophilic amino acid, and Xaa₂ is absent or is from one to four hydrophilic amino acids,
(bbb) 8 or more contiguous amino acid residues from the sequence

[SEQ ID NO: 62]
SKKKKLQGIYVLVMLVLLILAYRRRWRRLTVCGGIMFLACVLVLIVDAVL
QLSPLL,

(ccc) 8 or more contiguous amino acid residues from the sequence

[SEQ ID NO: 63]
LQGIYVLVMLVLLILAYRRRWRRLTVCGGIMFLACVLVLIVDAVLQLSPL
L,

(ddd) 8 or more contiguous amino acid residues from the sequence Xaa₁Xaa₂Xaa₃Xaa₄SGNRTYGPVFM(C)(S)LGGLLTMVAGAVWLTVMSNTLLSAWILTAGFLI FLIGFA [SEQ ID NO:64], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(eee) 8 or more contiguous amino acid residues from the sequence Xaa₁Xaa₂Xaa₃SGNRTYGPVFM(C)(S)LGGLLTMVAGAVWLTVMSNTLLSAWILTAGFLIFLIG FA [SEQ ID NO:65], wherein Xaa₁ is absent or is S or a hydrophilic amino acid, Xaa₂ is absent or is a hydrophilic amino acid, and Xaa₃ is absent or is from one to ten hydrophilic amino acids,
(fff) 8 or more contiguous amino acid residues from the sequence Xaa₁Xaa₂SGNRTYGPVFM(C)(S)LGGLLTMVAGAVWLTVMSNTLLSAWILTAGFLIFLIGFA [SEQ ID NO:66], wherein Xaa₁ is absent or is S or a hydrophilic amino acid, and Xaa₂ is absent or is from one to four hydrophilic amino acids,
(ggg) 8 or more contiguous amino acid residues from the sequence

[SEQ ID NO: 67]
SKKKKSGNRTYGPVFM(C)(S)LGGLLTMVAGAVWLTVMSNTLLSAWILT
AGFLIFLIGFA,

(hhh) 8 or more contiguous amino acid residues from the sequence

[SEQ ID NO: 68]
SGNRTYGPVFM[C][S]LGGLLTMVAGAVWLTVMSNTLLSAWILTAGFLI
FLIGFA,

(iii) 8 or more contiguous amino acid residues from the sequence Xaa₁Xaa₂Xaa₃Xaa₄SNEEPPPPYEDPYWGNGDRHSDYQPLGTQDQSLYLGLQHDGNDGLPP [SEQ ID NO:69], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(jjj) 8 or more contiguous amino acid residues from the sequence Xaa₁Xaa₂Xaa₃SNEEPPPPYEDPYWGNGDRHSDYQPLGTQDQSLYLGLQHDGNDGLPP [SEQ ID NO:70], wherein Xaa₁ is absent or is S or a hydrophilic amino acid, Xaa₂ is absent or is a hydrophilic amino acid, and Xaa₃ is absent or is from one to ten hydrophilic amino acids,
(kkk) 8 or more contiguous amino acid residues from the sequence Xaa₁Xaa₂SNEEPPPPYEDPYWGNGDRHSDYQPLGTQDQSLYLGLQHDGNDGLPP [SEQ ID NO:71], wherein Xaa₁ is absent or is S or a hydrophilic amino acid, and Xaa₂ is absent or is from one to four hydrophilic amino acids,
(lll) 8 or more contiguous amino acid residues from the sequence

[SEQ ID NO: 72]
SKKKKSNEEPPPPYEDPYWGNGDRHSDYQPLGTQDQSLYLGLQHDGNDGL
PP,

(mmm) 8 or more contiguous amino acid residues from the sequence

[SEQ ID NO: 73]
SNEEPPPPYEDPYWGNGDRHSDYQPLGTQDQSLYLGLQHDGNDGLPP,

(nnn) 8 or more contiguous amino acid residues from the sequence Xaa₁Xaa₂Xaa₃Xaa₄GNDGLPPPPYSPRDDSSQHIYEEAGRGSMNPVCLPVIVAPYLFWLAAIAA S [SEQ ID NO:74], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(ooo) 8 or more contiguous amino acid residues from the sequence Xaa₁Xaa₂Xaa₃GNDGLPPPPYSPRDDSSQHIYEEAGRGSMNPVCLPVIVAPYLFWLAAIAAS [SEQ ID NO:75], wherein Xaa₁ is absent or is S or a hydrophilic amino acid, Xaa₂ is absent or is a hydrophilic amino acid, and Xaa₃ is absent or is from one to ten hydrophilic amino acids,
(ppp) 8 or more contiguous amino acid residues from the sequence Xaa₁Xaa₂GNDGLPPPPYSPRDDSSQHIYEEAGRGSMNPVCLPVIVAPYLFWLAAIAAS [SEQ ID NO:76], wherein Xaa₁ is absent or is S or a hydrophilic amino acid, and Xaa₂ is absent or is from one to four hydrophilic amino acids,
(qqq) 8 or more contiguous amino acid residues from the sequence

[SEQ ID NO: 77]
SKKKKGNDGLPPPPYSPRDDSSQHIYEEAGRGSMNPVCLPVIVAPYLFWL
AAIAAS,

(rrr) 8 or more contiguous amino acid residues from the sequence

[SEQ ID NO: 78]
GNDGLPPPPYSPRDDSSQHIYEEAGRGSMNPVCLPVIVAPYLFWLAAIAA
S,

(sss) 8 or more contiguous amino acid residues from the sequence Xaa₁Xaa₂Xaa₃Xaa₄AAIAASCFTASVSTVVTATGLALSLLLLAAVASSYAAAQRKLLTPVTVLT [SEQ ID NO:79], wherein Xaa₁ is absent or is S or a hydrophilic amino acid, Xaa₂ is absent or is a hydrophilic amino acid, Xaa₃ is absent or is a hydrophilic amino acid, and Xaa₄ is absent or is one or more hydrophilic amino acids,
(ttt) 8 or more contiguous amino acid residues from the sequence Xaa₁Xaa₂Xaa₃AAIAASCFTASVSTVVTATGLALSLLLLAAVASSYAAAQRKLLTPVTVLT [SEQ ID NO:80], wherein Xaa₁ is absent or is S or a hydrophilic amino acid, Xaa₂ is absent or is a hydrophilic amino acid, and Xaa₃ is absent or is from one to ten hydrophilic amino acids,
(uuu) 8 or more contiguous amino acid residues from the sequence Xaa₁Xaa₂AAIAASCFTASVSTVVTATGLALSLLLLAAVASSYAAAQRKLLTPVTVLT [SEQ ID NO:81], wherein Xaa₁ is absent or is S or a hydrophilic amino acid, and Xaa₂ is absent or is from one to four hydrophilic amino acids,
(vvv) 8 or more contiguous amino acid residues from the sequence

[SEQ ID NO: 82]
SKKKKAAIAASCFTASVSTVVTATGLALSLLLLAAVASSYAAAQRKLLTP
VTVLT,

(www) 8 or more contiguous amino acid residues from the sequence

[SEQ ID NO: 83]
AAIAASCFTASVSTVVTATGLALSLLLLAAVASSYAAAQRKLLTPVTVLT,

(xxx) the sequence of any one of SEQ ID NOs: 9 to 83,
(yyy) 8 or more contiguous amino acid residues from the sequence of any one of

[SEQ ID NO: 84]
ESNEEPPPPY,
[SEQ ID NO: 85]
SNEEPPPPY,
[SEQ ID NO: 86]
HSDYQPLGT,
[SEQ ID NO: 87]
PLGTQDQSL,
[SEQ ID NO: 88]
PLGTQDQSLY,
[SEQ ID NO: 89]
LGTQDQSLY,
[SEQ ID NO: 90]
GTQDQSLYL,
[SEQ ID NO: 91]
GTQDQSLYL,
[SEQ ID NO: 92]
GTQDQSLYLG,
[SEQ ID NO: 93]
QSLYLGLQH,
[SEQ ID NO: 94]
SLYLGLQHD,
[SEQ ID NO: 95]
GLQHDGNDGL,
[SEQ ID NO: 96]
GNDGLPPPPY,
[SEQ ID NO: 97]
GLPPPPYSP,
[SEQ ID NO: 98]
GLPPPPYSPR,
[SEQ ID NO: 99]
PRDDSSQHIY,
[SEQ ID NO: 100]
RDDSSQHIY,
[SEQ ID NO: 101]
HIYEEAGRG,
[SEQ ID NO: 102]
ILLARLFLY,
[SEQ ID NO: 103]
SSCSSCPLSKI,
[SEQ ID NO: 104]
LLWTLVVLL,
[SEQ ID NO: 105]
FLYALALLL,
[SEQ ID NO: 106]
CLGGLLTMV,
[SEQ ID NO: 107]
LIVDAVLQL,
[SEQ ID NO: 108]
LTAGFLIFL,
[SEQ ID NO: 109]
TVCGGIMFL,

(zzz) the sequence of any one of SEQ ID NOs: 83-109,
(aaaa) or any combination of two or more of (a) to (zzz) above.

In one exemplary embodiment, the peptide comprises one or more epitopes derived from Latent Membrane Protein 2 (LMP2), for example, from full-length EBV LMP2 (amino acids 1-497). In one specifically contemplated embodiment, the peptide comprises, consists essentially of, or consists of an amino acid sequence selected from the group consisting of 8 or more contiguous amino acid residues from any one of SEQ ID NOs: 12, 13, 17, 18, 22, 23, 27, 28, 32, 33, 37, 38, 42, 43, 47, 48, 52, 53, 57, 58, 62, 63, 67, 68, 72, 73, 77, 78, 82, or 83.

In another specifically contemplated embodiment, the peptide comprises, consists essentially of, or consists of an amino acid sequence selected from the group consisting of 12 or more contiguous amino acid residues from any one of SEQ ID NOs: 12, 13, 17, 18, 22, 23, 27, 28, 32, 33, 37, 38, 42, 43, 47, 48, 52, 53, 57, 58, 62, 63, 67, 68, 72, 73, 77, 78, 82, or 83.

In another specifically contemplated embodiment, the peptide comprises, consists essentially of, or consists of an amino acid sequence selected from the group consisting of 15 or more, 18 or more, 20 or more, or 25 or more contiguous amino acid residues from any one of SEQ ID NOs: 12, 13, 17, 18, 22, 23, 27, 28, 32, 33, 37, 38, 42, 43, 47, 48, 52, 53, 57, 58, 62, 63, 67, 68, 72, 73, 77, 78, 82, or 83.

In one embodiment, the peptide comprises, consists essentially of, or consists of an amino acid sequence selected from the group consisting of any one of SEQ ID NOs: 12, 13, 17, 18, 22, 23, 27, 28, 32, 33, 37, 38, 42, 43, 47, 48, 52, 53, 57, 58, 62, 63, 67, 68, 72, 73, 77, 78, 82, or 83.

In another specifically contemplated embodiment, the peptide comprises, consists essentially of, or consists of an amino acid sequence selected from the group consisting of 15 or more, 18 or more, 20 or more, or 25 or more contiguous amino acid residues from any one of SEQ ID NOs: 9 to 83.

In one embodiment, the peptide comprises, consists essentially of, or consists of an amino acid sequence selected from the group consisting of any one of SEQ ID NOs: 9 to 83.

In one embodiment, the peptide comprises an amino acid sequence selected from the group consisting of any one of SEQ ID NOs: 84 to 109. In one example, the peptide comprises an amino acid sequence selected from the group consisting of any one of SEQ

ID NOs: 84 to 101.

In one embodiment, the peptide comprises an amino acid sequence selected from the group consisting of any two or more of SEQ ID NOs: 84 to 109. In one example, the peptide comprises an amino acid sequence selected from the group consisting of any two or more of SEQ ID NOs: 84 to 101.

In various embodiments the peptide comprises, consists of, or consists essentially of an amino acid sequence selected from the group consisting of
(a) 8 or more contiguous amino acid residues from the sequence Xaa₁Xaa₂Xaa₃Xaa₄GARGPESRLLEFYLAMPFATPMEAELARRSLAQDAPPL [SEQ ID NO:110], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids,
(b) 8 or more contiguous amino acid residues from the sequence Xaa₁Xaa₂Xaa₃GARGPESRLLEFYLAMPFATPMEAELARRSLAQDAPPL [SEQ ID NO:111], wherein Xaa₁ is absent or is S, Xaa₂ is absent or is a hydrophilic amino acid, and Xaa₃ is absent or is from one to ten hydrophilic amino acids,
(c) 8 or more contiguous amino acid residues from the sequence Xaa₁Xaa₂GARGPESRLLEFYLAMPFATPMEAELARRSLAQDAPPL [SEQ ID NO:112], wherein Xaa₁ is absent or is S, and Xaa₂ is absent or is from one to four hydrophilic amino acids,
(d) 8 or more contiguous amino acid residues from the sequence

[SEQ ID NO: 113]
SKKKKGARGPESRLLEFYLAMPFATPMEAELARRSLAQDAPPL,

(e) the sequence of any one of SEQ ID NOs: 110 to 113,
(f) 8 or more contiguous amino acid residues from the sequence

[SEQ ID NO: 114]
GARGPESRLLEFYLAMPFATPMEAELARRSLAQDAPPL,

(g) the sequence of SEQ ID NO: 114,
(h) 8 or more contiguous amino acid residues from the sequence LAMPFATPM [SEQ ID NO:115],
(i) the sequence of SEQ ID NO: 115,
(j) 8 or more contiguous amino acid residues from the sequence FATPMEAEL [SEQ ID NO:116],
(k) the sequence of SEQ ID NO: 116,
(l) 8 or more contiguous amino acid residues from the sequence Xaa₁Xaa₂Xaa₃Xaa₄VPGVLLKEFTVSGNILTIRLTAADHR [SEQ ID NO:117], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids,
(m) 8 or more contiguous amino acid residues from the sequence Xaa₁Xaa₂Xaa₃VPGVLLKEFTVSGNILTIRLTAADHR [SEQ ID NO:118], wherein Xaa₁ is absent or is S, Xaa₂ is absent or is a hydrophilic amino acid, and Xaa₃ is absent or is from one to ten hydrophilic amino acids,
(n) 8 or more contiguous amino acid residues from the sequence Xaa₁Xaa₂VPGVLLKEFTVSGNILTIRLTAADHR [SEQ ID NO:119], wherein Xaa₁ is absent or is S, and Xaa₂ is absent or is from one to four hydrophilic amino acids,
(o) 8 or more contiguous amino acid residues from the sequence

[SEQ ID NO: 120]
SKKKKVPGVLLKEFTVSGNILTIRLTAADHR,

(p) the sequence of any one of SEQ ID NOs: 117 to 120,
(q) 8 or more contiguous amino acid residues from the sequence

[SEQ ID NO: 121]
VPGVLLKEFTVSGNILTIRLTAADHR,

(r) the sequence of SEQ ID NO: 121,
(s) 8 or more contiguous amino acid residues from the sequence EFTVSGNIL [SEQ ID NO:122],
(t) the sequence of SEQ ID NO: 122,
(u) 8 or more contiguous amino acid residues from the sequence Xaa₁Xaa₂Xaa₃Xaa₄LQQLSLLMWITQCFLPVFLAQPPSGQRR [SEQ ID NO:123], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids
(v) 8 or more contiguous amino acid residues from the sequence Xaa₁Xaa₂Xaa₃LQQLSLLMWITQCFLPVFLAQPPSGQRR [SEQ ID NO:124], wherein Xaa₁ is absent or is S, Xaa₂ is absent or is a hydrophilic amino acid, and Xaa₃ is absent or is from one to ten hydrophilic amino acids,
(w) 8 or more contiguous amino acid residues from the sequence Xaa₁Xaa₂LQQLSLLMWITQCFLPVFLAQPPSGQRR [SEQ ID NO:125], wherein Xaa₁ is absent or is S, and Xaa₂ is absent or is from one to four hydrophilic amino acids,
(x) 8 or more contiguous amino acid residues from the sequence

[SEQ ID NO: 126]
SKKKKLQQLSLLMWITQCFLPVFLAQPPSGQRR,

(y) the sequence of any one of SEQ ID NOs: 123 to 126,
(z) 8 or more contiguous amino acid residues from the sequence

[SEQ ID NO: 127]
LQQLSLLMWITQCFLPVFLAQPPSGQRR,

(aa) the sequence of SEQ ID NO: 127,
(bb) 8 or more contiguous amino acid residues from the sequence SLLMWITQCFLPVF [SEQ ID NO:128],
(cc) the sequence of SEQ ID NO: 128,
(dd) 8 or more contiguous amino acid residues from the sequence SLLMWITQC [SEQ ID NO:129],
(ee) the sequence of SEQ ID NO: 129,
(ff) or any combination of two or more of (a) to (ee) above.

In one exemplary embodiment, the peptide epitope is derived from NY-ESO-1. In one specifically contemplated embodiment, the peptide comprises, consists essentially of, or consists of an amino acid sequence selected from the group consisting of 8 or more contiguous amino acid residues from any one of SEQ ID NO: 114, 115, 116, 121, 122, 127, 128, and 129.

In one embodiment, the peptide comprises, consists essentially of, or consists of an amino acid sequence selected from the group consisting of any one of SEQ ID NO: 114, 115, 116, 121, 122, 127, 128, and 129.

In one embodiment, the peptide comprises, consists essentially of, or consists of an amino acid sequence selected from the group consisting of any one of SEQ ID NO: 114, 121, and 127.

In one embodiment, the peptide comprises, consists essentially of, or consists of an amino acid sequence selected from the group consisting of any one of SEQ ID NO: 113, 120, and 126.

In various embodiments, the peptide comprises, consists essentially of, or consists of one or more ovalbumin protein epitopes. In various embodiments, the one or more ovalbumin protein are MHCI epitopes. In various embodiments, the one or more ovalbumin protein are MHCII epitopes.

In various embodiments, the peptide comprises, consists essentially of, or consists of:
(a) 8 or more contiguous amino acid residues from the sequence Xaa₁Xaa₂Xaa₃Xaa₄KISQAVHAAHAEINEAGRESIINFEKLTEWT [SEQ ID NO:130], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids
(b) 8 or more contiguous amino acid residues from the sequence Xaa₁Xaa₂Xaa₃ KISQAVHAAHAEINEAGRESIINFEKLTEWT [SEQ ID NO:131], wherein Xaa₁ is absent or is S, Xaa₂ is absent or is a hydrophilic amino acid, and Xaa₃ is absent or is from one to ten hydrophilic amino acids,
(c) 8 or more contiguous amino acid residues from the sequence Xaa₁Xaa₂ KISQAVHAAHAEINEAGRESIINFEKLTEWT [SEQ ID NO:132], wherein Xaa₁ is absent or is S, and Xaa₂ is absent or is from one to four hydrophilic amino acids,
(d) 8 or more contiguous amino acid residues from the sequence

[SEQ ID NO: 133]
SKKKKKISQAVHAAHAEINEAGRESIINFEKLTEWT,

(e) the sequence of any one of SEQ ID NOs: 130 to 133,
(f) 8 or more contiguous amino acid residues from the sequence

[SEQ ID NO: 134]
KISQAVHAAHAEINEAGRESIINFEKLTEWT,

(g) the sequence of SEQ ID NO: 134,
(h) 8 or more contiguous amino acid residues from the sequence SIINFEKL [SEQ ID NO: 135],
(i) the sequence of SEQ ID NO: 135,
(j) 8 or more contiguous amino acid residues from the sequence ISQAVHAAHAEINEAGR [SEQ ID NO: 136],
(k) the sequence of SEQ ID NO: 136,
(l) or any combination of any two or more of (a) to (k) above.

In various embodiments, the peptide comprises one or more ovalbumin protein epitopes selected from the group consisting of any one of SEQ ID NOs 130-136. In various embodiments, the peptide comprises a peptide comprising or consisting of 8 or more contiguous amino acids from the amino acid sequence of any one of SEQ ID NOs 130-136.

In various embodiments, the peptide comprises, consists of, or consists essentially of an amino acid sequence selected from the group consisting of any one of SEQ ID NOs 130-136.

In various embodiments, the peptide comprises one or more immunodominant A*0200 restricted epitopes derived from the cytomeglovirus (CMV) ppUL83 protein (‘NLV peptide’) consisting of
(gg) 8 or more contiguous amino acid residues from the sequence NLVPMVATV [SEQ ID NO:137],
(hh) the sequence of SEQ ID NO: 137,
(ii) 8 or more contiguous amino acid residues from the sequence CSKKKKNLVPMVATV [SEQ ID NO:138],
(jj) the sequence of SEQ ID NO: 138.

In various embodiments, the peptide comprises, consists essentially of, or consists of an amino acid sequence selected from the group consisting of 8 or more contiguous amino acids from the amino acid sequence of any one of SEQ ID NOs 9-129.

In various embodiments, the peptide comprises, consists essentially of, or consists of one or more amino acid sequences selected from the group consisting of those defined in proviso (1) of the first aspect.

In various embodiments, Xaa₄ in the sequences referred to herein is absent or is from 1 to 17 hydrophilic amino acids, for example, from 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2 hydrophilic amino acids, or is a hydrophilic amino acid.

In one embodiment, the peptide conjugate comprises two or more epitopes, such as two or more peptide epitopes.

In some embodiments, the peptide conjugate comprises an antigenic peptide.

In specifically contemplated embodiments, the peptide is a synthetic peptide.

In various embodiments, the compound of formula (I) is an isolated compound of formula (I).

In various embodiments, the compound of formula (I) is a pure, purified or substantially pure compound of formula (I).

In various embodiments, the compound of formula (I) of the invention is a compound selected from the group consisting of compounds 910, 911, 912, 913, 930, 931, and 932 of the Examples herein.

In another aspect, the present invention broadly consists in a method of making a peptide conjugate of the formula (IF) or a pharmaceutically acceptable salt or solvate thereof of the present invention, the method comprising:

(A) reacting
an epoxide of the formula (XVI):

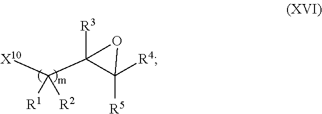

and
an amino acid-comprising conjugation partner comprising a thiol of the formula (III):

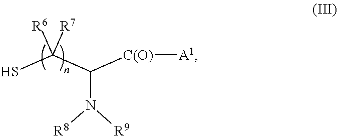

under conditions effective to conjugate the epoxide and amino acid-comprising conjugation partner and provide a compound of formula (XV):

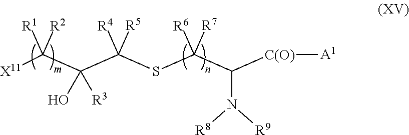

wherein
X10 is L1-Z1-, —OH, —SH, —NHR, HNRC(O)O—, P10-O—, P11-S—, P12-NR—, or P12-NRC(O)O—;
X11 is X10 or —OH, —SH, —NHR, or HNRC(O)O— when X10 is P10-O—, P11-S—, P12-NR—, or P12-NRC(O)O— and said conditions are effective to remove P10, P11, or P12;
P10, P11, and P12 are each independently a protecting group;
m, n, L1, Z1, R, R1, R2, R3, R4, R5, R6, R7, R8, R9, and A1 are as defined in the compound of formula (IF) of the invention (including provisos (1) and/or (2) of the first aspect); and
converting the compound of formula (XV) to the peptide-conjugate of the formula (IF) of the invention (including provisos (1) and/or (2) of the first aspect) or a pharmaceutically acceptable salt or solvate thereof by one or more additional synthetic steps:

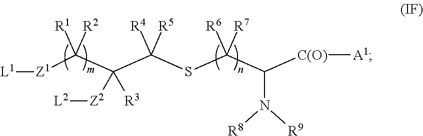

or
(B) reacting
an epoxide of the formula (XVI):

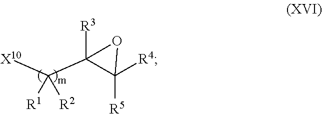

and
an amino acid-comprising conjugation partner comprising a thiol of the formula (III):

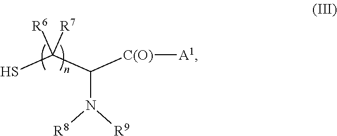

under conditions effective to conjugate the epoxide and amino acid-comprising conjugation partner and provide a compound of formula (XV):

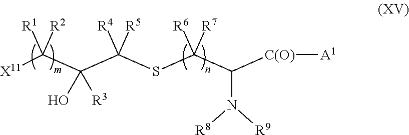

wherein
X10 is L1-Z1-, —OH, —SH, —NHR, HNRC(O)O—, P10-O—, P11-S—, P12-NR—, or P12-NRC(O)O—;
X11 is X10 or —OH, —SH, —NHR, or HNRC(O)O— when X10 is P10-O—, P11-S—, P12-NR—, or P12-NRC(O)O— and said conditions are effective to remove P10, P11, or P12;
P10, P11, and P12 are each independently a protecting group;
m, n, L1, Z1, R, R1, R2, R3, R4, R5, R6, R7, R8, R9, and A1 are as defined in the compound of formula (IF) but excluding provisos (1) and (2) of the first aspect; and
converting the compound of formula (XV) to an amino acid- or peptide-conjugate of the formula (IF) but excluding provisos (1) and (2) of of the first aspect or a salt or solvate thereof by one or more additional synthetic steps:

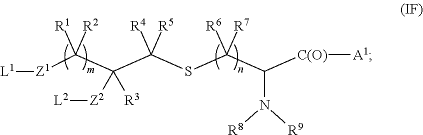

and
coupling the amino acid of the amino acid conjugate or an amino acid of the peptide conjugate to an amino acid or an amino acid of a peptide to provide the peptide-conjugate of formula (IF) of the present invention (including provisos (1) and/or (2) of the first aspect) or pharmaceutically acceptable salt or solvate thereof.

In various embodiments m is from 2 to 5, 2 to 4, or 2 to 3. In exemplary embodiments, m is 2. In other exemplary embodiments, m is from 3 to 5.

In various embodiments, X10 is L1-Z1- or —OH, —SH, —NHR, P10-O—, P11-S—, or P12-NR—; and X11 is X10 or —OH, —SH, or —NHR.

In various embodiments, X10 is L1-Z1-, —OH, or P10-O—; and X11 is X10 or —OH.

In various embodiments, X10 is L1-C(O)O—, OH, or P10-O—; and X11 is L1-C(O)O—, P10-O—, or OH.

In various embodiments, X10 is L1-C(O)O— or P10-O—; and X11 is L1-C(O)O—, P10-O—, or OH.

In exemplary embodiments, X10 is P10-O—; and X11 is P10-O— or OH.

In various embodiments, R9 is not hydrogen and/or A1 is not OH.

In various embodiments, the amino acid-comprising conjugation partner is a peptide containing conjugation partner comprising 15 or less, 14 or less, 13 or less, 12 or less, 11 or less, 10 or less, 9 or less, 8 or less, 7 or less, 6 or less, 5 or less, 4 or less, or 3 or less amino acid residues.

In various embodiments, the C-terminus of the amino acid comprising conjugation partner is protected with a carboxyl protecting group or a carboxamide protecting group and/or the Nα-amino group of the amino acid comprising conjugation partner is protected with an amino protecting group.

In exemplary embodiments, R9 is an amino protecting group.

In various embodiments, A1 is OP1 or NHP2. In certain embodiments, A1 is OP1.

In exemplary embodiments, R9 is an amino protecting group and A1 is OP1 in the amino acid comprising conjugation partner.

In various embodiments, the method comprises reacting the epoxide and amino acid-comprising conjugation partner in the presence of an acid, for example a strong acid.

In certain embodiments, the acid comprises hydrochloric acid, sulfuric acid, or a mixture thereof.

In certain embodiments, the acid comprises a lewis acid, for example BF₃.

In other embodiments, the method comprises reacting the epoxide and amino acid-comprising conjugation partner under neutral conditions.

In various embodiments, the neutral conditions comprise a protic solvent, such as an alcohol, for example ethanol.

In other embodiments, the method comprises reacting the epoxide and amino acid-comprising conjugation partner in the presence of a base, for example a mild base.

In some embodiments, the base is an organic amine, for example triethylamine.

In various embodiments, the method comprises providing the epoxide by reacting an alkene of the formula (XVII):

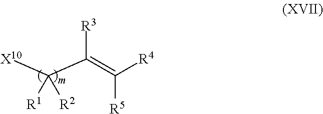

and an oxidant under conditions effective to epoxidise the alkene.

In various embodiments, the oxidant is a peroxide, such as an organic peroxide, for example m-chloro peroxybenzoic acid, or an organic N-oxide, for example pyridine N-oxide.

In various embodiments, the method comprises providing the epoxide by reacting an compound of the formula (XVII-A) wherein LG is a leaving group:

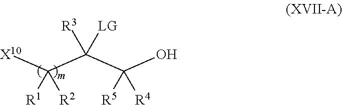

and a base under conditions effective for epoxidation.

In various embodiments, the compound of formula (XVII-A) is prepared from L-aspartic acid.

In various embodiments, the method further comprises providing a single stereoisomer or a stereoisomerically enriched mixture of the epoxide of formula (XVI).

In various embodiments, providing the single stereoisomer or a stereoisomerically enriched mixture of the epoxide of formula (XVI) comprises resolving a racemic mixture of the epoxide.

In various embodiments, the method comprises providing a single stereoisomer or a stereoisomerically enriched mixture of the compound of formula (XVII-A).

In various embodiments, the method comprises converting the compound of formula (XV) to an amino acid- or peptide conjugate of the formula (IF-1) or a pharmaceutically acceptable salt or solvate thereof by one or more additional synthetic steps:

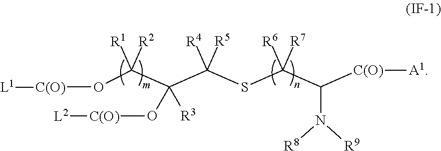

In various embodiments, the one or more synthetic steps comprises converting the hydroxyl group bound to the carbon to which R3 is attached to L2-Z2-.

In various embodiments, the one or more synthetic steps comprises acylating the compound of formula (XV) so as to replace the hydrogen atom of the hydroxyl group bound to the carbon to which R3 is attached with L2-C(O)—.

In various embodiments, X11 is P10-O— or OH; and the one or more synthetic steps comprise acylating the compound of formula (XV) so as to replace P10 or the hydrogen atom of the hydroxyl group of X11 with L1-C(O)—; and/or acylating the compound of formula (XV) so as to replace the hydrogen atom of the hydroxyl group bound to the carbon to which R3 is attached with L2-C(O)—.

In another aspect, the present invention broadly consists in a compound of the formula (XV):

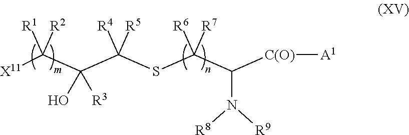

wherein
X11 is L1-Z1-, —OH, —SH, —NHR, HNRC(O)O—, P10-O—, P11-S—, P12-NR—, or P12-NRC(O)O—;
P10, P11, and P12 are each independently a protecting group;
m is an integer from 2 to 6; and
n, L1, Z1, R, R1, R2, R3, R4, R5, R6, R7, R8, R9, and A1 are as defined in the compound of formula (I) of the invention (including provisos (1) and/or (2) of the first aspect) or any embodiment thereof; or a salt or solvate thereof.

In another aspect, the present invention broadly consists in a method of making a compound of the formula (XV) or a salt or solvate thereof, the method comprising:

(A) reacting
an epoxide of the formula (XVI):

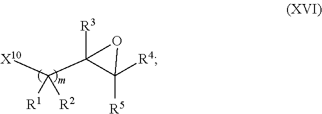

and
an amino acid-comprising conjugation partner comprising a thiol of the formula (III):

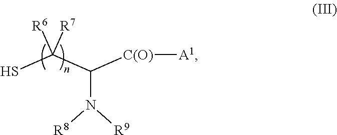

under conditions effective to conjugate the epoxide and amino acid-comprising conjugation partner and provide a compound of formula (XV):

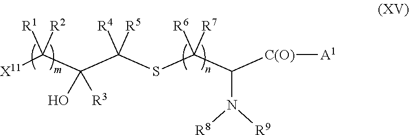

wherein
X10 is L1-Z1-, —OH, —SH, —NHR, HNRC(O)O—, P10-O—, P11-S—, P12-NR—, or P12-NRC(O)O—;
X11 is X10 or —OH, —SH, —NHR, or HNRC(O)O— when X10 is P10-O—, P11-S—, P12-NR—, or P12-NRC(O)O— and said conditions are effective to remove P10, P11, or P12;
P10, P11, and P12 are each independently a protecting group;
m, n, L1, Z1, R, R1, R2, R3, R4, R5, R6, R7, R8, R9, and A1 are as defined in the compound of formula (IF) of the invention (including provisos (1) and/or (2) of the first aspect).

In another aspect, the present invention broadly consists in the use of a compound of the formula (XV) or (XVI) in the synthesis of a peptide-conjugate of the formula (IF) of the present invention (including provisos (1) and/or (2) of the first aspect) or a pharmaceutically acceptable salt or solvate thereof.

In another aspect, the present invention broadly consists in a method of making a peptide-conjugate of the formula (I) or a pharmaceutically acceptable salt or solvate thereof of the present invention, the method comprising:

(A) reacting
a compound of the formula (XXI):

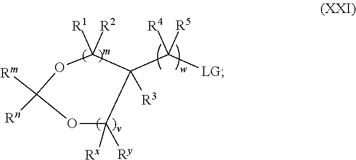

and
an amino acid-comprising conjugation partner comprising a thiol of the formula (III):

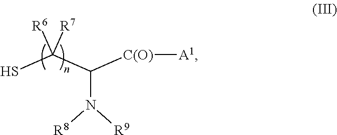

under conditions effective to conjugate the compound of formula (XXI) and amino acid-comprising conjugation partner and provide a compound of formula (XX):

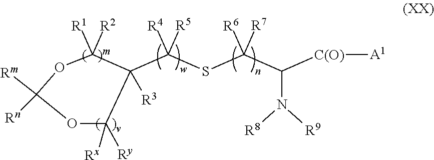

wherein
Rm and Rn are each independently hydrogen, C1-6alkyl, aryl, or heteroaryl;
LG is a leaving group; and
   m, w, v, n, Rx, Ry, R1, R2, R3, R4, R5, R6, R7, R8, R9, and A1 are as defined in the compound of formula (I) of the present invention (including provisos (1) and/or (2) of the first aspect); and
converting the compound of formula (XX) to a peptide conjugate of the formula (I) of the present invention (including provisos (1) and/or (2) of the first aspect) or a pharmaceutically acceptable salt or solvate thereof by one or more additional synthetic steps:

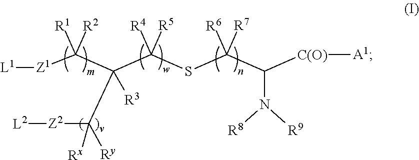

or
(B) reacting
a compound of the formula (XXI):

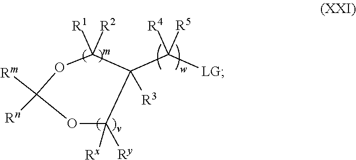

and
an amino acid-comprising conjugation partner comprising a thiol of the formula (III):

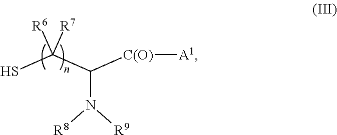

under conditions effective to conjugate the compound of formula (XXI) and amino acid-comprising conjugation partner and provide a compound of formula (XX):

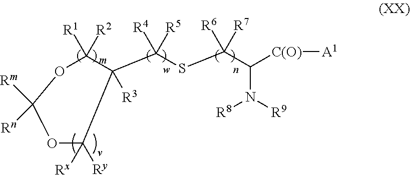

wherein
Rm and Rn are each independently hydrogen, C1-6alkyl, aryl, or heteroaryl;
LG is a leaving group; and
m, w, v, n, Rx, Ry, R1, R2, R3, R4, R5, R6, R7, R8, R9, and A1 are as defined in the compound of formula (I) but excluding provisos (1) and (2) of the first aspect; and
converting the compound of formula (XX) to an amino acid- or peptide conjugate of the formula (I) but excluding provisos (1) and (2) of the first aspect or a salt or solvate thereof by one or more additional synthetic steps:

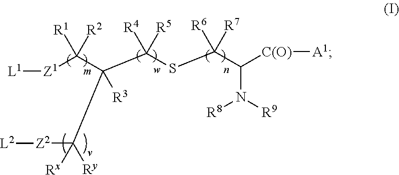

and
coupling the amino acid of the amino acid conjugate or an amino acid of the peptide conjugate to an amino acid or an amino acid of a peptide to provide the peptide-conjugate of formula (I) of the present invention (including provisos (1) and/or (2) of the first aspect) or pharmaceutically acceptable salt or solvate thereof.

In various embodiments, Rm and Rn are each independently selected from hydrogen, C1-6alkyl, or aryl.

In certain embodiments, Rm is hydrogen, C1-6alkyl, or aryl; and Rn is C1-6alkyl or aryl.

In various embodiments, the leaving group is a halo (for example chloro, bromo, or iodo) or sulfonate (for example a tosylate or mesylate).

In various embodiments, m and v are such that the compound of formula (XXI) comprises a 5-7-membered cyclic acetal.

In certain embodiment, the cyclic acetal is a 6-membered cyclic acetal.

In various embodiments, the cyclic acetal is a 5-membered cyclic acetal and w is an integer greater than 1.

In various embodiments, m is 2 and v is 1.

In various embodiments, R9 is not hydrogen and/or A1 is not OH.

In various embodiments, the amino acid-comprising conjugation partner is a peptide containing conjugation partner comprising 15 or less, 14 or less, 13 or less, 12 or less, 11 or less, 10 or less, 9 or less, 8 or less, 7 or less, 6 or less, 5 or less, 4 or less, or 3 or less amino acid residues.

In various embodiments, the C-terminus of the amino acid comprising conjugation partner is protected with a carboxyl protecting group or a carboxamide protecting group and/or the Nα-amino group of the amino acid comprising conjugation partner is protected with an amino protecting group.

In exemplary embodiments, R9 is an amino protecting group.

In various embodiments, A1 is OP1 or NHP2. In certain embodiments, A1 is OP1.

In exemplary embodiments, R9 is an amino protecting group and A1 is OP1 in the amino acid comprising conjugation partner.

In various embodiments, the method comprises reacting the compound of formula (XXI) and the amino acid-comprising conjugation partner of formula (III) in the presence of a base.

In various embodiments, the base comprises an organic amine, for example triethylamine, N-methylmorpholine, or collidine.

In various embodiments, the cyclic acetal of formula (XXI) is provided in the form of a single stereoisomer or a stereoisomerically enriched mixture.

In various embodiments, the method comprises converting the compound of formula (XX) to an amino acid- or peptide conjugate of the formula (IA) or a pharmaceutically acceptable salt or solvate thereof by one or more synthetic steps:

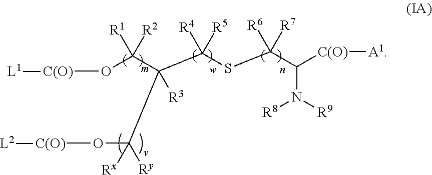

In various embodiments, the one or more synthetic steps comprises removing the acetal in the compound of formula (XX) to provide a compound of the formula (XXIII-1):

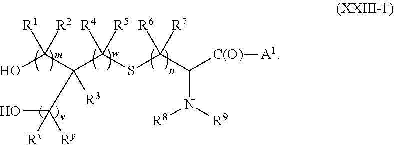

In various embodiments, wherein Rm is optionally substituted aryl, for example phenyl or methoxy substituted phenyl, the method comprises removing the acetal in the compound of formula (XX) to provide a compound of the formula (XXIII-2) or (XXIII-3):

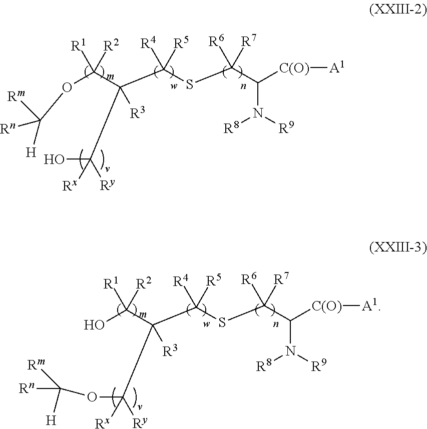

In various embodiments, the one or more synthetic steps comprise converting the hydroxyl group bound to the carbon to which R1 and R2 are attached in the compound of formula (XXIII-1) to L1-Z1-, and/or converting the hydroxyl group bound to the carbon to which Rx and Ry are attached to L2-Z2.

In various embodiments, the one or more synthetic steps comprise
converting the hydroxyl group bound to the carbon atom to which Rx and Ry are attached in the compound of formula (XXIII-2) to L2-Z2-, removing the RmRnCH- group to provide a hydroxyl group, and converting the hydroxyl group to L1-Z1; or
converting the hydroxyl group bound to the carbon to which Rx and Ry are attached in the compound of formula (XXIII-2) to L1-Z1-, removing the RmRnCH- group to provide a hydroxyl group, and converting the hydroxyl group to L2-Z2-.

In various embodiments, converting said hydroxyl group to L1-Z1- or L2-Z2- comprises acylating so as to replace the hydrogen atom of the hydroxyl group with L1-C(O)— or L2-C(O)—.

In another aspect, the present invention broadly consists in a compound of the formula (XX):

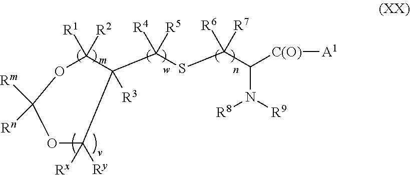

wherein:
Rm and Rn are each independently hydrogen, C1-6alkyl, aryl, or heteroaryl;
m and w are each independently an integer from 0 to 7 and v is an integer from 0 to 5,
   provided that:
      the sum of m, v, and w is at least 3; and
      the sum of m and w is from 0 to 7; and
n, Rx, Ry, R1, R2, R3, R4, R5, R6, R7, R8, R9, and A1 are as defined in the compound of formula (I) of the present invention (including provisos (1) and/or (2) of the first aspect) or any embodiment thereof; or a salt or solvate thereof.

In another aspect, the present invention broadly consists in a method of making a compound of the formula (XX) or a salt or solvate thereof, the method comprising:

(A) reacting
a compound of the formula (XXI):

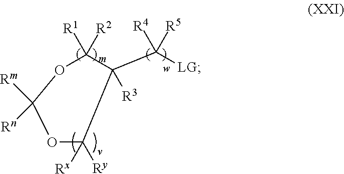

and
an amino acid-comprising conjugation partner comprising a thiol of the formula (III):

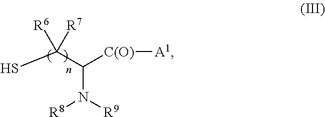

under conditions effective to conjugate the compound of formula (XXI) and amino acid-comprising conjugation partner and provide a compound of formula (XX):

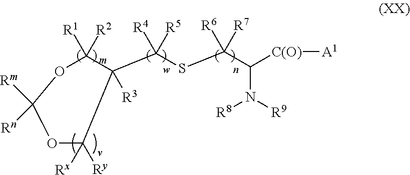

wherein
Rm and Rn are each independently hydrogen, C1-6alkyl, aryl, or heteroaryl;
LG is a leaving group; and
   m, w, v, n, Rx, Ry, R1, R2, R3, R4, R5, R6, R7, R8, R9, and A1 are as defined in the compound of formula (I) of the present invention (including provisos (1) and/or (2) of the first aspect).

In another aspect, the present invention broadly consists in the use of a compound of the formula (XX) or (XXI) in the synthesis of a peptide-conjugate of the formula (IA) of the present invention (including provisos (1) and/or (2) of the first aspect) or a pharmaceutically acceptable salt or solvate thereof.

In another aspect, the present invention broadly consists in a method of making a peptide conjugate of the formula (I) or a pharmaceutically acceptable salt or solvate thereof of the present invention, the method comprising:

(A) reacting
a first lipid-containing conjugation partner comprising a carbon-carbon double bond,
a second lipid-containing conjugation partner comprising a carbon-carbon double bond, and
an amino acid-comprising conjugation partner comprising a thiol
under conditions effective to conjugate the first lipid-containing conjugation partner and the second lipid-containing conjugation partner to the amino acid-comprising conjugation partner and provide the peptide-conjugate of formula (I) or salt or solvate thereof,
wherein in the amino acid- or peptide conjugate the sulfur atom from the thiol of the amino acid-comprising conjugation partner is conjugated to a carbon atom from the carbon-carbon double bond of the first lipid-containing conjugation partner, and a carbon atom from the carbon-carbon double bond of the first lipid-containing conjugation partner is conjugated to a carbon atom from the carbon-carbon double bond of the second lipid-containing conjugation partner; or
   (B) reacting
a first lipid-containing conjugation partner comprising a carbon-carbon double bond,
a second lipid-containing conjugation partner comprising a carbon-carbon double bond, and
an amino acid-comprising conjugation partner comprising a thiol
under conditions effective to conjugate the first lipid-containing conjugation partner and the second lipid-containing conjugation partner to the amino acid-comprising conjugation partner and provide an amino acid- or peptide-conjugate,
wherein in the amino acid- or peptide conjugate the sulfur atom from the thiol of the amino acid-comprising conjugation partner is conjugated to a carbon atom from the carbon-carbon double bond of the first lipid-containing conjugation partner, and a carbon atom from the carbon-carbon double bond of the first lipid-containing conjugation partner is conjugated to a carbon atom from the carbon-carbon double bond of the second lipid-containing conjugation partner; and
coupling the amino acid of the amino acid conjugate or an amino acid of the peptide conjugate to an amino acid or an amino acid of a peptide to provide the peptide-conjugate of formula (I) or salt or solvate thereof.

In one embodiment, the amino acid-comprising conjugation partner is a peptide-containing conjugation partner, and the lipid-containing conjugation partners are coupled to the peptide of the peptide-containing conjugation partner.

In some embodiments, the lipid-containing conjugation partners are conjugated to the or an amino acid of the amino acid-comprising conjugation partner or the peptide of the peptide-containing conjugation partner.

In certain embodiments, the lipid-containing conjugation partners are conjugated to the or an amino acid of the amino acid-comprising conjugation partner.

Accordingly, in another aspect, the present invention broadly consists in a method of making a peptide conjugate of formula (I) or a pharmaceutically acceptable salt or solvate thereof of the present invention, the method comprising reacting
a first lipid-containing conjugation partner comprising a carbon-carbon double bond,
a second lipid-containing conjugation partner comprising a carbon-carbon double bond, and
peptide-containing conjugation partner comprising a thiol
under conditions effective to conjugate the first lipid-containing conjugation partner and the second lipid-containing conjugation partner to the peptide-containing conjugation partner and provide the peptide conjugate of formula (I) or salt or solvate thereof,
wherein in the peptide conjugate the sulfur atom from the thiol of the peptide-containing conjugation partner is conjugated to a carbon atom from the carbon-carbon double bond of the first lipid-containing conjugation partner, and a carbon atom from the carbon-carbon double bond of the first lipid-containing conjugation partner is conjugated to a carbon atom from the carbon-carbon double bond of the second lipid-containing conjugation partner.

In various embodiment, the conjugate is a lipopeptide, such that the method is for making a lipopeptide.

In various embodiments, the first and second lipid-containing conjugation partners have the same structure (that is, the first and second lipid-containing conjugation partners are identical).

In various embodiments, the method comprises conjugating the sulfur atom of the thiol to a carbon atom of the carbon-carbon double bond of the first lipid containing conjugation partner and then conjugating a carbon atom from the carbon-carbon double bond to which the thiol is conjugated to a carbon atom of the carbon-carbon double bond of the second lipid-containing conjugation partner.

In various embodiments, the first lipid-containing conjugation partner is a compound of the formula (IIA):

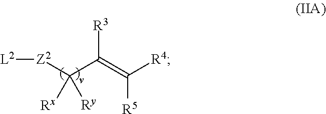

the second lipid-containing conjugation partner is a compound of the formula (IIB):

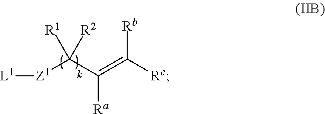

and
the amino acid-comprising conjugation partner comprises a structure of the formula (III):

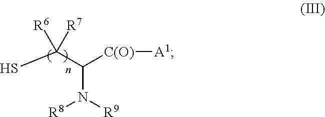

wherein:
when the method is (A), Ra, Rb, Rc, L1, L2, Z1, Z2, R1, R2, Rx, Ry, R3, R4, R5, R6, R7, R8, R9, A1, k, v, and n are as defined in the compound of formula (IB) of the present invention (including provisos (1) and/or (2) of the first aspect); and
when the method is (B), Ra, Rb, Rc, L1, L2, Z1, Z2, R1, R2, Rx, Ry, R3, R4, R5, R6, R7, R8, R9, A1, k, v, and n are as defined in the compound of formula (IB) but excluding provisos (1) and (2) of the first aspect.

In various embodiments, the amino acid- or peptide conjugate is a compound of the formula (IB):

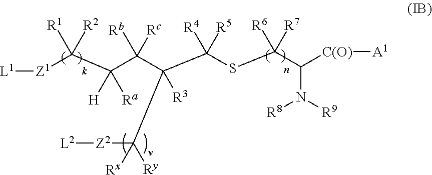

wherein:
when the method is (A), Ra, Rb, Rc, L1, L2, Z1, Z2, R1, R2, Rx, Ry, R3, R4, R5, R6, R7, R8, R9, A1, k, v, and n are as defined in the compound of formula (IB) of the present invention (including provisos (1) and/or (2) of the first aspect); and
when the method is (B), Ra, Rb, Rc, L1, L2, Z1, Z2, R1, R2, Rx, Ry, R3, R4, R5, R6, R7, R8, R9, A1, k, v, and n are as defined in the compound of formula (IB) but excluding provisos (1) and (2) of the first aspect.

In various embodiments, the lipid containing conjugation partners are in stoichiometric excess to the amino acid-comprising conjugation partner.

In various embodiments, the mole ratio of the lipid containing conjugation partners (combined) to amino acid-comprising conjugation partner is at least 7:1.

In various embodiments, the first lipid-containing conjugation partner is a compound of the formula (IIA-1):

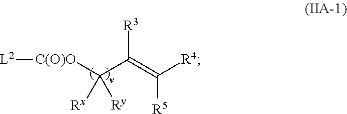

the second lipid-containing conjugation partner is a compound of the formula (IIB):

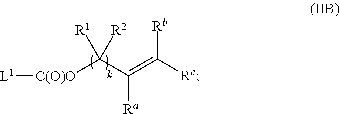

the amino acid-comprising conjugation partner comprises a structure of the formula (III):

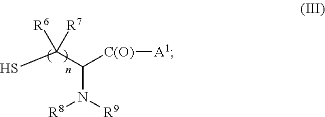

and
the conjugate is a compound of the formula (IC):

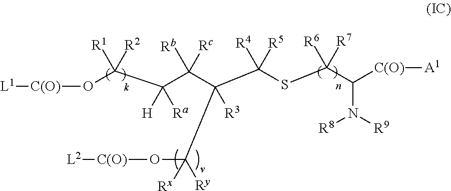

wherein:
when the method is (A), Ra, Rb, Rc, L1, L2, R1, R2, Rx, Ry, R3, R4, R5, R6, R7, R8, R9, A1, k, v, and n are as defined in the compound of formula (IC) of the present invention (including provisos (1) and/or (2) of the first aspect); and
when the method is (B), Ra, Rb, Rc, L1, L2, R1, R2, Rx, Ry, R3, R4, R5, R6, R7, R8, R9, A1, k, v, and n are as defined in the compound of formula (IC) but excluding provisos (1) and (2) of the first aspect.

In various embodiments, L1 is C11-21alkyl; k is 0-3, preferably 0; and Ra, Rb, and Rc are each hydrogen.

In various embodiments, L1 is C11-21alkyl; k is 1-3; and Ra, Rb, and Rc are each hydrogen.

In various embodiments, L2 is C11-21alkyl; v is 0-3, preferably 0; and R3, R4, and R5 are each hydrogen.

In various embodiments, n is 1; R6, R7, and R8 are each hydrogen; and R9 is hydrogen, an amino protecting group, L3-C(O), or A2.

In various embodiments, n is 1; R6, R7, and R8 are each hydrogen; and R9 is hydrogen, an amino protecting group, or L3-C(O), wherein L3 is linear C15alkyl or methyl.

In various embodiments, the compounds of formula (IIA) and (IIB) are each vinyl palmitate.

In various embodiments, the amino-acid comprising conjugation partner is cysteine, a protected cysteine (including Nα-amine and/or carboxyl protected cysteine), or a peptide comprising a cysteine residue (including an Nα-amine or carboxyl protected cysteine residue), for example, an N-terminal cysteine residue (including an Nα-amine protected cysteine residue).

In some embodiments, the method comprises reacting vinyl palmitate and an Nα-amino protected cysteine, such as Fmoc-Cys-OH, Boc-Cys-OH, Fmoc-Cys-OP1, or Boc-Cys-OP1. In some embodiments, the carboxyl group of the Nα-amino protected cysteine is protected.

In one embodiment, the conditions effective to conjugate the lipid-containing conjugation partners to the amino acid-comprising conjugation partner comprises the generation of one or more free radicals. In one embodiment, the conditions effective to conjugate the lipid-containing conjugation partners to the peptide-containing conjugation partner comprises the generation of one or more free radicals.

In some embodiments, the generation of one or more free radicals is initiated thermally and/or photochemically. In certain embodiments, the generation of one or more free radicals is initiated by the thermal and/or photochemical degradation of a free radical initiator. In exemplary embodiments, the generation of one or more free radicals is initiated by the thermal degradation of a thermal initiator or the photochemical degradation of a photochemical initiator.

In some embodiments, thermal degradation of the free radical initiator comprises heating the reaction mixture at a suitable temperature. In some embodiments, the reaction mixture is heated at a temperature is from about 40° C. to about 200° C., from about 50° C. to about 180° C., from about 60° C. to about 150° C., from about 65° C. to about 120° C., from about 70° C. to about 115° C., from about 75° C. to about 110° C., or from about 80° C. to about 100° C. In other embodiments, the reaction mixture is heated at a temperature of at least about 40° C., at least about 50° C., at least about 60° C., or at least about 65° C. In one specifically contemplated embodiment, the reaction mixture is heated at a temperature of about 90° C.

In some embodiments, photochemical degradation of the free radical initiator comprises irradiation with ultraviolet light, preferably having a frequency compatible with the side chains of naturally occurring amino acids. In a specifically contemplated embodiment, the ultraviolet light has a wavelength of about 365 nm. In exemplary embodiments, photochemical degradation of the free radical initiator is carried out at about ambient temperature.

In one specifically contemplated embodiment, the thermal initiator is 2,2′-azobisisobutyronitrile (AIBN). In one specifically contemplated embodiment, the photoinitiator is 2,2-dimethoxy-2-phenylacetophenone (DMPA).

In certain embodiments, the reaction is carried out in a liquid medium. In one embodiment, the liquid medium comprises a solvent. In one embodiment, the solvent is selected from the group consisting of N-methylpyrrolidone (NMP), dimethylsulfoxide (DMSO), N,N-dimethylformamide (DMF), dichloromethane (DCM), 1,2-dichloroethane, and mixtures thereof. In one specifically contemplated embodiment, the solvent comprises NMP, DMF, DMSO, or a mixture thereof.

In one specifically contemplated embodiment, the solvent comprises DMSO or NMP. In exemplary embodiments, the solvent comprises NMP.

In some embodiments, the reaction is carried out in the presence of one or more additives that inhibit the formation of by-products and/or that improve the yield of or conversion to the desired conjugate.

In various embodiments, the one or more additive is an extraneous thiol, an acid, an organosilane, or a combination of any two or more thereof.

In some exemplary embodiments, the extraneous or exogenous thiol is selected from the group consisting of reduced glutathione (GSH), 2,2′-(ethylenedioxy)diethanethiol (DODT), 1,4-dithiothreitol (DTT), protein, and sterically hindered thiols. In a specifically contemplated embodiment, the extraneous or exogenous thiol is DTT. In some embodiments, the extraneous or exogenous thiol is a sterically hindered thiol, for example tert-butyl mercaptan.

In various embodiments, the acid additive is a strong inorganic or organic acid. In various embodiments, the acid is a strong organic acid. In various embodiments, the acid is TFA.

In various embodiments, the organosilane is a trialkylsilane, for example TIPS.

In some embodiments, the one or more additive is selected from the group consisting of TFA, tert-butyl mercaptan, TIPS, and combinations of any two or more thereof.

In certain embodiments, the one or more additive is a combination of an acid and an extraneous thiol, for example TFA and tert-butyl mercaptan.

In other embodiments, the one or more additive is a combination of an acid and an organosilane, for example TFA and TIPS.

In other embodiments, the one or more additive is a combination of an extraneous thiol and an organosilane, and optionally an acid, for example a combination of t-BuSH and TIPS, and TFA.

In some embodiments, the reaction is carried out for a period of time from about 5 minutes to about 48 h, 5 minutes to about 24 h, from about 5 minutes to about 12 hours, from about 5 minutes to about 6 hours, from about 5 minutes to about 3 hours, 5 minutes to 2 hours, or form about 5 minutes to about 1 hour. In exemplary embodiments, the reaction is carried out for a period of time from about 5 minutes to about 1 h. In some embodiments, the reaction is carried out until one of the conjugation partners is at least about 70%, 80%, 90%, 95%, 97%, 99%, or 100% consumed.

In certain embodiments, the reaction is carried out under substantially oxygen free conditions.

In various embodiments, the amino acid-comprising conjugation partner is a peptide-containing conjugation partner.

In one embodiment, the amino acid-comprising conjugation partner comprises an epitope. In one embodiment, the peptide-containing conjugation partner comprises an epitope, such as a peptide epitope.

In one embodiment, the amino acid-comprising conjugation partner comprises two or more epitopes. In one embodiment, the peptide-containing conjugation partner comprises two or more epitopes.

In one embodiment, the amino acid-comprising conjugation partner consists of a peptide.

In one embodiment, the amino acid-comprising conjugation partner consists of a peptide comprising a peptide epitope. In one embodiment, the peptide-containing conjugation partner consists of a peptide. In one embodiment, the peptide-containing conjugation partner consists of a peptide comprising a peptide epitope.

In some embodiments, the amino acid-comprising conjugation partner comprises an epitope bound to the or an amino acid of the conjugation partner. In some embodiments, the peptide-containing conjugation partner comprises an epitope bound to the peptide of the peptide containing conjugation partner. In some embodiments, the epitope is bound to the peptide via linker group.

In some embodiments, the amino acid-comprising conjugation partner comprises a peptide epitope bound to the or an amino acid of the conjugation partner via a linker group. In some embodiments, the peptide-containing conjugation partner comprises a peptide epitope bound to the peptide via a linker group.

In some embodiments, the amino acid-comprising conjugation partner and/or the peptide-containing conjugation partner comprises an antigenic peptide.

In one embodiment, the amino acid-comprising conjugation partner and/or peptide conjugate comprises a synthetic peptide. In some embodiments, the synthetic peptide is a peptide prepared by a method comprising solid phase peptide synthesis (SPPS).

In various embodiments, the method comprises coupling the amino acid of the amino acid conjugate or an amino acid of the peptide conjugate to an amino acid or an amino acid of a peptide to provide a peptide conjugate.

In various embodiments, the method comprises coupling the amino acid of the amino acid conjugate to an amino acid or an amino acid of a peptide to provide a peptide conjugate of the present invention.

In various embodiments, the peptide comprises an epitope. In various embodiments, the epitope is a peptide epitope.

In some embodiments, the method further comprises coupling the amino acid of the amino acid conjugate to an amino acid or a peptide to provide a peptide conjugate of the present invention.

In some embodiments, coupling a peptide comprises individually coupling one or more amino acids and/or one or more peptides.

In some embodiments, the method further comprises coupling the amino acid of the amino acid conjugate or an amino acid of the peptide conjugate to an amino acid or a peptide so as to provide a peptide conjugate of the invention comprising a linker group or one or more amino acids thereof.

In some embodiments, the method further comprises coupling an amino acid of the peptide conjugate comprising a linker group or one or more amino acids thereof to an amino acid or a peptide so as to provide a peptide conjugate of the invention comprising a peptide epitope bound to the amino acid to which lipid moieties are conjugated via a linker group.

In some embodiments, the amino acid of the peptide conjugate to which the lipid moeities are conjugated is an N-terminal amino acid residue.

In some embodiments, the method further comprises coupling the amino acid of the amino acid conjugate or an amino acid of the peptide conjugate to an amino acid or a peptide so as to provide a peptide conjugate of the invention comprising a peptide epitope.

In some embodiments, the method further comprises coupling an epitope to the amino acid of the amino acid conjugate or an amino acid of the peptide conjugate.

In some embodiments, the method further comprises coupling a peptide epitope to the amino acid of the amino acid conjugate or an amino acid of the peptide conjugate.

In some embodiments, the epitope is coupled or bound via a linker group.

In some embodiments, the method further comprises coupling an epitope to the peptide of the peptide conjugate.

In some embodiments, the method further comprises coupling a peptide epitope to the peptide of the peptide conjugate.

In some embodiments, the epitope is bound to the peptide via a linker group.

In various embodiments, the method is (B) and the amino acid-comprising conjugation partner consists of an amino acid, for example cysteine (including Nα-amino and/or C-terminus protected cysteines).

In various embodiments, the amino acid- or peptide-conjugate compound of formula (I) but excluding provisos (1) and (2) of the first aspect is an amino acid-conjugate.

In some embodiments, A1 is OH, OP1, NH2, or NHP2 and/or R9 is hydrogen, C1-6alkyl, C3-6cycloalkyl, an amino protecting group, or L3-C(O) in the amino acid- or peptide-conjugate compound of formula (I) but excluding provisos (1) and (2) of the first aspect.

In some of such embodiments, A1 is OP1 or OH and/or R9 is hydrogen, an amino protecting group or L3-C(O) in the amino acid- or peptide-conjugate compound of formula (I) but excluding provisos (1) and (2) of the first aspect.

In various of such embodiments, A1 is OH, OP1, NH2, or NHP2 and R9 is hydrogen, C1-6alkyl, C3-6cycloalkyl, an amino protecting group, or L3-C(O) in the amino acid- or peptide-conjugate compound of formula (I) but excluding provisos (1) and (2) of the first aspect.

In various embodiments, A1 is OH, or OP1, and R9 is hydrogen, an amino protecting group, or L3-C(O) in the amino acid- or peptide-conjugate compound of formula (I) but excluding provisos (1) and (2) of the first aspect.

In various embodiments, the C-terminus of the amino acid comprising conjugation partner is protected with a protecting group and/or the Nα-amino group of the amino acid comprising conjugation partner is protected with a protecting group.

In various embodiments, the carboxyl group of the C-terminus of the amino acid is protected with a carboxyl protecting group or a carboxamide protecting group and/or the Nα-amino group of the amino acid is protected with an amino protecting group.

In various embodiments, the carboxyl group of the C-terminus of the amino acid is protected with a carboxyl protecting group and/or the Nα-amino group of the amino acid is protected with an amino protecting group.

In some embodiments, the carboxyl group of the C-terminus of the peptide is protected with a carboxyl protecting group and/or the Nα-amino group of the peptide is protected with an amino protecting group.

In some embodiments, the amino acid residue comprising the thiol is a terminal amino acid residue. In some embodiments, the amino acid residue comprising the thiol is an N-terminal residue.

In some embodiments, A1 and/or R9 is a group other than an amino acid or a peptide, and the method comprises coupling an amino acid or a peptide so as to replace A1 and/or R9 with the amino acid or peptide.

In some embodiments, A1 a group other than an amino acid or a peptide, and the method comprises coupling an amino acid or a peptide so as to replace A1 with the amino acid or peptide.

In some embodiments, A1 is a OH, OP1, NH2, or NHP2 and/or R9 is hydrogen, an amino protecting group or L3-C(O), and the method comprises coupling an amino acid or a peptide so as to replace A1 and/or R9 with the amino acid or peptide.

In some embodiments, A1 is a OH, OP1, NH2, or NHP2 and R9 is hydrogen, an amino protecting group or L3-C(O) and the method further comprises coupling an amino acid or a peptide so as to replace A1 and/or R9 with the amino acid or peptide.

In some embodiments, coupling a peptide comprises individually coupling one or more amino acids and/or one or more peptides.

In some embodiments, coupling the amino acid or peptide provides a peptide conjugate comprising a peptide epitope. In some embodiments, the coupling the amino acid or peptide provides a peptide conjugate comprising a linker group or one or more amino acids thereof. In some embodiments, coupling the amino acid or peptide provides a peptide conjugate comprising a peptide epitope bound to the amino acid to which the lipid moieties are conjugated via a linker group.

In some embodiments, the Nα-amino group of the amino acid comprising the thiol to which the lipid moieties are conjugated is acylated. In some embodiments, R9 in the amino acid comprising conjugation partner comprising the thiol is L3-C(O)—, for example Me-C(O)—.

In certain embodiments, the method further comprises acylating the Nα-amino group of the amino acid of the amino acid conjugate or the amino acid residue of the peptide conjugate to which the lipid moeities are conjugated. In certain embodiments, the method further comprises acylating the Nα-amino group with a C2-20 fatty acid, such as acetyl.

In some embodiments, R9 is hydrogen or an amino protecting group, and the method further comprises acylating the amino acid conjugate or peptide conjugate so as to replace the hydrogen or amino protecting group at R9 with L3-C(O).

In some embodiments, acylating the amino acid conjugate or peptide conjugate so as to replace the amino protecting group at R9 with L3-C(O) comprises removing the amino protecting group at R9 to provide a hydrogen at R9.

In certain embodiments, the or an amino acid of the amino acid-comprising conjugation partner comprises the thiol. In certain embodiments, an amino acid residue of the peptide of the peptide-containing conjugation partner comprises the thiol.

In certain embodiments, the thiol is the thiol of a cysteine residue.

In certain embodiments, the cysteine residue is a terminal residue. In certain embodiments, the cysteine residue is an N-terminal residue.

In some embodiments, the amino group of the cysteine residue is acylated.

In one embodiment, the amino group is acylated with a C2-20 fatty acid.

In one exemplary embodiment, the C2-20 fatty acid is acetyl or palmitoyl. In another exemplary embodiment, the C2-20 fatty acid is acetyl.

In some embodiments, the amino acid-comprising conjugation partner and/or peptide conjugate comprises from 8 to 220, 8 to 200, 8 to 175, 8 to 150, 8 to 125, 8 to 100, 8 to 90, 8 to 80, 8 to 70, 8 to 60, 8 to 50, 8 to 40, 8 to 30, 8 to 25, 8 to 20, or 8 to 15 amino acids. In some embodiments, the peptide-containing conjugation partner comprises from 8 to 220, 8 to 200, 8 to 175, 8 to 150, 8 to 125, 8 to 100, 8 to 90, 8 to 80, 8 to 70, 8 to 60, 8 to 50, 8 to 40, 8 to 30, 8 to 25, 8 to 20, or 8 to 15 amino acids.

In one exemplary embodiment, the amino acid-comprising conjugation partner and/or peptide conjugate comprises a peptide comprising from 8 to 60 amino acids. In one exemplary embodiment, the peptide comprises from 8 to 60 amino acids.

In other embodiments, the amino acid-comprising conjugation partner and/or peptide conjugate comprises from 5 to 220, 8 to 220, 5 to 175, 8 to 175, 8 to 150, 10 to 150, 15 to 125, 20 to 100, 20 to 80, 20 to 60, 25 to 100, 25 to 80, 25 to 60, 30 to 80, 40 to 60, or 50 to 60 amino acids. In other embodiments, the peptide-containing conjugation partner comprises from 5 to 220, 8 to 220, 5 to 175, 8 to 175, 8 to 150, 10 to 150, 15 to 125, 20 to 100, 20 to 80, 20 to 60, 25 to 100, 25 to 80, 25 to 60, 30 to 80, 40 to 60, or 50 to 60 amino acids.

In other embodiments, the amino acid comprising conjugation partner and/or peptide conjugate comprises from 5 to 150, 5 to 125, 5 to 100, 5 to 75, 5 to 60, 5 to 50, 5 to 40, 5 to 30, 5 to 25, 5 to 20, 8 to 150, 8 to 125, 8 to 100, 8 to 75, 8 to 60, 8 to 50, 8 to 40, 8 to 30, 8 to 25, or 8 to 20 amino acids. In other embodiments, the peptide-containing conjugation partner comprises from 5 to 150, 5 to 125, 5 to 100, 5 to 75, 5 to 60, 5 to 50, 5 to 40, 5 to 30, 5 to 25, 5 to 20, 8 to 150, 8 to 125, 8 to 100, 8 to 75, 8 to 60, 8 to 50, 8 to 40, 8 to 30, 8 to 25, or 8 to 20 amino acids.

In various embodiments, the amino acid comprising conjugation partner is a short peptide. In some embodiments, the short peptide comprises less than 10, 9, 8, 7, 6, 5, 4, or 3 amino acids.

In one embodiment, the amino acid-comprising conjugation partner and/or peptide conjugate comprises one or more solubilising groups. In one embodiment, the peptide-containing conjugation partner comprises one or more solubilising groups.

In certain embodiments, the solubilising group is an amino acid sequence comprising two or more hydrophilic amino acid residues in the peptide chain. In certain embodiments, the solubilising group is an amino acid sequence comprising a sequence of two or more consecutive hydrophilic amino acid residues in the peptide chain. In one embodiment, the hydrophilic amino acid residues are cationic amino acid residues. In one embodiment, the cationic amino acid residues are arginine or lysine residues. In one specifically contemplated embodiment, the cationic amino acid residues are lysine residues. In one embodiment, the sequence comprises from 2 to 20, 2 to 15, 2 to 10, 3 to 7, or 3 to 5 amino acids. In one embodiment, the solubilising group is a tri-, tetra-, penta-, hexa-, or hepta- lysine sequence. In one specifically contemplated embodiment, the solubilising group is a tetralysine sequence.

In some embodiments, the peptide conjugate and/or amino-acid comprising conjugation partner comprises a serine residue adjacent to the amino acid residue to which the lipid moeities are conjugated. In a specifically contemplated embodiment, the peptide of the peptide-containing conjugation partner comprises a serine residue adjacent to the amino acid residue to which the lipid moeities are conjugated. In an exemplary embodiment, the amino acid residue to which the lipid moeities are conjugated is N-terminal. In a specifically contemplated embodiment, the peptide further comprises a consecutive sequence of two or more hydrophilic amino acid residues adjacent to the serine residue.

In certain embodiments, the peptide conjugate and/or amino-acid comprising conjugation partner comprises a consecutive sequence of two or more hydrophilic amino acid residues adjacent to the serine residue.

In certain embodiments, the peptide conjugate and/or amino acid-comprising conjugation partner comprises only naturally occurring amino acids. In certain embodiments, the peptide-containing conjugation partner comprises only naturally occurring amino acids. In other embodiments, 75% or more, 80% or more, 85% or more, 90% or more, 95% or more, 97% or more, or 99% or more of the amino acid residues in the peptide are naturally occurring amino acids.

In other embodiments, 75% or more, 80% or more, 85% or more, 90% or more, 95% or more, 97% or more, or 99% or more of the amino acid residues in the peptide conjugate and/or amino acid-comprising conjugation partner are naturally occurring amino acids.

In exemplary embodiments, the peptide conjugate and/or amino acid-comprising conjugation partner comprises a peptide comprising a peptide epitope. In exemplary embodiments, the peptide of the peptide-containing conjugation partner comprises one or more peptide epitopes.

In various embodiments, the peptide comprises, consists essentially of, or consists of an amino acid sequence selected from the group consisting of those defined in proviso (1) of the first aspect.

In various embodiments, the peptide comprises, consists essentially of, or consists of one or more EBV LMP2 epitopes. In various embodiments, the one or more EBV LMP2 epitopes are MHCI epitopes. In various embodiments, the peptide comprises one or more EBV LMP2 epitopes selected from the group consisting of any one of SEQ ID NOs 84-109. In various embodiments, the peptide comprises a peptide comprising or consisting of 12 or more contiguous amino acids from the amino acid sequence of any one of SEQ ID NOs 9-83. In various embodiments, the peptide comprises a peptide comprising or consisting of 15 or more contiguous amino acids from the amino acid sequence of any one of SEQ ID NOs 9-83, or comprising or consisting of 20 or more contiguous amino acids from the amino acid sequence of any one of SEQ ID NOs 9-83.

In various embodiments, the peptide comprises a recombinant peptide comprising or consisting of 12 or more contiguous amino acids from the amino acid sequence of any one of SEQ ID NOs 9-83. In various embodiments, the recombinant peptide comprises or consists of 15 or more contiguous amino acids from the amino acid sequence of any one of SEQ ID NOs 9-83, or comprises or consists of 20 or more contiguous amino acids from the amino acid sequence of any one of SEQ ID NOs 9-83.

In one exemplary embodiment, the peptide epitope is derived from NY-ESO-1. In one specifically contemplated embodiment, the peptide comprises, consists essentially of, or consists of an amino acid sequence selected from the group consisting of 8 or more contiguous amino acid residues from any one of SEQ ID NO: 114, 115, 116, 121, 122, 127, 128, and 129.

In various embodiments, the peptide comprises, consists essentially of, or consists of one or more NY-ESO-1 epitopes. In various embodiments, the one or more NY-ESO-1 epitopes are MHCI epitopes. In various embodiments, the the peptide comprises, consists essentially of, or consists of an amino acid sequence selected from the group consisting of 8 or more contiguous amino acid residues from any one of SEQ ID NO: 114, 115, 116, 121, 122, 127, 128, and 129. In various embodiments, the peptide comprises a peptide comprising or consisting of 12 or more contiguous amino acids from the amino acid sequence of any one of SEQ ID NO: 114, 115, 116, 121, 122, 127, 128, and 129. In various embodiments, the peptide comprises a peptide comprising or consisting of 15 or more contiguous amino acids from the amino acid sequence of any one of SEQ ID NO: 114, 115, 116, 121, 122, 127, 128, and 129, or comprising or consisting of 20 or more contiguous amino acids from the amino acid sequence of any one of SEQ ID NO: 114, 115, 116, 121, 122, 127, 128, and 129.

In one specifically contemplated embodiment, the reactive functional groups of the amino acids of the peptide-containing conjugation partner are unprotected.

In certain embodiments, one or more reactive functional groups of one or more amino acids of the peptide conjugate are unprotected.

In certain embodiments, one or more reactive functional groups of the amino acid of the amino acid conjugate are unprotected.

In certain embodiments, one or more reactive functional groups of one or more amino acids of the amino acid-comprising conjugation partner are unprotected.

In certain embodiments, the amino acid-comprising conjugation partner comprises a peptide, wherein the reactive functional groups of the side chains of the amino acids of the peptide are unprotected, with the exception of any thiols other than the thiol to be reacted.

In certain specifically contemplated embodiments, the reactive functional groups of the amino acids of the peptide of the peptide-containing conjugation partner are unprotected.

In certain specifically contemplated embodiments, the reactive functional groups of the amino acids of the peptide of the peptide-containing conjugation partner are unprotected, with the exception of any thiols other than the thiol to be reacted.

Those skilled in the art will appreciate that the peptide of the peptide conjugate and/or peptide-containing conjugation partner may, as described herein, be optionally substituted, modified, or bound to various other moieties as described herein to provide the peptide conjugate and/or peptide containing conjugation partner.

In some embodiments, the method comprises
synthesising the amino acid sequence of a peptide by solid phase peptide synthesis (SPPS);
coupling the amino acid of an amino acid conjugate or an amino acid of a peptide conjugate to the solid phase bound peptide by SPPS so as to provide a peptide conjugate comprising a peptide epitope, a peptide conjugate comprising a linker group or one or more amino acids thereof, or a peptide conjugate comprising a peptide epitope bound to the amino acid to which lipid moeities are conjugated via a linker group.

In some embodiments, the method comprises
reacting the lipid-containing conjugation partners and an amino acid-comprising conjugation partner to provide an amino acid or peptide conjugate;
synthesising the amino acid sequence of a peptide by solid phase peptide synthesis (SPPS);
coupling the amino acid of the amino acid conjugate or an amino acid of the peptide conjugate to the solid phase bound peptide by SPPS so as to provide a peptide conjugate comprising a peptide epitope, a peptide conjugate comprising a linker group or one or more amino acids thereof, or a peptide conjugate comprising a peptide epitope bound to the amino acid to which lipid moeities are conjugated via a linker group.

In some embodiments, the method further comprises acylating the Nα-amino group of the amino acid of the amino acid conjugate or the amino acid to which the lipid-moieties are conjugated of any one of the peptide conjugates.

In some embodiments, the method comprises cleaving the peptide conjugate from the solid phase support.

In some embodiments, the method comprises
synthesising the amino acid sequence of the peptide of the peptide-containing conjugation partner by solid phase peptide synthesis (SPPS); and
reacting the lipid-containing conjugation partners and peptide-containing conjugation partner in accordance with any of the embodiments described herein.

In exemplary embodiments, the method comprises
synthesising the amino acid sequence of the peptide of the peptide-containing conjugation partner by SPPS,
cleaving the peptide from the solid phase support; and
reacting the lipid-containing conjugation partners and peptide-containing conjugation partner in accordance with any of the embodiments described herein.

In one embodiment, the peptide-containing conjugation partner is not purified prior to reaction with the lipid-containing conjugation partners.

In some embodiments, one or more protecting groups are removed on cleaving the peptide from the solid phase support. In certain embodiments, all of the protecting groups present in the peptide are removed.

In one embodiment, the SPPS is Fmoc-SPPS.

In some embodiments, the amino acid residue in the peptide of the peptide-containing conjugation partner bearing the thiol to be reacted is an N-terminal amino acid residue and the method comprises acylating the N-terminal amino group prior to cleaving the peptide from the solid phase. In specifically contemplated embodiments, the N-terminal residue is a cysteine residue.

In one embodiment, the method further comprises separating the peptide conjugate from the reaction medium and optionally purifying the peptide conjugate.

In another aspect, the present invention broadly consists in a method of making a peptide conjugate, the method comprising
providing an amino acid- or peptide conjugate of the formula (I) but excluding provisos (1) and (2) of the first aspect or a salt or solvate thereof, and
coupling the amino acid of the amino acid conjugate or an amino acid of the peptide conjugate to an amino acid or an amino acid of a peptide to provide a peptide conjugate of the formula (I) of the invention (including provisos (1) and/or (2) of the first aspect) or a salt or solvate thereof.

In various embodiments, the product peptide conjugate is a compound of the formula (I) or a pharmaceutically acceptable salt thereof of the present invention.

In various embodiments, the amino acid of the amino acid conjugate is coupled under conditions that reduce epimerisation at the α-carbon of the amino acid. In various embodiments, the conditions are such that less than about 35, 30, 25, 20, 15, 10, 5, 3, 2, or 1% by mol of the amino acid is epimerised. In various embodiments, the conditions that reduce epimerisation comprise the use of PyBOP or BOP as the coupling reagent. In various embodiments, the conditions that reduce epimerisation comprise the use of PyBOP as the coupling reagent. In various embodiments, the conditions comprise the use of PyBOP or BOP; and 2,4,6-trimethylpyridine. In various embodiments, the conditions comprise the use of PyBOP and 2,4,6-trimethylpyridine.

In another aspect, the present invention broadly consists in use of an amino acid- or peptide-conjugate of the formula (I) of the present invention (including provisos (1) and/or (2) of the first aspect) or a salt or solvate thereof in the synthesis of an immunogenic peptide-conjugate.

In various embodiments, the immunogenic peptide conjugate is a compound of the formula (I) of the present invention or a pharmaceutically acceptable salt thereof.

In another aspect, the present invention broadly consists in a peptide conjugate of the present invention produced by a method of the present invention.

In another aspect, the present invention broadly consists in a composition comprising a peptide conjugate of formula (I) of the present invention or a salt or solvate thereof.

In various embodiments, the composition comprises isolated, pure, purified or substantially purified compound of formula (I) of the present invention or a salt or solvate thereof.

In various embodiments, the composition comprises at least about 60, 70, 75, 80, 85, 90, 95, 97, 98, or 99% by weight compound of formula (I) of the present invention or a salt or solvate thereof.

In various embodiments, the composition is free of substantially free of amino acid- or peptide containing compounds other than compounds of formula (I) of the present invention.

In another aspect, the present invention broadly consists in a pharmaceutical composition comprising an effective amount of a peptide conjugate compound of the formula (I) of the present invention or a pharmaceutically acceptable salt or solvate thereof, and a pharmaceutically acceptable carrier.

In various embodiments, the pharmaceutical composition comprises an effective amount of two or more peptide conjugate compounds of the formula (I) of the present invention.

In one embodiment, the pharmaceutical composition is an immunogenic composition.

In one embodiment, the pharmaceutical composition does not include an extrinsic adjuvant.

In some embodiments, the pharmaceutical composition is a vaccine.

In one embodiment, the pharmaceutical composition comprises an effective amount of two or more peptide conjugates of the present invention, for example the pharmaceutical composition comprises an effective amount of three or more peptide conjugates of the present invention.

In one embodiment, the pharmaceutical composition comprises an effective amount of one or more peptide conjugates of the present invention together with one or more peptides described herein, or any combination thereof. For example, the pharmaceutical composition comprises an effective amount of two or more peptide conjugates of the present invention and one or more peptides described herein, or an effective amount of one or more peptide conjugates of the present invention and two or more peptides described herein.

In another aspect, the present invention broadly consists in a method of vaccinating or eliciting an immune response in a subject comprising administering to the subject an effective amount of one or more peptide conjugate compounds of the formula (I) of the invention or a pharmaceutically acceptable salt or solvate thereof, or an effective amount of a pharmaceutical composition of of the present invention.

In another aspect, the present invention broadly consists in use of one or more peptide conjugate compounds of formula (I) of the present invention or a pharmaceutically acceptable salt or solvate thereof or a pharmaceutical composition of the present invention in the manufacture of a medicament for vaccinating or eliciting an immune response in a subject.

In another aspect, the present invention broadly consists in one or more peptide conjugate compounds of the formula (I) of the present invention or a pharmaceutically acceptable salt or solvate thereof or a pharmaceutical composition of the present invention for vaccinating or eliciting an immune response in a subject.

In another aspect, the present invention broadly consists in use of one or more peptide conjugate compounds of the formula (I) of the invention or a pharmaceutically acceptable salt or solvate thereof or a pharmaceutical composition of the present invention for vaccinating or eliciting an immune response in a subject.

In another aspect, the present invention broadly consists in a method of activating TLR2 in a subject, the method comprising administering to the subject an effective amount of one or more peptide conjugate of the invention or a pharmaceutically acceptable salt or solvate thereof, or an effective amount of a pharmaceutical composition of the invention.

Use of one or more peptide conjugate compounds of the invention or a pharmaceutically acceptable salt or solvate thereof or a pharmaceutical composition of the invention in the manufacture of a medicament for activating TLR2 in a subject.

One or more peptide conjugate compounds of the invention or a pharmaceutically acceptable salt or solvate thereof or a pharmaceutical composition of the invention for activating TLR2 in a subject.

In various embodiments, the method, use, one or more compounds, or pharmaceutical composition is for eliciting an immune response in a subject.

In various embodiments, the method, use, one or more compounds, or pharmaceutical composition is for vaccinating a subject.

In some embodiments, the method comprises the administration of one or more peptides described herein and one or more peptide conjugates of the present invention or two or more peptide conjugates of the present invention, for example one or more peptides in combination with one or more peptide conjugates to the subject.

In some embodiments, one or more peptides described herein and one or more peptide conjugates of the present invention or two or more peptide conjugates of the present invention, for example one or more peptides in combination with one or more peptide conjugates, are used for vaccinating or eliciting an immune response in the subject or in the manufacture of a medicament for vaccinating or eliciting an immune response in the subject.

In some embodiment, two or more peptide conjugates are used or administered.

In some embodiments the two or more peptide conjugates, or one or more peptides and one or more peptide conjugates are used or administered simultaneously, sequentially, or separately.

In some embodiments, the subject is in need thereof.

Asymmetric centers may exist in the compounds described herein. The asymmetric centers may be designated as (R) or (S), depending on the configuration of substituents in three dimensional space at the chiral carbon atom. All stereochemical isomeric forms of the compounds, including diastereomeric, enantiomeric, and epimeric forms, as well as d-isomers and I-isomers, and mixtures thereof, including enantiomerically enriched and diastereomerically enriched mixtures of stereochemical isomers, are within the scope of the invention.

Individual enantiomers can be prepared synthetically from commercially available enantiopure starting materials or by preparing enantiomeric mixtures and resolving the mixture into individual enantiomers. Resolution methods include conversion of the enantiomeric mixture into a mixture of diastereomers and separation of the diastereomers by, for example, recrystallization or chromatography, and any other appropriate methods known in the art. Starting materials of defined stereochemistry may be commercially available or made and, if necessary, resolved by techniques well known in the art.

The compounds described herein may also exist as conformational or geometric isomers, including cis, trans, syn, anti, entgegen (E), and zusammen (Z) isomers. All such isomers and any mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol, imine/enamine, and thioketone/enethiol tautomerism.

The compounds described herein may also exist as isotopologues and isotopomers, wherein one or more atoms in the compounds are replaced with different isotopes. Suitable isotopes include, for example, ¹H, ²H (D), ³H (T), ¹²C, ¹³C, ¹⁴C, ¹⁶O, and ¹⁸O. Procedures for incorporating such isotopes into the compounds described herein will be apparent to those skilled in the art. Isotopologues and isotopomers of the compounds described herein are also within the scope of the invention.

Also within the scope of the invention are salts of the compounds described herein, including pharmaceutically acceptable salts. Such salts include, acid addition salts, base addition salts, and quaternary salts of basic nitrogen-containing groups.

Acid addition salts can be prepared by reacting compounds, in free base form, with inorganic or organic acids. Examples of inorganic acids include, but are not limited to, hydrochloric, hydrobromic, nitric, sulfuric, and phosphoric acid. Examples of organic acids include, but are not limited to, acetic, trifluoroacetic, propionic, succinic, glycolic, lactic, malic, tartaric, citric, ascorbic, maleic, fumaric, pyruvic, aspartic, glutamic, stearic, salicylic, methanesulfonic, benzenesulfonic, isethionic, sulfanilic, adipic, butyric, and pivalic.

Base addition salts can be prepared by reacting compounds, in free acid form, with inorganic or organic bases. Examples of inorganic base addition salts include alkali metal salts, alkaline earth metal salts, and other physiologically acceptable metal salts, for example, aluminium, calcium, lithium, magnesium, potassium, sodium, or zinc salts. Examples of organic base addition salts include amine salts, for example, salts of trimethylamine, diethylamine, ethanolamine, diethanolamine, and ethylenediamine.

Quaternary salts of basic nitrogen-containing groups in the compounds may be may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like.

The compounds described herein may form or exist as solvates with various solvents. If the solvent is water, the solvate may be referred to as a hydrate, for example, a mono-hydrate, a di-hydrate, or a tri-hydrate. All solvated forms and unsolvated forms of the compounds described herein are within the scope of the invention.

The general chemical terms used in the formulae herein have their usual meaning.

The term “aliphatic” is intended to include saturated and unsaturated, nonaromatic, straight chain, branched, acyclic, and cyclic hydrocarbons. Those skilled in the art will appreciate that aliphatic groups include, for example, alkyl, alkenyl, alkynyl, cycloalkyl, and cycloalkenyl groups, and hybrids thereof such as (cycloalkyl)alkyl, (cycloalkenyl)alkyl and (cycloalkyl)alkenyl groups. In various embodiments, aliphatic groups comprise from 1-12, 1-8, 1-6, or 1-4 carbon atoms. In some embodiments, aliphatic groups comprise 5-21, from 9-21, or from 11-21 carbon atoms, such as from 11, 13, 15, 17, or 19 carbon atoms. In some embodiments, the aliphatic group is saturated.

The term “heteroaliphatic” is intended to include aliphatic groups, wherein one or more chain and/or ring carbon atoms are independently replaced with a heteroatom, preferably a heteroatom selected from oxygen, nitrogen and sulfur. In some embodiments, the heteroaliphatic is saturated. Examples of heteroaliphatic groups include linear or branched, heteroalkyl, heteroalkenyl, and heteroalkynyl groups.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” is intended to include straight and branched chain alkyl groups having at least one double bond between two carbon atoms. In some embodiments, alkenyl groups have from 2 to 12, from 2 to 10, from 2 to 8, from 2 to 6, or from 2 to 4 carbon atoms. In some embodiments, alkenyl groups have from 5-21, from 9-21, or from 11-21 carbon atoms, such as from 11, 13, 15, 17, or 19 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds. Examples include, but are not limited to, —C≡CH, —C≡CH₃, —CH₂C≡CH₃, and —C≡CH₂CH(CH₂CH₃)₂.

The term “heteroalkyl” is intended to include alkyl groups, wherein one or more chain carbon atoms are replaced with a heteroatom, preferably a heteroatom selected from the group consisting of oxygen, nitrogen, and sulfur. In some embodiments, the heteroalkyl is saturated. Heteroalkyl groups include, for example, polyethylene glycol groups and polyethylene glycol ether groups, and the like.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 14, from 6 to 12, or from 6 to 10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” is intended to include non-aromatic ring systems containing 3 or more ring atoms, of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 ring atoms. Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl.

The term “heteroaryl” is intended to include aromatic ring systems containing 5 or more ring atoms, of which, one or more is a heteroatom. In some embodiments, the heteroatom is nitrogen, oxygen, or sulfur. In some embodiments, heteroaryl groups include mono-, bi- and tricyclic ring systems having from 5 to 16, from 5 to 14, from 5 to 12, from 5 to 10, from 5 to 8, or from 5 to 6 ring atoms. Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl.

The term “halo” or “halogen” is intended to include F, Cl, Br, and I.

The term “heteroatom” is intended to include oxygen, nitrogen, sulfur, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent/s are attached is not exceeded, and that the substitution results in a stable compound. In various embodiments, optional substituents in the compounds described herein include but are not limited to halo, CN, NO₂, OH, NH₂, NHR10, NR10R20, C1-6haloalkyl, C1-6haloalkoxy, C(O)NH₂, C(O)NHR10, C(O)NR10R20, SO₂R10, OR10, SR10, S(O)R10, C(O)R10, and C1-6aliphatic; wherein R10 and R20 are each independently C1-6aliphatic, for example C1-6alkyl.

The term “carboxyl protecting group” as used herein is means a group that is capable of readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “amine protecting group” as used herein means a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

The term “carboxamide protecting group” as used herein means a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

As used herein, the term “and/or” means “and”, or “or”, or both.

The term “(s)” following a noun contemplates the singular and plural form, or both.

The term “comprising” as used in this specification, including the claims, means “consisting at least in part of”. When interpreting each statement in this specification, including the claims, that includes the term “comprising”, features other than that or those prefaced by the term may also be present. Related terms such as “comprise” and “comprises” are to be interpreted in the same manner. The “containing” is also to be interpreted in the same manner.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The invention will be described with reference to the accompanying figures in which:

FIG. 1 is a graph showing the results of a representative TLR agonism assay in HEK-Blue™-hTLR2 cells using titrated concentrations of agonist constructs: 910 (dotted white bars); 930 (grey bars); 931 (striped bars); 932 (square hatched bars); and (R)-Pam2Cys-SK₄-SLLMWITQV (black bars). Data presented as mean+/−SD ABS (635 nm) values for triplicate wells following background subtraction. Dotted lines indicate ABS in PBS-only wells.

FIG. 2A is a bar graph showing the results of a human TLR2 agonism assay in HEK-Blue™-hTLR2 cells using titrated concentrations of the following agonist constructs: A) (from left to right): 45a, 45b, 46a, 46b, 47b, 910, 911, 912 and 913 (structures depicted in Table 3); B) (from left to right): 45b, 910 and chain elongated structures 930, 931, and 932 (structures depicted in Table 4).

FIG. 3 is a bar graph showing the results of a murine TLR2 agonism assay using titrated concentrations of the following agonist constructs: A) (from left to right): 45a, 45b, 46a, 46b, 47b, 910, 911, 912 and 913 (structures depicted in Table 3); B) (from left to right): 45b, 910 and chain elongated structures 930, 931, and 932 (structures depicted in Table 4).

### DETAILED DESCRIPTION OF THE INVENTION

The present invention provides peptide conjugate compounds of the formula (I) as defined herein in the first aspect. The inventors have advantageously found that such conjugates have surprising immunogenic activity.

The peptide conjugate compounds of formula (I) may be prepared using the methods and procedures described herein.

Starting materials and/or intermediates useful in the methods may be prepared using known synthetic chemistry techniques (for example, the methods generally described in Louis F Fieser and Mary F, Reagents for Organic Synthesis v. 1-19, Wiley, New York (1967-1999 ed.) or Beilsteins Handbuch der organischen Chemie, 4, Aufl. Ed. Springer-Verlag Berlin, including supplements (also available via the Beilstein online database)) or, in some embodiments, may be commercially available.

Preparation of the compounds may involve the protection and deprotection of various chemical groups. The need for protection and deprotection, and the selection of appropriate protecting groups, can be readily determined by a person skilled in the art. Protecting groups and methods for protection and deprotection are well known in the art (see e.g. T. W. Greene and P. G. M. Wuts, Protective Groups in Organic Synthesis, 3^{rd} Ed., Wiley & Sons, Inc., New York (1999)).

As shown in Scheme A1 and described below, compounds of formula (IF) that are compounds of formula (I) wherein w is 1, v is 0, and m is from 2 to 6, preferably 2 or 3 to 5, may be prepared via a method of the present invention involving the conjugation of an epoxide to an amino acid-comprising conjugation partner.

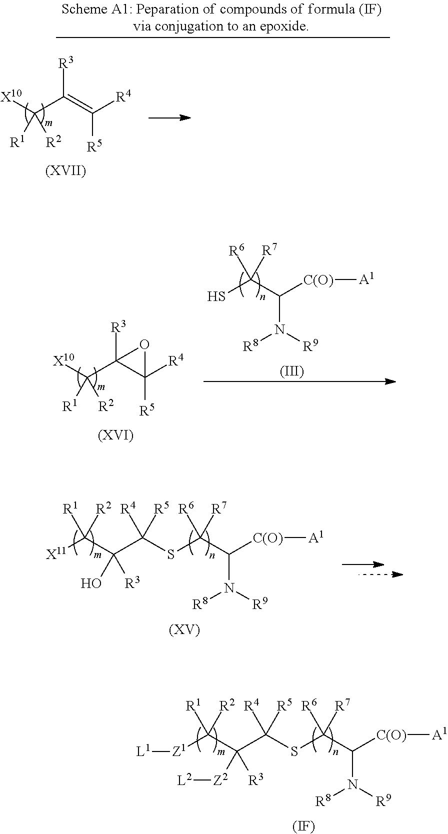

The method comprises reacting an epoxide of the formula (XVI) and an amino acid-comprising conjugation partner comprising a thiol of the formula (III) under conditions effective to provide the compound of formula (XV) by conjugation of the thiol to the epoxide.

In one embodiment of the method, method (A), the variables m, n, L1, Z1, R, R1, R2, R3, R4, R5, R6, R7, R8, R9, and A1 in the compound of formula (XV) are as defined in the compound of formula (IF) of the invention (including provisos (1) and/or (2) of the first aspect); and the method further comprises converting the compound of formula (XV) to the compound of formula (IF) of the invention by one or more additional synthetic steps. In this embodiment, the amino acid-comprising conjugation partner may comprise a peptide that corresponds the peptide present in the compound of the formula (IF) of the invention produced by the method.

In another embodiment of the method, method (B), the variables m, n, L1, Z1, R, R1, R2, R3, R4, R5, R6, R7, R8, R9, and A1 in the compound of formula (XV) are as defined in the compound of formula (IB) but excluding provisos (1) and (2) of the first aspect; and the method further comprises converting the compound of formula (XV) to a compound of formula (IF) but excluding provisos (1) and (2) of the first aspect by one or more additional synthetic steps; and coupling the compound to an amino acid or peptide to provide the compound of formula (IF) of the invention (including proviso (1) and/or (2) of the first aspect). In this embodiment, the amino acid-comprising conjugation partner may consist of an amino acid or may comprise a peptide that corresponds to a portion of the peptide present in the compound of formula (IF) of the invention produced by the method.

In some embodiments, the amino acid comprising conjugation partner reacted with the epoxide consists of an amino acid, for example an Nα-amine protected and/or C-terminus protected cysteine. In other embodiments, the amino acid comprising conjugation partner comprises a peptide, for example a short peptide. In such embodiments, the amino acid comprising conjugation partner may comprise about 15 amino acid residues or less, for example 5, 4, or 3 amino acid residues.

The Nα-amino group of the amino acid comprising conjugation partner is preferably protected or otherwise substituted (i.e. is not in the form of a free amine —NH2 group) to prevent reaction during the conjugation reaction. The C-terminus of the amino acid comprising conjugation partner may also be protected.

X10 in the compound of formula (XVI) may be a protected hydroxyl, thiol, amine, or carbamate group (P10-O—, P11-S—, P12-NR—, or P12-NRC(O)O—, respectively) from which L1-Z1- and L2-Z2- may subsequently be formed. Where X10 is a protected group, the protecting group may be removed in the conjugation reaction to provide a compound of the formula (XV) wherein X11 is the corresponding deprotected group. For example, where X10 is a P10-O— group conjugation may provide the corresponding hydroxyl group as X11 in the compound of formula (XV).

The epoxide of formula (XVI) comprises a stereogenic centre at the carbon atom to which R3 is attached. Thus, a single stereoisomer of the epoxide or a stereoisomerically enriched mixture of the epoxide may used in the reaction to control the stereochemistry of the carbon atom to which R3 is attached in the compound of formula (XV) and subsequent products formed, including the compound of formula (IF). Various methods for providing enantiopure or enantioenriched mixtures of epoxides are known in the art. In various embodiments, providing the single stereoisomer or a stereoisomerically enriched mixture of the epoxide of formula (XVI) comprises resolving a racemic mixture of the epoxide. For example, resolving a racemic epoxide mixture by kinetic hydrolysis, as described by Jacobsen et al, Science, 1997, 277, 936-938.

The epoxide of formula (XVI) may be provided by reacting an alkene of the formula (XVII) with an oxidant under conditions effective to epoxidise the alkene. Numerous methods for epoxidising alkenes are known in the art. In certain embodiments, the epoxidation is carried out by reacting the alkene with a peroxide or an organic N-oxide as the oxidant. Examples of suitable peroxides include organic peroxides, for example m-chloro peroxybenzoic acid. Examples of N-oxides include, for example, pyridine N-oxide and the like. Other suitable oxidants will be apparent to those skilled in the art. The reaction may be carried out in a liquid reaction medium comprising a suitable solvent, for example dichloromethane. Alkenes of the formula (XVII) may be commercially available or prepared from commercially available precursors using standard synthetic chemistry techniques.

Those skilled in the art will appreciate that certain X10 groups may be susceptible to oxidation in the epoxidation reaction, for example when X10 comprises an amine group (which may form an N-oxide) or thioether group (which may form e.g. sulfoxides or sulfones). Such groups may be protected during the reaction to prevent oxidation or reduced back to the desired group at an appropriate point in the synthetic sequence after the epoxidation reaction has been carried out.

Alternatively, the epoxide of formula (XVI) may be prepared by treating a compound of formula (XVII-A), wherein LG is a suitable leaving group such as a halogen, with a base in a suitable solvent to displace the leaving group as shown in scheme A2.

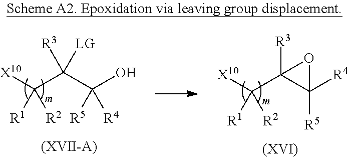

Compounds of the formula (XVII-A) may be commercially available or may be prepared from commercially available precursors. Advantageously, in some embodiments, the compound of formula (XVII-A) may be prepared from an enantiopure α-amino acid. The epoxidation reaction proceeds stereospecifically with inversion of stereochemistry at the carbon to which R3 is attached.

For example, as shown in scheme A2-1, the compound of formula (XVII-A1), which corresponds to a compound of formula (XVII-A) wherein m is 2 and R1 and R2, and R3, R4, and R5 are hydrogen, X10 is —OH, and LG is bromo, may be prepared from L-aspartic acid (see Volkmann, R. A. et al. J. Org. Chem., 1992, 57, 4352-4361).

L-Aspartic acid may be converted to be bromosuccinic acid (AA-1) by, for example, treatment with sodium nitrite and a strong acid such as sulfuric acid, to generate nitrous acid in situ, in the presence of sodium bromide at a temperature from −10 to 0° C. The reaction proceeds stereospecifically with overall retention of stereochemistry.

Reduction of bromosuccinic acid (AA-1) to bromodiol (XVII-A1) may be carried out using a suitable reductant, for example by treatment with borane or borane-dimethyl sulfide complex in THF at −78° C. allowing the reaction mixture to warm to room temperature. Epoxidation to provide the compound of formula (XVI-1a) may be carried out by reacting bromodiol (XVII-A1) with a base, for example cesium carbonate in dichloromethane at room temperature. As noted above, the reaction proceeds stereospecifcally with overall inversion of stereochemistry.

The opposite enantiomer of epoxide (XVI-1a) can be prepared from D-aspartic acid by the same procedure.

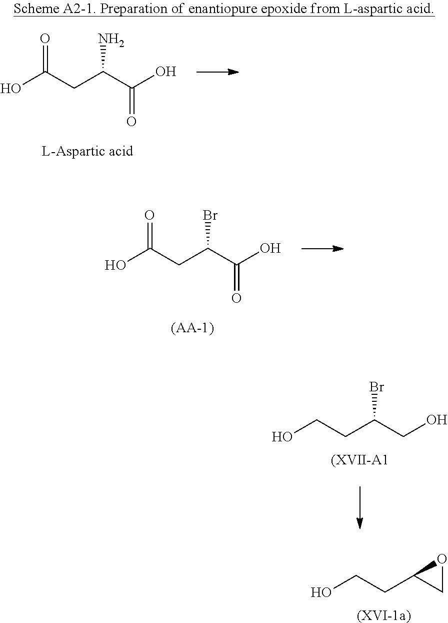

Referring again to Scheme A1, the compound of formula (XV) may be subsequently converted by one or more synthetic steps to compound of the formula (IF) as defined in either method (A) or (B). In the one or more steps, the hydroxyl group bound to the carbon to which R3 is attached is converted to an L2-Z2- group.

If X11 is not L1-Z1-, then the one or more steps also comprises converting X11 to L1-Z1-. The L1-Z1- and L2-Z2- groups may be introduced simultaneously or sequentially in any order.

In certain embodiments, the one or more steps comprises acylating the compound of formula (XV) so as to replace the hydrogen atom of the hydroxyl group bound to the carbon to which R3 is attached with L2-C(O)—.

In exemplary embodiments, X10 is P10-O— or OH; and X11 is P10-O— or OH.

In various embodiments, X11 is P10-O— or OH; and the one or more synthetic steps comprise acylating the compound of formula (XV) so as to replace P10 or the hydrogen atom of the hydroxyl group of X11 with L1-C(O)—; and/or the hydrogen atom of the hydroxyl group bound to the carbon to which R3 is attached with L2-C(O)—.

In certain embodiments, as shown below in Scheme A3 and described herein, the method comprises reacting an epoxide of formula (XVI-1) bearing a protected hydroxyl group with an amino acid comprising conjugation partner of the formula (III) to provide a compound of the formula (XV-1a).

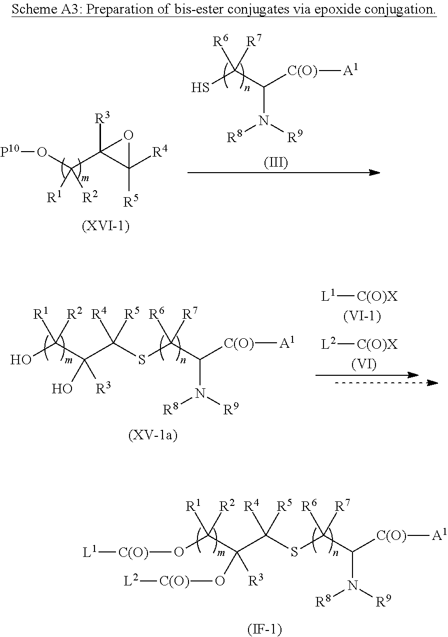

The conjugation reaction may be carried out under acidic conditions by reacting the epoxide and thiol in the presence of an acid, for example hydrochloric acid, sulfuric acid, or a mixture thereof. The reaction may be carried out in a liquid reaction medium comprising a suitable solvent, such as dichloromethane, at a temperature from about −10 to about 50° C., for example from 0 to 40° C.

The hydroxyl protecting group P10 is selected such that it is removable under the conditions effective for conjugation and is therefore removed during the conjugation reaction to provide the desired diol of formula (XV-1a). Suitable protecting groups will be apparent to those skilled in the art and may include, for example, acid labile silyl protecting groups.

Alternatively, the conjugation reaction may be carried using an epoxide of the formula (XVI) wherein X10 is a hydroxyl group, such as the epoxide of formula (XVI-1a).

The diol of the formula (XV-1a) may be converted to the compound of formula (IF-1) by reaction with the compounds of formula (VI-1) and (VI), wherein X is OH or a suitable leaving group (for example a halide, such as chloro or bromo), under conditions effective for esterification.

The conditions effective for esterification depend on the nature of the compound of formula (IV) and/or (VI-1). For example, where X is OH, the reaction may be carried out in the presence of a base, such as DMAP, and activating agent, such as N,N′-diisopropylcarbodiinnide (DIC) in a liquid medium comprising a suitable solvent, such as THF.

In various embodiments, the compound of formula (VI) and (VI-1) are identical. For example, the compound of formula (VI) and (VI-1) may each be palmitic acid. In such embodiments, conversion of the diol of formula (XV-1a) to the compound of formula (IF-1) may be accomplished in a single step.

In certain embodiments, different L1 and L2 groups may be introduced by reacting the diol with a stoichiometric amount of a compound of formula (VI-1) or (VI) to esterify the more reactive of the two alcohols, and then reacting the resultant ester with the other a compound of formula (VI) or (VI-1) to esterify the second alcohol of the diol.

In other embodiments, the method comprises reacting an epoxide of formula (XVI-1) and an amino acid comprising conjugation partner of the formula (III) to provide a compound of the formula (XV-1b) as shown in Scheme A4 below. In such embodiments, the hydroxyl protecting group P10 is stable and is not removed under the conjugation reaction conditions.

The protected alcohol of the formula (XV-1b) provides ready access to compounds of formula (IF-1) wherein L1 and L2 are different. Using the compound of formula (XV-1b) to access such compounds, rather than the diol of formula (XV-1a), may be more convenient in certain embodiments, for example where there is poor selectivity between the alcohols of the diol of formula (XV-1a).

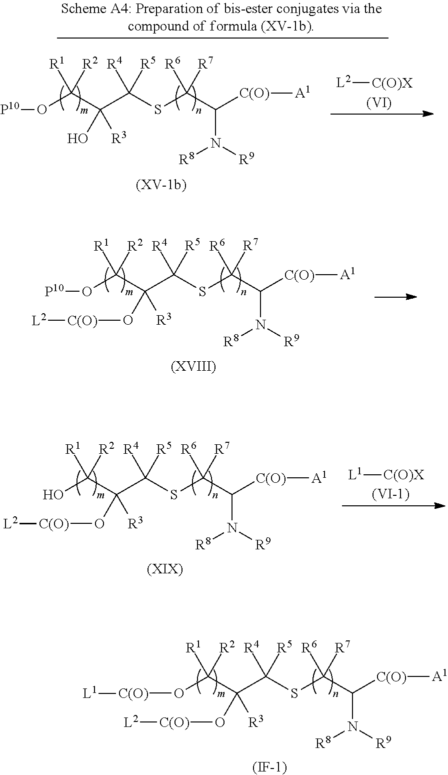

The β-sulfanylhydroxyl group of the compound of formula (XV-1b) may be acylated with a compound of formula (VI) under conditions effective for esterification to provide protected ester (XVIII), then the protecting group P10 removed to provide the alcohol of formula (XIX). The conditions for removal of the protecting group depend on the protecting group used. For example, dilute HF may be used to remove silyl protecting groups, such as TBDMS, TBDPS, and the like. The alcohol of formula (XIX) may then be acylated with a compound of formula (VI-1) under conditions effective for esterification to provide the desired compound of formula (IF-1).

Those skilled in the art will appreciate that hydroxyl groups, for example those in the compounds of formulae (XV-1a), (XV-1b), and (XIX), may be converted to various other functional groups, such as thiols and amines, to provide access compounds of formula (I) bearing L1-Z1- and L2-Z2- groups other than esters.

For example, the compound of formula (XV-1b) can be used to prepare thioester and amide analogues of the compound of formula (IF-1), as shown below in Scheme A5. To prepare amide analogue (IF-3), the hydroxyl group in the compound of formula (XV-1b) may first be converted to an azide and then reduced to the corresponding amine. The reaction may be carried out under modified Mitsunobu conditions (e.g. L. Rokhum et al, J. Chem. Sci, 2012, 124, 687-691) using PPh₃, I₂, imidazole, and NaN₃ to provide the azide, and then PPh₃ to reduce azide to the amine. Alternatively, the azide may be obtained by first converting the hydroxyl group to a suitable leaving group, for example a tosyl or mesyl group, and then treating with NaN₃.

Acylation of the amine with a compound of formula (VI) provides the amide of formula (XVIII-2). The acylation reaction may be carried out by reacting a carboxylic acid of the formula (VI) in the presence of a base, for example DMAP, and an activating agent, for example DIC, in a suitable solvent such as THF. Deprotection of the protecting group P10 and esterification of the resultant alcohol (XIX-2) provides the compound of the formula (IF-3).

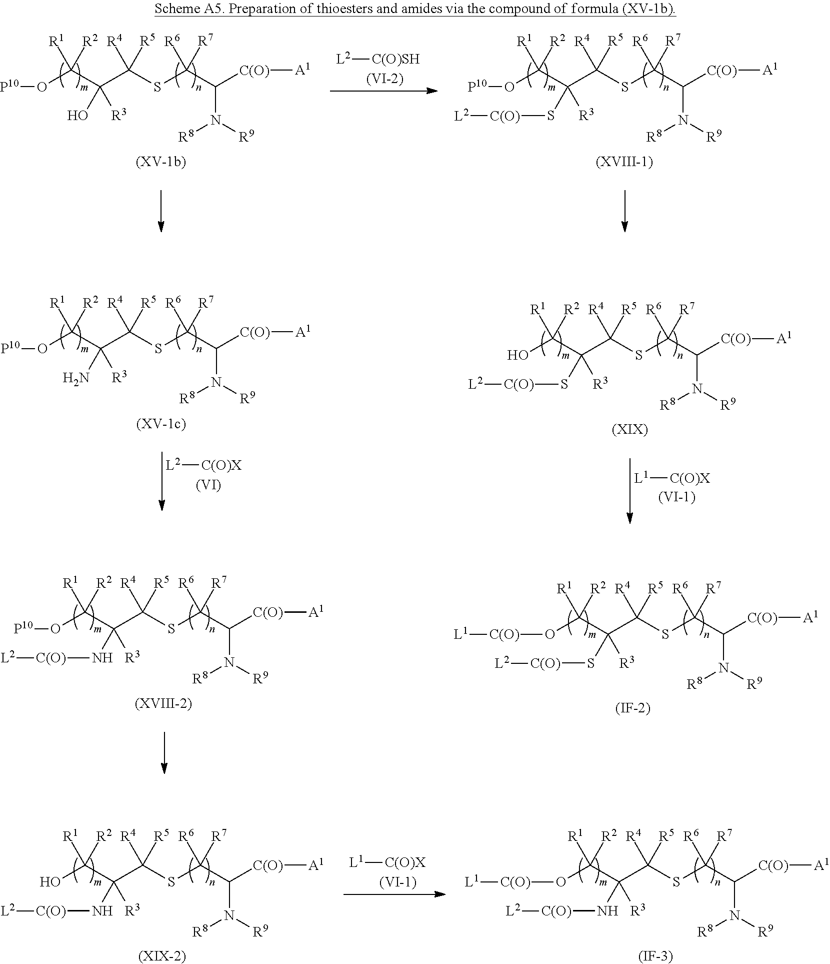

Thioester analogue (IF-2) may be prepared by first reacting the compound of formula (XV-1b) under Mitsunobu conditions (e.g. PPh₃, diethylazodicarboxylate (DEAD)) and trapping with the desired thioacid of formula (VI-2), for example thiopalmitic acid, to provide the compound of formula (XVIII-1)(see e.g. O. Schulze et al, Carbohydrate Res., 2004, 339, 1787-1802). Deprotection of the protecting group P10 and esterification of the resultant alcohol (XIX-1) provides the compound of the formula (IF-2).

Thioester and amide analgoues of bis-ester (IF-1) may also be prepared from the compound of formula (XIX), as shown in Scheme A6. The compound of formula (XIX) may be converted to the compound of formula (IF-4) by methods analogous to those described above for the conversion of the compound formula (XV-1b) to the compound of formula (XVIII-1).

Similarly, the compound of formula (XIX) may be converted to the compound of formula (IF-5) by methods analogous to those described above for the conversion of the compound of formula (XV-1b) to the compound of formula (XVIII-2).

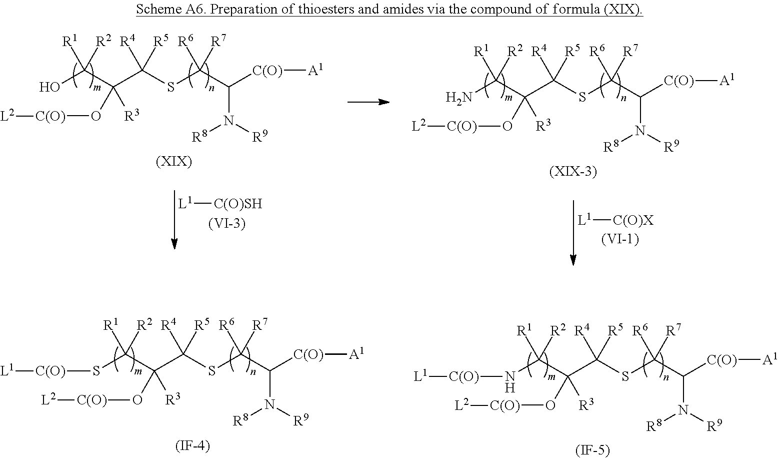

Further analogues of bis-ester (IF-1) may be prepared by replacing the compound of formula (XIX) in Scheme A6 with a compound of formula (XIX-1) or (XIX-2) and then following the synthetic sequences described.

Numerous other compounds of formula (IF) may be prepared by analogous methods, as will be appreciated by those skilled in the art.

Compounds of formula (VI), (VI-1), (VI-2), and (VI-3) may be commercially available or prepared from commercially available precursors using standard synthetic chemistry techniques.

Compounds of formula (I) may also be prepared by a method of the present invention comprising the conjugation of an amino acid comprising conjugation partner and an acetal, as shown in Scheme B1.

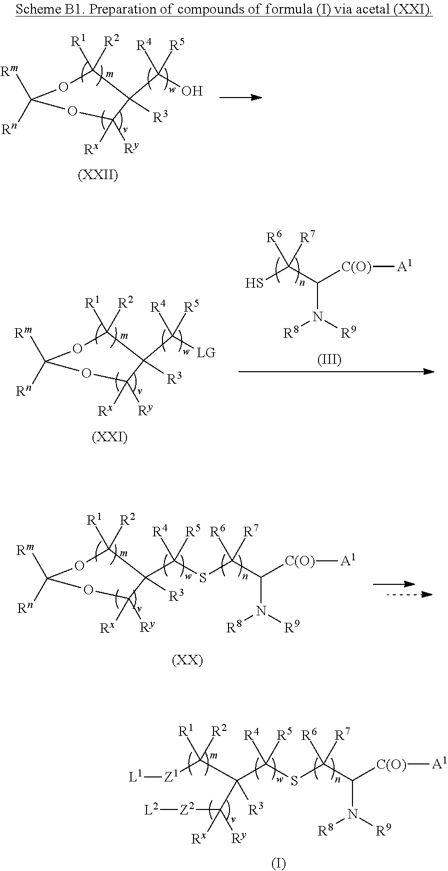

The method comprises reacting an amino acid comprising conjugation partner of the formula (III) and an acetal of the formula (XXI), wherein LG is a suitable leaving group, under conditions effective to provide a compound of the formula (XX).

In one embodiment of the method, method (A), the variables m, w, v, n, Rx, Ry, R1, R2, R3, R4, R5, R6, R7, R8, R9, and A1 in the compound of formula (XX) are as defined in the compound of formula (I) of the invention (including provisos (1) and/or (2) of the first aspect); and the method further comprises converting the compound of formula (XX) to the compound of formula (I) of the invention by one or more additional synthetic steps. In this embodiment, the amino acid-comprising conjugation partner may comprise a peptide that corresponds the peptide present in the compound of the formula (I) of the invention produced by the method.

In another embodiment of the method, method (B), the variables m, w, v, n, Rx, Ry, R1, R2, R3, R4, R5, R6, R7, R8, R9, and A1 in the compound of formula (XX) are as defined in the compound of formula (I) but excluding provisos (1) and (2) of the first aspect; and the method further comprises converting the compound of formula (XX) to a compound of formula (I) but excluding provisos (1) and (2) of the first aspect by one or more additional synthetic steps; and coupling the compound to an amino acid or peptide to provide the compound of formula (I) of the invention (including proviso (1) and/or (2) of the first aspect). In this embodiment, the amino acid-comprising conjugation partner may consist of an amino acid or may comprise a peptide that corresponds to a portion of the peptide present in the compound of formula (I) of the invention produced by the method.

In some embodiments, the amino acid comprising conjugation partner reacted with the acetal consists of an amino acid, for example an Nα-amine protected and/or C-terminus protected cysteine. In other embodiments, the amino acid comprising conjugation partner comprises a peptide, for example a short peptide. In such embodiments, the amino acid comprising conjugation partner may comprise about 15 amino acid residues or less, for example 5, 4, or 3 amino acid residues.

The Nα-amino group of the amino acid comprising conjugation partner is preferably protected or otherwise substituted (i.e. is not in the form of a free amine —NH2 group) to prevent reaction during the conjugation reaction. The C-terminus of the amino acid comprising conjugation partner may also be protected.

In the reaction, the thiol of the compound of formula (III) displaces the leaving group (LG) in the acetal of formula (XXI). Suitable leaving groups include but are not limited to halo (for example chloro, bromo, or iodo) or sulfonate (for example a tosylate or mesylate). Other suitable leaving groups will be apparent to those skilled in the art.

The size of the acetal ring in the compound of formula (XXI) may vary. The acetal ring may comprise from 5 to 7 ring atoms (i.e. may be a 5-7-membered cyclic acetal). In certain embodiments, the cyclic acetal is 6-membered. It will be appreciated that when the cyclic acetal is a 5-membered cyclic acetal, in order to provide a compound of the formula (I), w is at least 2 (such that the sum of m, v, and w is at least 3).

The conjugation reaction may be carried out in the presence of a base. For example, the reaction may be carried out in the presence of organic amine, in a suitable solvent, for example DMF, at a temperature of about 50° C. Suitable organic amines include but are not limited to triethylamine, N-methylmorpholine, collidine, and the like.

The compound of formula (XXI) may be provided in stereoisomerically pure form or a stereoisomerically enriched mixture by reacting stereoisomerically pure or a stereoisomerically enriched mixture of the compound of the compound of formula (XXII). Advantageously, stereoisomerically pure compounds of formula (XXII) are readily commercially available, such as (4R)- or (4S)-(2,2-dimethyl-1,3-dioxan-4-yl)-methanol.

Other compounds of formula (XXII) may be prepared by routine methods known in the art. As shown in Scheme B1-1, a compound of formula (XXII-B), wherein Pg is a suitable hydroxyl protecting group, may be reacted with a compound of the formula (XXII-C1) to provide the acetal of formula (XXII-D), which may then be converted to the compound of formula (XXII) by removal of the protecting group Pg. Alternatively, the compound of formula (XXII-B) may be reacted with an acyclic acetal of the formula (XXII-C2), wherein Ro and Rp are each independently C1-4alkyl. The acetylisation reaction may be carried out using an acid, such as camphorsulfonic acid, in a suitable solvent, such as dichloromethane.

The conditions for removal of the protecting group Pg, depend on the protecting group used. For example, a silyl ether protecting group, such as TBDMS, may be removed by treatment with a source of fluorine, such as tetrabutylammonium fluoride (TBAF) in suitable solvent, such as THF. See, for example C. R. Reddy et al, (Tetrahedron Letters, 2010, 51(44) 5840-5842); and Sauret-Cladiére et al (Tetrahedron Asymmetry, 1997, 8(3), 417-423).

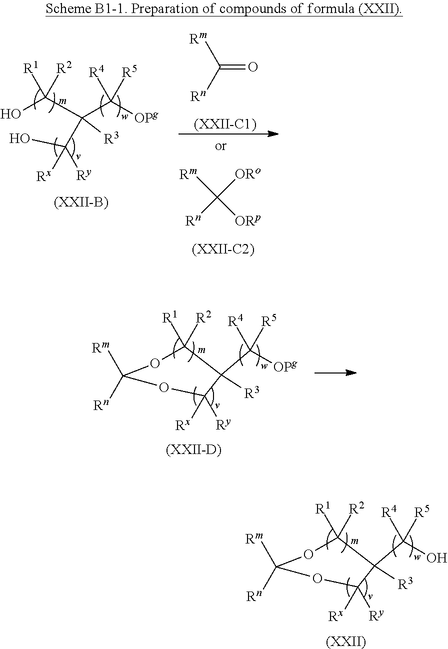

Referring again to Scheme B1, compounds of formula (XXI) may be prepared from compounds of formula (XXII) by reaction with a suitable precursor of the leaving group. For example, tosylate or mesylate leaving groups may be prepared by reaction with tosyl chloride or mesyl chloride in the presence of a base and a suitable solvent, and an iodo leaving group may be prepared by reaction with PPh₃ and I₂.

The compound of formula (XX) may subsequently be converted by one or more synthetic steps to a compound of the formula (I) as defined in either method (A) or (B), for example a compound of the formula (IA).

The one or more synthetic steps may comprise removing the acetal to provide a diol of the formula (XXIII-1). The hydroxyl group bound to the carbon to which R1 and R2 are attached in the compound of formula (XXIII-1) may be converted to L1-Z1-, and/or the hydroxyl group bound to the carbon to which Rx and Ry are attached may be converted to L2-Z2.

For example, as shown in Scheme B2, the acetal in the compound of formula (XX) may be removed to provide the diol of formula (XXIII-1) by treatment with an acid such as p-toluene sulfonic acid in a solvent such as dichloromethane. The diol of formula (XXIII-1) may be converted to the bis-ester compound of formula (IA) via one or more acylation steps in a manner analogous to that described for the conversion of the compound of formula (XV-1a) to the compound of formula (IF-1).

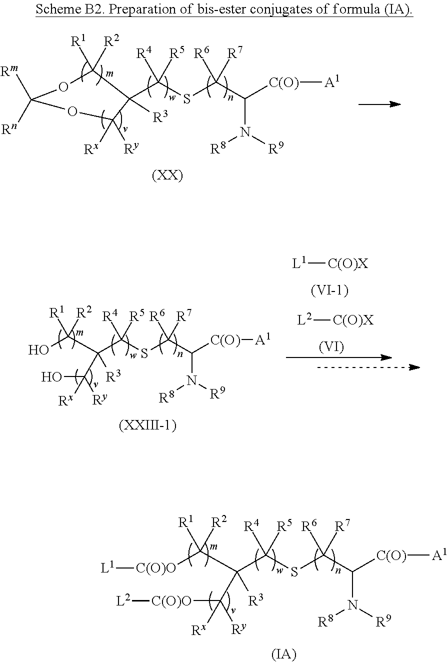

Alternatively, in various embodiments wherein Rm is optionally substituted aryl, for example phenyl or methoxy substituted phenyl, the one or more synthetic steps may comprise removing the acetal to provide a compound of the formula (XXIII-2) or (XXIII-3). The one or more steps may comprise converting the hydroxyl group bound to the carbon atom to which Rx and Ry are attached in the compound of formula (XXIII-2) to L2-Z2-, removing the RmRnCH- group to provide a hydroxyl group, and converting the hydroxyl group to L1-Z1; or converting the hydroxyl group bound to the carbon to which Rx and Ry are attached in the compound of formula (XXIII-2) to L1-Z1-, removing the RmRnCH- group to provide a hydroxyl group, and converting the hydroxyl group to L2-Z2-. Such methods advantageously allows allow the introduction of different L1-Z1 and L2-Z2- groups.

As illustrated in Scheme B3, the acetal in the compound of formula (XX) may be removed by, for example, treatment with a suitable reducing agent, for example diisobutylaluminium hydride (DIBAL). The resulting compound of formula compound of formula (XXIII-2) may then be acylated with the compound of formula (VI) to introduce the desired L2-C(O)O— group. Removal of the RmRnCH- group to provide the compound of formula (XXV-2) may be carried out by hydrogenolysis (e.g. for a benzyl or p-methoxybenzyl group) or any other suitable method having regard to the nature of RmRnCH- group. The compound of formula (XXV-2) may then be converted to the compound of formula (IA) by acylating with the compound of formula (IV-1). The acylation steps may be carried out as described herein with respect to the preparation of the compound of formula (IF-1).

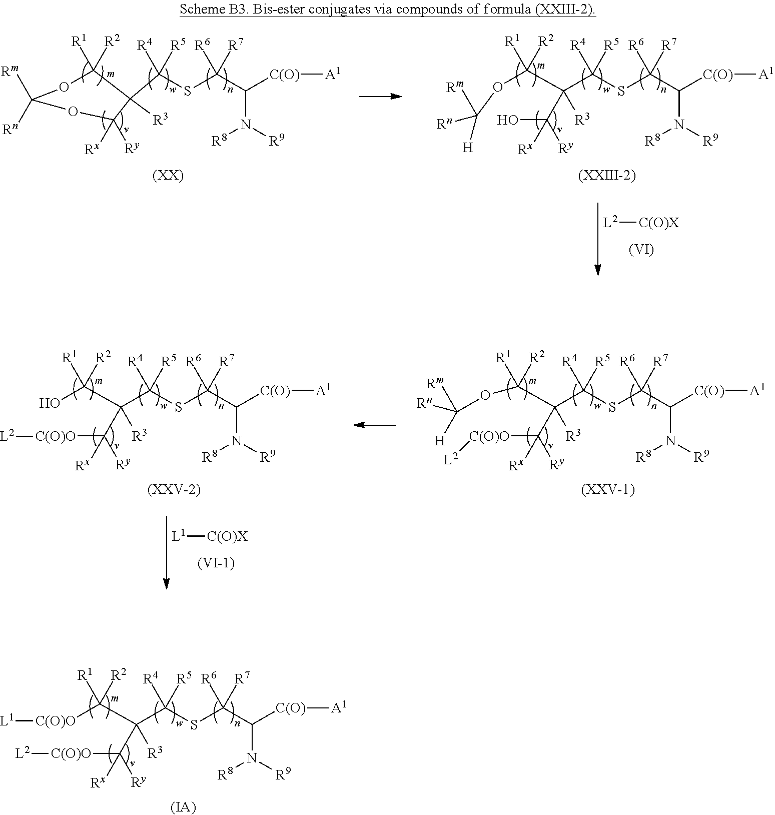

It will be apparent to those skilled in the art that compounds of formula (IA) may be prepared from compounds of formula (XXIII-3) by a replacing the compounds of formulae (XXIII-2), (VI) and (VI-1) in Scheme B3 with the compounds of formulae (XXIII-3), (VI-1), and (VI), respectively, and then following the synthetic sequence described.

Hydroxyl groups produced on removal of the acetal or RmRnCH- group, such as those in the compounds formulae (XXIII-1), (XXIII-2), (XXIII-3), and (XXV-2), may be converted to various other functional groups, such as thiols and amines, to provide access compounds of formula (I) bearing other Z1 and Z2 groups.

It will be appreciated that amide and thioester analogues of the bis-ester compound of formula (IA) may be prepared by methods analogous to those described above with respect to the amide and thioester analogues of the bis-ester compound of formula (IF-1).

The present invention also provides a method for preparing compounds of formula (I) of the invention via a thiol-ene reaction. The method comprises reacting a first lipid-containing conjugation partner comprising a carbon-carbon double bond, a second lipid-containing conjugation partner a carbon-carbon double bond, and an amino acid-comprising conjugation partner comprising a thiol, under conditions effective to conjugate the first and second lipid-containing conjugation partners to the amino acid-comprising conjugation partner. Each lipid containing conjugation partner comprises and therefore in the reaction provides to the compound of formula (I) a lipid moiety one comprising L1, the other comprising L2.

The thiol-ene reaction involves the addition of a thiol across a non-aromatic carbon-carbon double bond (i.e. hydrothiolation of the carbon-carbon double bond). The reaction proceeds via a free radical mechanism. There are three distinct phases in the reaction: initiation, coupling, and termination.

Typically, radical generation gives rise to an electrophilic thiyl radical which propagates across the ene group of an alkene, forming a carbon-centred radical and chain transfer from an additional thiol molecule quenches the radical on carbon to give the final product.

Without wishing to be bound by theory, the inventors believe that in the method of the present invention, the thiol is conjugated to a carbon atom of the carbon-carbon double bond of the first lipid containing conjugation partner to form a carbon-centred radical, and that this carbon-centred radical, instead of being quenched, is then conjugated with a carbon atom of the carbon-carbon double bond of the second lipid-containing conjugation partner.

The method thus provides amino acid- and peptide conjugates in which the sulfur atom from the thiol is conjugated to a carbon atom from the carbon-carbon double bond of the first lipid-containing conjugation partner, and a carbon atom from the carbon-carbon double bond of the first lipid-containing conjugation partner is conjugated to a carbon atom from the carbon-carbon double bond of the second lipid-containing conjugation partner.

In one embodiment of the method, method (A), conjugation of the first and second lipid-containing conjugation partners to the amino acid-comprising conjugation partner provides the peptide conjugate of the formula (I) of the invention. In this embodiment, the amino acid-comprising conjugation partner may comprise a peptide that corresponds the peptide present in the peptide conjugate of the formula (I) of the invention produced by the method.

In another embodiment of the method, method (B), conjugation of the first and second lipid-containing conjugation partners to the amino acid-comprising conjugation partner provides an amino acid- or peptide-conjugate (of the formula (I) but excluding provisos (1) and (2) of the first aspect); and the method further comprises coupling the amino acid- or peptide-conjugate to an amino acid or peptide to provide the peptide conjugate of formula (I) of the invention (including proviso (1) and/or (2) of the first aspect). In this embodiment, the amino acid-comprising conjugation partner may consist of an amino acid or may comprise a peptide that corresponds to a portion of the peptide present in the compound of formula (I) of the invention produced by the method.

The first and second lipid containing conjugation partners may be the same or different. Those skilled in the art will appreciate that reacting different lipid containing conjugation partners at the same time may provide a mixture of (potentially up to four different) conjugates. Accordingly, in certain exemplary embodiments, the first and second lipid containing conjugation partners are the same.

The thiolene reaction may be regioselective with respect to which carbon atom of the carbon-carbon double bond of the first lipid-containing conjugation partner is conjugated to the thiol and also with respect to which carbon atom of the carbon-carbon double bond of the second lipid-containing conjugation partner is conjugated to which carbon atom of the carbon-carbon double bond from the first lipid-containing conjugation partner. Those skilled in the art will appreciate that various regioisomers may be formed in the reaction.

In certain embodiments, the method comprises reacting a first lipid containing conjugation partner of the formula (IIA) and a second lipid containing conjugation partner of the formula (IIB) with a thiol containing amino acid comprising conjugation partner (III) under conditions effective to provide a compound of the formula (IB) (Scheme C1). When the method is (A), Ra, Rb, Rc, L1, L2, Z1, Z2, R1, R2, Rx, Ry, R3, R4, R5, R6, R7, R8, R9, A1, k, v, and n are as defined in the compound of formula (IB) of the present invention (including provisos (1) and/or (2) of the first aspect); and when the method is (B), Ra, Rb, Rc, L1, L2, Z1, Z2, R1, R2, Rx, Ry, R3, R4, R5, R6, R7, R8, R9, A1, k, v, and n are as defined in the compound of formula (IB) but excluding provisos (1) and (2) of the first aspect.

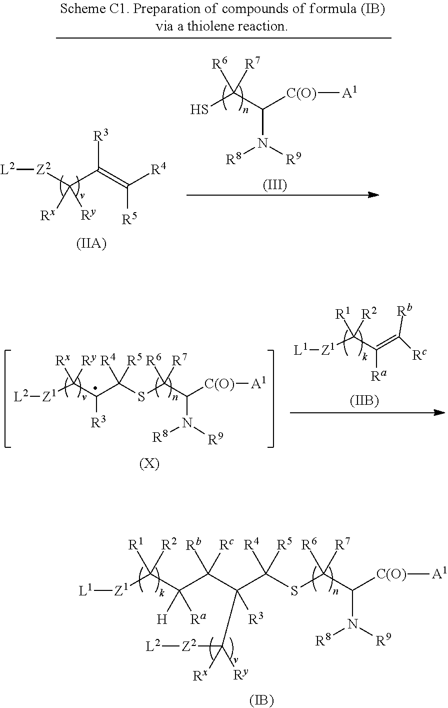

The conditions effective for formation of the compound of formula (IB) may vary. In various embodiments, the conditions effective for formation of the compound of formula (IB) may comprise carrying out the reaction with a stoichiometric excess of lipid containing conjugation partner to thiol, such as a stoichiometric ratio of the lipid containing conjugation partners (IIA) and (IIB) (combined) to amino acid-comprising conjugation partner of at least 7:1, for example 8:1, 9:1, 10:1, 20:1, 30:1, 40:1, 50:1, 60:1, or 70:1.

The degree of conversion of the amino acid-comprising conjugation partner to the product compound of formula (IB) may vary. Preferably, at least 5, 10, 15, 20, 25, 30, 35, 40, 45, 50, 60, or 70% of the amino acid-comprising conjugation partner is converted to the compound of formula (IB). Conversion may be determined by HPLC.

As noted above, without wishing to be bound by theory, the inventors believe that under such conditions reaction of the alkene of formula (IA) with the thiol of formula (III) results in the formation of a carbon-centred radical of the formula (X), which is trapped with the second alkene of the formula (IIB), rather than quenched by abstraction of a proton from the thiol of another molecule of the formula (III), to provide the desired amino acid- or peptide conjugate.

The reaction may result in the production of a mixture of stereoisomers as it may not be possible to control or influence the stereochemistry of bond formation between the carbon atom to which R3 is bound and the carbon atom to which Rb and Rc are bound owing to the radical intermediate generated in the course of the reaction. The reaction typically produces a mixture of epimers with respect to the carbon atom to which R3 is bound.

In certain embodiments, the Z1 and Z2 in the lipid containing-conjugation partners are each —C(O)O—, and the compound of formula (I) formed in the thiolene method is a compound of formula (IC) as defined herein.

In exemplary embodiments, the thiolene method of the present invention comprises reacting an amino acid-comprising conjugation partner comprising a structure of the formula (III) with lipid containing-conjugation partners of the formula (IIA) and (IIB) that are vinyl esters to provide a compound of the formula (ID). The reaction may be carried out, for example as described herein, by irradiating a reaction mixture comprising the amino acid comprising conjugation partner; lipid containing-conjugation partners; a photochemical initiator, such as DMPA. One or more additives may be included that reduce the formation of by products, such as a sterically hindered thiol (for example tert-butylmercaptan), an acid (for example TFA), or an organosilane (for example triisopropylsilane), or a combination of any two or more thereof. The reaction may be carried out in a suitable solvent, such as NMP, at ambient temperature for a suitable period of time, such as 30 minutes.

The reaction is typically initiated by the generation of one or more free radicals in the reaction mixture. One or more free radicals may be generated in the method by any method known in the art. The free radicals may be generated thermally and/or photochemically. One or more free radical initiators may be used to initiate the generation of free radicals. Suitable free radical initiators include thermal initiators and photoinitiators.

Free radicals are generated from thermal initiators by heating. The rate of degradation of the thermal initiator and resulting free radical formation depends on the initiator and the temperature at which the initiator is heated. Higher temperatures generally result in faster decomposition. A person skilled in the art will be able to select an appropriate temperature for heating the initiator without undue experimentation.

Numerous thermal initiators are commercially available. Examples of thermal initiators include but are not limited to tert-amyl peroxybenzoate, 1,1′-azobis(cyclohexanecarbonitrile), 2,2′-azobisisobutyronitrile (AIBN), benzoyl peroxide, tert-butyl hydroperoxide, tert-butyl peracetate, tert-butyl peroxide, tert-butyl peroxybenzoate, tert-butylperoxy isopropyl carbonate, lauroyl peroxide, peracetic acid, and potassium persulfate.

Free radicals may be generated from photoinitiators by irradiation with light. The frequency of light necessary to induce degradation of the photoinitiators and free radical formation depends on the initiator. Many photoinitiators can be initiated with ultraviolet light.

Light of a specific wavelength or wavelength range may be used to selectively irradiate the initiator, where the lipid-containing conjugation partners or amino acid-comprising conjugation partner, for example a peptide-containing conjugation partner, comprises photosensitive groups. In certain embodiments, a frequency of about 365 nm is used. Light of this frequency is generally compatible with the side chains of naturally occurring amino acids.

A wide range of photoinitiators are commercially available. Examples of photoinitiators include but are not limited to acetophenone, anisoin, anthraquinone, anthraquinone-2-sulfonic acid, benzil, benzoin, benzoin ethyl ether, benzoin isobutyl ether, benzoin methyl ether, benzophenone, 3,3′,4,4′-benzophenonetetracarboxylic dianhydride, 4-benzoylbiphenyl, 2-benzyl-2-(dimethylamino)-4′-morpholinobutyrophenone, 4′-bis(diethylamino)benzophenone, 4,4′-bis(dimethylamino)benzophenone, camphorquinone, 2-chlorothioxanthen-9-one, dibenzosuberenone, 2,2-diethoxyacetophenone, 4,4′-dihydroxybenzophenone, 2,2-dimethoxy-2-phenylacetophenone (DMPA), 4-(dimethylamino)benzophenone, 4,4′-dimethylbenzil, 2,5-dimethylbenzophenone, 3,4-dimethylbenzophenone, 4′-ethoxyacetophenone, 2-ethylanthraquinone, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, and thioxanthen-9-one.

A person skilled in the art will be able to select appropriate free radical initiators for use in the method having regard to, for example, the nature of the lipid-containing conjugation partners, amino acid-comprising conjugation partner, for example a peptide-containing conjugation partner, and any other components present in the reaction mixture. In some embodiments, the initiator is present in the reaction in a stoichiometric ratio relative to the starting material comprising the thiol of from about 20:1 to about 0.05:1, from about 10:1 to about 0.05:1, from about 5:1 to about 0.05:1, from about 3:1 to about 0.5:1.

The lipid-containing conjugation partners and amino acid-comprising conjugation partner, for example a peptide-containing conjugation partner, may be prepared using known synthetic chemistry techniques (for example, the methods generally described in Louis F Fieser and Mary F, Reagents for Organic Synthesis v. 1-19, Wiley, New York (1967-1999 ed.) or Beilsteins Handbuch der organischen Chemie, 4, Aufl. Ed. Springer-Verlag Berlin, including supplements (also available via the Beilstein online database)) or, in some embodiments, may be commercially available.

For example, lipid-containing conjugation partner compounds of the formula (IIA-1) may be prepared by reacting a compound of the formula (VI) wherein X is OH or a suitable leaving group with a compound of the formula (VII) wherein Y is H, a metal or metalloid, or acyl (for example, alkylcarbonyl) under conditions effective for esterification (or transesterification where Y is an acyl group) (Scheme C2).

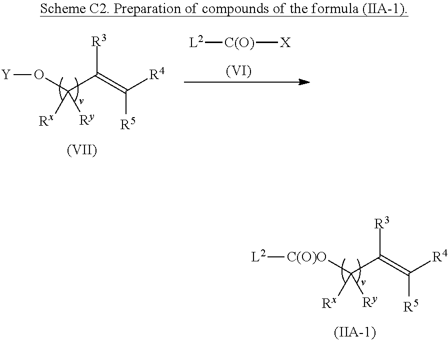

Methods for esterification (or transesterification) are well known in the art. For example, when X is chloro and Y is H, the reaction may be carried out in the presence of a base, such as pyridine or triethylamine, in a suitable solvent. The acid chloride may be converted in situ to a more reactive species (e.g. to the corresponding iodide, using sodium iodide). The temperature at which the reaction is carried out depends on the reactivity of the acid species and the solvent used.

For example, vinyl esters of the formula (IIA-1) may be produced by transesterification with vinyl acetate (itself produced industrially by the reaction of acetic acid and acetylene or acetic acid and ethylene over a suitable catalyst) using an acid or metal catalyst. See, for example, EP0376075A2 and S. K. Karmee, J. Oil Palm Res., 2012, 1518-1523.

Vinyl esters of the formula (IIA-1) may also be prepared by the addition a carboxylic acid to a terminal acetylene in the presence of a catalyst (usually a palladium or ruthenium complex). See, for example, V. Cadierno, J. Francos, J. Gimeno Organometallics, 2011, 30, 852-862; S. Wei, J. Pedroni, A. Meissner, A. Lumbroso, H.-J. Drexler, D. Heller, B. Breit, Chem. Eur. J., 2013, 19, 12067-12076. Non-terminal acetylenes may also be reacted. See, for example, N. Tsukada, A. Takahashi, Y. Inoue, Tetrahedron Lett., 2011, 52, 248-250 and M. Rotem, Y. Shvo, J. Organometallic Chem. 1993, 448, 159-204.

Further examples of methods for preparing vinyl esters of formula (IIA-I) include: reaction of divinylmercury with aromatic and aliphatic acids [see, for example, D. J. Foster, E. Tobler, J. Am. Chem. Soc. 1961, 83, 851]; Cu(II)-catalyzed esterification of arene carboxylic acids with trimethoxy(vinyl)silane in the presence of AgF [see, for example, F. Luo, C. Pan, P. Qian, J. Cheng, Synthesis 2010, 2005]; vinyl transfer reactions from vinyl acetate to primary and secondary alcohols, and also to carboxylic acids with a catalyst system consisting of 2 mol-% of [AuCl(PPh₃)] and 2 mol-% of AgOAc [see, for example, A. Nakamura, M. Tokunaga, Tetrahedron Lett. 2008, 49, 3729]; and Ir complex ([Ir(cod)Cl]₂/P(OMe)₃)-catalyzed transvinylation [see, for example, H. Nakagawa, Y. Okimoto, S. Sakaguchi, Y. Ishii, Tetrahedron Lett. 2003, 44, 103].

Other suitable methods for preparing compounds of formula (II-A) will be apparent to those skilled in the art.

Lipid containing conjugation partner compounds of the formula (IIB-1) may be prepared in an analogous fashion, where the compounds of formula (IIA-1) and (IIB-1) are different.

Numerous compounds of formula (VI) are commercially available. Others may be prepared using standard synthetic chemistry techniques from commercially available precursors. For example, compounds of formula (VI) wherein X is chloro may be prepared treating the corresponding carboxylic acid with thionyl chloride in a suitable solvent or mixture of solvents.

Similarly, compounds of formula (VII) are also commercially available or may be prepared from commercially available precursors using standard synthetic chemistry techniques.

The order in which the lipid-containing conjugation partners and amino acid-comprising conjugation partner, for example a peptide-containing conjugation partner, and any other components present in the reaction mixture are introduced into the reaction vessel may vary. The reaction may be carried out as a one-pot procedure.

The ratio of the lipid-containing conjugation partners to amino acid-comprising conjugation partner, for example a peptide-containing conjugation partner, in the reaction may vary. In some embodiments, the mole ratio of the first lipid-containing conjugation partner and second lipid-containing conjugation partner combined (i.e. in total) to the amino acid-comprising conjugation partner is at least 7:1, for example 8:1, 9:1, 10:1, 20:1, 30:1, 40:1, 50:1, 60:1, or 70:1.

The reaction may be carried out at any suitable temperature. In some embodiments, the reaction is carried out at a temperature from about −25° C. to about 200° C., from about −10° C. to about 150° C., from about 0° C. to about 125° C., from about ambient temperature to about 100° C. In some embodiments, the reaction is carried out at a temperature of less than about 200° C., less than about 175° C., less than about 150° C., less than about 125° C., or less than about 100° C.

In some embodiments, the reaction is carried out at a temperature above ambient temperature. In one embodiment, the reaction is carried out at a temperature from 40 to 200° C., from 50 to 150° C., from 60 to 100° C., from 65 to 90° C., or from 70 to 80° C. In some embodiments, the reaction is carried out at a temperature greater than 40° C., greater than 50° C., greater than 75° C., greater than 100° C., or greater than 150° C.

The temperature at which the reaction is carried out may depend on how free radicals are generated in the reaction. The temperature used may be selected to control the rate of the reaction. The temperature may be adjusted during the course of the reaction to control the rate of the reaction.

If free radicals are generated thermally (e.g. using a thermal initiator), the reaction will generally be carried out at a temperature above ambient temperature. The temperature will depend on the reactivity of the species from which free radicals are generated.

If free radicals are generated photochemically the reaction may be carried out, advantageously, at ambient temperature. In certain embodiments, it may be desirable to cool the reaction mixture to slow the rate of reaction or conversely heat the reaction mixture to increase the rate of reaction.

A person skilled in the art will be able to select appropriate temperatures for carrying out the method having regard to the reactivity of the starting materials and other reactants present.

The temperature at which the reaction is carried out may be controlled by heating or cooling the reaction mixture by suitable method known in the art. Heat may be applied to the reaction mixture, for example, using a heat exchanger within the reaction vessel, a heating jacket surrounding the reaction vessel, or by immersing the reaction vessel in a heated liquid (e.g. an oil or sand bath). In certain exemplary embodiments, the reaction mixture is heated by microwave irradiation.

The progress of the reaction may be monitored by any suitable means, for example, by thin layer chromatography (TLC) or high performance liquid chromatorgraphy (HPLC). The reaction may be allowed to proceed to substantial completion, as monitored by the consumption of at least one of the starting materials. In some embodiments, the reaction is allowed to proceed for a period of time from 1 minute to 7 days, 5 minutes to 72 hours, 10 minutes to 48 hours, 10 minutes to 24 hours. In other embodiments, the reaction is allowed to proceed for a period of time less than 72 h, less than 48 h, less than 24 h, less than 12 h, less than 6 h, less than 4 h, less than 2 h, or less than 1 h.

In some embodiments, the reaction is carried out until at least about 50%, at least about 60%, at least about 70%, at least about 75%, at least about 80%, at least about 85%, at least about 90%, at least about 95%, at least about 97%, at least about 99% of the amino acid-comprising conjugation partner has been consumed. The consumption of starting materials may be monitored by any suitable method, for example, HPLC.

The reaction mixture may be mixed by any suitable method known in the art, for example, using a magnetic or mechanical stirrer. The method used may depend on the scale on which the reaction is carried out.

The reaction is generally carried out in a liquid reaction medium. The liquid reaction medium may comprise a solvent. Examples of suitable solvents include N-methylpyrrolidone (NMP), dimethylformamide, dichloromethane, 1,2-dichloroethane, chloroform, carbon tetrachloride, water, methanol, ethanol, dimethylsulfoxide, trifluoroacetic acid, acetic acid, acetonitrile, and mixtures thereof.

The solvent may be selected based on the solubility of the starting materials and other reactants present, for example the free radical initiator. In some embodiments, the lipid-containing conjugation partners are hydrophobic. The hydrophobicity or hydrophilicity of an amino acid-comprising conjugation partner may vary depending on, for example, the amino acid sequence of the peptide of a peptide-containing conjugation partner. The presence of a solubilising group in the peptide-containing conjugation partner may increase solubility in polar solvents, such as water. A person skilled in the art will be able to select an appropriate solvent without undue experimentation.

The reaction may be carried out under substantially oxygen-free conditions. Oxygen may quench free radicals formed in the reaction. The reaction mixture may be degassed with an inert gas (e.g. nitrogen or argon) that is substantially oxygen-free to remove any dissolved oxygen before free radicals are generated. Alternatively, individual components of the reaction mixture may be degassed with inert gas that is substantially oxygen-free prior to being combined in the reaction vessel. The reaction may be carried out under an atmosphere of inert gas that is substantially oxygen-free.

The method of the present invention may be carried out at ambient pressure.

An additive that inhibits the formation of undesirable by-products and/or that improves the yield of or conversion to the desired product may be included in the reaction mixture in the thiolene method of the present invention. The one or more additive may be an extraneous thiol, an acid, an organosilane, or a combination of any two or more thereof.

The inventors have found that in some embodiments the inclusion of an extraneous or exogenous thiol as an additive in the reaction mixture reduces the formation of undesirable by products. The extraneous thiol may, in some embodiments, increase the efficiency or conversion of the desired thiolene reaction. Examples of suitable extraneous thiols include but are not limited to reduced glutathione, DODT, DTT, protein, sterically hindered thiols, and the like.

In some embodiments, the extraneous thiol is DTT.

In other embodiments, the extraneous thiol is a sterically hindered thiol. Non-limiting examples of a suitable sterically hindered extraneous thiol include tert-butyl mercaptan and 1-methylpropyl mercaptan.

Without wishing to be bound by theory, the inventors believe that in certain embodiments an extraneous thiol such as tert-butylmercaptan can provide a proton to quench the radical intermediate formed on propagation of the radical of formula (X) with the alkene of formula (IIB) to provide the desired compound of formula (IB) and the resulting thiyl radical can propagate the reaction by generating another mole of thiyl radical from the amino acid comprising conjugation partner of formula (III).

It will be apparent that extraneous thiols may in certain embodiments also be capable of prematurely quenching the reaction by providing a proton radical of formula (X). In such embodiments, the extraneous thiol and the amount in which it is used may be selected such that the yield of or conversion to (as determined by HPLC) the compound of formula (IB) is optimised.

In various embodiments, the extraneous thiol is present in the reaction in a stoichiometric ratio relative to the amino acid comprising conjugation partner of from about 200:1 to about 0.05:1, 100:1 to 0.05:1, 80:1 to 0.05:1, 60:1 to 0.05:1, 40:1 to 0.05:1, 20:1 to about 0.05:1, 10:1 to about 0.5:1, 5:1 to about 1:1, or 3:1 to about 1:1. In certain embodiments, a sterically hindered thiol such as t-BuSH is present in the reaction in a stoichiometric ratio relative to the amino acid comprising conjugation partner of from about 100:1 to 0.05:1, for example about 80:1, about 40:1, or about 3:1.

The inclusion of an acid in some embodiments may also reduce the formation of undesirable by-products. The acid may be a strong inorganic acid, for example HCl, or organic acid, for example TFA. In certain embodiments, the additive is TFA. Without wishing to be bound by theory, the inventors believe that decreasing the pH of the reaction mixture may result in the protonation of electron rich side chains of residues such as lysine, etc. which could otherwise participate in single electron transfers and form radical species in the reaction. In various embodiments, the reaction mixture comprises from about 0.01 to 25, 0.01 to 15, 0.01 to 10, or 1 to 10% v/v acid additive. In certain embodiments, the reaction mixture comprises from 1-10% v/v TFA, for example 5% v/v TFA.

The inventors have found that in some embodiments including both tert-butyl mercaptan and TFA as additives in the reaction mixture can reduce the the formation of undesirable by products and increase the conversion of starting material to the desired product. Accordingly, in certain exemplary embodiments, the reaction mixture comprises a combination of an acid and an exogenous thiol, such as a combination of a strong organic acid and a sterically hindered thiol, for example a combination of TFA and tert-butyl mercaptan.

An organosilane may also be included as an additive in the thiolene reaction. Organosilanes are radical-based reducing agents, the activity of which can be modulated by varying the substituents on the silicon atom. In various embodiments, the organosilane is a compound of the formula (R^{q})₃SiH, wherein Rq at each instance is independently hydrogen or an organic group, for example alkyl or aryl, provided that at least one Rq is not hydrogen. Examples of organosilanes include but are not limited to triethylsilane (TES), triphenylsilane, diphenylsilane, triisopropylsilane (TIPS), and the like. In various embodiments, the organosilane is a trialkylsilane, for example TIPS or TES.

Without wishing to be bound by theory, the inventors believe that, as with an extraneous thiol, in certain embodiments an organosilane such as TIPS can act as a hydrogen donor to provide the desired compound of formula (IB) and promote propagation of the reaction.

In various embodiments, the organosilane is present in the reaction in a stoichiometric ratio relative to the amino acid comprising conjugation partner of from about 200:1 to about 0.05:1, 100:1 to 0.05:1, 80:1 to 0.05:1, 60:1 to 0.05:1, 40:1 to 0.05:1, 20:1 to 0.05:1, 10:1 to 0.5:1, 5:1 to about 1:1, or 3:1 to about 1:1. In certain embodiments, a trialkylsilane such as TIPS is present in the reaction in a stoichiometric ratio relative to the amino acid comprising conjugation partner of from about 100:1 to 0.05:1, for example about 80:1 or about 40:1.

The organosilane may be used as an additive in combination with an extraneous thiol. Alternatively, the organosilane may be used instead of an extraneous thiol. An acid, such as TFA, may also be present. The inventors have found that in certain embodiments using TIPS in the reaction together with TFA but without any extraneous thiol can provide higher conversion to the desired compound of formula (IB) than when a combination of TIPS, t-BuSH, and TFA are used.

The additive is generally used in an amount sufficient to minimise the formation of undesirable by products without adversely affecting the reaction or any, optional, subsequent steps in the method.

The products formed in the reaction and conversion to the desired product may be determined by, for example, HPLC.

The concentration of the lipid-containing conjugation partners and amino acid-comprising conjugation partner, for example a peptide-containing conjugation partner, respectively, in the reaction mixture may also affect the reaction. Those skilled in the art will be able to vary the concentration of the lipid-containing conjugation partners and peptide-containing conjugation partner in the reaction mixture to e.g. optimise yield and purity without undue experimentation.

In some embodiments, the starting material comprising the thiol is present in a concentration from about 0.05 mM to about 1 M, from about 0.5 mM to about 1 M, from about 1 mM to about 1 M. In some embodiments, the concentration is at least about 0.05 mM, 0.5 mM, or 1 mM.

In some embodiments, the concentration of the starting materials comprising the alkenes is at least about 0.05 mM, 0.5 mM, or 1 mM.

In some embodiments, the amino acid conjugate or peptide conjugate is separated from the reaction medium after the reaction and optionally purified. The conjugate may be separated from the reaction medium using any suitable method known in the art, for example, by precipitation.

In some embodiments, the amino acid or peptide conjugate is purified after separating it from the reaction medium. For example, the conjugate may be purified by HPLC using one or more suitable solvents.

The present invention also provides a method of making a peptide conjugate, the method comprising
providing an amino acid- or peptide conjugate of the formula (I) but excluding provisos (1) and (2) of the first aspect or a salt or solvate thereof, and
coupling the amino acid of the amino acid conjugate or an amino acid of the peptide conjugate to an amino acid or an amino acid of a peptide to provide a peptide conjugate of the formula (I) of the invention or a salt or solvate thereof.

The amino acid- or peptide conjugate of the formula (I) but excluding provisos (1) and (2) of the first aspect or a salt or solvate thereof may be provided by the methods described herein.

The peptide conjugate produced by and/or the peptide-containing conjugation partner and/or the peptides coupled in the methods of the present invention may comprise a synthetic peptide. Synthetic peptides may be prepared using solid phase peptide synthesis (SPPS).

The basic principle for solid phase peptide synthesis (SPPS) is a stepwise addition of amino acids to a growing polypeptide chain anchored via a linker molecule to a solid phase support, typically a resin particle, which allows for cleavage and purification once the polypeptide chain is complete. Briefly, a solid phase resin support and a starting amino acid are attached to one another via a linker molecule. Such resin-linker-acid matrices are commercially available.

The amino acid to be coupled to the resin is protected at its Na-terminus by a chemical protecting group.

The amino acid may also have a side-chain protecting group. Such protecting groups prevent undesired or deleterious reactions from taking place during the process of forming the new peptide bond between the carboxyl group of the amino acid to be coupled and the unprotected Nα-amino group of the peptide chain attached to the resin.

The amino acid to be coupled is reacted with the unprotected Nα-amino group of the N-terminal amino acid of the peptide chain, increasing the chain length of the peptide chain by one amino acid. The carboxyl group of the amino acid to be coupled may be activated with a suitable chemical activating agent to promote reaction with the Nα-amino group of the peptide chain. The Nα-protecting group of N-terminal amino acid of the peptide chain is then removed in preparation for coupling with the next amino acid residue. This technique consists of many repetitive steps making automation attractive whenever possible. Those skilled in the art will appreciate that peptides may be coupled to the Na-amino group of the solid phase bound amino acid or peptide instead of an individual amino acid, for example where a convergent peptide synthesis is desired.

When the desired sequence of amino acids is achieved, the peptide is cleaved from the solid phase support at the linker molecule.

SPPS may be carried out using a continuous flow method or a batch flow method. Continuous flow permits real-time monitoring of reaction progress via a spectrophotometer, but has two distinct disadvantages—the reagents in contact with the peptide on the resin are diluted, and scale is more limited due to physical size constraints of the solid phase resin. Batch flow occurs in a filter reaction vessel and is useful because reactants are accessible and can be added manually or automatically.

Two types of protecting groups are commonly used for protecting the N-alpha-amino terminus: “Boc” (tert-butyloxycarbonyl) and “Fmoc” (9-fluorenylmethyloxycarbonyl). Reagents for the Boc method are relatively inexpensive, but they are highly corrosive and require expensive equipment and more rigorous precautions to be taken. The Fmoc method, which uses less corrosive, although more expensive, reagents is typically preferred.

For SPPS, a wide variety of solid support phases are available. The solid phase support used for synthesis can be a synthetic resin, a synthetic polymer film or a silicon or silicate surface (e.g. controlled pore glass) suitable for synthesis purposes. Generally, a resin is used, commonly polystyrene suspensions, or polystyrene-polyethyleneglycol, or polymer supports for example polyamide. Examples of resins functionalized with linkers suitable for Boc-chemistry include PAM resin, oxime resin SS, phenol resin, brominated Wang resin and brominated PPOA resin. Examples of resins suitable for Fmoc chemistry include amino-methyl polystyrene resins, AMPB-BHA resin, Sieber amide resin, Rink acid resin, Tentagel S AC resin, 2-chlorotrityl chloride resin, 2-chlorotrityl alcohol resin, TentaGel S Trt-OH resin, Knorr-2-chlorotrityl resin, hydrazine-2-chlorotrityl resin, ANP resin, Fmoc photolable resin, HMBA-MBHA resin, TentaGel S HMB resin, Aromatic Safety Catch resinBAl resin and Fmoc-hydroxylamine 2 chlorotrityl resin. Other resins include PL Cl-Trt resin, PL-Oxime resin and PL-HMBA Resin. Generally resins are interchangeable.

For each resin appropriate coupling conditions are known in the literature for the attachment of the starting monomer or sub-unit.

Preparation of the solid phase support includes solvating the support in an appropriate solvent (e.g. dimethylformamide). The solid phase typically increases in volume during solvation, which in turn increases the surface area available to carry out peptide synthesis.

A linker molecule is then attached to the support for connecting the peptide chain to the solid phase support. Linker molecules are generally designed such that eventual cleavage provides either a free acid or amide at the C-terminus. Linkers are generally not resin-specific. Examples of linkers include peptide acids for example 4-hydroxymethylphenoxyacetyl-4′-methylbenzyhydrylamine (HMP), or peptide amides for example benzhydrylamine derivatives, or the hydroxymethylphenoxypropionyl (HMPP) linker.

The first amino acid of the peptide sequence may be attached to the linker after the linker is attached to the solid phase support or attached to the solid phase support using a linker that includes the first amino acid of the peptide sequence. Linkers that include amino acids are commercially available.

The next step is to deprotect the Nα-amino group of the first amino acid. For Fmoc SPPS, deprotection of the Nα-amino group may be carried out with a mild base treatment (piperazine or piperidine, for example). Side-chain protecting groups may be removed by moderate acidolysis (trifluoroacetic acid (TFA), for example). For Boc SPPS, deprotection of the Nα-amino group may be carried out using for example TFA.

Following deprotection, the amino acid chain extension, or coupling, proceeds by the formation of peptide bonds. This process requires activation of the C-α-carboxyl group of the amino acid to be coupled. This may be accomplished using, for example, in situ reagents, preformed symmetrical anhydrides, active esters, acid halides, or urethane-protected N-carboxyanhydrides. The in situ method allows concurrent activation and coupling. Coupling reagents include carbodiimide derivatives, for example N,N′-dicyclohexylcarbodiinnide or N,N-diisopropylcarbodiimide. Coupling reagents also include uronium or phosphonium salt derivatives of benzotriazol. Examples of such uronium and phosphonium salts include HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), and HAPyU (O-(benzotriazol-1-yl)oxybis-(pyrrolidino)-uronium hexafluorophosphate. In some embodiments, the coupling reagent is HBTU, HATU, BOP, or PyBOP.

After the desired amino acid sequence has been synthesized, the peptide is cleaved from the resin. The conditions used in this process depend on the sensitivity of the amino acid composition of the peptide and the side-chain protecting groups. Generally, cleavage is carried out in an environment containing a plurality of scavenging agents to quench the reactive carbonium ions that originate from the protective groups and linkers. Common cleaving agents include, for example, TFA and hydrogen fluoride (HF). In some embodiments, where the peptide is bound to the solid phase support via a linker, the peptide chain is cleaved from the solid phase support by cleaving the peptide from the linker.

The conditions used for cleaving the peptide from the resin may concomitantly remove one or more side-chain protecting groups.

The use of protective groups in SPPS is well established. Examples of common protective groups include but are not limited to acetamidomethyl (Acm), acetyl (Ac), adamantyloxy (AdaO), benzoyl (Bz), benzyl (Bzl), 2-bromobenzyl, benzyloxy (BzlO), benzyloxycarbonyl (Z), benzyloxymethyl (Bonn), 2-bromobenzyloxycarbonyl (2-Br-Z), tert-butoxy (tBuO), tert-butoxycarbonyl (Boc), tert-butoxymethyl (Bum), tert-butyl (tBu), tert-buthylthio (tButhio), 2-chlorobenzyloxycarbonyl (2-Cl-Z), cyclohexyloxy (cHxO), 2,6-dichlorobenzyl (2,6-DiCI-Bzl), 4,4′-dimethoxybenzhydrol (Mbh), 1-(4,4-dimethyl-2,6-dioxo-cyclohexylidene)3-methyl-butyl (ivDde), 4-{N-[1-(4,4-dimethyl-2,6-dioxo-cyclohexylidene)3-methylbutyl]-amino) benzyloxy (ODmab), 2,4-dinitrophenyl (Dnp), fluorenylmethoxycarbonyl (Fmoc), formyl (For), mesitylene-2-sulfonyl (Mts), 4-methoxybenzyl (MeOBzl), 4-methoxy-2,3,6-trimethyl-benzenesulfonyl (Mtr), 4-methoxytrityl (Mmt), 4-methylbenzyl (MeBzI), 4-methyltrityl (Mtt), 3-nitro-2-pyridinesulfenyl (Npys), 2,2,4,6,7-pentamethyldihydrobenzofurane-5-sulfonyl (Pbf), 2,2,5,7,8-pentamethyl-chromane-6-sulfonyl (Pmc), tosyl (Tos), trifluoroacetyl (Tfa), trimethylacetamidomethyl (Tacm), trityl (Trt) and xanthyl (Xan).

Where one or more of the side chains of the amino acids of the peptide contains functional groups, such as for example additional carboxylic, amino, hydroxy or thiol groups, additional protective groups may be necessary. For example, if the Fmoc strategy is used, Mtr, Pmc, Pbf may be used for the protection of Arg; Trt, Tmob may be used for the protection of Asn and Gln; Boc may be used for the protection of Trp and Lys; tBu may be used for the protection of Asp, Glu, Ser, Thr and Tyr; and Acm, tBu, tButhio, Trt and Mmt may be used for the protection of Cys. A person skilled in the art will appreciate that there are numerous other suitable combinations.

The methods for SPPS outlined above are well known in the art. See, for example, Atherton and Sheppard, “Solid Phase Peptide Synthesis: A Practical Approach,” New York: IRL Press, 1989; Stewart and Young: “Solid-Phase Peptide Synthesis 2nd Ed.,” Rockford, Ill.: Pierce Chemical Co., 1984; Jones, “The Chemical Synthesis of Peptides,” Oxford: Clarendon Press, 1994; Merrifield, J. Am. Soc. 85:2146-2149 (1963); Marglin, A. and Merrifield, R. B. Annu. Rev. Biochem. 39:841-66 (1970); and Merrifield R. B. JAMA. 210(7):1247-54 (1969); and “Solid Phase Peptide Synthesis—A Practical Approach” (W. C. Chan and P. D. White, eds. Oxford University Press, 2000). Equipment for automated synthesis of peptides or polypeptides is readily commercially available from suppliers such as Perkin Elmer/Applied Biosystems (Foster City, Calif.) and may be operated according to the manufacturer's instructions.

Following cleavage from the resin, the peptide may be separated from the reaction medium, e.g. by centrifugation or filtration. The peptide may then be subsequently purified, e.g. by HPLC using one or more suitable solvents.

Advantageously, the inventors have found that in some embodiments the peptide-containing conjugation partner may be used in the methods of the present invention without purification following cleavage of the peptide from the resin.

The inventors have also advantageously found that in some embodiments the thiolene method of the present invention can be carried out using a peptide-containing conjugation partner, wherein the peptide does not contain an Nα-amino group protecting group or any side chain protecting groups. The reaction is generally selective for reaction of a thiol and a non-aromatic carbon-carbon double bond.

It may be necessary to protect thiol groups present in the peptide-containing conjugation partner (e.g. in cysteine residues of the peptide) with a protective group to prevent undesirable competing reactions in the methods of the present invention. The thiol groups may be protected with a protective group that is not removable under the conditions used to remove one or more other protecting groups present in the peptide or to cleave the peptide from the resin.

Typically, the peptide will be synthesised using amino acids bearing the appropriate protecting groups. A person skilled in the art will be able to select appropriate protecting groups without undue experimentation.

The amino acid-comprising conjugation partner and/or lipid-containing conjugation partners may comprise one or more unsaturated carbon-carbon bonds in addition to the carbon-carbon double bonds of the lipid containing conjugation partners to be reacted. Those skilled in the art will appreciate that the selectivity of the thiol for the carbon-carbon double bond to be reacted in such embodiments may depend on, for example, the steric and/or electronic environment of the carbon-carbon double bond relative to the one or more additional unsaturated carbon-carbon bonds. In certain embodiments, the carbon-carbon double bonds to be reacted are activated relative to any other unsaturated carbon-carbon bonds in the amino acid-comprising conjugation partner and lipid-containing conjugation partner. In certain embodiments, the carbon-carbon double bonds to be reacted are activated relative to any other unsaturated carbon-carbon bonds in the peptide-containing conjugation partner and lipid-containing conjugation partner.

In some embodiments, the Nα-amino group of the amino acid of the amino acid-comprising conjugation partner comprising the thiol is acylated, for example acetylated. In some embodiments, the methods of the present invention may comprise acylating, for example acetylating, the Nα-amino group of the amino acid of the amino acid-comprising conjugation partner comprising the carbon-carbon double bond or thiol to be reacted.

Where a peptide-containing conjugation partner has been synthesised by SPPS, acylation may be carried out prior to or after cleavage from the resin. In some embodiments, the amino acid residue of the peptide-containing conjugation partner bearing the thiol to be reacted is an N-terminal amino acid residue, for example cysteine, and the method comprises acylating the N-terminal amino group prior to cleaving the peptide.

In some embodiments, the method further comprises acylating, for example acetylating, the Nα-amino group of the amino acid of the amino acid conjugate or the amino acid residue of the peptide conjugate to which the lipid moieties are conjugated.

Acylation of the Nα-amino group of an amino acid may be carried out by reacting an amino acid or peptide with an acylating agent in the presence of base in a suitable solvent, for example DMF. Non-limiting examples of acylating agents include acid halides, for example acid chlorides such as acetyl chloride, and acid anhydrides, for example acetic anhydride. Such agents maybe commercially available or may be prepared by methods well known in the art. Non-limiting examples of suitable bases include triethylamine, diisopropylethylamine, 4-methylmorpholine, and the like.

In other embodiments, the synthesising the peptide of the peptide-containing conjugation partner comprises coupling an amino acid or a peptide comprising an amino acid that is acylated, for example acetylated, at the Nα-amino group and comprises the thiol to be reacted to one or more amino acids and/or one or more peptides.

In some embodiments, the method comprises coupling the amino acid of the amino acid conjugate to an amino acid or a peptide to provide a peptide conjugate. In some embodiments, the method comprises coupling the amino acid of the amino acid conjugate to an amino acid or peptide bound to a solid phase resin support by SPPS. In some embodiments, the method comprises coupling the amino acid of the amino acid conjugate to a peptide bound to a solid phase resin support by SPPS. The method may comprise synthesising the peptide bound to the solid phase resin support by SPPS.

In some embodiments, the method further comprises coupling the amino acid of the amino acid conjugate or an amino acid of the peptide conjugate to an amino acid or a peptide so as to provide a peptide conjugate comprising a peptide epitope. In some embodiments, the peptide to be coupled comprises a peptide epitope. In other embodiments, a peptide epitope is formed on coupling. The coupling may be carried out by SPPS as described herein.

In some embodiments, the method comprises coupling the amino acid of the amino acid conjugate to a peptide bound to a solid phase resin support by SPPS so as to provide a peptide conjugate comprising a peptide epitope.

In one embodiment, the peptide of the peptide conjugate to be coupled is bound to a solid phase resin support, and the method comprises coupling an amino acid of the peptide conjugate to be coupled to an amino acid or a peptide so as to provide a peptide conjugate comprising a peptide epitope.

In an alternate embodiment, the method comprises coupling an amino acid of the peptide conjugate to an amino acid or peptide bound to a solid phase resin support by SPPS so as to provide peptide conjugate comprising a peptide epitope.

In some embodiments, the method further comprises coupling an epitope, for example a peptide epitope, to the amino acid conjugate or peptide conjugate. Where the method comprises coupling a peptide epitope, the coupling may be carried out by SPPS as described herein.

In certain embodiments, the epitope, for example a peptide epitope, is coupled or bound via a linker group. In certain embodiments, the linker group is an amino sequence, for example a sequence of two or more, three or more, or four or more contiguous amino acids. In certain embodiments, the linker comprises from about 2 to 20, 2 to 18, 2 to 16, 2 to 14, 2 to 12, 2 to 10, 4 to 20, 4 to 18, 4 to 16, 4 to 14, 4 to 12, or 4 to 10 amino acids.

It will be appreciated by those skilled in the art that coupling an amino acid or a peptide to another amino acid or peptide as described herein may comprise forming a peptide bond between the Nα-terminus of the amino acid or an amino acid of the peptide of one coupling partner and the C-terminus of the amino acid or an amino acid of the peptide of the other coupling partner.

In some embodiments, the method of the present invention comprises synthesising the amino acid sequence of the peptide of the peptide-containing conjugation partner by SPPS; and reacting the peptide-containing conjugation partner.

In some embodiments, the method of the present invention comprises synthesising the amino acid sequence of the peptide of the peptide-containing conjugation partner by SPPS; and reacting the lipid-containing conjugation partners with the peptide-containing conjugation partner.

In some embodiments, synthesising the amino acid sequence of the peptide of the peptide-containing conjugation partner by SPPS comprises coupling an amino acid or peptide to an amino acid or peptide bound to a solid phase resin support to provide the amino acid sequence of the peptide or a portion thereof. In certain embodiments, the amino acid sequence of the entire peptide of the peptide-containing conjugation partner is synthesised by SPPS.

The peptide-containing conjugation partner may be reacted, for example with the lipid-containing conjugation partners in the thiolene method, while bound to a solid phase resin support. Alternatively, the peptide may be cleaved from the solid phase resin support, and optionally purified, prior to reaction, for example with the lipid-containing conjugation partners.

The peptide conjugate and/or amino acid-comprising conjugation partner, for example a peptide-containing conjugation partner, may comprise one or more solubilising groups. The one or more solubilising groups increase the solubility of, for example, the peptide-containing conjugation partner in polar solvents, such as water. In exemplary embodiments, the solubilising group does not adversely affect the biological activity of the peptide conjugate.

The presence of a solubilising group may be advantageous for formulation and/or administration of the peptide conjugate as a pharmaceutical composition.

In some embodiments, the solubilising group is bound to the peptide of the peptide conjugate and/or peptide-containing conjugation partner. In some embodiments, the solubilising group is bound to the peptide of the peptide-containing conjugation partner. In some embodiments, the peptide of the peptide conjugate and/or the peptide of the peptide-containing partner comprises a solubilising group. In some embodiments, the peptide of the peptide-containing partner comprises a solubilising group.

In some embodiments, the solubilising group is bound to the side chain of an amino acid in the peptide chain. In some embodiments, the solubilising group is bound to the C- or N-terminus of the peptide chain. In some embodiments, the solubilising group is bound between two amino acid residues in the peptide chain. In some embodiments, the solubilising group is bound to the Nα-amino group of one amino acid residue in the peptide chain and the carboxyl group of another amino acid residue in the peptide chain. Examples of suitable solubilising groups include, but are not limited to, hydrophilic amino acid sequences or polyethylene glycols (PEGs).

In one embodiment, the solubilising group is a hydrophilic amino acid sequence comprising two or more hydrophilic amino acid residues in the peptide chain. In some embodiments, the solubilising group is an amino acid sequence comprising a sequence of two or more consecutive hydrophilic amino acid residues in the peptide chain. Such solubilising groups may be formed by adding each amino acid of the solubilising group to the peptide chain by SPPS.

In another embodiment, the solubilising group is a polyethylene glycol. In some embodiments, the polyethylene glycol is bound to the Nα-amino group of one amino acid residue in the peptide chain and the carboxyl group of another amino acid residue in the peptide chain.

In some embodiments, the polyethylene glycol comprises from about 1 to about 100, about 1 to about 50, about 1 to about 25, about 1 to about 20, about 1 to about 15, about 1 to about 15, about 1 to about 10, about 2 to about 10, or about 2 to about 4 ethylene glycol monomer units. Methods for coupling polyethylene glycols to peptides are known.

In some embodiments, the peptide conjugate and/or peptide-containing conjugation partner comprises an antigen, for example, an antigenic peptide. In one embodiment, the peptide of the peptide conjugate or peptide-containing conjugation partner is or comprises an antigen; or an antigen is bound to peptide, optionally via a linker. In some embodiments, the peptide-containing conjugation partner comprises an antigen, for example, an antigenic peptide. In one embodiment, the peptide of the peptide-containing conjugation partner is or comprises an antigen; or an antigen is bound to peptide, optionally via a linker.

In one embodiment, the antigen comprises a peptide comprising an epitope. In one embodiment, the peptide comprising an epitope is a glycopeptide comprising an epitope. In one embodiment, the antigen comprises a glycopeptide comprising an epitope.

In some embodiments, the peptide conjugate and/or peptide-containing conjugation partner comprises an epitope. In some embodiments, the peptide of the peptide conjugate and/or peptide-containing conjugation partner comprises an epitope. In some embodiments, the peptide-containing conjugation partner comprises an epitope. In some embodiments, the peptide of the peptide-containing conjugation partner comprises an epitope.

In some embodiments, the peptide conjugate and/or peptide-containing conjugation partner comprises two or more epitopes, for example, the peptide of the peptide conjugate and/or peptide-containing conjugation partner comprises two or more epitopes.

In some embodiments, the peptide conjugate and/or peptide-containing conjugation partner is or comprises a glycopeptide comprising an epitope. In some embodiments, the peptide of the peptide conjugate and/or peptide-containing conjugation partner is a glycopeptide. In some embodiments, the peptide conjugate and/or peptide-containing conjugation partner comprises a glycopeptide comprising an epitope bound to the peptide of the peptide conjugate and/or peptide-containing conjugation partner. In some embodiments, the peptide-containing conjugation partner is or comprises a glycopeptide comprising an epitope. In some embodiments, the peptide of the peptide-containing conjugation partner is a glycopeptide. In some embodiments, the peptide-containing conjugation partner comprises a glycopeptide comprising an epitope bound to the peptide of the peptide-containing conjugation partner.

In some embodiments, the peptide conjugate and/or peptide-containing conjugation partner comprises a proteolytic cleavage site. In some embodiments, the peptide of the peptide conjugate and/or peptide-containing conjugation partner comprises a proteolytic cleavage site. In some embodiments, the peptide-containing conjugation partner comprises a proteolytic cleavage site. In some embodiments, the peptide of the peptide-containing conjugation partner comprises a proteolytic cleavage site.

In some embodiments, the peptide of the peptide conjugate and/or peptide-containing conjugation partner comprises one or more linker groups. In some embodiments, the peptide of the peptide-containing conjugation partner comprises one or more linker groups.

In some embodiments, the peptide conjugate and/or peptide-containing conjugation partner comprises a linker group. In some embodiments, the peptide-containing conjugation partner comprises a linker group.

In some embodiments, the peptide conjugate and/or peptide-containing conjugation partner comprises an epitope bound to the peptide of the peptide conjugate and/or peptide-containing conjugation partner via a linker group. In some embodiments, the peptide-containing conjugation partner comprises an epitope bound to the peptide of the peptide-containing conjugation partner via a linker group.

Examples of linker groups include but are not limited to amino acid sequences (for example, a peptide), polyethylene glycol, alkyl amino acids, and the like. In some embodiments, the linker is or comprises a proteolytic cleavage site. In some embodiments, the linker is or comprises a solubilising group.

In some embodiments, the linker is bound between two amino acid residues in the peptide chain.

In some embodiments, the linker group is bound to the Nα-amino group of one amino acid residue in the peptide conjugate and/or peptide-containing conjugation partner and the carboxyl group of another amino acid residue in the peptide-containing conjugation partner. In some embodiments, the linker group is bound to the Nα-amino group of one amino acid residue in the peptide-containing conjugation partner and the carboxyl group of another amino acid residue in the peptide-containing conjugation partner.

In certain embodiments, the linker group is cleavable in vivo from the amino acids to which it is bound. In certain embodiments, the linker group is cleavable by hydrolysis in vivo. In certain embodiments, the linker group is cleavable by enzymatic hydrolysis in vivo. Linker groups may be introduced by any suitable method known in the art.

The method may further comprise coupling an epitope to the amino acid of the amino acid conjugate or the peptide of the peptide conjugate. The epitope may be bound via a linker group, as described above. In some embodiments, the epitope is a peptide epitope. In some embodiments, the method comprises coupling a glycopeptide comprising an epitope.

It will be appreciated that in certain desirable embodiments, the peptide conjugates of the invention maintain appropriate uptake, processing, and presentation by antigen presenting cells. Desirably, the lipid-containing conjugate does not interfere with presentation of any antigenic peptide present in the conjugate by antigen presenting cells.

Confirmation of the identity of the peptides synthesized may be conveniently achieved by, for example, amino acid analysis, mass spectrometry, Edman degradation, and the like.

The method of the present invention may further comprise separating the amino acid conjugate from the liquid reaction medium. Alternatively, the method of the present invention may further comprise separating the peptide conjugate from the liquid reaction medium. Any suitable separation methods known in the art may be used, for example, precipitation and filtration. The conjugate may be subsequently purified, for example, by HPLC using one or more suitable solvents.

The present invention also relates to peptide conjugates made by the methods of the present invention.

The peptide conjugates may be pure or purified, or substantially pure.

As used herein “purified” does not require absolute purity; rather, it is intended as a relative term where the material in question is more pure than in the environment it was in previously. In practice the material has typically, for example, been subjected to fractionation to remove various other components, and the resultant material has substantially retained its desired biological activity or activities. The term “substantially purified” refers to materials that are at least about 60% free, preferably at least about 75% free, and most preferably at least about 90% free, at least about 95% free, at least about 98% free, or more, from other components with which they may be associated during manufacture.

The term “α-amino acid” or “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

In certain embodiments the peptide-containing conjugation partner comprises only natural amino acids. The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The term “amino acid analog” or “non-naturally occurring amino acid” refers to a molecule which is structurally similar to an amino acid and which can be substituted for an amino acid. Amino acid analogs include, without limitation, compounds which are structurally identical to an amino acid, as defined herein, except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine, or substitution or the carboxy group with an ester or carboxamide).

Unless otherwise indicated, conventional techniques of molecular biology, microbiology, cell biology, biochemistry and immunology, which are within the skill of the art may be employed in practicing the methods described herein. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press NY, 1994); Antibodies: A Laboratory Manual (Harlow et al., eds., 1987); and Methods of Immunological Analysis (R. Masseyeff, W. H. Albert, and N. A. Staines, eds., Weinheim: VCH Verlags gesellschaft mbH, 1993).

The term “peptide” and the like is used herein to refer to any polymer of amino acid residues of any length. The polymer can be linear or non-linear (e.g., branched), it can comprise modified amino acids or amino acid analogs. The term also encompasses amino acid polymers that have been modified naturally or by intervention, for example, by disulfide bond formation, glycosylation, lipidation, acetylation, phosphorylation, or any other modification or manipulation, for example conjugation with labeling or bioactive components.

The inventors have found that peptide conjugates of the present invention have immunological activity.

Cell-mediated immunity is primarily mediated by T-lymphocytes. Pathogenic antigens are expressed on the surface of antigen presenting cells (such as macrophages, B-lymphocytes, and dendritic cells), bound to either major histocompatibility MHC Class I or MHC Class II molecules. Presentation of pathogenic antigen coupled to MHC Class II activates a helper (CD4+) T-cell response. Upon binding of the T-cell to the antigen-MHC II complex, CD4+ T-cells, release cytokines and proliferate.

Presentation of pathogenic antigens bound to MHC Class I molecules activates a cytotoxic (CD8+) T-cell response. Upon binding of the T-cell to the antigen-MHC I complex, CD8+ cells secrete perforin and other mediators, resulting in target cell death. Without wishing to be bound by any theory, the applicants believe that in certain embodiments an enhanced response by CD8+ cells is achieved in the presence of one or more epitopes recognised by CD4+ cells.

Methods to assess and monitor the onset or progression of a cell-mediated response in a subject are well known in the art. Convenient exemplary methods include those in which the presence of or the level of one or more cytokines associated with a cell-mediated response, such as those identified herein, is assessed. Similarly, cell-based methods to assess or monitor the onset and progression of a cell-mediated response are amenable to use in the present invention, and may include cell proliferation or activation assays, including assays targeted at identifying activation or expansion of one or more populations of immune cells, such as T-lymphocytes.

In certain embodiments, methods of the invention elicit both a cell-mediated immune response and a humoral response.

The humoral immune response is mediated by secreted antibodies produced by B cells. The secreted antibodies bind to antigens presented on the surface of invading pathogens, flagging them for destruction.

Again, methods to assess and monitor the onset or progression of a humoral response are well known in the art. These include antibody binding assays, ELISA, skin-prick tests and the like.

Without wishing to be bound by theory, the inventors believe that the peptide conjugates in some embodiments stimulate Toll like receptors (TLRs).

Toll-like receptors (TLRs) are highly conserved pattern recognition receptors (PRRs) that recognise pathogen-associated molecular patterns and transmit danger signals to the cell (Kawai, T., Akira, S., Immunity 2011, 34, 637-650). TLR2 is a cell-surface receptor expressed on a range of different cell types, including dendritic cells, macrophages and lymphocytes (Coffman, R. L., Sher, A., Seder, R. A., Immunity 2010, 33, 492-503).

TLR2 recognises a wide range of microbial components including lipopolysaccharides, peptidoglycans and lipoteichoic acid. It is unique amongst TLRs in that it forms heterodimers, with either TLR1 or TLR6; the ability to form complexes with other PRRs may explain the wide range of agonists for TLR2 (Feldmann, M., Steinman, L., Nature 2005, 435, 612-619). Upon ligand binding and heterodimerisation, signalling takes place via the MyD88 pathway, leading to NFκB activation and consequent production of inflammatory and effector cytokines.

Di- and triacylated lipopeptides derived from bacterial cell-wall components have been extensively studied as TLR2 agonists (Eriksson, E. M. Y., Jackson, D. C., Curr. Prot. and Pept. Sci. 2007, 8, 412-417). Lipopeptides have been reported to promote dendritic cell maturation, causing the up-regulation of co-stimulatory molecules on the cell surface and enhanced antigen-presentation. Lipopeptides have also been reported to stimulate macrophages to release cytokines and promote the activation of lymphocytes including B cells and CD8+ T cells.

In some embodiments, the peptide conjugate has TLR2 agonist activity. S-(2,3-bis(palmitoyloxy)-(2RS)-propyl)-N-palmitoyl-(R-)-Cys-Lys-Lys-Lys-Lys-OH (Pam3CSK4) is a potent TLR2 agonist and may be selected as benchmark against which the TLR2 agonism of the peptide conjugate compounds of the invention may be compared. In some embodiments, the peptide conjugate has a TLR2 agonist potency from 1,000, 100, or 10 fold less than of the potency of Pam3CSK4 to 1,000, 100, or 10 fold more than the potency of Pam3CSK4. In some embodiments, the peptide conjugate has TLR2 agonist activity comparable to Pam3CSK4. In some embodiments, the peptide conjugate has TLR2 agonist activity at least about 50%, about 60%, about 70%, about 80%, about 90% that of Pam3CSK4. In some embodiments, the peptide conjugate has TLR2 agonist activity greater than that of Pam3CSK4. For example, in some embodiments, the peptide conjugate has TLR2 agonist activity greater than 100%, 150%, 200%, or 500%. In other embodiments, the peptide conjugate has TLR2 agonist activity 10 fold or 100 fold greater than that of Pam3CSK4. In some embodiments, for example in embodiments where a modulated immune response is desirable, the peptide conjugate has TLR2 agonist activity less than that of Pam3CSK4. For example, the peptide conjugate has TLR2 agonist activity less than about 50%, less than about 40%, less than about 30%, less than about 20%, less than about 10%, less than about 1%, or less than 0.1% that of Pam3CSK4.

In some embodiments, the TLR2 agonist activity is determined using a Hek-Blue™ cell assay (for example, by following a procedure analogous to that described in the Examples herein).

In some embodiments, the TLR2 is murine TLR2 or human TLR2. In certain exemplary embodiments, the TLR2 (mTLR2) is human TLR2 (hTLR2).

In some embodiments, the peptide conjugate has an EC₅₀ for TLR2 agonism (preferably hTLR2) of less than about 500 nM as determined using a HEK-Blue™ cell assay (for example, by following a procedure analogous to that described in the Examples herein), for example less than about 400, 300, 250, 200, 175, 150, 125, 100, 75, 50, 25, 20, 15, 10, 5, 4, 3, 2.5, 2, 1.5, 1, 0.9, 0.8, 0.7, 0.6, 0.5, 0.4, 0.3, 0.2 or 0.1 nM, and useful ranges may be selected from any two of these values, for example from about 0.1 nM to 500 nM, 0.1 nM to 150 nM, or 0.1 nM to 10 nM. In some embodiments, the peptide conjugate has an EC₅₀ for TLR2 agonism (preferably hTLR2) of at least about 0.01 nM as determined using a HEK-Blue™ cell assay (for example, by following a procedure analogous to that described in the Examples herein), for example at least 0.05, 0.1, 0.5, 1, 1.5, or 2 nM, and useful ranges may be selected from any two of these values, for example from about 0.01 nM to 2 nM, 0.01 nM to 1.5 nM, or 0.01 nM to 1 nM. In some embodiments, the peptide of the peptide conjugate and/or peptide-containing conjugation partner comprises a serine amino acid residue adjacent to the amino acid through which the lipid moieties are conjugated to the peptide. In some embodiments, the serine is bound to the C-termini of the amino acid. The presence of the serine amino acid residue in this position may enhance TLR2 binding.

As will be appreciated by those skilled in the art on reading this disclosure, the peptide conjugate may comprise an epitope, including, for example two or more epitopes. The epitope may be coupled or bound to the peptide via a linker group. In some embodiments, the epitope is a peptide epitope. A person skilled in the art will appreciate that a wide range of peptide epitopes may be employed in the present invention.

### Antigens

It will be appreciated that a great many antigens, for example tumour antigens or antigens from various pathogenic organisms, have been characterised and are suitable for use in the present invention. All antigens, whether or not presently characterized, that are capable of eliciting an immune response are contemplated.

Accordingly, depending on the choice of antigen the conjugates of the present invention find application in a wide range of immunotherapies, including but not limited to the treatment and prevention of infectious disease, the treatment and prevention of cancer, and the treatment of viral re-activation during or following immunosuppression, for example in patients who have had bone marrow transplants or haematopoietic stem cell transplants.

Also contemplated are antigens comprising one or more amino acid substitutions, such as one or more conservative amino acid substitutions.

A “conservative amino acid substitution” is one in which an amino acid residue is replaced with another residue having a chemically similar or derivatised side chain. Families of amino acid residues having similar side chains, for example, have been defined in the art. These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid analogs (e.g., phosphorylated or glycosylated amino acids) are also contemplated in the present invention, as are peptides substituted with non-naturally occurring amino acids, including but not limited to N-alkylated amino acids (e.g. N-methyl amino acids), D-amino acids, 3-amino acids, and γ-amino acids.

Fragments and variants of antigens are also specifically contemplated.

A “fragment” of a peptide, is a subsequence of the peptide that performs a function that is required for the enzymatic or binding activity and/or provides three dimensional structure of the peptide, such as the three dimensional structure of a polypeptide.

The term “variant” as used herein refers to peptide sequences, including for example peptide sequences different from the specifically identified sequences, wherein one or more amino acid residues is deleted, substituted, or added. Variants are naturally-occurring variants, or non-naturally occurring variants. Variants are from the same or from other species and may encompass homologues, paralogues and orthologues. In certain embodiments, variants of peptides including peptides possess biological activities that are the same or similar to those of the wild type peptides. The term “variant” with reference to peptides encompasses all forms of peptides as defined herein.

Those of skill in the art will appreciate that the conjugates of the present invention are in certain embodiments particularly suited for stimulating T-cell responses, for example in the treatment of neoplastic diseases, including cancer. Conjugates of the present invention comprising one or more tumour antigens are specifically contemplated. It will be appreciated that tumour antigens contemplated for use in the preparation of peptide conjugates of the invention will generally comprise one or more peptides. In certain embodiments of the invention, including for example pharmaceutical compositions of the invention, one or more additional tumour antigens may be present, wherein the one or more tumour antigens does not comprise peptide. Tumour antigens are typically classified as either unique antigens, or shared antigens, with the latter group including differentiation antigens, cancer-specific antigens, and over-expressed antigens. Examples of each class of antigens are amenable to use in the present invention. Representative tumour antigens for use in the treatment, for example immunotherapeutic treatment, or vaccination against neoplastic diseases including cancer, are discussed below. Compounds, vaccines and compositions comprising one or more antigens prepared using those methods of immunisation are specifically contemplated.

In certain embodiments, the tumour antigen is a peptide-containing tumour antigen, such as a polypeptide tumour antigen or glycoprotein tumour antigens. In certain embodiments, the tumour antigen is a saccharide-containing tumour antigen, such as a glycolipid tumour antigen or a ganglioside tumour antigen. In certain embodiments, the tumour antigen is a polynucleotide-containing tumour antigen that expresses a polypeptide-containing tumour antigen, for instance, an RNA vector construct or a DNA vector construct, such as plasmid DNA.

Tumour antigens appropriate for the use in the present invention encompass a wide variety of molecules, such as (a) peptide-containing tumour antigens, including peptide epitopes (which can range, for example, from 8-20 amino acids in length, although lengths outside this range are also common), lipopolypeptides and glycoproteins, (b) saccharide-containing tumour antigens, including poly-saccharides, mucins, gangliosides, glycolipids and glycoproteins, including and (c) polynucleotides that express antigenic polypeptides. Again, those skilled in the art will recognise that a tumour antigen present in a conjugate or composition of the present invention will typically comprise peptide. However, embodiments of the invention where one or more conjugates comprises a tumour antigen that does not itself comprise peptide, but for example is bound to the amino acid-comprising or peptide-containing conjugation partner, are contemplated. Similarly, compositions of the invention in which one or more tumour antigens that does not itself comprise peptide is present are contemplated.

In certain embodiments, the tumour antigens are, for example, (a) full length molecules associated with cancer cells, (b) homologues and modified forms of the same, including molecules with deleted, added and/or substituted portions, and (c) fragments of the same, provided said fragments remain antigenic or immunogenic. In certain embodiments, the tumour antigens are provided in recombinant form. In certain embodiments, the tumour antigens include, for example, class I-restricted antigens recognized by CD8+ lymphocytes or class II-restricted antigens recognized by CD4+ lymphocytes. In certain embodiments, tumor antigens include synthetic peptides comprising class I-restricted antigens recognized by CD8+ lymphocytes or class II-restricted antigens recognized by CD4+ lymphocytes.

Shared tumour antigens are generally considered to be native, unmutated sequences that are expressed by tumours due to epigenetic changes that allow de-repression of developmentally-repressed genes. Accordingly, shared antigens are typically considered preferable to over-expressed or differentiation-associated antigens because there is no expression in normal tissues. Also, the same antigens can be targeted in a number of cancer patients. For example, the cancer-testis antigen NY-ESO-1 is present in the majority of patients with many tumours, and a sizeable minority of patients with other tumours. In another example, breast differentiation tumour antigens NYBR-1 and NYBR-1.1 are found in a proportion of breast cancer sufferers. Shared tumour antigens thus represent an attractive target for development.

The use of shared tumour antigens, such cancer-testis antigens including NY-ESO-1, CTSP-1, CTSP-2, CTSP-3, CTSP-4, SSX2, and SCP1, and breast cancer antigens NYBR-1 and NYBR-1.1, in conjugates of the present invention is specifically contemplated herein.

In one exemplary embodiment, the peptide of the peptide-containing conjugation partner or of the peptide conjugate comprises one or more epitopes derived from NY-ESO-1. In one embodiment, the peptide comprises one or more epitopes derived from NY-ESO-1 residues 79-116. In one embodiment, the peptide comprises one or more epitopes derived from NY-ESO-1 residues 118-143. In one embodiment, the peptide comprises one or more epitopes derived from NY-ESO-1 residues 153-180.

In one specifically contemplated embodiment, the peptide of the peptide-containing conjugation partner or of the peptide conjugate, comprises, consists essentially of, or consists of an amino acid sequence selected from the group consisting of 8 or more contiguous, 10 or more contiguous, 12 or more contiguous, 15 or more contiguous, 20 or more contiguous, or 25 or more contiguous amino acids from any one of SEQ ID NOs: 9 to 28.

In various embodiments, the peptide comprises more that one amino acid sequence selected from the group consisting of any one of SEQ ID NOs: 9 to 28. In one embodiment, the peptide comprises one or more amino acid sequences selected from the group consisting of SEQ ID NOs: 12-15, 20, 21, and 26-28.

Similarly, the prostate vaccine Sipuleucel-T (APC8015, Provenge™), which comprises the antigen prostatic acid phosphatase (PAP), is present in 95% of prostate cancer cells. At least in part due to this potential for efficacy in a significant proportion of prostate cancer sufferers, Sipuleucel-T was approved by the FDA in 2010 for use in the treatment of asymptomatic, hormone-refractory prostate cancer. The use of PAP antigen in conjugates of the present invention is specifically contemplated in the present invention.

Unique antigens are considered to be those antigens that are unique to an individual or are shared by a small proportion of cancer patients, and typically result from mutations leading to unique protein sequences. Representative examples of unique tumour antigens include mutated Ras antigens, and mutated p53 antigens. As will be appreciated by those skilled in the art having read this specification, the methods of the present invention enable the ready preparation of conjugates comprising one or more unique tumour antigens, for example to elicit specific T-cell responses to one or more unique tumour antigens, for example in the preparation of patient-specific therapies.

Accordingly, representative tumour antigens include, but are not limited to, (a) antigens such as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours), (b) mutated antigens, for example, p53 (associated with various solid tumours, for example, colorectal, lung, head and neck cancer), p21/Ras (associated with, for example, melanoma, pancreatic cancer and colorectal cancer), CDK4 (associated with, for example, melanoma), MUM1 (associated with, for example, melanoma), caspase-8 (associated with, for example, head and neck cancer), CIA 0205 (associated with, for example, bladder cancer), HLA-A2-R1701, beta catenin (associated with, for example, melanoma), TCR (associated with, for example, T-cell non-Hodgkins lymphoma), BCR-abl (associated with, for example, chronic myelogenous leukemia), triosephosphate isomerase, MA 0205, CDC-27, and LDLR-FUT, (c) over-expressed antigens, for example, Galectin 4 (associated with, for example, colorectal cancer), Galectin 9 (associated with, for example, Hodgkin's disease), proteinase 3 (associated with, for example, chronic myelogenous leukemia), Wilm's tumour antigen-1 (WT 1, associated with, for example, various leukemias), carbonic anhydrase (associated with, for example, renal cancer), aldolase A (associated with, for example, lung cancer), PRAME (associated with, for example, melanoma), HER-2/neu (associated with, for example, breast, colon, lung and ovarian cancer), alpha-fetoprotein (associated with, for example, hepatoma), KSA (associated with, for example, colorectal cancer), gastrin (associated with, for example, pancreatic and gastric cancer), telomerase catalytic protein, MUC-1 (associated with, for example, breast and ovarian cancer), G-250 (associated with, for example, renal cell carcinoma), p53 (associated with, for example, breast, colon cancer), and carcinoembryonic antigen (associated with, for example, breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, for example, melanoma), (e) prostate associated antigens such as PAP, prostatic serum antigen (PSA), PSMA, PSH-P1, PSM-P1, PSM-P2, associated with for example, prostate cancer, (f) immunoglobulin idiotypes (associated with myeloma and B cell lymphomas, for example), and (g) other tumour antigens, such as polypeptide- and saccharide-containing antigens including (i) glycoproteins such as sialyl Tn and sialyl Le.sup.x (associated with, for example, breast and colorectal cancer) as well as various mucins; glycoproteins are coupled to a carrier protein (for example, MUC-1 are coupled to KLH); (ii) lipopolypeptides (for example, MUC-1 linked to a lipid moiety); (iii) polysaccharides (for example, Globo H synthetic hexasaccharide), which are coupled to a carrier proteins (for example, to KLH), (iv) gangliosides such as GM2, GM12, GD2, GD3 (associated with, for example, brain, lung cancer, melanoma), which also are coupled to carrier proteins (for example, KLH).

Other representative tumour antigens amenable to use in the present invention include TAG-72, (See, e.g., U.S. Pat. No. 5,892,020; human carcinoma antigen (See, e.g., U.S. Pat. No. 5,808,005); TP1 and TP3 antigens from osteocarcinoma cells (See, e.g., U.S. Pat. No. 5,855,866); Thomsen-Friedenreich (TF) antigen from adenocarcinoma cells (See, e.g., U.S. Pat. No. 5,110,911); KC-4 antigen from human prostrate adenocarcinoma (See, e.g., U.S. Pat. No. 4,743,543); a human colorectal cancer antigen (See, e.g., U.S. Pat. No. 4,921,789); CA125 antigen from cystadenocarcinoma (See, e.g., U.S. Pat. No. 4,921,790); DF3 antigen from human breast carcinoma (See, e.g., U.S. Pat. Nos. 4,963,484 and 5,053,489); a human breast tumour antigen (See, e.g., U.S. Pat. No. 4,939,240); p97 antigen of human melanoma (See, e.g., U.S. Pat. No. 4,918,164); carcinoma or orosomucoid-related antigen (CORA) (See, e.g., U.S. Pat. No. 4,914,021); T and Tn haptens in glycoproteins of human breast carcinoma, MSA breast carcinoma glycoprotein; MFGM breast carcinoma antigen; DU-PAN-2 pancreatic carcinoma antigen; CA125 ovarian carcinoma antigen; YH206 lung carcinoma antigen, Alphafetoprotein (AFP) hepatocellular carcinoma antigen; Carcinoembryonic antigen (CEA) bowel cancer antigen; Epithelial tumour antigen (ETA) breast cancer antigen; Tyrosinase; the raf oncogene product; gp75; gp100; EBV-LMP 1 & 2; EBV-EBNA 1, 2 & 3C; HPV-E4, 6, 7; C017-1A; GA733; gp72; p53; proteinase 3; telomerase; and melanoma gangliosides. These and other tumour antigens, whether or not presently characterized, are contemplated for use in the present invention.

In certain embodiments, the tumour antigens are derived from mutated or altered cellular components. Representative examples of altered cellular components include, but are not limited to ras, p53, Rb, altered protein encoded by the Wilms' tumour gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor.

Polynucleotide-containing antigens used in the present invention include polynucleotides that encode polypeptide tumour antigens such as those listed above. In certain embodiments, the polynucleotide-containing antigens include, but are not limited to, DNA or RNA vector constructs, such as plasmid vectors (e.g., pCMV), which are capable of expressing polypeptide tumour antigens in vivo.

The present invention also contemplates the preparation of conjugates comprising viral antigens that are capable of stimulating T-cell to elicit effective anti-viral immunity in patients who are or have been immunosuppressed, for example patients who have had bone marrow transplants, haematopoietic stem cell transplants, or are otherwise undergoing immunosuppression.

Similarly, antigens derived from viruses associated with increased incidence of cancer, or that are reported to be cancer-causing, such as human papillomavirus, hepatitis A virus, and hepatitis B virus, are contemplated for use in the present invention.

For example, in certain embodiments, the tumour antigens include, but are not limited to, p15, Hom/Mel-40, H-Ras, E2A-PRL, H4-RET, IGH-IGK, MYL-RAR, Epstein Barr virus antigens, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

In certain embodiments, the tumour antigens include viral proteins implicated in oncogenesis, such as antigens from Epstein Barr virus, human papillomavirus (HPV), including E6 and E7, and hepatitis B and C, and human T-cell lymphotropic virus.

It will be appreciated that such viral proteins, as well as various other viral proteins can also be targets for T cell activity in, for example, treatment against viral disease. In fact, the present invention may be useful in any infection where T cell activity is known to play a role in immunity (effectively all virus infections and many bacterial infections as well, such as tuberculosis). The infectious diseases described herein are provided by way of example only and are in no way intended to limit the scope of the invention. It will be appreciated that the present invention may be useful in the treatment of various other diseases and conditions.

Representative antigens for use in vaccination against pathogenic organisms are discussed below. Compounds, vaccines and compositions comprising one or more antigens prepared using those methods of immunisation are specifically contemplated.

### Tuberculosis Antigens

It will be appreciated that a great many M. tuberculosis antigens have been characterised and are suitable for use in the present invention. All M. tuberculosis antigens, whether or not presently characterized, that are capable of eliciting an immune response are contemplated.

Exemplary M. tuberculosis antigens suitable for use include early secretary antigen target (ESAT)-6, Ag85A, Ag85B (MPT59), Ag85B, Ag85C, MPT32, MPT51, MPT59, MPT63, MPT64, MPT83, MPB5, MPB59, MPB64, MTC28, Mtb2, Mtb8.4, Mtb9.9, Mtb32A, Mtb39, Mtb41, TB10.4, TB10C, TB11B, TB12.5, TB13A, TB14, TB15, TB15A, TB16, TB16A, TB17, TB18, TB21, TB20.6, TB24, TB27B, TB32, TB32A, TB33, TB38, TB40.8, TB51, TB54, TB64, CFP6, CFP7, CFP7A, CFP7B, CFP8A, CFP8B, CFP9, CFP10, CFP11, CFP16, CFP17, CFP19, CFP19A, CFP19B, CFP20, CFP21, CFP22, CFP22A, CFP23, CFP23A, CFP23B, CFP25, CFP25A, CFP27, CFP28, CFP28B, CFP29, CFP30A, CFP30B, CFP50, CWP32, hspX (alpha-crystalline), APA, Tuberculin purified protein derivative (PPD), ST-CF, PPE68, LppX, PstS-1, PstS-2, PstS-3, HBHA, GroEL, GroEL2, GrpES, LHP, 19 kDa lipoprotein, 71 kDa, RD1-ORF2, RD1-ORF3, RD1-ORF4, RD1-ORF5, RD1-ORF8, RD1-ORF9A, RD1-ORF9B, Rv1984c, Rv0577, Rv1827, BfrB, Tpx. Rv1352, Rv1810, PpiA, Cut2, FbpB, FbpA, FbpC, DnaK, FecB, Ssb, RpIL, FixA, FixB, AhpC2, Rv2626c, Rv1211, Mdh, Rv1626, Adk, CIpP, SucD (Belisle et al, 2005; U.S. Pat. No. 7,037,510; US 2004/0057963; US 2008/0199493; US 2008/0267990), or at least one antigenic portion or T-cell epitope of any of the above mentioned antigens.

### Hepatitis Antigens

A number of hepatitis antigens have been characterised and are suitable for use in the present invention. Exemplary hepatitis C antigens include C—p22, E1—gp35, E2—gp70, NS1—p7, NS2—p23, NS3—p70, NS4A—p8, NS4B—p27, NS5A—p56/58, and NS5B—p68, and together with one or more antigenic portions or epitopes derived therefrom are each (whether alone or in combination) suitable for application in the present invention. All hepatitis antigens, whether or not presently characterized, that are capable of eliciting an immune response are contemplated.

### Influenza Antigens

Many influenza antigens have been characterised and are suitable for use in the present invention. Exemplary influenza antigens suitable for use in the present invention include PB, PB2, PA, any of the hemagglutinin (HA) or neuraminidase (NA) proteins, NP, M, and NS, and together with one or more antigenic portions or epitopes derived therefrom are each (whether alone or in combination) suitable for application in the present invention. All influenza antigens, whether or not presently characterized, that are capable of eliciting an immune response are contemplated.

### Anthrax Antigens

A number of B. anthracis antigens have been identified as potential candidates for vaccine development and are useful in the present invention. For example, PA83 is one such antigen for vaccine development. Currently, only one FDA licensed vaccine for anthrax is available called “Anthrax Vaccine Adsorbed” (AVA) or BioThrax®. This vaccine is derived from the cell-free supernatant of a non-encapsulated strain of B. anthracis adsorbed to aluminum adjuvant. PA is the primary immunogen in AVA. Other exemplary anthrax antigens suitable for use in the present invention include Protective antigen (PA or PA63), LF and EF (proteins), poly-gamma-(D-glutamate) capsule, spore antigen (endospore specific components), BcIA (exosporium specific protein), BxpB (spore-associated protein), and secreted proteins. All anthrax antigens together with one or more antigenic portions or epitopes derived therefrom, whether or not presently characterized, that are capable of eliciting an immune response are contemplated.

### Tularemia Antigens

A number of F. tularensis antigens have been identified as potential candidates for vaccine development and are useful in the present invention. For example, AcpA and IgIC are antigens suitable for vaccine development. Other exemplary Tularemia antigens suitable for use in the present invention include 0-antigen, CPS, outer membrane proteins (e.g. FopA), lipoproteins (e.g. Tul4), secreted proteins and lipopolysaccharide. All tularemia antigens together with one or more antigenic portions or epitopes derived therefrom, whether or not presently characterized, that are capable of eliciting an immune response are contemplated.

### Brucellosis Antigens

A number of B. abortusis antigens have been identified as potential candidates for vaccine development and are useful in the present invention. For example, Omp16 is one such antigen for vaccine development. Other exemplary Brucellosis antigens suitable for use in the present invention include O-antigen, lipopolysaccharide, outer membrane proteins (e.g. Omp16), secreted proteins, ribosomal proteins (e.g. L7 and L12), bacterioferritin, p39 (a putative periplasmic binding protein), groEL(heat-shock protein), lumazine synthase, BCSP31 surface protein, PAL16.5 OM lipoprotein, catalase, 26 kDa periplasmic protein, 31 kDa Omp31, 28 kDa Omp, 25 kDa Omp, and 10 kDA Om lipoprotein. All brucellosis antigens together with one or more antigenic portions or epitopes derived therefrom, whether or not presently characterized, that are capable of eliciting an immune response are contemplated.

### Meningitis Antigens

A number of N. meningitidis antigens have been identified as potential candidates for vaccine development and are useful in the present invention. For example, Cys6, PorA, PorB, FetA, and ZnuD are antigens suitable for vaccine development. Other exemplary Meningitis antigens suitable for use in the present invention include 0-antigen, factor H binding protein (fHbp), TbpB, NspA, NadA, outer membrane proteins, group B CPS, secreted proteins and lipopolysaccharide. All meningitis antigens together with one or more antigenic portions or epitopes derived therefrom, whether or not presently characterized, that are capable of eliciting an immune response are contemplated.

### Dengue Antigens

A number of Flavivirus antigens have been identified as potential candidates for vaccine development to treat dengue fever and are useful in the present invention. For example, dengue virus envelope proteins E1-E4 and the membrane proteins M1-M4 are antigens suitable for vaccine development. Other exemplary dengue antigens suitable for use in the present invention include C, preM, 1, 2A, 2B, 3, 4A, 4B and 5. All dengue antigens together with one or more antigenic portions or epitopes derived therefrom, whether or not presently characterized, that are capable of eliciting an immune response are contemplated.

### Ebola Antigens

A number of ebola virus antigens have been identified as potential candidates for vaccine development to treat ebola infection and are useful in the present invention. For example, Filoviridae Zaire ebolavirus and Sudan ebolavirus virion spike glycoprotein precursor antigens ZEBOV-GP, and SEBOV-GP, respectively, are suitable for vaccine development. Other exemplary ebola antigens suitable for use in the present invention include NP, vp35, vp40, GP, vp30, vp24 and L. All ebola antigens together with one or more antigenic portions or epitopes derived therefrom, whether or not presently characterized, that are capable of eliciting an immune response are contemplated.

### West Nile Antigens

A number of West Nile virus antigens have been identified as potential candidates for vaccine development to treat infection and are useful in the present invention. For example, Flavivirus envelope antigen (E) from West Nile virus (WNV) is a non-toxic protein expressed on the surface of WNV virions (WNVE) and are suitable for vaccine development. Other exemplary WNV antigens suitable for use in the present invention include Cp, Prm, NS1, NS2A, NS2B, NS3, NS4A, NS4B and NS5.

All West Nile antigens together with one or more antigenic portions or epitopes derived therefrom, whether or not presently characterized, that are capable of eliciting an immune response are contemplated.

The above-listed or referenced antigens are exemplary, not limiting, of the present invention.

The present invention also relates to pharmaceutical composition comprising an effective amount of a peptide conjugate of the present invention or a pharmaceutically acceptable salt or solvent thereof, and a pharmaceutically acceptable carrier.

The pharmaceutical compositions may comprise an effective amount of two or more peptide conjugates of the invention in combination. In some embodiments, the pharmaceutical compositions may comprise one or more peptide conjugates of the invention and one or more peptides as described herein.

The term “pharmaceutically acceptable carrier” refers to a carrier (adjuvant or vehicle) that may be administered to a subject together with the peptide conjugate of the present invention, or a pharmaceutically acceptable salt or solvent thereof, and a pharmaceutically acceptable carrier.

Pharmaceutically acceptable carriers that may be used in the compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-O-cyclodextrins, or other solubilized derivatives may also be advantageously used to enhance delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

The compositions are formulated to allow for administration to a subject by any chosen route, including but not limited to oral or parenteral (including topical, subcutaneous, intramuscular and intravenous) administration.

For example, the compositions may be formulated with an appropriate pharmaceutically acceptable carrier (including excipients, diluents, auxiliaries, and combinations thereof) selected with regard to the intended route of administration and standard pharmaceutical practice. For example, the compositions may be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions may be formulated to optimize bioavailability, immunogenicity, or to maintain plasma, blood, or tissue concentrations within the immunogenic or therapeutic range, including for extended periods. Controlled delivery preparations may also be used to optimize the antigen concentration at the site of action, for example.

The compositions may be formulated for periodic administration, for example to provide continued exposure. Strategies to elicit a beneficial immunological response, for example those that employ one or more “booster” vaccinations, are well known in the art, and such strategies may be adopted.

The compositions may be administered via the parenteral route. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the composition. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard shell tablet or a capsule containing a binder, e.g., lactose or mannitol, a conventional filler, and a tabletting agent.

Examples of dosage forms suitable for transdermal administration include, but are not limited, to transdermal patches, transdermal bandages, and the like.

Examples of dosage forms suitable for topical administration of the compositions include any lotion, stick, spray, ointment, paste, cream, gel, etc., whether applied directly to the skin or via an intermediary such as a pad, patch or the like.

Examples of dosage forms suitable for suppository administration of the compositions include any solid dosage form inserted into a bodily orifice particularly those inserted rectally, vaginally and urethrally.

Examples of dosage of forms suitable for injection of the compositions include delivery via bolus such as single or multiple administrations by intravenous injection, subcutaneous, subdermal, and intramuscular administration or oral administration.

Examples of dosage forms suitable for depot administration of the compositions and include pellets of the peptide conjugates or solid forms wherein the peptide conjugates are entrapped in a matrix of biodegradable polymers, microemulsions, liposomes or are microencapsulated.

Examples of infusion devices for the compositions include infusion pumps for providing a desired number of doses or steady state administration, and include implantable drug pumps.

Examples of implantable infusion devices for compositions include any solid form in which the peptide conjugates are encapsulated within or dispersed throughout a biodegradable polymer or synthetic, polymer such as silicone, silicone rubber, silastic or similar polymer.

Examples of dosage forms suitable for transmucosal delivery of the compositions include depositories solutions for enemas, pessaries, tampons, creams, gels, pastes, foams, nebulised solutions, powders and similar formulations containing in addition to the active ingredients such carriers as are known in the art to be appropriate. Such dosage forms include forms suitable for inhalation or insufflation of the compositions, including compositions comprising solutions and/or suspensions in pharmaceutically acceptable, aqueous, or organic solvents, or mixture thereof and/or powders. Transmucosal administration of the compositions may utilize any mucosal membrane but commonly utilizes the nasal, buccal, vaginal and rectal tissues. Formulations suitable for nasal administration of the compositions may be administered in a liquid form, for example, nasal spray, nasal drops, or by aerosol administration by nebulizer, including aqueous or oily solutions of the polymer particles. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Examples of dosage forms suitable for buccal or sublingual administration of the compositions include lozenges, tablets and the like. Examples of dosage forms suitable for opthalmic administration of the compositions include inserts and/or compositions comprising solutions and/or suspensions in pharmaceutically acceptable, aqueous, or organic solvents.

Examples of formulations of compositions, including vaccines, may be found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

While the composition may comprise one or more extrinsic adjuvants, advantageously in some embodiments this is not necessary. In some embodiments, the peptide conjugate comprises an epitope and is self adjuvanting.

The present invention provides a method of vaccinating or eliciting an immune response in a subject comprising administering to the subject an effective amount of a peptide conjugate of the present invention. The present invention also relates to a peptide conjugate of the invention for vaccinating or eliciting an immune response in a subject, and to use of a peptide conjugate of the invention in the manufacture of a medicament for vaccinating or eliciting an immune response in a subject.

The present invention also provides a method of vaccinating or eliciting an immune response in a subject comprising administering to the subject an effective amount of the pharmaceutical composition of the present invention. The present invention also relates to a pharmaceutical composition of the invention for vaccinating or eliciting an immune response in a subject, and to the use of one or more peptide conjugates of the present invention in the manufacture of a medicament for vaccinating or eliciting an immune response in a subject.

The present invention also provides a method of activating TLR2 in a subject comprising administering to the subject an effective amount of one or more peptide conjugate of the invention or a pharmaceutically acceptable salt or solvate thereof, or an effective amount of a pharmaceutical composition of the invention. The present invention also provides use of one or more peptide conjugate compounds of the invention or a pharmaceutically acceptable salt or solvate thereof or a pharmaceutical composition of the invention in the manufacture of a medicament for activating TLR2 in a subject and one or more peptide conjugate compounds of the invention or a pharmaceutically acceptable salt or solvate thereof or a pharmaceutical composition of the invention in a subject for activating TLR2 in a subject. Activating TLR2 can stimulate and/or elicit an immune response in the subject, and, in some embodiments, provide immunity.

The administration or use of one or more peptides described herein and/or one or more peptide conjugates of the present invention, for example one or more peptide described herein in together with one or more peptide conjugates, for vaccinating or eliciting an immune response in the subject is contemplated herein.

Where two or more peptide conjugates, or one or more peptides and one or more peptide conjugates are administered or used, the two or more peptide conjugates, or one or more peptides and one or more peptide conjugates may be administered or used simultaneously, sequentially, or separately.

A “subject” refers to a vertebrate that is a mammal, for example, a human. Mammals include, but are not limited to, humans, farm animals, sport animals, pets, primates, mice and rats. The subject may be in need of said vaccinating, eliciting an immune response, or activating TLR2.

An “effective amount” is an amount sufficient to effect beneficial or desired results including clinical results. An effective amount can be administered in one or more administrations by various routes of administration.

The effective amount will vary depending on, among other factors, the disease indicated, the severity of the disease, the age and relative health of the subject, the potency of the compound administered, the mode of administration and the treatment desired. A person skilled in the art will be able to determine appropriate dosages having regard to these any other relevant factors.

The efficacy of a composition can be evaluated both in vitro and in vivo. For example, the composition can be tested in vitro or in vivo for its ability to induce a cell-mediated immune response. For in vivo studies, the composition can be fed to or injected into an animal (e.g., a mouse) and its effects on eliciting an immune response are then assessed. Based on the results, an appropriate dosage range and administration route can be determined.

The composition may be administered as a single dose or a multiple dose schedule. Multiple doses may be used in a primary immunisation schedule and/or in a booster immunisation schedule.

In certain embodiments, eliciting an immune response comprises raising or enhancing an immune response. In exemplary embodiments, eliciting an immune response comprises eliciting a humoral and a cell mediated response.

In certain embodiments, eliciting an immune response provides immunity.

The immune response is elicited for treating a disease or condition. A person skilled in the art will appreciate that the peptide conjugates described herein are useful for treating a variety of diseases and conditions, depending, for example, on the nature of epitope.

In some embodiments, the diseases or conditions are selected from those associated with the various antigens described herein.

In some embodiments the disease or condition is an infectious disease, cancer, or viral re-activation post-bone marrow transplant or following induction of profound immunosuppression for any other reason.

The term “treatment”, and related terms such as “treating” and “treat”, as used herein relates generally to treatment, of a human or a non-human subject, in which some desired therapeutic effect is achieved. The therapeutic effect may, for example, be inhibition, reduction, amelioration, halt, or prevention of a disease or condition.

The compositions may be used to elicit systemic and/or mucosal immunity. Enhanced systemic and/or mucosal immunity may be reflected in an enhanced TH1 and/or TH2 immune response. The enhanced immune response may include an increase in the production of IgG1 and/or IgG2a and/or IgA.

### Examples

### 1. Example 1

This example describes the synthesis of diastereomerically pure amino acid conjugates 6A and 6B.

### 1.1 Preparation and Use of Enantiopure Epoxides 102A and 102B

Diastereomerically pure amino acid conjugates 6A and 6B may be prepared using enantiopure epoxide 102A or enantiopure epoxide 102B produced stereospecifically from an enantiomerically pure starting material.

Enantiopure epoxide 102A and enantiopure epoxide 102B were prepared from L-aspartic acid and D-aspartic acid, respectively, by following the procedure described in Volkmann, R. A. et al. J. Org. Chem., 1992, 57, 4352-4361 for the preparation of (R)-(2-hydroxyethyl)oxirane (102A) from L-aspartic acid.

### (S)-2-Bromosuccinic Acid

To a solution of sodium bromide (15.46 g, 150.24 mmol) in 6N H₂SO₄ (33 mL) at 0° C. was added L-aspartic acid (5.00 g, 37.56 mmol). To the resultant mixture was added sodium nitrite (3.11 g, 45.07 mmol) portionwise over 90 min. The reaction mixture was allowed to stir at 0° C. for a further 2 h. The mixture was then diluted with H₂O (17 mL) and extracted with Et₂O (100 mL). The aqueous layer was diluted with brine (20 mL) and further extract with Et₂O (3×100 mL). The combined organic extracts were dried over anhydrous Na₂SO₄ and concentrated in vacuo to give (S)-2-bromosuccinic acid (2.98 g, 41%) as a white solid. The crude was used in subsequent synthetic steps without further purification. [α]_{D}^{19.7} −71.5 (c 0.46 in EtOAc) (lit −73.5 (c 6.0 in EtOAc); δ_{H} (400 MHz; DMSO) 12.8 (2H, br s, 2×CO₂H), 4.54 (1H, dd, J=8.5, 6.4 Hz, H-1), 3.10 (1H, dd, J=17.2, 8.6 Hz, H-2), 2.90 (1H, dd, J=17.1, 6.4 Hz, H-2); δ_{C} (100 MHz; DMSO) 171.0 (C, CO₂H), 170.1 (C, CO₂H), 40.5 (CH, C-1), 39.5 (CH₂, C-2). Spectroscopic data were consistent with those reported in literature.

### (R)-2-Bromosuccinic Acid

(R)-2-Bromosuccinic acid was prepared by following the procedure described above for the preparation of (S)-2-bromosuccinic acid, but using D-aspartic acid instead of L-aspartic acid. [α]_{D}^{20.2} +66.5 (c 0.2 in EtOAc). The remaining spectroscopic data was identical to that observed for (S)-2-bromosuccinic acid.

### (S)-2-Bromo-1,4-butanediol

To a solution of (S)-2-bromosuccinic acid (2.98 g, 15.20 mmol) in THF (35 mL) at −78° C. was added BH₃.DMS complex (4.33 mL, 45.61 mmol) dropwise over 90 min. The reaction was allowed to stir at −78° C. for 2 h and then warmed to r.t. and allowed to stir for a further 60 h. The reaction was then cooled to 0° C. and MeOH (15 mL) was added slowly. The mixture was then concentrated in vacuo and the residue diluted with MeOH (15 mL). This process was repeated 3 times to give the 2-bromo-1,4-butanediol (2.55 g, quant.) as a yellow oil. The crude was used in subsequent synthetic steps without further purification. [α]_{D}^{19.6} −36.8 (c 0.5 in CHCl₃); δ_{H} (400 MHz, CDCl₃) 4.34 (1H, dq, J=7.7, 5.3 Hz, H-2), 3.92-3.78 (4H, m, H-1, H-4), 2.40 (2H, br s, 2×OH), 2.20-2.06 (2H, m, H-3); δ_{C} (100 MHz; CDCl₃) 67.1 (CH₂, C-1), 60.1 (CH₂, C-4), 55.2 (CH, C-2), 37.8 (CH₂, C-3). Spectroscopic data were consistent with those reported in literature.

### (R)-2-Bromo-1,4-butanediol

(R)-2-Bromo-1,4-butanediol was prepared by following the procedure described above for the preparation of (S)-2-bromo-1,4-butanediol, but using (R)-2-bromosuccinic acid instead of (S)-2-bromosuccinic acid. [α]_{D}^{21.3} +20.0 (c 0.17 in CHCl₃). The remaining spectroscopic data was identical to that observed for (S)-2-bromo-1,4-butanediol.

### (R)-(2-Hydroxyethyl)oxirane (102A)

To a solution of (S)-2-bromo-1,4-butanediol (2.31 g, 13.76 mmol) in CH₂Cl₂ (46 mL) at r.t. was added Cs₂CO₃ (8.74 g, 24.77 mmol). The resultant mixture was allowed to stir at r.t. for 72 h. The reaction was then filtered through a pad of Celite® and concentrated in vacuo to give (R)-(2-hydroxyethyl)oxirane (102A) as a yellow oil with quantitative conversion. The crude material was used in subsequent synthetic steps without further purification. [α]_{D}^{22.9} +35.0 (c 0.61 in CHCl₃); δ_{H} (400 MHz; CDCl₃) 3.83-3.79 (2H, m, H-1), 3.12-3.08 (1H, m, H-3), 2.81 (1H, dd, J=4.8, 4.1 Hz, H-4), 2.60 (1H, dd, J=4.8, 2.8 Hz, H-4), 2.03-1.95 (1H, m, H-2), 1.78 (1H, t, J=5.4 Hz, OH), 1.71 (1H, dq, J=14.6, 5.9 Hz, H-2); δ_{C} (100 MHz; CDCl₃) 60.0 (CH₂, C-1), 50.5 (CH, C-3), 46.5 (CH₂, C-4), 34.6 (CH₂, C-2). Spectroscopic data were consistent with those reported in literature.

### (S)-(2-hydroxyethyl)oxirane (102B)

(S)-(2-Hydroxyethyl)oxirane (102B) was prepared by following the procedure described above for the preparation of (R)-(2-hydroxyethyl)oxirane (102A), but using (R)-2-bromo-1,4-butanediol instead of (S)-2-bromo-1,4-butanediol. [α]_{D}^{22.9} −35.2 (c 0.23 in CHCl₃). The remaining spectroscopic data was identical to that observed for (S)-2-bromo-1,4-butanediol.

### Preparation of Diastereomerically Pure 6A

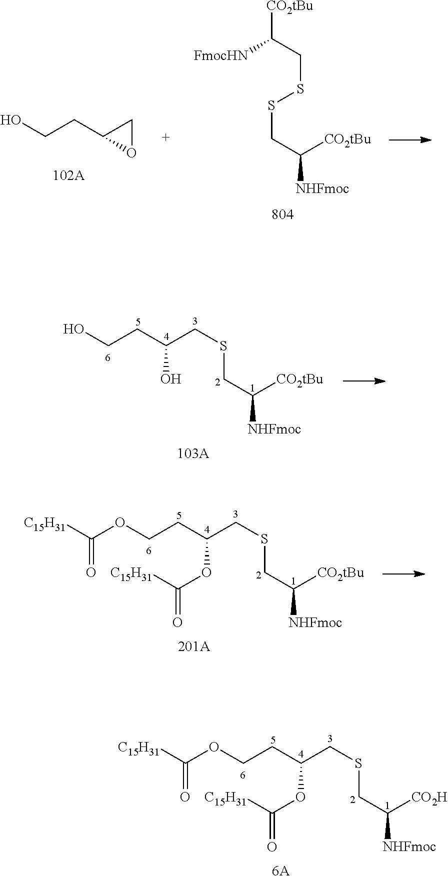

To a stirred solution of disulfide 804 (1.59 g, 2.06 mmol) in CH₂Cl₂ (10 mL) at 0° C. was added zinc powder (0.94 g, 14.42 mmol) and a freshly prepared mixture of methanol, conc. hydrochloric acid and conc. sulfuric acid (100:7:1, 5 mL). The resultant mixture was allowed to stir at 0° C. for 30 min after which was added epoxide 102A (0.73 g, 8.24 mmol). The reaction mixture was allowed to stir at 55° C. or refluxed at 70° C. for 17 h. The mixture was then diluted with CH₂Cl₂ (30 mL), filtered through a pad of Celite® and washed with brine (50 mL). The aqueous layer was extracted with CH₂Cl₂ (3×50 mL) and the combined organic extracts were dried over anhydrous MgSO₄ and concentrated in vacuo. The crude was purified by flash column chromatography (hexanes-EtOAc, 1:3) to give 103A (1.72 g, 88%) as a colourless oil.

R_{f} 0.15 (hexanes-EtOAc 1:3); [α]_{D}^{20.2} −3.5 (c 0.32 in CHCl₃); νₘₐₓ(neat)/cm⁻¹ 3347, 2976, 1703, 1518, 1449, 1413, 1369, 1335, 1249, 1151; 6 H (400 MHz; CDCl₃) 7.77 (2H, d, J=7.5, FmocH), 7.61 (2H, d, J=7.2 Hz, FmocH), 7.40 (2H, t, J=7.4 Hz, FmocH), 7.32 (2H, t, J=7.5 Hz, FmocH), 5.81 (1H, d, J=8.0 Hz, NH), 4.53-4.50 (1H, m, H-1), 4.40 (2H, d, J=6.8 Hz, FmocCH₂), 4.23 (1H, t, J=7.0 Hz, FmocCH), 3.94-3.88 (1H, m, H-4), 3.85-3.81 (2H, m, H-6), 3.03 (1H, dd, J=14.0, 4.2 Hz, H-2), 2.94 (1H, dd, J=14.3, 6.1 Hz, H-2), 2.82 (1H, dd, J=14.0, 2.9 Hz, H-3), 2.56 (1H, dd, J=14.0, 9.0 Hz, H-3), 1.74-1.71 (1H, m, H-5), 1.50 (9H, s, C(CH₃)₃); δ_{C} (100 MHz; CDCl₃) 169.7 (C, COOtBu), 156.0 (C, C(O)Fmoc), 143.6 (C, Fmoc), 141.0 (C, Fmoc), 127.5 (CH, Fmoc), 126.9 (CH, Fmoc), 125.0 (CH, Fmoc), 120.0 (CH, Fmoc), 82.6 (C, C(CH₃)₃), 69.5 (CH, C-4), 67.2 (CH₂, FmocCH₂), 60.2 (CH₂, C-6), 54.5 (CH, C-1), 46.9 (CH, FmocCH), 40.5 (CH₂, C-3), 37.5 (CH₂, C-5), 35.2 (CH₂, C-2), 27.8 (3×CH₃, C(CH₃)₃); HRMS (ESI+) [M+Na]⁺ 510.1921 talc for C₂₅H₃₃NNaO₆S 510.1921.

### Synthesis of Compound 6A (Procedure A)

To a stirred solution of diol 103A (1.52 g, 3.12 mmol) and palmitic acid (2.40 g, 9.35 mmol) in THF (45 mL) at r.t. was added N,N′-diisopropylcarbodiimide (1.93 mL, 12.46 mmol) and 4-dimethylaminopyridine (0.04 g, 0.31 mmol). The reaction mixture was allowed to stir at r.t. for 19 h. The mixture was then filtered through a pad of Celite®, diluted with EtOAc (50 mL), washed with 1M aq. citric acid (30 mL) and brine (30 mL) and concentrated in vacuo. The residue was then redissolved in TFA (3 mL) and allowed to stir at r.t. for 30 min. The reaction mixture was again concentrated in vacuo. The crude was purified by flash column chromatography (hexanes-EtOAc, 9:1→0:1) to give the title compound 6A (1.98 g, 70%) as a colorless oil. [α]_{D}^{23.9} +8.4 (c 0.44 in CHCl₃); νₘₐₓ (neat)/cm⁻¹ 2922, 2852, 1733, 1525, 1450, 1168, 1110; ¹H NMR (400 MHz, CDCl₃) δ 7.75 (2H, d, J=7.5 Hz), 7.61 (2H, d, J=7.2 Hz), 7.38 (2H, t, J=7.4 Hz), 7.30 (2H, t, J=7.4 Hz), 5.89 (1H, d, J=7.8 Hz), 5.13-5.06 (1H, m), 4.69-4.63 (1H, m), 4.39 (2H, d, J=6.5 Hz), 4.23 (1H, t, J=7.0 Hz), 4.16-4.06 (2H, m), 3.16 (1H, dd, J=13.8, 4.0 Hz), 3.03 (1H, dd, J=13.8, 6.1 Hz), 2.81-2.70 (2H, m), 2.33-2.26 (4H, m), 2.10-1.89 (2H, m), 1.60-1.58 (4H, m,), 1.35-1.20 (48H, m), 0.89 (6H, t, J=6.8 Hz); ¹³C NMR (100 MHz, CDCl₃) δ 174.0, 173.6, 156.0, 143.6, 141.2, 127.6, 127.0, 125.1, 120.0, 69.5, 67.3, 60.3, 53.6, 47.0, 36.3, 34.5, 34.3, 34.1, 32.0, 31.8, 29.6, 29.4, 29.3, 29.2, 29.1, 25.0, 22.6, 14.0; HRMS (ESI+) [M+H]⁺ 908.6069 calc for C₅₄H₈₆NO₆S 908.6065, [M+Na]⁺ 930.5888 calc for C₅₄H₈₅NNaO₈S 930.5875.

### Synthesis of Compound 6A (Procedure B)

Diastereomerically pure diol 103A was also converted to diastereomerically pure conjugate 6A by following procedures analogous to the representative procedures described below.

### Representative Procedure for Conversion of 103A to 201A

To a stirred solution of diol 103A (0.327 g, 0.67 mmol) and palmitic acid (0.516 g, 2.01 mmol) in THF (9 mL) at r.t. is added diisopropylcarbodiimide (0.414 mL, 2.68 mmol) and 4-dimethylaminopyridine (0.01 g, 0.07 mmol). The reaction mixture is allowed to stir at r.t. for 19 h. The mixture is then diluted with EtOAc (30 mL), filtered through a bed of Celite® and concentrated in vacuo. The crude is purified by flash column chromatography (CH₂Cl₂) to give 201A as yellow oil.

### Representative Procedure for Conversion of 201A to 6A

A solution of diester 201A (0.35 g, 0.364 mmol) in trifluoroacetic acid (2 mL) is allowed to stir at r.t. for 1 h after which the mixture is concentrated in vacuo. The crude is purified by flash column chromatography (hexanes-EtOAc, 9:1→0:1) to give 6A as a colourless oil.

Fmoc-Cys-OH is described in the literature: H.-K. Cui, Y. Guo, Y. He, F.-L. Wang, H.-H. Chang, Y. J. Wang, F.-M. Wu, C.-L. Tian, L. Lu, Angew. Chem. Int. Eng., 2013, 52(36), 9558-9562.

### Preparation of Diastereomerically Pure 6B

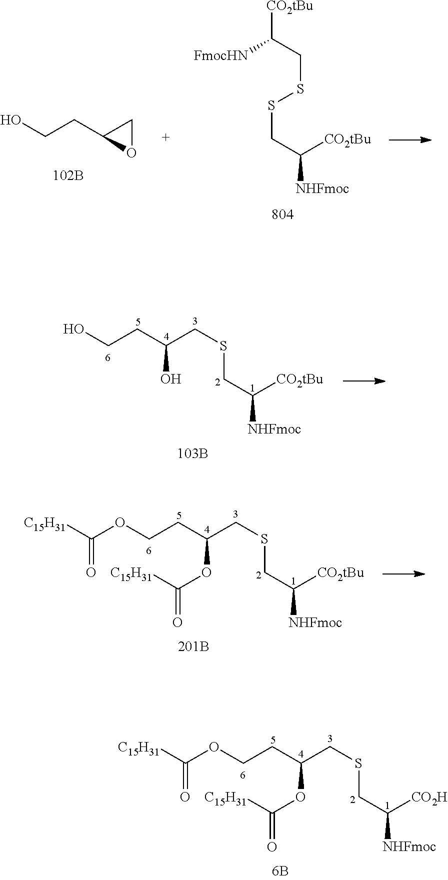

### Synthesis of Compound 103B

To a stirred solution of disulfide 804 (2.01 g, 2.53 mmol) in CH₂Cl₂ (14 mL) at 0° C. was added zinc powder (1.15 g, 17.51 mmol) and a freshly prepared mixture of methanol, conc. hydrochloric acid and conc. sulfuric acid (100:7:1, 7 mL). The resultant mixture was allowed to stir at 0° C. for 30 min after which was added epoxide 102B (0.89 g, 10.11 mmol). The reaction mixture was allowed to stir at 55° C. or refluxed at 70° C. for 17 h. The mixture was then diluted with CH₂Cl₂ (30 mL), filtered through a pad of Celite® and washed with brine (50 mL). The aqueous layer was extracted with CH₂Cl₂ (3×50 mL) and the combined organic extracts were dried over anhydrous MgSO₄ and concentrated in vacuo. The crude was purified by flash column chromatography (hexanes-EtOAc, 3:1) to give the 103B (2.17 g, 88%) as a colourless oil.

R_{f} 0.15 (hexanes-EtOAc 1:3); [α]_{D}²² +8.5 (c 0.3 in CHCl₃); νₘₐₓ(neat)/cm⁻¹ 3347, 2976, 1703, 1518, 1449, 1413, 1369, 1335, 1249, 1151; 6 H (400 MHz; CDCl₃) 7.77 (2H, d, J=7.5 Hz, FmocH), 7.61 (2H, d, J=7.4 Hz, FmocH), 7.40 (2H, t, J=7.4 Hz, FmocH), 7.32 (2H, t, J=7.5 Hz, FmocH), 5.74 (1H, d, J=7.0 Hz, NH), 4.51-4.47 (1H, m, H-1), 4.42-4.39 (2H, m, FmocCH₂), 4.24 (1H, t, J=7.0 Hz, FmocCH), 3.93 (1H, br s, H-4), 3.85-3.81 (2H, m, H-6), 3.31 (1H, br s, OH-4), 3.00-2.78 (2H, m, H-2), 2.80 (1H, dd, J=13.5, 3.2 Hz, H-3), 2.55 (1H, dd, J=13.8, 8.4, Hz, H-3), 2.36 (1H, br s, OH-6) 1.73 (2H, q, J=5.3, H-5), 1.50 (9H, s, C(CH₃)₃); HRMS δ_{C} (100 MHz; CDCl₃) 169.8 (C, COOtBu), 156.1 (C, C(O)Fmoc), 143.8 (C, Fmoc), 141.3 (C, Fmoc), 127.7 (CH, Fmoc), 127.1 (CH, Fmoc), 125.1 (CH, Fmoc), 120.0 (CH, Fmoc), 83.0 (C, C(CH₃)₃), 69.9 (CH, C-4), 67.2 (CH₂, FmocCH₂), 60.7 (CH₂, C-6), 54.7 (CH, C-1), 47.1 (CH, FmocCH), 40.9 (CH₂, C-3), 37.6 (CH₂, C-5), 35.5 (CH₂, C-2), 28.0 (3×CH₃, C(CH₃)₃); HRMS (ESI+) [M+Na]⁺ 510.1921 calc for C₂₆H₃₃NNaO₆S 510.1921.

### Synthesis of Compound 6B (Procedure A)

To a stirred solution of diol 103B (1.68 g, 3.44 mmol) and palmitic acid (2.65 g, 10.31 mmol) in THF (50 mL) at r.t. was added N,N′-diisopropylcarbodiimide (2.13 mL, 13.75 mmol) and 4-dimethylaminopyridine (0.04 g, 0.34 mmol). The reaction mixture was allowed to stir at r.t. for 19 h. The mixture was then filtered through a pad of Celite®, diluted with EtOAc (50 mL), washed with 1M aq. citric acid (30 mL) and brine (30 mL) and concentrated in vacuo. The residue was then redissolved in TFA (3 mL) and allowed to stir at r.t. for 30 min. The reaction mixture was again concentrated in vacuo. The crude was purified by flash column chromatography (hexanes-EtOAc, 9:1→0:1) to give the title compound 6B (2.02 g, 65%) as a colorless oil. [α]_{D}^{23.4} −4.0 (c 0.2 in CHCl₃); νₘₐₓ(neat)/cm⁻¹ 2922, 2852, 1733, 1525, 1450, 1168, 1110; ¹H NMR (400 MHz, CDCl₃) δ 7.75 (2H, d, J=7.5 Hz), 7.61 (2H, d, J=7.4 Hz), 7.39 (2H, t, J=7.4 Hz), 7.40 (2H, t, J=7.4 Hz), 5.86 (1H, d, J=7.5), 5.13-5.03 (1H, m), 4.72-4.62 (1H, m), 4.40 (2H, d, J=6.9 Hz), 4.24 (1H, t, J=7.0 Hz), 4.11 (2H, t, J=6.5 Hz), 3.14 (1H, dd, J=12.9, 3.5 Hz), 3.08 (1H, dd, J=13.8, 4.4 Hz), 2.82-2.69 (2H, m), 2.31-2.26 (4H, m), 2.10-1.87 (2H, m), 1.65-1.54 (4H, m), 1.33-1.22 (48H, m), 0.89 (6H, t, J=6.8 Hz); ¹³C NMR (100 MHz, CDCl₃) δ 174.1, 173.7, 155.0, 143.7, 141.3, 127.8, 127.1, 125.2, 120.0, 69.7, 67.5, 60.5, 53.7, 47.1, 36.5, 34.8, 34.4, 34.3, 32.2, 32.0, 29.8, 29.7, 29.6, 29.4, 29.2, 25.0, 22.8, 14.1; HRMS (ESI+) [M+H]⁺ 908.6069 calc for C₅₄H₈₆NO₆S 908.6065, [M+Na]⁺ 930.5888 calc for C₅₄H₈₅NNaO₈S 930.5875.

### Synthesis of Compound 6B (Procedure B)

Diastereomerically pure diol 103B was also converted to diastereomerically pure conjugate 6B by following procedures analogous to the representative procedures described below.

### Representative Procedure for Conversion of 103B to 201B

To a stirred solution of diol 103B (0.327 g, 0.67 mmol) and palmitic acid (0.516 g, 2.01 mmol) in THF (9 mL) at r.t. is added diisopropylcarbodiimide (0.414 mL, 2.68 mmol) and 4-dimethylaminopyridine (0.01 g, 0.07 mmol). The reaction mixture is allowed to stir at r.t. for 19 h. The mixture is then diluted with EtOAc (30 mL), filtered through a bed of Celite® and concentrated in vacuo. The crude is purified by flash column chromatography (CH₂Cl₂) to give 201B as yellow oil.

### Representative Procedure for Conversion of 201B to 6B

A solution of diester 201B (0.35 g, 0.364 mmol) in trifluoroacetic acid (2 mL) is allowed to stir at r.t. for 1 h after which the mixture is concentrated in vacuo. The crude is purified by flash column chromatography (hexanes-EtOAc, 9:1→0:1) to give 6B as a colourless oil.

### 2. Example 2

This example demonstrates the synthesis of amino acid conjugates from various starting materials.

### 7.1 Synthesis of Amino Acid Conjugate 806 from Alcohol 800

### Step i

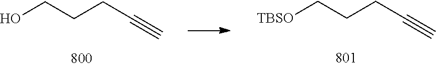

To a stirred solution of 4-pentyn-1-ol 800 (5 mL, 53.72 mmol) in CH₂Cl₂ (150 mL) at r.t. was added imidazole (3.66 g, 53.72 mmol) and tert-butyldimethylsilyl chloride (8.10 g, 53.72 mmol). The reaction mixture was allowed to stir at r.t. for 24 h. The mixture was then diluted with Et₂O (200 mL) and washed with water (3×100 mL) and brine (100 mL). The organic layer was dried over anhydrous Na₂SO₄ and concentrated in vacuo. The crude was purified by filtration through silica gel to give 801 (10.64 g, quant.) as a colourless liquid. Alkyne 801 was used in subsequent synthetic steps without characterisation.

### Step ii

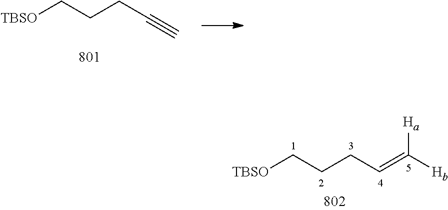

To a stirred solution of alkyne 801 (14.08 g, 70.00 mmol) in hexanes (150 mL) at r.t. was added quinoline (11.75 mL, 100.00 mmol) and Lindar's catalyst (1.408 g). The reaction mixture was connected to a Hz-filled balloon (1 atm) and allowed to stir at r.t. for 5 h. The mixture was then filtered through a pad of Celite® and concentrated in vacuo. The crude product was purified by flash column chromatography (petroleum ether-EtOAc, 9:1) to give 802 (14.09 g, 99%) as a colourless liquid.

R_{f} 0.88 (petroleum ether-EtOAc 9:1); δ_{H} (400 MHz; CDCl₃) 5.82 (1H, ddt, J=17.0, 10.2, 6.7 Hz, H-4), 5.02 (1H, d, J=17.1 Hz, Ha-5), 4.95 (1H, d, J=10.4 Hz, H_{b}-5), 3.62 (2H, t, J=6.5 Hz, H-1), 2.10 (2H, q, J=7.2 Hz, H-3), 1.61 (2H, p, J=7.0 Hz, H-2), 0.90 (9H, s, SiC(CH₃)₃), 0.05 (6H, s, Si(CH₃)₂); δ_{C} (100 MHz; CDCl₃) 138.6 (CH, C-4), 114.5 (CH₂, C-5), 62.6 (CH₂, C-1), 32.0 (CH₂, C-2), 30.5 (CH₂, C-3), 26.0 (3×CH₃, SiC(CH₃)₃), 18.4 (C, SiC(CH₃)₃), −5.3 (2×CH₃, Si(CH₃)₂). Spectroscopic data were consistent with those reported in literature.

### Step iii

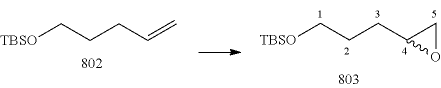

To a stirred solution of alkene 802 (8.646 g, 43.16 mmol) in CH₂Cl₂ (100 mL) at r.t. was added mCPBA (8.191 g, 47.47 mmol). The reaction mixture was allowed to stir at r.t. for 15 h. The mixture was then filtered through Celite®, diluted with Et₂O (100 mL) and washed with sat. aq. NaHCO₃ (3×100 mL) and brine (100 mL). The organic layer was dried over anhydrous Na₂SO₄ and concentrated in vacuo. The crude product was purified by flash column chromatography (petroleum ether-EtOAc, 9:1) to give 803 (8.09 g, 87%) as a colourless liquid.

R_{f} 0.51 (petroleum ether-EtOAc 9:1); δ_{H} (400 MHz; CDCl₃) 3.70-3.60 (2H, m, H-1), 2.96-2.92 (1H, m, H-4), 2.75 (1H, dd, J=5.0, 4.0 Hz, H-5), 2.47 (1H, dd, J=5.0, 2.8 Hz, H-5), 1.73-1.53 (4H, m, H-2, H-3), 0.89 (9H, s, SiC(CH₃)₃), 0.04 (6H, s, Si(CH₃)₂); ac (100 MHz; CDCl₃) 62.7 (CH₂, C-1), 52.2 (CH, C-4), 47.1 (CH₂, C-5), 29.1 (CH₂, C-2), 29.0 (CH₂, C-3), 25.9 (3×CH₃, SiC(CH₃)₃), 18.3 (C, SiC(CH₃)₃), −5.3 (2×CH₃, Si(CH₃)₂). Spectroscopic data were consistent with those reported in literature.

### Step iv

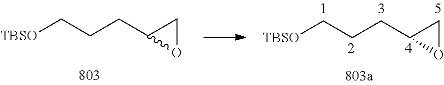 [SMILES: CC(C)(C)[Si](C)(C)OCCCC1CO1 ; CC(C)(C)[Si](C)(C)OCCC[C@@H]1CO1]

To a stirred solution of racemic epoxide 803 (8.272 g, 38.24 mmol), (R,R)-(+)-N,N′-bis(3,5-di-tert-butylsalicylidene)-1,2-cyclohexanediaminocobalt(II) (0.121 g, 0.19 mmol) and glacial acetic acid (0.04 mL, 0.76 mmol) in THF (0.35 mL) at 0° C. was added water (0.38 mL) dropwise. The reaction mixture was allowed to stir at r.t. for 48 h. The mixture was then concentrated in vacuo. The crude product was purified by flash column chromatography (petroleum ether-EtOAc, 9:1) to give 803a (4.12 g, 49%) as a yellow oil.

R_{f}0.51 (petroleum ether-EtOAc 9:1); [α]_{D}^{21.4} +4.65 (c 1.15 in CHCl₃); δ_{H} (400 MHz; CDCl₃) 3.61 (2H, t, J=6.0 Hz, H-1), 2.93-2.88 (1H, m, H-4), 2.74 (1H, dd, J=5.0, 4.0 Hz, H-5), 2.46 (1H, dd, J=5.0, 3.0 Hz, H-5), 1.63-1.46 (4H, m, H-2, H-3), 0.89 (9H, s, SiC(CH₃)₃), 0.04 (6H, s, Si(CH₃)₂); δ_{C} (100 MHz; CDCl₃) 63.0 (CH₂, C-1), 52.3 (CH, C-4), 47.1 (CH₂, C-5), 32.6 (CH₂, C-2), 32.3 (CH₂, C-3), 26.0 (3×CH₃, SiC(CH₃)₃), 18.4 (C, SiC(CH₃)₃), −5.3 (2×CH₃, Si(CH₃)₂). Spectroscopic data were consistent with those reported in literature.

### Step v

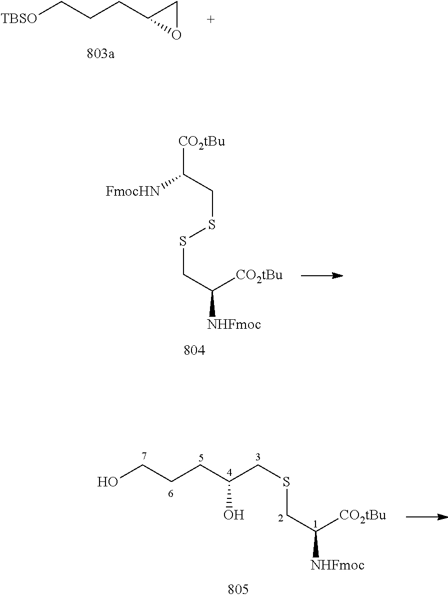

To a stirred solution of disulfide 804 (0.751 g, 0.94 mmol), which is commercially available, in CH₂Cl₂ (5 mL) at 0° C. was added zinc powder (0.508 g, 7.78 mmol) and a freshly prepared mixture of methanol, conc. hydrochloric acid and conc. sulfuric acid (100:7:1, 2 mL). The resultant mixture was allowed to stir at 0° C. for 30 min. The mixture was then allowed to stir at 70° C. or 65° C. for 5 min after which was added epoxide 803a (0.839 g, 3.88 mmol). The reaction mixture was allowed to stir at 65° C. for 19 h. The mixture was then diluted with EtOAc (50 mL), filtered through a pad of Celite® and washed with brine (50 mL). The aqueous layer was extracted with EtOAc (3×50 mL) and the combined organic extracts were dried over anhydrous Na₂SO₄ and concentrated in vacuo. The crude product was purified by flash column chromatography (hexanes-EtOAc, 1:3) to give 805 (0.568 g, 60%) as a colourless oil.

R_{f} 0.34 (hexane-EtOAc 1:3); [α]_{D}^{21.0} −26.7 (c 0.03 in CHCl₃); νₘₐₓ(neat)/cm⁻¹ 3321, 2931, 1706, 1532, 1450, 1369, 1248, 1152, 1050; 6 H (400 MHz; CHCl₃) 7.76 (2H, d, J=7.5 Hz, FmocH), 7.61 (2H, d, J=7.2 Hz, FmocH), 7.40 (2H, t, J=7.4 Hz, FmocH), 7.31 (2H, t, J=7.4 Hz, FmocH), 5.90 (1H, d, J=7.8 Hz, NH), 4.51 (1H, dd, J=12.3, 5.2 Hz, H-1), 4.39 (2H, d, J=7.1 Hz, FmocCH₂), 4.23 (1H, t, J=7.1 Hz, FmocCH), 3.73-3.58 (3H, m, H-4, H-7), 3.03 (1H, dd, J=13.9, 4.4 Hz, H-2), 2.95 (1H, dd, J=13.9, 5.7 Hz, H-2), 2.80 (1H, dd, J=13.6, 2.9 Hz, H-3), 2.53 (1H, dd, J=13.6, 8.9 Hz, H-3), 1.72-1.61 (4H, m, H-5, H-6), 1.49 (9H, s, C(CH₃)₃)); δ_{C} (100 MHz; CHCl₃) 169.8 (C, CO₂tBu), 156.1 (C, FmocCO), 143.9 (C, Fmoc), 141.1 (C, Fmoc), 127.9 (CH, Fmoc), 127.2 (CH, Fmoc), 125.3 (CH, Fmoc), 120.1 (CH, Fmoc), 83.2 (C, C(CH₃)₃), 70.1 (CH, C-4), 67.3 (CH₂, FmocCH₂), 62.8 (CH₂, C-7), 54.7 (CH, C-1), 47.2 (CH, FmocCH), 41.2 (CH₂, C-3), 35.5 (CH₂, C-2), 33.4 (CH₂, C-5), 29.2 (CH₂, C-6), 28.1 (3×CH₃, C(CH₃)₃); HRMS (ESI+) [M+Na]⁺ 524.2077 talc for C₂₇H₃₅NNaO₆S 524.2075.

### Step vi

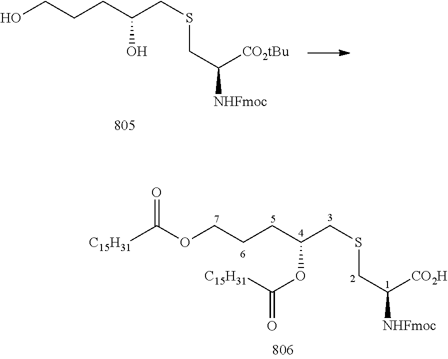

To a stirred solution of diol 805 (0.114 g, 0.243 mmol) and palmitic acid (0.180 g, 0.702 mmol) in THF (3 mL) at r.t. was added N,N′-diisopropylcarbodiimide (0.145 mL, 0.936 mmol) and 4-dimethylaminopyridine (0.011 g, 0.094 mmol). The reaction mixture was allowed to stir at r.t. for 17 h. The mixture was then filtered through a pad of Celite®, diluted with EtOAc (30 mL), washed with 1M aq. citric acid (30 mL) and brine (30 mL) and concentrated in vacuo. The residue was then redissolved in TFA (3 mL) and allowed to stir at r.t for 45 min. The reaction mixture was again concentrated in vacuo. The crude product was purified by flash column chromatography (hexanes-EtOAc, 9:1→0:1) to give 806 (0.220 g, 98%) as a colourless oil.

R_{f} 0.15 (petroleum ether-EtOAc 1:1); [α]_{D}^{21.3} +10.0 (c 0.08 in CHCl₃); νₘₐₓ(neat)/cm⁻¹ 2919, 2851, 1723, 1521, 1521, 1221, 1108, 1054; 6 H (400 MHz; CHCl₃) 7.76 (2H, d, J=7.5 Hz, FmocH), 7.62 (2H, d, J=7.4 Hz, FmocH), 7.39 (2H, t, J=7.4 Hz, FmocH), 7.30 (2H, td, J=11.2, 0.9 Hz, FmocH), 5.78 (1H, d, J=7.6 Hz, NH), 5.04-4.95 (1H, m, H-4), 4.60 (1H, dd, J=12.2, 5.2 Hz, H-1), 4.38 (2H, d, J=7.2 Hz, FmocCH₂), 4.24 (2H, t, J=7.1 Hz, FmocCH), 4.13-3.99 (2H, m, H-7), 3.16 (1H, dd, J=13.9, 4.5 Hz, H-2), 3.04 (1H, dd, J=14.0, 5.3 Hz, H-2), 2.78-2.70 (2H, m, H-3), 2.34-2.25 (4H, m, 2×PamCH_{2α}alkyl), 1.74-1.56 (8H, m, 2×PamCH_{2β}alkyl, H-5, H-6), 1.32-1.22 (48H, m, 24×PamCH₂alkyl), 0.88 (6H, t, J=6.9 Hz, 2×PamCH₃alkyl); δ_{C} (100 MHz; CHCl₃) 174.3 (C, CO₂H), 174.0 (C, PamCO₂), 173.5 (C, PamCO₂), 156.0 (C, FmocCO), 143.7 (C, Fmoc), 141.3 (C, Fmoc), 127.8 (CH, Fmoc), 127.1 (CH, Fmoc), 121.2 (CH, Fmoc), 120.0 (CH, Fmoc), 72.1 (CH, C-4), 67.5 (CH₂, FmocCH₂), 63.8 (CH₂, C-7), 53.6 (CH, C-1), 47.1 (CH, FmocCH), 36.5 (CH₂, C-3), 34.6 (CH₂, PamCH_{2α}alkyl), 34.5 (CH₂, PamCH_{2α}alkyl), 34.3 (CH₂, C-2), 31.9 (2×CH₂, PamCH₂alkyl), 29.7-29.2 (21×CH₂, PamCH₂alkyl, C-5), 25.0 (2×CH₂, PamCH_{2β}alkyl), 24.6 (CH₂, C-6), 22.7 (2×CH₂, PamCH₂alkyl), 14.1 (2×CH₃, PamCH₃alkyl); HRMS (ESI+) [M+Na]⁺ 944.6045 calc for C₅₅H₈₇NNaO₈S 944.6028.

### 7.1.2 Synthesis of Amino Acid Conjugate 811 from Alcohol 807

### Step i

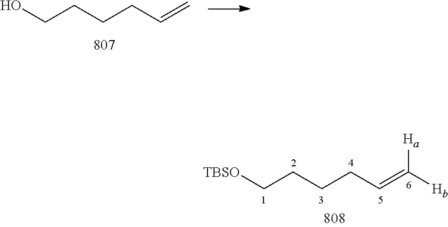

To a stirred solution of 5-hexen-1-ol 807 (5.00 mL, 41.64 mmol) in CH₂Cl₂ (150 mL) at r.t. was added imidazole (2.86 g, 43.06 mmol) and tert-butyldimethylsilyl chloride (6.34 g, 42.06 mmol). The reaction mixture was allowed to stir at r.t. for 19 h. The mixture was then diluted with EtOAc (400 mL), washed with water (200 mL) and brine (200 mL), dried over anhydrous Na₂SO₄and concentrated in vacuo. The crude product was purified by flash column chromatography (petroleum ether) to give 808 (8.846 g, quant.) as a colourless oil.

R_{f} 0.90 (petroleum ether-EtOAc 9:1); δ_{H} (400 MHz; CDCl₃) 5.81 (1H, ddt, J=17.1, 10.1, 6.7 Hz, H-5), 5.00 (1H, dq, J=17.2, 1.7 Hz, Hₐ-6), 4.94 (1H, d, J=10.5 Hz, H_{b}-6), 3.61 (2H, t, J=6.2 Hz, H-1), 2.06 (2H, q, J=7.1 Hz, H-4), 1.59-1.50 (2H, m, H-2), 1.47-1.39 (2H, m, H-3), 0.89 (9H, s, SiC(CH₃)₃), 0.05 (6H, s, Si(CH₃)₂); δ_{C} (100 MHz; CDCl₃) 139.0 (CH, C-5), 114.3 (CH₂, C-6), 63.1 (CH₂, C-1), 33.5 (CH₂, C-4), 32.3 (CH₂, C-2), 26.0 (3×CH₃, SiC(CH₃)₃), 25.2 (CH₂, C-3), 18.4 (C, SiC(CH₃)₃), −5.3 (2×CH₃, Si(CH₃)₂). Spectroscopic data were consistent with those reported in literature.

### Step ii

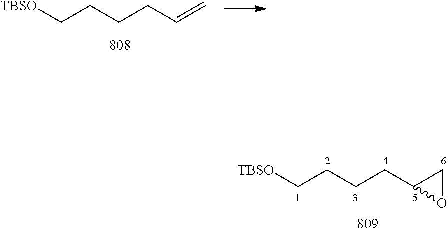

To a stirred solution of alkene 808 (7.58 g, 35.35 mmol) in CH₂Cl₂ (150 mL) at r.t. was added mCPBA (9.15 g, 53.05 mmol) portionwise. The reaction mixture was allowed to stir at r.t. for 18 h. The mixture was then diluted with Et₂O (200 mL), filtered through Celite®, washed with 2M aq. NaOH (200 mL) and brine (200 mL), dried over anhydrous Na₂SO₄ and concentrated in vacuo. The crude product was purified by flash column chromatography (petroleum ether-EtOAc, 9:1) to give 809 (6.91 g, 85%) as a colourless oil.

R_{f} 0.60 (petroleum ether-EtOAc 9:1); δ_{H} (400 MHz; CDCl₃) 3.61 (2H, t, J=6.0 Hz, H-1), 2.93-2.88 (1H, m, H-5), 2.74 (1H, dd, J=5.0, 4.0 Hz, H-6), 2.46 (1H, dd, J=5.0, 3.0 Hz, H-6), 1.63-1.46 (6H, m, H-2, H-3, H-4), 0.89 (9H, s, SiC(CH₃)₃), 0.04 (6H, s, Si(CH₃)₂); δ_{C} (100 MHz; CDCl₃) 63.0 (CH₂, C-1), 52.3 (CH, C-5), 47.1 (CH₂, C-6), 32.6 (CH₂, C-4), 32.3 (CH₂, C-2), 26.0 (3×CH₃, SiC(CH₃)₃), 22.3 (CH₂, C-3), 18.4 (C, SiC(CH₃)₃), −5.3 (2×CH₃, Si(CH₃)₂). Spectroscopic data were consistent with those reported in literature.

### Step iii

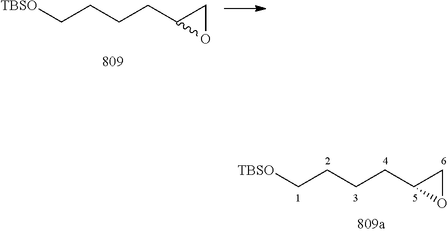 [SMILES: CC(C)(C)[Si](C)(C)OCCCCC1CO1 ; CC(C)(C)[Si](C)(C)OCCCC[C@@H]1CO1]

To a stirred solution of racemic epoxide 809 (5.887 g, 25.56 mmol), (R,R)-(+)-N,N′-bis(3,5-di-tert-butylsalicylidene)-1,2-cyclohexanediaminocobalt(II) (0.083 g, 0.13 mmol) and glacial acetic acid (0.03 mL, 0.51 mmol) in THF (0.3 mL) at 0° C. was added water (0.253 mL) dropwise. The reaction mixture was allowed to stir at r.t. for 48 h. The mixture was then concentrated in vacuo. The crude product was purified by flash column chromatography (petroleum ether-EtOAc, 9:1) to give 809a (2.913 g, 49%) as a yellow oil.

R_{f} 0.60 (petroleum ether-EtOAc 9:1); [α]_{D}^{20.4} +5.0 (c 0.02 in CHCl₃); δ_{H} (400 MHz; CDCl₃) 3.61 (2H, t, J=6.0 Hz, H-1), 2.93-2.88 (1H, m, H-5), 2.74 (1H, dd, J=5.0, 4.0 Hz, H-6), 2.46 (1H, dd, J=5.0, 3.0 Hz, H-6), 1.63-1.46 (6H, m, H-2, H-3, H-4), 0.89 (9H, s, SiC(CH₃)₃), 0.04 (6H, s, Si(CH₃)₂); δ_{C} (100 MHz; CDCl₃) 63.0 (CH₂, C-1), 52.3 (CH, C-5), 47.1 (CH₂, C-6), 32.6 (CH₂, C-4), 32.3 (CH₂, C-2), 26.0 (3×CH₃, SiC(CH₃)₃), 22.3 (CH₂, C-3), 18.4 (C, SiC(CH₃)₃), −5.3 (2×CH₃, Si(CH₃)₂). Spectroscopic data were consistent with those reported in literature.

### Step iv

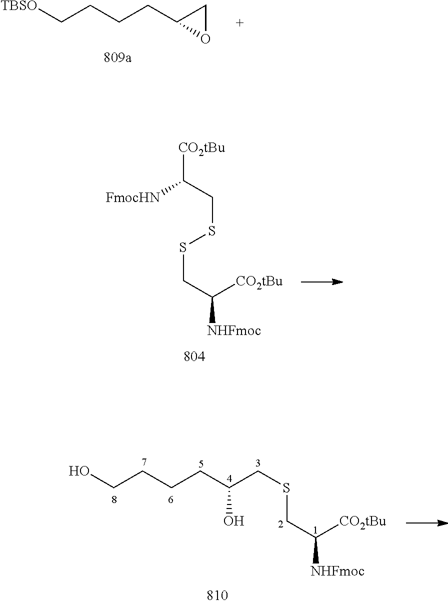

To a stirred solution of disulfide 804 (0.500 g, 0.649 mmol) in CH₂Cl₂ (5 mL) at 0° C. was added zinc powder (0.300 g, 4.54 mmol) and a freshly prepared mixture of methanol, conc. hydrochloric acid and conc. sulfuric acid (100:7:1, 2 mL). The resultant mixture was allowed to stir at 0° C. for 30 min. The mixture was then allowed to stir at 65° C. for 5 min after which was added epoxide 809a (0.600 g, 2.60 mmol). The reaction mixture was allowed to stir at 65° C. for 19 h. The mixture was then diluted with EtOAc (50 mL), filtered through a pad of Celite® and washed with brine (50 mL). The aqueous layer was extracted with EtOAc (3×50 mL) and the combined organic extracts were dried over anhydrous Na₂SO₄ and concentrated in vacuo. The crude product was purified by flash column chromatography (hexanes-EtOAc, 4:1→1:3) to give 810 (0.553 g, 83%) as a colourless oil.

R_{f} 0.39 (hexane-EtOAc 1:3); [α]_{D}^{21.2} −25.0 (c 0.07 in CHCl₃); νₘₐₓ(neat)/cm⁻¹ 3343, 2934, 2862, 1705, 1513, 1450, 1369, 1344, 1248, 1152; δ_{H} (400 MHz; CHCl₃) 7.76 (2H, d, J=7.5 Hz, FmocH), 7.61 (2H, d, J=7.0 Hz, FmocH), 7.40 (2H, t, J=7.4 Hz, FmocH), 7.30 (2H, td, J=11.2, 1.1 Hz, FmocH), 5.88 (1H, d, J=7.8 Hz, NH), 4.52 (1H, dd, J=12.5, 5.2 Hz, H-1), 4.39 (2H, d, J=8.1 Hz, FmocCH₂), 4.23 (1H, t, J=7.1 Hz, FmocCH), 3.70-3.59 (3H, m, H-4, H-8), 3.03 (1H, dd, J=13.7, 4.7 Hz, H-2), 2.94 (1H, dd, J=13.7, 5.4 Hz, H-2), 2.80 (1H, dd, J=13.6, 3.4 Hz, H-3), 2.51 (1H, dd, J=13.4, 8.7 Hz, H-3), 1.60-1.38 (15H, m, H-5, H-6, H-7, C(CH₃)₃)); δ_{C} (100 MHz; CHCl₃) 169.7 (C, CO₂tBu), 156.0 (C, FmocCO), 143.8 (C, Fmoc), 141.3 (C, Fmoc), 127.8 (CH, Fmoc), 127.1 (CH, Fmoc), 125.2 (CH, Fmoc), 120.0 (CH, Fmoc), 83.1 (C, C(CH₃)₃), 69.8 (CH, C-4), 67.2 (CH₂, FmocCH₂), 62.5 (CH₂, C-8), 54.6 (CH, C-1), 47.1 (CH, FmocCH), 41.1 (CH₂, C-3), 35.8 (CH₂, C-5), 35.4 (CH₂, C-2), 32.4 (CH₂, C-7), 28.0 (3×CH₃, C(CH₃)₃), 21.9 (CH₂, C-6); HRMS (ESI+) [M+Na]⁺ 538.2226 talc for C₂₈H₃₇NNaO₆S 538.2234.

### Step v

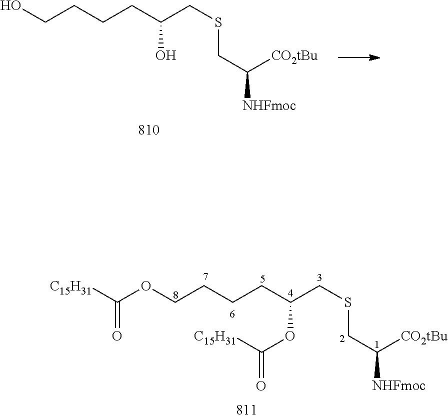

To a stirred solution of diol 810 (0.190 g, 0.370 mmol) and palmitic acid (0.284 g, 1.10 mmol) in THF (3 mL) at r.t. was added N,N′-diisopropylcarbodiimide (0.226 mL, 1.47 mmol) and 4-dimethylaminopyridine (0.018 g, 0.147 mmol). The reaction mixture was allowed to stir at r.t. for 17 h. The mixture was then filtered through a pad of Celite®, diluted with EtOAc (50 mL), washed with 1M aq. citric acid (30 mL) and brine (30 mL) and concentrated in vacuo. The residue was then redissolved in TFA (3 mL) and allowed to stir at r.t. for 45 min. The reaction mixture was again concentrated in vacuo. The crude product was purified by flash column chromatography (hexanes-EtOAc, 9:1→0:1) to give 811 (0.301 g, quant.) as a colourless oil.

R_{f} 0.20 (petroleum ether-EtOAc 1:1); [α]_{D}^{21.2} +10.0 (c 0.07 in CHCl₃); νₘₐₓ(neat)/cm⁻¹ 3331, 2917, 2850, 1728, 1692, 1532, 1467, 1451, 1244, 1221, 1198, 1175; 6 H (400 MHz; CHCl₃) 7.76 (2H, d, J=7.5 Hz, FmocH), 7.62 (2H, d, J=7.2 Hz, FmocH), 7.40 (2H, t, J=7.4 Hz, FmocH), 7.30 (2H, td, J=11.2, 1.0 Hz, FmocH), 5.82 (1H, d, J=7.9 NH), 5.03-4.92 (1H, m, H-4), 4.71-4.60 (1H, m, H-1), 4.40 (2H, d, J=7.0 Hz, FmocCH₂), 4.24 (1H, t, J=7.1 Hz, FmocCH), 4.11-4.00 (2H, m, H-8), 3.15 (1H, dd, J=13.9, 4.4 Hz, H-2), 3.04 (1H, dd, J=13.8, 5.8 Hz, H-2), 2.78-2.65 (2H, m, H-3), 2.31 (2H, t, J=7.6 Hz, PamCH_{2α}alkyl), 2.28 (2H, t, J=7.6 Hz, PamCH_{2α}alkyl), 1.74-1.55 (8H, m, 2×PamCH_{2β}alkyl, H-5, H-7), 1.45-1.17 (50H, m, 24×PamCH₂alkyl, H-6), 0.88 (6H, t, J=6.8 Hz, 2×PamCH₃alkyl); δ_{C} (100 MHz; CHCl₃) 174.3 (C, CO₂H), 174.0 (C, PamCO₂), 173.9 (C, PamCO₂), 156.1 (C, FmocCO), 143.7 (C, Fmoc), 141.3 (C, Fmoc), 127.8 (CH, Fmoc), 127.1 (CH, Fmoc), 125.2 (CH, Fmoc), 120.0 (CH, Fmoc), 72.4 (CH, C-4), 67.4 (CH₂, FmocCH₂), 64.0 (CH₂, C-8), 53.6 (CH, C-1), 47.1 (CH, FmocCH), 36.6 (CH₂, C-3), 34.6 (CH₂, PamCH_{2α}alkyl), 34.5 (CH₂, PamCH_{2α}alkyl), 34.4 (CH₂, C-2), 32.7 (CH₂, C-5), 32.0 (2×CH₂, PamCH₂alkyl), 29.7-29.3 (20×CH₂, PamCH₂alkyl), 28.3 (CH₂, C-7), 25.0 (2×CH₂, PamCH_{2β}alkyl), 25.0 (2×CH₂, PamCH_{2β}alkyl), 22.7 (2×CH₂, PamCH₂alkyl), 21.7 (CH₂, C-6), 14.4 (2×CH₃, PamCH₃alkyl); HRMS (ESI+) [M+Na]⁺ 958.6239 talc for C₅₆H₈₉NNaO₈S 958.6238.

### 7.1.3 Synthesis of Amino Acid Conjugate 820 from Alkene 814

### A) Synthesis of Alkene 814 from Alcohol 812

### Step i

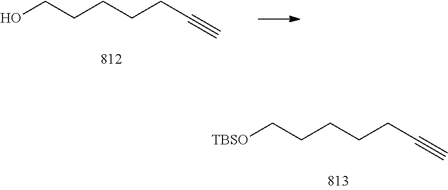

To a stirred solution of 6-heptyn-1-ol 812 (3.33 mL, 26.75 mmol) in CH₂Cl₂ (80 mL) at r.t. was added imidazole (1.76 g, 27.01 mmol) and tert-butyldimethylsilyl chloride (4.07 g, 27.01 mmol). The reaction mixture was allowed to stir at r.t. for 24 h. The mixture was then diluted with Et₂O (100 mL) and washed with water (3×100 mL) and brine (100 mL). The organic layer was dried over anhydrous Na₂SO₄ and concentrated in vacuo. The crude product was purified by filtration through silica gel to give alkyne 813 (5.68 g, quant.) as a colourless liquid. Alkyne 813 was used in subsequent synthetic steps without characterisation.

### Step ii

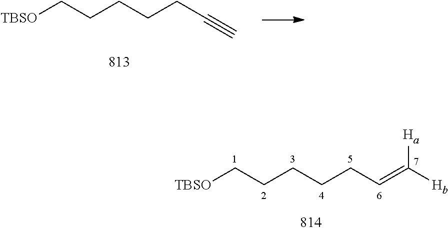

To a stirred solution of alkyne 813 (5.34 g, 25.18 mmol) in hexanes (140 mL) at r.t. was added quinoline (4.18 mL, 35.26 mmol) and Lindar's catalyst (0.53 g). The reaction mixture was connected to a Hz-filled balloon (1 atm) and allowed to stir at r.t. for 2 h. The mixture was then filtered through a pad of Celite® and concentrated in vacuo. The crude product was purified by flash column chromatography (petroleum ether-EtOAc, 9:1) to give 814 (5.34 g, quant.) as a colourless liquid.

R_{f} 0.91 (petroleum ether-EtOAc 9:1); δ_{H} (400 MHz; CDCl₃) 5.81 (1H, ddt, J=17.0, 10.3, 6.7 Hz, H-6), 4.99 (1H, dd, J=17.0 Hz, Ha-7) 4.93 (1H, dd, J=10.1 Hz, H_{b}-7), 3.60 (2H, t, J=6.6 Hz, H-1), 2.05 (2H, q, J=7.0 Hz, H-5), 1.56-1.31 (6H, m, H-2, H-3, H-4), 0.89 (9H, s, SiC(CH₃)₃), 0.05 (6H, s, Si(CH₃)₂); δ_{C} (100 MHz; CDCl₃) 139.1 (CH, C-6), 114.2 (CH₂, C-7), 63.2 (CH₂, C-1), 33.8 (CH₂, C-5), 33.7 (CH₂, C-4), 28.7 (CH₂, C-3), 26.0 (3×CH₃, SiC(CH₃)₃), 25.3 (CH₂, C-2), 18.4 (C, SiC(CH₃)₃), −5.3 (2×CH₃, Si(CH₃)₂). Spectroscopic data were consistent with those reported in literature.

### B) Synthesis of Alkene 814 from Alcohol 815

### Step i

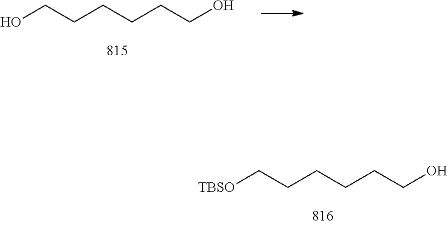 [SMILES: OCCCCCCO ; CC(C)(C)[Si](C)(C)OCCCCCCO]

To a stirred solution of 1,6-hexanediol (815) (16.00 g, 135.39 mmol) in CH₂Cl₂ (150 mL) at r.t. was added imidazole (9.22 g, 135.39 mmol) and tert-butyldimethylsilyl chloride (20.41 g, 135.39 mmol). The reaction mixture was allowed to stir at r.t. for 19 h. The mixture was then filtered, washed with H₂O (100 mL) and brine (100 mL), dried over anhydrous Na₂SO₄ and concentrated in vacuo. The crude product was purified by flash column chromatography (petroleum ether-EtOAc, 4:1) to give 816 (25.13 g, 80%) as a colourless liquid. Alcohol 816 was used in subsequent synthetic steps without characterisation.

### Step ii

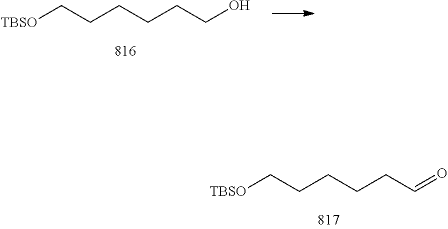 [SMILES: CC(C)(C)[Si](C)(C)OCCCCCCO ; CC(C)(C)[Si](C)(C)OCCCCCC=O]

To a stirred solution of alcohol 816 (4.90 g, 21.10 mmol) in CH₂Cl₂ (11 mL) at 0° C. was added dimethylsulfoxide (11.08 mL, 154.05 mmol), Et₃N (14.71 mL, 105.52 mmol) and sulfur trioxide pyridine complex (9.89 g, 63.31 mmol). The reaction mixture was allowed to stir at 0° C. for 30 min. The mixture was then quenched with water (20 mL) and extracted with EtOAc (2×50 mL). The combined organic extracts were washed with water (50 mL) and brine (50 mL), dried over anhydrous Na₂SO₄ and concentrated in vacuo. The crude product was purified by flash column chromatography (petroleum ether-EtOAc, 9:1) to give 817 (4.71 g, 97%) as a colourless oil. Aldehyde 817 was used in subsequent synthetic steps without characterization.

### Step iii

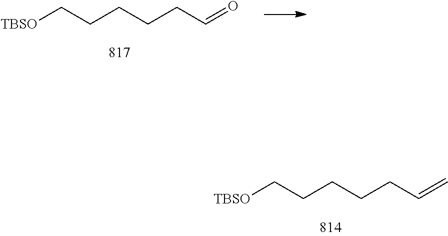

To a stirred solution of methyltriphenylphosphonium bromide (4.60 g, 12.89 mmol) in THF (30 mL) at −78° C. was added a solution of n-butyllithium (7.16 mL, 1.8 M, 12.89 mmol) dropwise. The resultant mixture was warmed to r.t. and allowed to stir for 1 h. The reaction mixture was then cooled to −78° C. and aldehyde 817 (2.56 g, 11.21 mmol) in THF (6 mL) was added dropwise. The reaction mixture was allowed to stir at −78° C. for 3 h and then warmed to r.t. and allowed to stir for a further 15 h. The mixture was then quenched with sat. aq. NH₄Cl (10 mL) and extracted with EtOAc (3×70 mL). The combined organic extracts were washed with water (2×50 mL) and brine (50 mL), dried over anhydrous Na₂SO₄ and concentrated in vacuo. The crude product was purified by flash column chromatography (petroleum ether-EtOAc, 99:1) to give 814(2.50 g, 98%) as a colourless liquid.

R_{f} 0.91 (petroleum ether-EtOAc 9:1); δ_{H} (400 MHz; CDCl₃) 5.81 (1H, ddt, J=17.0, 10.3, 6.7 Hz, H-6), 4.99 (1H, dd, J=17.0 Hz, Ha-7) 4.93 (1H, dd, J=10.1 Hz, H_{b}-7), 3.60 (2H, t, J=6.6 Hz, H-1), 2.05 (2H, q, J=7.0 Hz, H-5), 1.56-1.31 (6H, m, H-2, H-3, H-4), 0.89 (9H, s, SiC(CH₃)₃), 0.05 (6H, s, Si(CH₃)₂); δ_{C} (100 MHz; CDCl₃) 139.1 (CH, C-6), 114.2 (CH₂, C-7), 63.2 (CH₂, C-1), 33.8 (CH₂, C-5), 33.7 (CH₂, C-4), 28.7 (CH₂, C-3), 26.0 (3×CH₃, SiC(CH₃)₃), 25.3 (CH₂, C-2), 18.4 (C, SiC(CH₃)₃), −5.3 (2×CH₃, Si(CH₃)₂). Spectroscopic data were consistent with those reported in literature.

### C) Synthesis of Amino Acid Conjugate 820 from Alkene 814

### Step i

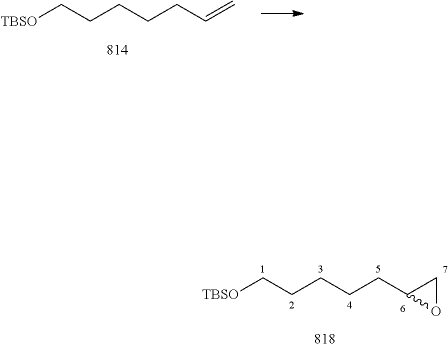 [SMILES: C=CCCCCCO[Si](C)(C)C(C)(C)C ; CC(C)(C)[Si](C)(C)OCCCCCC1CO1]

To a stirred solution of alkene 814 (4.30 g, 18.40 mmol) in CH₂Cl₂ (40 mL) at r.t. was added mCPBA (4.46 g, 25.84 mmol). The reaction mixture was allowed to stir at r.t. for 7 h. The mixture was then filtered through Celite®, diluted with Et₂O (60 mL) and washed with sat. aq. NaHCO₃ (3×100 mL) and brine (100 mL). The organic layer was dried over anhydrous Na₂SO₄ and concentrated in vacuo. The crude product was purified by flash column chromatography (petroleum ether-EtOAc, 9:1) to give 818 (4.30 g, 96%) as a colourless liquid.

R_{f} 0.63 (petroleum ether-EtOAc 9:1); δ_{H} (400 MHz; CDCl₃) 3.60 (2H, t, J=6.5 Hz, H-1), 2.92-2.88 (1H, m, H-6), 2.74 (1H, t, J=4.5 Hz, H-7), 2.46 (1H, dd, J=5.0, 2.8 Hz, H-7), 1.56-1.36 (8H, m, H-2, H-3, H-4, H-5), (9H, s, SiC(CH₃)₃), 0.04 (6H, s, Si(CH₃)₂); δ_{C} (100 MHz; CDCl₃) 63.1 (CH₂, C-1), 52.3 (CH, C-6), 47.1 (CH₂, C-7), 32.8 (CH₂, C-5), 32.5 (CH₂, C-2), 26.0 (3×CH₃, SiC(CH₃)₃), 25.8 (CH₂, C-4), 25.7 (CH₂, C-3), 18.4 (C, SiC(CH₃)₃), −5.3 (2×CH₃, Si(CH₃)₂). Spectroscopic data were consistent with those reported in literature.

### Step ii

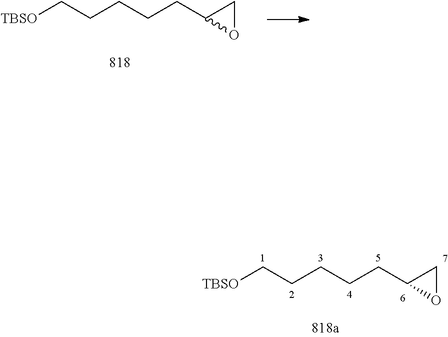 [SMILES: CC(C)(C)[Si](C)(C)OCCCCCC1CO1 ; CC(C)(C)[Si](C)(C)OCCCCC[C@@H]1CO1]

To a stirred solution of racemic epoxide 818 (2.23 g, 9.13 mmol), (R,R)-(+)-N,N′-bis(3,5-di-tert-butylsalicylidene)-1,2-cyclohexanediaminocobalt(II) (0.03 g, 0.05 mmol) and glacial acetic acid (0.01 mL, 0.18 mmol) in THF (0.1 mL) at 0° C. was added water (0.09 mL) dropwise. The reaction mixture was allowed to stir at r.t. for 48 h. The mixture was then concentrated in vacuo. The crude product was purified by flash column chromatography (petroleum ether-EtOAc, 9:1) to give 818a (1.09 g, 49%) as a yellow oil.

R_{f} 0.63 (petroleum ether-EtOAc 9:1); [α]_{D}^{21.3} +4.2 (c 0.90 in CHCl₃); δ_{H} (400 MHz; CDCl₃) 3.60 (2H, t, J=6.5 Hz, H-1), 2.92-2.88 (1H, m, H-6), 2.74 (1H, t, J=4.5 Hz, H-7), 2.46 (1H, dd, J=5.0, 2.8 Hz, H-7), 1.56-1.36 (8H, m, H-2, H-3, H-4, H-5), (9H, s, SiC(CH₃)₃), 0.04 (6H, s, Si(CH₃)₂); δ_{C} (100 MHz; CDCl₃) 63.1 (CH₂, C-1), 52.3 (CH, C-6), 47.1 (CH₂, C-7), 32.8 (CH₂, C-5), 32.5 (CH₂, C-2), 26.0 (3×CH₃, SiC(CH₃)₃), 25.8 (CH₂, C-4), 25.7 (CH₂, C-3), 18.4 (C, SiC(CH₃)₃), −5.3 (2×CH₃, Si(CH₃)₂). Spectroscopic data were consistent with those reported in literature.

### Step iii

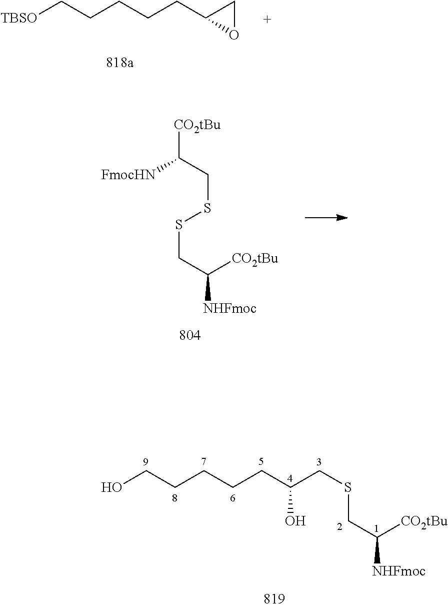

To a stirred solution of disulfide 804 (0.30 g, 0.375 mmol) in CH₂Cl₂ (1 mL) at 0° C. was added zinc powder (0.20 g, 3.01 mmol) and a freshly prepared mixture of methanol, conc. hydrochloric acid and conc. sulfuric acid (100:7:1, 1 mL). The resultant mixture was allowed to stir at 0° C. for 30 min after which was added epoxide 818a (0.344 g, 1.13 mmol). The reaction mixture was allowed to stir at 70° C. for 17 h. The mixture was then diluted with EtOAc (30 mL), filtered through a pad of Celite® and washed with brine (30 mL). The aqueous layer was extracted with EtOAc (3×30 mL) and the combined organic extracts were dried over anhydrous MgSO₄ and concentrated in vacuo. The crude product was purified by flash column chromatography (hexanes-EtOAc, 1:3) to give 819 (0.350 g, 88%) as a colourless oil.

R_{f} 0.4 (hexane-EtOAc 1:3); [α]_{D}^{20.8} −20.0 (c 0.03 in EtOAc); νₘₐₓ(neat)/cm⁻¹ 3365, 3933, 1703, 1514, 1450, 1369, 1343, 1248, 1151, 1046; δ_{H} (400 MHz; MeOD) 7.79 (2H, d, J=7.5 Hz, FmocH), 7.68 (2H, d, J=7.4 Hz, FmocH), 7.39 (2H, t, J=7.4 Hz, FmocH), 7.31 (2H, t, J=4.7 Hz, FmocH), 4.34 (2H, d, J=7.1 Hz, FmocCH), 4.28 (1H, dd, J=8.2, 5.1 Hz, H-1), 4.23 (1H, t, J=7.0 Hz, FmocCH₂), 3.72-3.61 (1H, m, H-4), 3.57-3.79 (2H, m, H-9), 3.01 (1H, dd, J=13.8, 5.0 Hz, H-2), 2.86 (1H, dd, J=13.7, 8.3 Hz, H-2), 2.69 (1H, dd, J=13.4, 4.9 Hz, H-3), 2.60 (1H, dd, J=13.4, 7.0 Hz, H-3), 1.57-1.34 (17H, m, H-5, H-6, H-7, H-8, C(CH₃)₃); δ_{C} (100 MHz; MeOD) 171.8 (C, CO₂tBu), 158.1 (C, FmocCO), 145.3 (C, Fmoc), 142.6 (C, Fmoc), 128.8 (CH, Fmoc), 128.2 (CH, Fmoc), 126.4 (CH, Fmoc), 121.0 (CH, Fmoc), 83.3 (C, C(CH₃)₃), 71.9 (CH, C-4), 68.2 (CH₂, FmocCH₂), 62.9 (CH₂, C-9), 56.5 (CH, C-1), 50.2 (CH, FmocCH), 40.8 (CH₂, C-3), 37.3 (CH₂, C-5), 35.5 (CH₂, C-2), 33.6 (CH₂, C-8), 28.3 (3×CH₃, C(CH₃)₃), 26.9 (CH₂, C-7), 26.6 (CH₂, C-6); HRMS (ESI+) [M+Na]⁺ 552.2390 calc for C₂₉H₃₉NNaO₆S 552.2393.

### Step iv

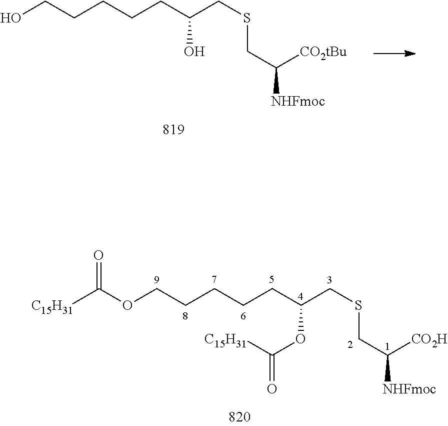

To a stirred solution of diol 819 (0.168 g, 0.317 mmol) and palmitic acid (0.244 g, 0.951 mmol) in THF (4.6 mL) at r.t. was added N,N′-diisopropylcarbodiimide (0.191 mL, 1.269 mmol) and 4-dimethylaminopyridine (0.016 g, 0.127 mmol). The reaction mixture was allowed to stir at r.t. for 17 h. The mixture was then filtered through a pad of Celite®, diluted with EtOAc (30 mL), washed with 1M aq. citric acid (30 mL) and brine (30 mL) and concentrated in vacuo. The residue was then redissolved in TFA (3 mL) and allowed to stir at r.t. for 45 min. The reaction mixture was again concentrated in vacuo. The crude product was purified by flash column chromatography (hexanes-EtOAc, 9:1→0:1) to give 820 (0.301 g, quant.) as a colourless oil.

R_{f} 0.21 (petroleum ether-EtOAc 1:1); [α]_{D}^{20.8} +7.5 (c 0.24 in CHCl₃); νₘₐₓ(neat)/cm⁻¹ 3319, 2919, 2851, 1722, 1521, 1471, 1450, 1221, 1055; 6 H (400 MHz; CDCl₃) 7.76 (2H, d, J=7.6 Hz, FmocH), 7.61 (2H, d, J=7.3 Hz, FmocH), 7.40 (2H, t, J=7.7 Hz, FmocH), 7.30 (2H, td, J=11.2, 1.1 Hz, FmocH), 5.82, (1H, d, J=7.7 Hz, NH), 5.00-4.94 (1H, m, H-4), 4.64 (1H, dd, J=12.3, 5.6 Hz, H-1), 4.40 (2H, d, J=7.1 Hz, FmocCH₂), 4.24 (1H, t, J=7.1 Hz, FmocCH), 4.10-4.00 (2H, m, H-9), 3.14 (1H, dd, J=13.8, 4.3 Hz, H-2), 3.04 (1H, dd, J=13.8, 5.6 Hz, H-2), 2.76-2.67 (2H, m H-3), 2.31 (2H, t, J=7.6 Hz, PamCH_{2α}alkyl), 2.28 (2H, t, J=7.6 Hz, PamCH_{2α}alkyl), 1.65-1.56 (8H, m, 2×PamCH_{2β}alkyl, H-8, H-5), 1.39-1.18 (52H, m, 24×PamCH₂alkyl, H-6, H-7), 0.88 (6H, t, J=6.9 Hz, 2×PamCH₃alkyl); δ_{C} (100 MHz; CDCl₃) 174.4 (C, CO₂H), 156.1 (C, FmocCO), 143.7 (C, Fmoc), 141.3 (C, Fmoc), 127.8 (CH, Fmoc), 127.1 (CH, Fmoc), 125.2 (CH, Fmoc), 120.0 (CH, Fmoc), 72.4 (CH, C-4), 67.5 (CH₂, FmocCH₂), 64.2 (CH₂, C-9), 53.6 (CH, C-1), 47.1 (CH, FmocCH), 36.5 (CH₂, C-3), 34.6 (CH₂, C-2), 34.3 (2×CH₂, PamCH_{2α}alkyl), 33.0 (CH₂, C-5), 31.9 (2×CH₂, PamCH₂alkyl) 29.7-28.4 (21×CH₂, PamCH₂alkyl, C-8), 25.5 (CH₂, C-7), 25.0 (2×CH₂, PamCH_{2β}alkyl), 24.8 (CH₂, C-6), 22.7 (2×CH₂, PamCH₂alkyl), 14.1 (2×CH₃, PamCH₃alkyl); HRMS (ESI+) [M+Na]⁺ 972.6358 calc for C₆₇H₉₁NNaO₈S 972.6392.

### 3. Example 3

This example investigates human TLR2 agonism of compounds of the present invention compared with a conjugate of a known TLR2 agonist ((R)-Pam2Cys).

### 3.1 Peptide Conjugate Synthesis

### 3.1.1 Synthesis of Compounds 910, 911, 912, and 913 Procedure A

Peptide conjugates of the invention 910, 911, 912 and 913 (depicted in Table 1) comprising the peptide sequence SKKKKSLLMWITQV [SEQ ID No: 139] were prepared as described and depicted below (Scheme 1).

The peptide sequence SKKKKSLLMWITQV [SEQ ID No: 139] includes an immunogenic peptide epitope (underlined), which is an analogue of sequence derived from the NY-ESO-1 protein (NY-ESO-1 157-165) and may be used to stimulate human NY-ESO-1-specific CD8+ T-cells as described in Chen, J-L et al. The Journal of Immunology, 2000, 165, 948-955.

The SKKKK solubilising tag is believed to improve handling and ease the purification of the potentially lipophilic peptide.

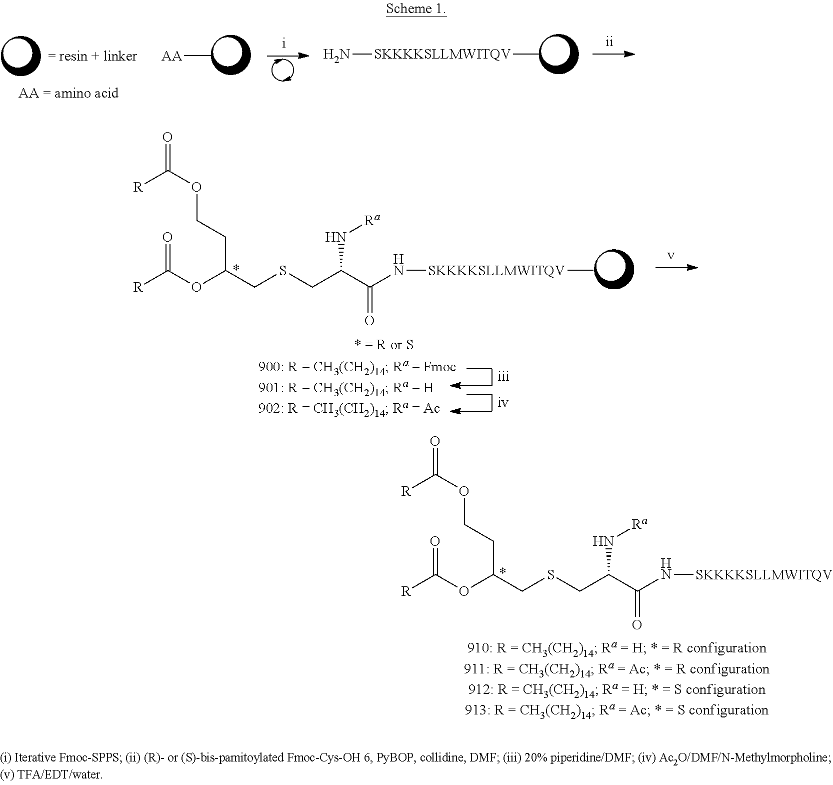

The desired peptide sequence was synthesised using standard iterative Fmoc Solid-Phase Peptide Synthesis techniques on a Tribute peptide synthesiser (Protein Technologies International, Tucson, Ariz.).

A typical deprotection and coupling cycle carried out on a 0.1 mmol scale entailed removal of the Fmoc protecting group from the resin-bound amino-acid using two treatments of 20% piperidine in DMF (4 mL×5 min) then washing the resin with DMF. In a separate vessel the Fmoc amino acid (0.5 mmol) and coupling agent (1-[bis(dimethylamino)methylene]-1H-1,2,3-triazolo[4,5-b]pyridinium 3-oxide hexafluorophosphate (HATU), 0.45 mmol) were dissolved in DMF (1.5 mL) and base (4-methylmorpholine (NMM), 1 mmol) added. After mixing for 1 minute, this solution was transferred to the resin, which was agitated at room temperature (RT) for 1 hour, drained and washed.

Reverse phase (RP)-HPLC was carried out using a Dionex Ultimate 3000 HPLC system. with UV detection at 210 nm or 225 nm. For semi-preparative purifications, a peptide sample was injected into a reverse-phase Phenomenex Gemini C18 column (5μ, 110 Å; 10×250 mm) equilibrated in a suitable mixture of eluent A (water/0.1% TFA) and eluent B (MeCN/0.1% TFA) then an increasing gradient of eluent B was generated to elute the constituent components. Analytical HPLC was performed similarly, using a Phenomenex Gemini C18 column (3μ, 110 Å; 4.6×150 mm). Low-resolution mass spectra were obtained using an Agilent Technologies 6120 Quadrapole mass spectrometer. After coupling the penultimate amino acid residue, the resin-bound peptide chain was then derivatised with the desired diastereomer of amino acid conjugate 6 using BOP (benzotriazole-1-yl-oxy-tris-(dimethylamino)-phosphonium hexafluorophosphate) and collidine in DMF. The conditions for coupling of the amino acid conjugate reduce the propensity of the α-carbon of the amino acid to epimerise on activation. The amino acid conjugate (0.1 mmol) and BOP (0.095 mmol) were combined and dissolved in DMF (1.0 mL). Neat 2,4,6-trimethylpyridine (0.2 mmol) was added. After mixing for 30 seconds the solution was transferred to 0.02 mmol of resin, which was then agitated for 60 minutes, drained and washed (DMF) to afford 900.

The Fmoc group was then removed using 20% piperidine in DMF to provide 901. The terminal amino group could then be acetylated using Acetic anhydride in DMF to afford 902. Resin (0.01 mmol) was suspended in DMF (2 mL) acetic anhydride (0.1 mL) and N-methylmorpholine (0.1 mL) were added, and the mixture agitated for 5 minutes. The resin was then drained and washed extensively with DMF.

Peptide 901 was cleaved from the resin to provide the peptide conjugate 910 with the R configuration at the indicated position and R^{a}=H or the peptide conjugate 912 with the S configuration at the indicated position and R^{a}=H (Scheme 1). Peptide 902 was cleaved from the resin to provide the peptide conjugate 911 with the R configuration at the indicated position and R^{a}=Acetyl or the peptide conjugate 913 with the S configuration at the indicated position and R^{a}=Acetyl (Scheme 1). Resin (0.01 mmol) in 1.0 mL of trifluoroacetic acid containing 5% (v/v) 2,2′-(ethylenedioxy)diethanethiol was agitated at room temperature for 2 hours. The supernatant was then drained through a sinter into chilled diethyl ether (10 mL). The resin was then washed with a further 1 mL of TFA, which was also added to the ether. The precipitated material was pelleted by centrifugation and the pellet washed once with ether (5 mL) before being dissolved in 1:1 MeCN/Water (+0.1% tfa) and lyophilised.

Purification of 910, 911, 912 and 913 was performed by semi-preparative HPLC using a Phenomenex Gemini C18 (5μ, 110 Å) 10×250 mm column with eluent A being water (+0.1% tfa) and eluent B being MeCN (+0.1% tfa). After injection of the crude peptide sample on to the column the following gradient was generated: 5% B to 50% B over 1.5 minutes followed by 50% B to 100% B over 23.5 minutes at a flow of 4 mL/min. The desired product material collected on elution from the column and freeze-dried.

910: m/z (ESI) 1179.9 [M+2H+]. HPLC analysis: Column: Phenomenex Gemini C18 (3μ, 110 Å, 4.6×150 mm); eluent A, water/0.1% TFA; eluent B: MeCN/0.1% TFA; gradient: 5-100% B over 25 min @ 1 mL/min. Retention time: 22.9 mins.

911: m/z (ESI) 1201.0 [M+2H+]. HPLC analysis: Column: Phenomenex Gemini C18 (3μ, 110 Å, 4.6×150 mm); eluent A, water/0.1% TFA; eluent B: MeCN/0.1% TFA; gradient: 5-100% B over 25 min @ 1 mL/min. Retention time: 24.3 mins.

912: m/z (ESI) 1179.9 [M+2H+]. HPLC analysis: Column: Phenomenex Gemini C18 (3μ, 110 Å, 4.6×150 mm); eluent A, water/0.1% TFA; eluent B: MeCN/0.1% TFA; gradient: 5-100% B over 30 min @ 1 mL/min. Retention time: 22.8 mins.

913: m/z (ESI) 1200.9 [M+2H+]. HPLC analysis: Column: Phenomenex Gemini C18 (3μ, 110 Å, 4.6×150 mm); eluent A, water/0.1% TFA; eluent B: MeCN/0.1% TFA; gradient: 5-100% B over 30 min @ 1 mL/min. Retention time: 24.1 mins.

TABLE 1
Structures of peptide conjugates 910-913. All structures comprise the
peptide sequence SKKKKSLLMWITQV [SEQ ID No: 139]

| No. | Structure |
|---|---|
| 910 | 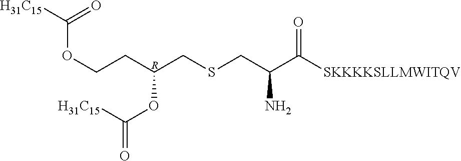 |
| 911 | 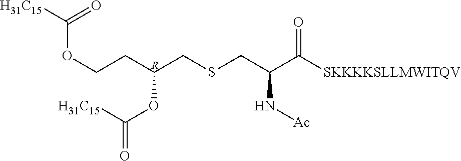 |
| 912 | 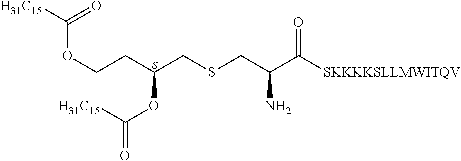 |
| 913 | 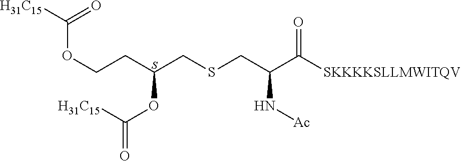 |

### Procedure B

Peptide conjugates of the invention 910, 911, 912 and 913 (depicted in Table 1) were also prepared by the following alternative procedure.

Fmoc-amino acids were supplied with the following side-chain protection: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Tyr(OtBu)-OH, Fmoc-Val-OH.

Purification was performed using semi-preparative Reverse Phase (RP) High-Performance Liquid Chromatography (HPLC) using a column and running a gradient as described per procedure for each peptide. Both semi-preparative and analytical RP-HPLC were conducted using a Dionex Ultimate 3000 HPLC system with the described solvent system. The purest fractions were combined and lyophilised. Liquid chromatography-mass spectrometry (LC-MS) chromatograms were acquired on an Agilent 1120 Compact LC system with a Hewlett Packard series 1100 MSD mass spectrometer.

### Preparation of Peptidyl Resin 390 Comprising Amino Acid Residue:

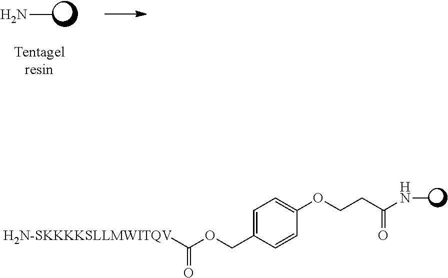

Tentagel resin (0.581 g, 0.25 mmol scale) was swollen in DMF (6 mL) for 10 min. Fmoc-L-Val-OCH₂-pC₅H₄—OCH₂CH₂CO₂H (0.155 g, 0.30 mmol) and HATU (0.114 g, 0.30 mmol) were dissolved in DMF (3 mL) and activated with N-methylmorpholine (0.055 mL, 0.50 mmol) for 1 min. The resultant mixture was added to the resin and agitated for 1 h at r.t. The resin was drained and washed with DMF (2×3 mL). The resin was then treated with 20% v/v piperidine in DMF (2 mL) and agitated for 10 min at r.t. The resin was drained and the process repeated. The resin was drained and washed with DMF (4×3 mL). The resin was transferred to a Tribute® automated peptide synthesiser reaction vessel. The peptide was then elongated using a Tribute® automatic peptide synthesiser. Automated synthesis was performed by alternating cycles Fmoc-AA-OH coupling and Fmoc deprotection. The resin was washed with DMF (5×4 mL) prior to addition of the coupling mixture, which consisted of Fmoc-AA-OH (5 eq.) and HATU (4.75 eq.) in DMF (2.5 mL) and 2M N-methylmorpholine in DMF (1.5 mL). Upon completion of coupling after agitation for 1 h at r.t., the resin was drained and washed with DMF (5×4 mL). The resin was treated with Ac₂O (4 mL) and agitated for 10 min at r.t. The resin was drained, washed with DMF (5×4 mL), treated with 20% v/v piperidine in DMF (4 mL) and agitated for 10 min. The resin was drained and the process was repeated. The next cycle of washing, coupling and deprotection was repeated until all amino acids in the sequence were coupled. The resin was then washed with DMF (2×4 mL) and CH₂Cl₂ (2×4 mL) to give the peptidyl resin 390 (0.957 g, 0.261 mmol g⁻¹ loading).

### Synthesis of Lipidated Peptides 910, 911, 912 and 913:

Peptidyl resin 390 (0.038 g, 0.01 mmol scale) was swollen in DMF (2 mL) for 10 min. Fmoc-(S)-homoPam₂Cys-OH (6B), for the preparation of 910 and 911, or Fmoc-(R)-homoPam₂Cys-OH (6A), for the preparation of 912 and 913, (0.045 g, 0.5 mmol) and PyBOP (0.025 g, 0.0475 mmol) were dissolved in DMF (1 mL) and activated with 2,4,6-collidine (0.013 mL, 0.10 mmol) for 1 min. The resultant mixture was added to the resin and agitated for 1 h at r.t. to give the protected peptidyl resin. The resin was drained and washed with DMF (3×2 mL), a subsequent Ninhydrin test was negative. The resin was then treated with 20% v/v piperidine in DMF (2 mL) and agitated for 10 min at r.t. The resin was drained and the process repeated. The resin was drained, washed with DMF (3×2 mL) and CH₂Cl₂ (3×2 mL). The resin was then either (a), for the preparation of 911 and 913, treated with Ac₂O (2 mL) and agitated for 10 min at r.t., then drained and washed with CH₂Cl₂ (2 mL); or (b), for the preparation of 910 and 913, drained and washed with CH₂Cl₂ (2 mL).

The peptidyl resin was cleaved and precipitated as follows to give the crude peptide. The resin-bound peptide was treated with a cleavage cocktail of 5% DODT:TFA and agitated for 3 h at r.t. The resin was separated from the cleavage cocktail solution by filtration, and the filtrate was treated with cold diethyl ether to precipitate the crude peptide followed by centrifugation at 4000 rpm for 5 min. The supernatant was discarded and the pellet washed with diethyl ether and centrifugation was repeated once more. Upon discarding the supernatant, the resulting peptide pellet was dried under a flow of N₂ and lyophilised from MeCN:H₂O (1:1)+0.1% TFA.

The crude peptide was purified by semi-preparative RP-HPLC on a Phenomenex Gemini C18 column (5μ, 110 Å, 10.0×250 mm) running a gradient of 5-50% (45% MeCN per min) and then 50-100% (2% MeCN per min) MeCN in H₂O+0.1% TFA at r.t. The purified peptides 910, 911, 912, and 913 were obtained as amorphous solids.

910: (6 mg, 22% with >98% purity). Rₜ 22.9 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1179.8 calc for C₁₁₇H₂₁₀N₂₁O₂₄S₂ 1179.9.

911: (3 mg, 12% with >98% purity). Rₜ 24.2 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1201.1 calc for C₁₁₉H₂₁₂N₂₁O₂₅S₂ 1201.3.

912: (5 mg, 26% with >98% purity). Rₜ 22.7 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1179.8 calc for C₁₁₇H₂₁₀N₂₁O₂₄S₂ 1179.9.

913: (3 mg, 12% with >98% purity). Rₜ 24.2 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1201.1 calc for C₁₁₉H₂₁₂N₂₁O₂₅S₂ 1201.3.

### 3.1.2 Synthesis of Compounds 930, 931, and 932

### Procedure A

Peptide conjugates of the invention 930, 931 and 932 (depicted in Table 2) comprising the peptide sequence SKKKKSLLMWITQV [SEQ ID No: 139] were prepared as described and depicted below (Scheme 2).

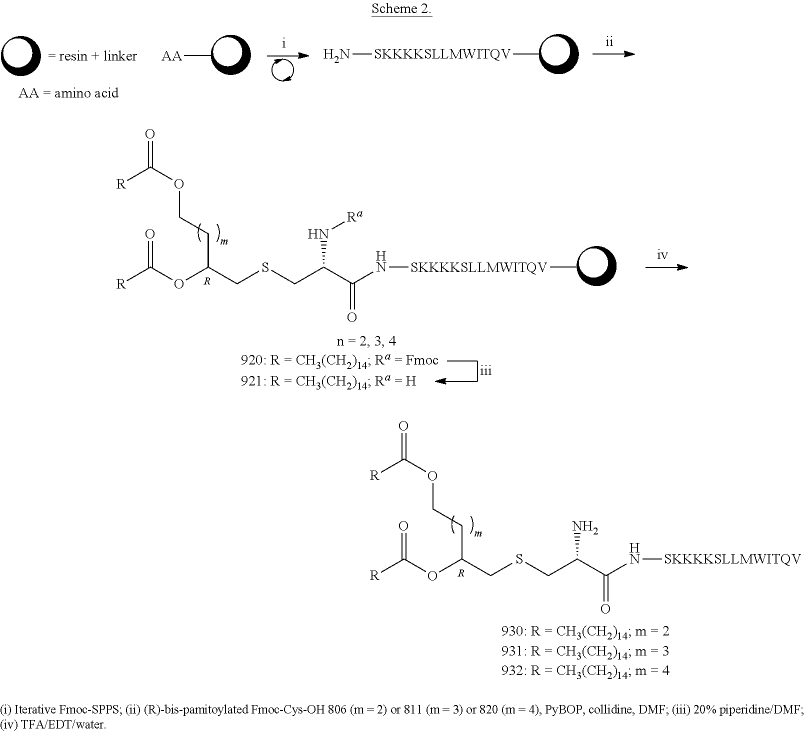

The desired peptide sequence was synthesised using standard iterative Fmoc SPPS techniques as described above.

After coupling the penultimate amino acid residue, the resin-bound peptide chain was then derivatised with either conjugate 806, 811 or 820 using PyBOP (benzotriazol-1-yl-oxytripyrrolidinophosphonium hexafluorophosphate) and collidine in DMF. The conditions for coupling of the amino acid conjugate reduce the propensity of the α-carbon of the amino acid to epimerise on activation. The amino acid conjugate (0.1 mmol) and BOP (0.095 mmol) were combined and dissolved in DMF (1.0 mL). Neat 2,4,6-trimethylpyridine (0.2 mmol) was added. After mixing for 60 seconds the solution was transferred to 0.02 mmol of resin, which was then agitated for 60 minutes, drained and washed (DMF) to afford 920 (m=2, 3, or 4).

The Fmoc group was then removed using 20% piperidine in DMF to provide 921 (m=2, 3, or 4).

Peptide 921 (m=2, 3, or 4) was cleaved from the resin to provide the peptide conjugate 930 (m=2) with the R configuration at the indicated position (Scheme 2), the peptide conjugate 931 (m=3) with the R configuration at the indicated position, or peptide conjugate 932 (m=4) with the R configuration at the indicated position. Resin (0.01 mmol) in 1.0 mL of trifluoroacetic acid containing 5% (v/v) 2,2′-(ethylenedioxy)diethanethiol was agitated at room temperature for 2 hours. The supernatant was then drained through a sinter into chilled diethyl ether (10 mL). The resin was then washed with a further 1 mL of TFA, which was also added to the ether. The precipitated material was pelleted by centrifugation and the pellet washed once with ether (5 mL) before being dissolved in 1:1 MeCN/Water (+0.1% tfa) and lyophilised.

Purification of 930, 931 and 932 was performed by semi-preparative HPLC using a Phenomenex Gemini C18 (5μ, 110 Å) 10×250 mm column with eluent A being water (+0.1% tfa) and eluent B being MeCN (+0.1% tfa). After injection of the crude peptide sample on to the column the following gradient was generated: 5% B to 45% B over 3 minutes followed by 45% B to 65% B over 16 minutes at a flow of 4 mL/min. The desired product material collected on elution from the column and freeze-dried.

930: m/z (ESI) 1186.6 [M+2H+]. HPLC analysis: Column: Phenomenex Jupiter C18 (5μ, 300 Å, 4.6×150 mm); eluent A, water/0.1% TFA; eluent B: MeCN/0.1% TFA; gradient: 5-95% B over 30 min @ 1 mL/min. Retention time: 25.3 mins.

931: m/z (ESI) 1193.6 [M+2H+]. HPLC analysis: Column: Phenomenex Jupiter C18 (5μ, 300 Å, 4.6×150 mm); eluent A, water/0.1% TFA; eluent B: MeCN/0.1% TFA; gradient: 5-95% B over 30 min @ 1 mL/min. Retention time: 25.7 mins.

932: m/z (ESI) 1200.8 [M+2H+]. HPLC analysis: Column: Phenomenex Jupiter C18 (5μ, 300 Å, 4.6×150 mm); eluent A, water/0.1% TFA; eluent B: MeCN/0.1% TFA; gradient: 5-95% B over 30 min @ 1 mL/min. Retention time: 26.2 mins.

TABLE 2
Structures of peptide conjugates 930-932. All structures comprise the
peptide sequence SKKKKSLLMWITQV [SEQ ID No: 139]

| No. | Structure |
|---|---|
| 930 | 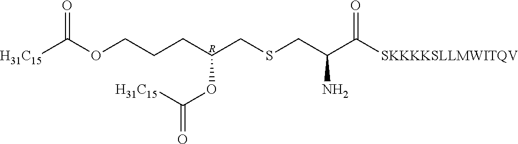 |
| 931 | 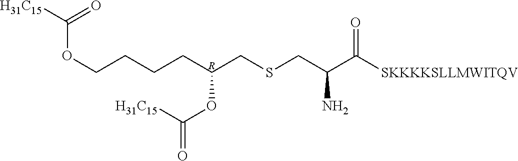 |
| 932 | 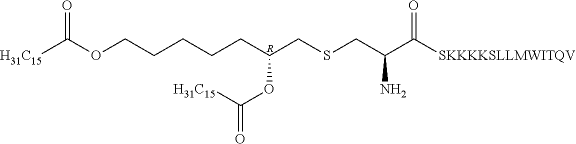 |

### Procedure B

Peptide conjugates of the invention 930, 931 and 932 (depicted in Table 2) were also prepared by the following alternative procedure.

Fmoc-amino acids, purification, and LCMS were as set forth in Procedure B of section 3.1.1 of this Example.

### Synthesis of Lipidated Peptides 930, 931, and 932:

Peptidyl resin 390, prepared as set forth in Procedure B of section 3.1.1 of this Example, (0.083 g, 0.02 mmol scale) was swollen in DMF (2 mL) for 10 min. Amino acid 806 (0.092 g, 0.10 mmol), 811 (0.093 g, 0.10 mmol), or 820 (0.095 g, 0.10 mmol), for the preparation of 930, 931, or 932, respectively, and BOP (0.042 g, 0.095 mmol) were dissolved in DMF (2 mL) and activated with collidine (0.026 mL, 0.20 mmol) for 1 min. The resultant mixture was added to the resin and agitated for 1 h at r.t. to give the protected peptidyl resin. The resin was drained and washed with DMF (3×2 mL), a subsequent Ninhydrin test was negative. The resin was then treated with 20% v/v piperidine in DMF (2 mL) and agitated for 10 min at r.t. The resin was drained and the process repeated. The resin was drained, washed with DMF (3×2 mL) and CH₂Cl₂ (3×2 mL).

The peptidyl resin was cleaved and precipitated as follows to give the crude peptide. The resin-bound peptide was treated with a cleavage cocktail of 5% DODT:TFA and agitated for 3 h at r.t. The resin was separated from the cleavage cocktail solution by filtration. All volatiles were removed using a flow of N₂ and the resulting residue was lyophilised from MeCN:H₂O (1:1)+0.1% TFA.

The crude peptide was purified by semi-preparative RP-HPLC on a Phenomenex Gemini C18 column (5μ, 110 Å, 10.0×250 mm) running a gradient of 5-50% (45% MeCN per min) and then 50-100% (2% MeCN per min) MeCN in H₂O+0.1% TFA at r.t. The purified peptides 930, 931, and 932 were obtained as amorphous solids.

930: (8 mg, 17% with >98% purity). Rₜ 20.9 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1187.1 calc for C₁₁₈H₂₁₃N₂₁O₂₄S₂ 1186.6.

931: (9 mg, 19% with >98% purity). Rₜ 25.7 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1194.1 calc for C₁₁₉H₂₁₅N₂₁O₂₄S₂ 1193.6.

932: (9 mg, 19% with >98% purity). Rₜ 25.7 min on a Phenomenex Gemini C18 3μ 110 Å 4.6×150 mm column using a 5-100% MeCN:H₂O+0.1% TFA, 3.8% MeCN per min gradient; LRMS (ESI+) [M+2H]²⁺1201.1 calc for C₁₂₀H₂₁₇N₂₁O₂₄S₂ 1200.8.

### 3.1.3 (R)-Pam2Cys-SK₄-SLLMWITQV

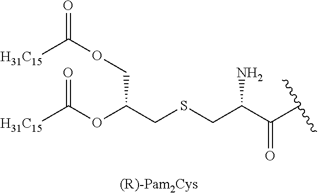

Peptide conjugate (R)-Pam2Cys-SK₄-SLLMWITQV was prepared using methods analogous to those described herein above.

### 3.2 Toll-Like Receptor 2 (TLR2) Agonism Using Hek-Blue Cells

HEK-Blue™ Detection medium and HEK-Blue™-hTLR2 cells were purchased from Invivogen. HEK-Blue™ cells express endogenous levels of human (h)TLR1 and hTLR6, and exhibit co-transfection of hTLR2 and the reporter gene SEAP (secreted embryonic alkaline phosphatase). The SEAP reporter gene is under the control of the IFN-8 minimal promoter fused to five AP-1 and five NFκB binding sites. Cells were cultured according to manufacturer's instructions.

On the day of the assay, TLR agonists: 910; 930; 931; 932; (R)-Pam2Cys-SK₄-SLLMWITQV; or PBS (negative control) were plated in 20p1 of endotoxin free water in a 96-well plate. All constructs tested were produced in-house as described above. HEK-Blue™-hTLR2 cells were resuspended at ˜2.78×10⁵ cells/ml in HEK-Blue™ Detection medium and 180p1 of the cell suspension added to each well (˜5×10⁴ cells) to give final agonist concentrations as indicated across a 7-log₁₀ dilution series (10⁻⁶ M to 10⁻¹² M). Cells were incubated for 16 h at 37° C. in 5% CO₂. SEAP expression was quantified using an EnSpire plate reader (PerkinElmer) at 635 nM. Data presented as mean+/−SD ABS (635 nm) values for triplicate wells following background subtraction.

### Results

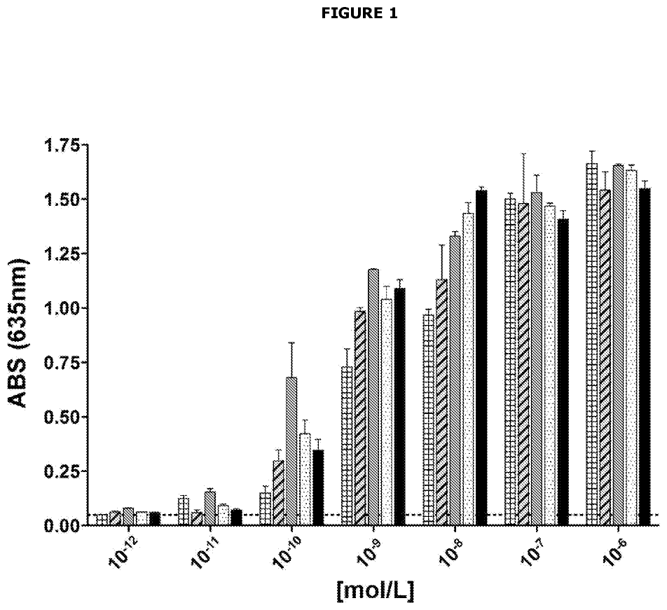

The results are shown in FIG. 1. All constructs analysed exhibited hTLR2 agonism at concentrations ranging from 10⁻⁶ to 10⁻¹¹M. Constructs 930 (grey bars), 931 (striped bars), and 932 (square hatched bars) exhibited comparable human TLR2 agonism to 910 (dotted white bars) and to Pam2Cys-SKKKK-SLLMWITQV (black bars) at each concentration (10⁻⁶ to 10⁻¹¹M), demonstrating that homologation extension does not inhibit binding to, or signalling through human TLR2 by diacylated lipopeptide constructs.

### 4. Example 4

This example investigates human TLR2 agonism of compounds of the present invention compared with conjugates of known TLR2 agonists ((R)-Pam2Cys, (S)-Pam2Cys, (R)-Pam3Cys, and (S)-Pam3Cys).

### 4.1 Peptide Conjugates

The peptide conjugates tested are listed in Tables 3 and 4 below. Peptide conjugates 910, 911, 912, 913, 930, 931, and 932 were prepared as described above in Example 3. Peptide conjugates 45a, 45b, 46a, 46b, 47a, 47b were prepared using methods analogous to those described herein above.

### 4.2 Toll-Like Receptor 2 (TLR2) Agonism Using Hek-Blue Cells

Human TLR2 agonism by the peptide conjugates was investigated using HEK-Blue™-hTLR2 cells and Hek-Blue™ Detection medium (Invivogen) across an 8-log₁₀ dilution series (10⁹ fM to 10² fM). Agonists were diluted and incubated with HEK-Blue™-hTLR2 cells for 16 h, and well absorbance (ABS) then determined at 655 nm using an Ultramark™ microplate reader (BioRad). EC₅₀ (nM) values were determined by non-linear regression curve fit of normalised ABS (655 nm) values using Prism 7 software (GraphPad).

### Results

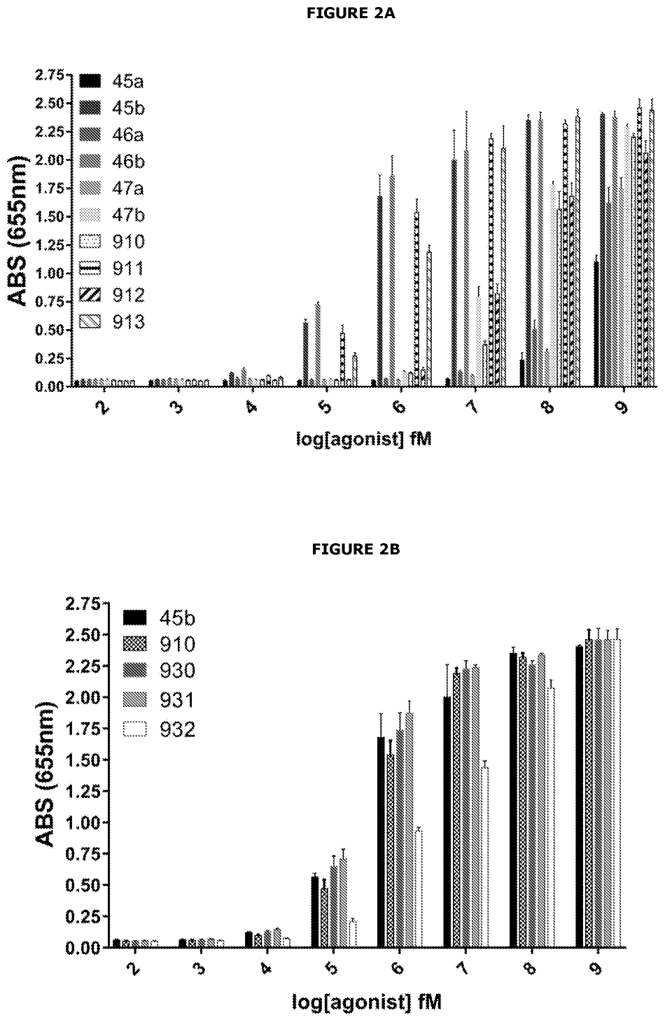

EC₅₀ values were determined for homoPam2Cys constructs 910, 911, 912 and 913 and compared with the EC₅₀ values for corresponding Pam2Cys constructs 45a and 45b, N-acetylated Pam2Cys constructs 46a and 46b and Pam3Cys constructs 47a and 47b (Table 3). The results for the hTLR agonism assay are shown in FIG. 2A. All constructs exhibited hTLR2 agonism. HomoPam2Cys constructs demonstrated comparable activity to the corresponding Pam2Cys and Pam3Cys constructs. (S)-homoPam2Cys constructs 912 and 913 showed improved activity over the (S)-Pam2Cys constructs 45a and 46a as well as comparable activity to the (R)-Pam3Cys construct 47b.

TABLE 3
Structures of peptide conjugates 45a, 45b, 46a, 46b, 47a, 47b, 910-913 and
EC₅₀ for human TLR2 agonism as determined by HEK-Blue ^{TM} assay.
X = -SKKKKSLLMWITQV [SEQ ID No: 139].

| No. | Structure | EC₅₀ (nM) |
|---|---|---|
| 45a | 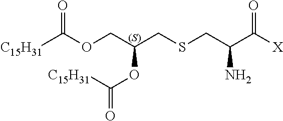 | 155.693 |
| 45b | 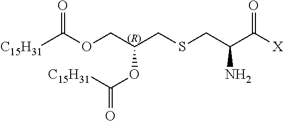 | 0.468 |
| 46a | 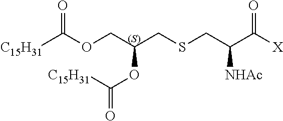 | 150.598 |
| 46b | 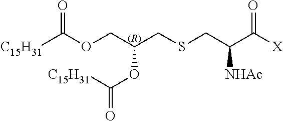 | 0.281 |
| 47a | 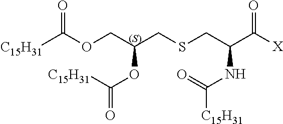 | 161.006 |
| 47b | 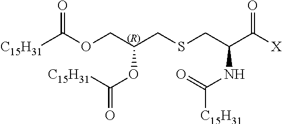 | 22.959 |
| 910 | 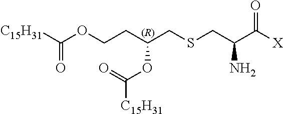 | 0.609 |
| 911 | 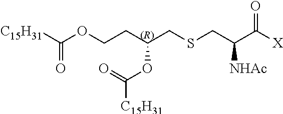 | 1.162 |
| 912 | 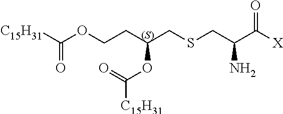 | 46.156 |
| 913 | 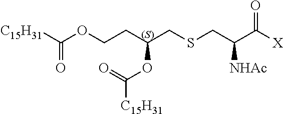 | 17.381 |

EC₅₀ values were then determined for chain extended constructs 930, 931 and 932 and compared with the EC₅₀ values for homoPam2Cys construct 910 and corresponding Pam2Cys construct 45b (Table 4). The hTLR agonism assay results are shown in FIG. 2B. All constructs analysed exhibited significant hTLR2 agonism. Constructs 930 and 931 had similar EC₅₀ values to 910 and 45b. Construct 932 exhibited slightly reduced activity compared with construct 910.

TABLE 4
Structures of peptide conjugates 45b, 910, 930-932 and EC₅₀ for human
TLR2 agonism as determined by HEK-Blue ^{TM} assay.
X = -SKKKKSLLMWITQV [SEQ ID No: 139].

| No. | Structure | EC₅₀ (nM) |
|---|---|---|
| 45b | 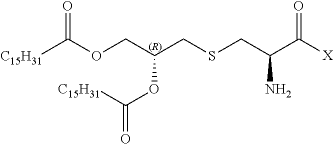 | 0.468 |
| 910 | 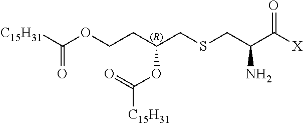 | 0.609 |
| 930 | 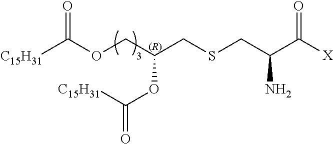 | 0.383 |
| 931 | 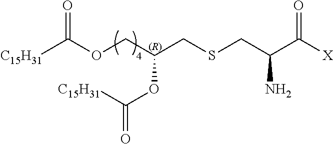 | 0.304 |
| 932 | 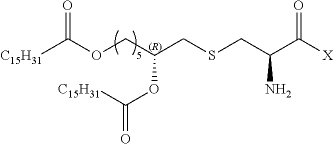 | 4.445 |

### 5. Example 5

This example investigates murine TLR2 agonism of compounds of the present invention compared with conjugates of known TLR2 agonists ((R)-Pam2Cys, (S)-Pam2Cys, (R)-Pam3Cys, and (S)-Pam3Cys).

### 5.1 Peptide Conjugates

The peptide conjugates tested are listed in Tables 3 and 4 above. Peptide conjugates 910, 911, 912, 913, 930, 931, and 932 were prepared as described above in Example 3. Peptide conjugates 45a, 45b, 46a, 46b, 47a, 47b were prepared using methods analogous to those described herein above.

### 5.2 Toll-Like Receptor 2 (TLR2) Agonism Using Hek-Blue Cells

Murine TLR2 agonism by the peptide conjugates was investigated using HEK-Blue™-mTLR2 cells and Hek-Blue™ Detection medium (Invivogen) across an 8-log₁₀ dilution series (10⁹ fM to 10² fM) as described above in Example 4.

### Results

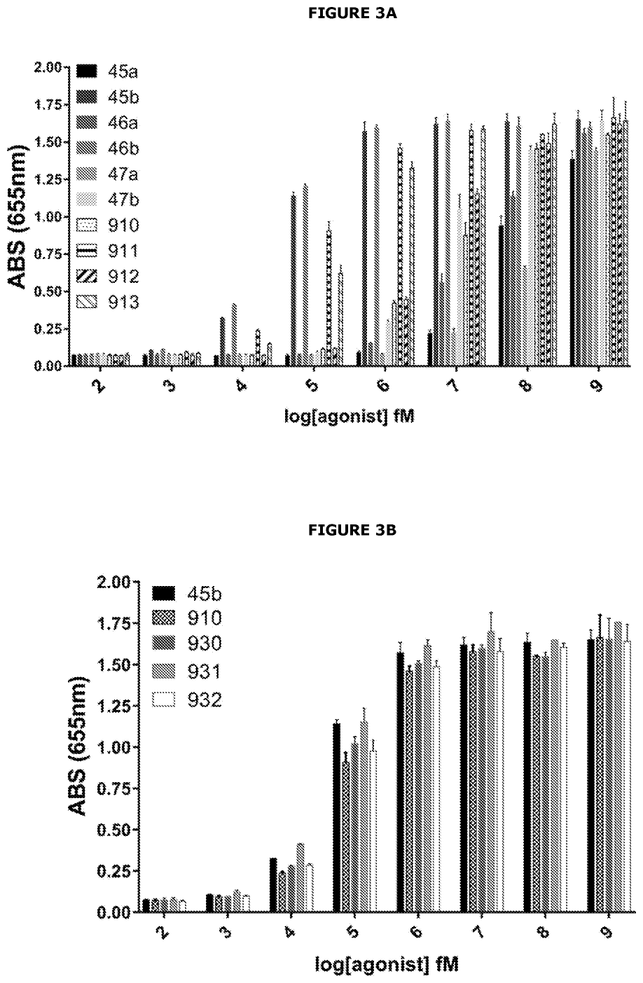

Results for homoPam2Cys constructs 910, 911, 912 and 913 compared with corresponding Pam2Cys constructs 45a and 45b, N-acetylated Pam2Cys constructs 46a and 46b and Pam3Cys constructs 47a and 47b (structures depicted in Table 3) are presented in FIG. 3A. All constructs exhibited murine TLR2 agonism.

Results for chain extended constructs 930, 931 and 932 compared with homoPam2Cys construct 910 and corresponding Pam2Cys construct 45b (structures depicted in Table 4) are presented in FIG. 3B. All constructs analysed exhibited murine TLR2 agonism.

It is not the intention to limit the scope of the invention to the abovementioned examples only. As would be appreciated by a skilled person in the art, many variations are possible without departing from the scope of the invention.

1. A compound of the formula (I): 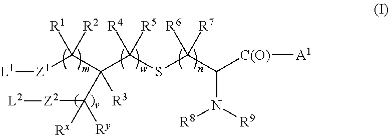
wherein
m and w are each independently an integer from 0 to 7 and v is an integer from 0 to 5,
provided that:
the sum of m, v, and w is at least 3; and
the sum of m and w is from 0 to 7;
n is 1 or 2;
Z1 and Z2 are each independently selected from the group consisting of —O—, —NR—, —S—, —S(O)—, —SO₂—, —C(O)O—, —OC(O)—, —C(O)NR—, —NRC(O)—, —C(O)S—, μSC(O)—, —OC(O)O—, —NRC(O)O—, —OC(O)NR—, and —NRC(O)NR—;
R1, R2, Rx, Ry, R4, R5, R6, and R7 at each instance of m, v, w, and n are each independently hydrogen or C1-6aliphatic;
R, R3, and R8 are each independently hydrogen or C1-6aliphatic;
R9 is hydrogen, C1-6aliphatic, an amino protecting group, L3-C(O)—, or A2;
L1 and L2 are each independently selected from C5-21aliphatic or C4-20heteroaliphatic;
L3 is C1-21aliphatic or C2-20heteroaliphatic;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any aliphatic or heteroaliphatic present in any of R, R1, R2, R3, R4, R5, R6, R7, R8, R9, Rx, Ry, L1, L2, and L3 is optionally substituted;
or a pharmaceutically acceptable salt or solvate thereof;
with the proviso that:
(1) at least one of R9 and A1 is a peptide comprising, consisting essentially of, or consisting of an amino acid sequence selected from the group consisting of:
(a) 8 or more contiguous amino acid residues from the sequence Xaa₁Xaa₂Xaa₃Xaa₄LQQLSLLMWITQXaa₂₂FLPVFLAQPPSGQRR [SEQ ID NO:1], wherein Xaa₁ is absent or is S, Xaa₂ is absent or is a hydrophilic amino acid, Xaa₃ is absent or is a hydrophilic amino acid, and Xaa₄ is absent or is one or more hydrophilic amino acids,
(b) 8 or more contiguous amino acid residues from the sequence Xaa₁Xaa₂Xaa₃LQQLSLLMWITQXaa₂₂FLPVFLAQPPSGQRR [SEQ ID NO:2], wherein Xaa₁ is absent or is S, Xaa₂ is absent or is a hydrophilic amino acid, and Xaa₃ is absent or is from one to ten hydrophilic amino acids,
(c) 8 or more contiguous amino acid residues from the sequence Xaa₁Xaa₂LQQLSLLMWITQXaa₂₂FLPVFLAQPPSGQRR [SEQ ID NO:3], wherein Xaa₁ is absent or is S, and Xaa₂ is absent or is from one to four hydrophilic amino acids,
(d) 8 or more contiguous amino acid residues from the sequence
[SEQ ID NO: 4]
SKKKKLQQLSLLMWITQXaa₂₂FLPVFLAQPPSGQRR,
(e) the sequence of any one of SEQ ID NOs: 1 to 4,
(f) 8 or more contiguous amino acid residues from the sequence
[SEQ ID NO: 5]
LQQLSLLMWITQXaa₂₂FLPVFLAQPPSGQRR,
(g) the sequence of SEQ ID NO: 5,
(h) 8 or more contiguous amino acid residues from the sequence
[SEQ ID NO: 6]
SLLMWITQXaa₂₂FLPVF,
(i) the sequence of SEQ ID NO: 6,
(j) 8 or more contiguous amino acid residues from the sequence
[SEQ ID NO: 7]
SKKKKSLLMWITQXaa₂₂,
(k) the sequence of SEQ ID NO: 7,
(l) 8 or more contiguous amino acid residues from the sequence SLLMWITQXaa₂₂ [SEQ ID NO:8],
(m) the sequence of SEQ ID NO: 8,
(n) or any combination of two or more of (a) to (m) above,
wherein Xaa₂₂ in each sequence is independently any naturally occurring amino acid except C (for example, V, I, or L), and any sequence of 8 or more contiguous amino acid residues from any of the above sequences comprises Xaa₂₂; or
(2) m is an integer from 3 to 7, and at least one of R9 and A1 is an amino acid or a peptide.

2. The compound of claim 1, wherein at least one of R9 and A1 is a peptide comprising, consisting essentially of, or consisting of one or more amino acid sequences selected from the group as defined in proviso (1) of claim 1.

3. The compound of claim 2, wherein m and w are each independently from 0 to 5.

4. The compound of claim 2 or 3, wherein m and w are each independently from 1 to 4.

5. The compound of any one of claims 2 to 4, wherein the sum of m and w is from 2 to 7.

6. The compound of any one of claims 2 to 5, wherein the sum of m and w is from 2 to 5.

7. The compound of any one of claims 2 to 6, wherein the sum of m and w is 3.

8. The compound of any one of claims 2 to 7, wherein m is from 1 to 6.

9. The compound of any one of claims 2 to 8, wherein m is from 1 to 5.

10. The compound of any one of claims 2 to 9, wherein m is from 1 to 3.

11. The compound of any one of claims 2 to 10, wherein m is 2.

12. The compound of claim 1, wherein m is an integer from 3 to 7 and at least one of R9 and A1 is an amino acid or a peptide as defined in proviso (2) of claim 1.

13. The compound of claim 12, wherein at least one of R9 and A1 is a peptide.

14. The compound of claim 12 or 13, wherein the peptide comprises, consists essentially of, or consists of an amino acid sequence selected from the group consisting of 8 or more contiguous amino acids from the amino acid sequence of any one of SEQ ID NOs 8-129.

15. The compound of claim 12 or 13, wherein the peptide comprises, consists essentially of, or consists of one or more amino acid sequences selected from the group defined in proviso (1) of claim 1.

16. The compound of any one of the preceding claims, wherein Xaa₂₂ in each sequence is V.

17. The compound of any one of the preceding claims, wherein m is from 3 to 6.

18. The compound of any one of the preceding claims, wherein m is from 3 to 5.

19. The compound of any one of the preceding claims, wherein
R1, R2, Rx, Ry, R4, R5, R6, and R7 at each instance of m, v, w, and n are each independently hydrogen, C1-6alkyl, or C3-6cycloalkyl;
R, R3, and R8 are each independently hydrogen, C1-6alkyl, or C3-6cycloalkyl;
R9 is hydrogen, C1-6alkyl, C3-6cycloalkyl, an amino protecting group, L3-C(O), or A2;
L1 and L2 are each independently selected from C5-21alkyl, C5-21alkenyl, or C4-20heteroalkyl;
L3 is C1-21alkyl, C2-21alkenyl, C3-6cycloalkyl, or C2-20heteroalkyl;
A1 is an amino acid, a peptide, OH, OP1, NH2, or NHP2, wherein P1 is a carboxyl protecting group, and wherein P2 is a carboxamide protecting group;
A2 is an amino acid or a peptide;
wherein any alkyl, alkenyl, cycloalkyl or heteroalkyl present in any of R, R1, R2, R3, R4, R5, R6, R7, R8, R9, Rx, Ry, L1, L2, and L3 is optionally substituted.

20. The compound of any one of the preceding claims, wherein Z1 and Z2 are each independently selected from the group consisting of —C(O)O—, —C(O)NR—, and —C(O)S—.

21. The compound of any one of the preceding claims, wherein the compound is a compound of the formula (IA):

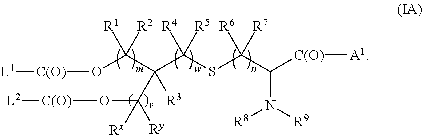

22. The compound of any one of the preceding claims, wherein v is from 0 to 3.

23. The compound of any one of the preceding claims, wherein v is 0.

24. The compound of any one of the preceding claims, wherein w is 1 or 2.

25. The compound of any one of the preceding claims, wherein w is 1.

26. The compound of any one of the preceding claims, wherein n is 1.

27. The compound of any one of the preceding claims, wherein L1 and L2 are each independently C5-21alkyl.

28. The compound of any one of the preceding claims, wherein L1 and L2 are each independently linear C15alkyl.

29. The compound of any one of the preceding claims, wherein L3 is methyl or linear C15alkyl.

30. The compound of any one of the preceding claims, wherein L3 is methyl.

31. The compound of any one of the preceding claims, wherein the amino protecting group is Boc or Fmoc.

32. The compound of any one of the preceding claims, wherein R1 and R2 at each instance of m are each independently C1-6alkyl or hydrogen, preferably hydrogen.

33. The compound of any one of the preceding claims, wherein R3 is C1-6alkyl or hydrogen, preferably hydrogen.

34. The compound of any one of the preceding claims, wherein R4 and R5 at each instance of w are each independently C1-6alkyl or hydrogen, preferably hydrogen.

35. The compound of any one of the preceding claims, wherein Rx and Ry at each instance of v are each independently C1-6alkyl or hydrogen, preferably hydrogen.

36. The compound of any one of the preceding claims, wherein R6 and R7 at each instance of n are each independently C1-6alkyl or hydrogen, preferably hydrogen.

37. The compound of any one of the preceding claims, wherein R8 is independently C1-6alkyl or hydrogen, preferably hydrogen.

38. The compound of any one of the preceding claims, wherein R9 is C1-6alkyl, hydrogen, an amino protecting group, L3-C(O), or A2, preferably hydrogen, an amino protecting group, L3-C(O), or A2.

39. The compound of any one of the preceding claims, wherein the compound is a compound of the formula (IF):

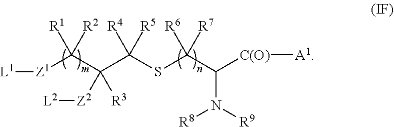

40. The compound of claim 39, wherein the compound is a compound of the formula (IF-1):

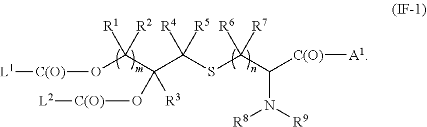

41. The compound of any one of claims 1 to 38, wherein the compound is a compound of the formula (IB): 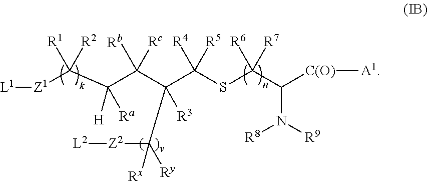
wherein
k is an integer from 0 to 4; and
Ra, Rb, and Rc are each independently hydrogen or C1-6aliphatic.

42. The compound of claim 41, wherein the compound of formula (IB) is a compound of the formula (IC):

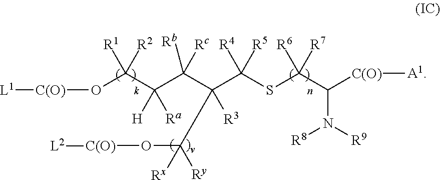

43. The compound of claim 41 or 42, wherein k is 0 to 3.

44. The compound of any one of claims 41 to 43, wherein k is 0.

45. The compound of any one of claims 41 to 43, wherein k is 1 to 3.

46. The compound of any one of claims 41 to 45, wherein Ra, Rb, and Rc are each independently hydrogen, C1-6alkyl, or C3-6cycloalkyl, preferably hydrogen.

47. The compound of any one of claims 41 to 46, wherein Ra, Rb, and Rc are each independently selected from hydrogen or C1-6alkyl, preferably hydrogen.

48. The compound of any one of the preceding claims, wherein the compound is a compound of the formula (ID-1):

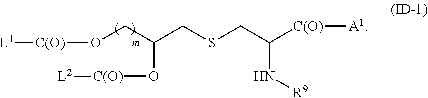

49. The compound of claim 48, wherein m is from 3 to 5.

50. The compound of claim 48, wherein the compound is a compound of the formula (ID):

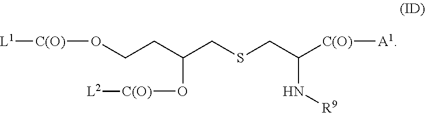

51. The compound of any one of the preceding claims, wherein the compound of formula (I) has the formula (IE):

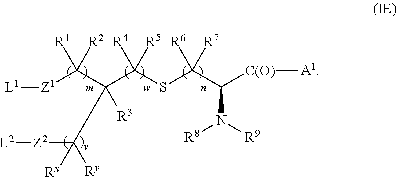

52. The compound of any one of the preceding claims, wherein the compound has the formula (IE-1):

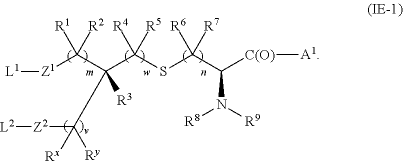

53. The compound of any one of the preceding claims, wherein the compound has the formula (IE-2):

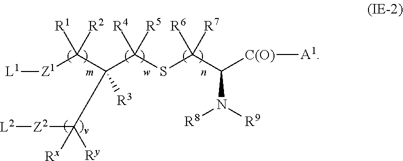

54. The compound of any one of the preceding claims, wherein the peptide comprises an epitope.

55. The compound of claim 54, wherein the epitope is a peptide epitope.

56. The compound of claim 54 or 55, wherein the epitope is coupled or bound via a linker group.

57. The compound of any one of the preceding claims, wherein the amino acid of the peptide conjugate to which the lipid moieties are conjugated is an N-terminal amino acid residue.

58. The compound of any one of the preceding claims, wherein A1 is serine or a peptide comprising serine as the first N-terminal amino acid residue.

59. The compound of any one of the preceding claims, wherein A1 and/or A2 is a peptide comprising a solubilising group.

60. The compound of claim 59, wherein the solubilising group comprises an amino acid sequence comprising two or more hydrophilic amino acid residues in the peptide chain.

61. The compound of claim 60, wherein the two or more hydrophilic amino acid residues are adjacent to the serine residue.

62. The compound of any one of the preceding claims, wherein A1 is a peptide and R9 is hydrogen or L3-C(O), for example Me-C(O).

63. The compound of any one of the preceding claims, wherein the optional substituents are selected from the group consisting of halo, CN, NO₂, OH, NH₂, NHR10, NR10R20, C1-6haloalkyl, C1-6haloalkoxy, C(O)NH₂, C(O)NHR10, C(O)NR10R20, SO₂R10, OR10, SR10, S(O)R10, C(O)R10, and C1-6aliphatic; wherein R10 and R20 are each independently C1-6aliphatic, for example C1-6alkyl.

64. A method of making a peptide conjugate of the formula (I) or a pharmaceutically acceptable salt or solvate thereof according to any one of the preceding claims, the method comprising:
(A) reacting
a first lipid-containing conjugation partner comprising a carbon-carbon double bond,
a second lipid-containing conjugation partner comprising a carbon-carbon double bond, and
an amino acid-comprising conjugation partner comprising a thiol
under conditions effective to conjugate the first lipid-containing conjugation partner and the second lipid-containing conjugation partner to the amino acid-comprising conjugation partner and provide the peptide-conjugate of formula (I) or salt or solvate thereof,
wherein in the amino acid- or peptide conjugate the sulfur atom from the thiol of the amino acid-comprising conjugation partner is conjugated to a carbon atom from the carbon-carbon double bond of the first lipid-containing conjugation partner, and a carbon atom from the carbon-carbon double bond of the first lipid-containing conjugation partner is conjugated to a carbon atom from the carbon-carbon double bond of the second lipid-containing conjugation partner; or
(B) reacting
a first lipid-containing conjugation partner comprising a carbon-carbon double bond,
a second lipid-containing conjugation partner comprising a carbon-carbon double bond, and
an amino acid-comprising conjugation partner comprising a thiol
under conditions effective to conjugate the first lipid-containing conjugation partner and the second lipid-containing conjugation partner to the amino acid-comprising conjugation partner and provide an amino acid- or peptide-conjugate,
wherein in the amino acid- or peptide conjugate the sulfur atom from the thiol of the amino acid-comprising conjugation partner is conjugated to a carbon atom from the carbon-carbon double bond of the first lipid-containing conjugation partner, and a carbon atom from the carbon-carbon double bond of the first lipid-containing conjugation partner is conjugated to a carbon atom from the carbon-carbon double bond of the second lipid-containing conjugation partner; and
coupling the amino acid of the amino acid conjugate or an amino acid of the peptide conjugate to an amino acid or an amino acid of a peptide to provide the peptide-conjugate of formula (I) or salt or solvate thereof.

65. The method of claim 64, wherein the first and second lipid-containing conjugation partners have the same structure.

66. The method of claim 64 or 65, wherein the method comprises conjugating the sulfur atom of the thiol to a carbon atom of the carbon-carbon double bond of the first lipid containing conjugation partner and then conjugating a carbon atom from the carbon-carbon double bond to which the thiol is conjugated to a carbon atom of the carbon-carbon double bond of the second lipid-containing conjugation partner.

67. The method of any one of claims 64 to 66, wherein:
the first lipid-containing conjugation partner is a compound of the formula (IIA): 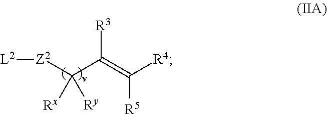
the second lipid-containing conjugation partner is a compound of the formula (IIB): 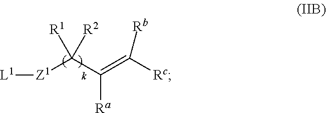
and
the amino acid-comprising conjugation partner comprises a structure of the formula (III): 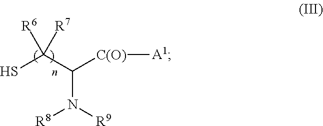
wherein:
when the method is (A), Ra, Rb, Rc, L1, L2, Z1, Z2, R1, R2, Rx, Ry, R3, R4, R5, R6, R7, R8, R9, A1, k, v, and n are as defined in the compound of formula (IB) according to any one of the preceding claims (including provisos (1) and/or (2) of claim 1); and
when the method is (B), Ra, Rb, Rc, L1, L2, Z1, Z2, R1, R2, Rx, Ry, R3, R4, R5, R6, R7, R8, R9, A1, k, v, and n are as defined in the compound of formula (IB) according to any one of the preceding claims but excluding provisos (1) and (2) of claim 1.

68. The method of any one of claims 64 to 67, wherein the amino acid- or peptide conjugate is a compound of the formula (IB): 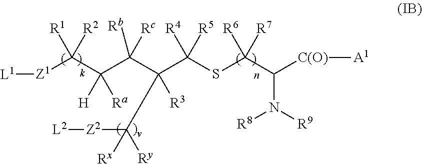
wherein:
when the method is (A), Ra, Rb, Rc, L1, L2, Z1, Z2, R1, R2, Rx, Ry, R3, R4, R5, R6, R7, R8, R9, A1, k, v, and n are as defined in the compound of formula (IB) according to any one of the preceding claims (including provisos (1) and (2) of claim 1); and
when the method is (B), Ra, Rb, Rc, L1, L2, Z1, Z2, R1, R2, Rx, Ry, R3, R4, R5, R6, R7, R8, R9, A1, k, v, and n are as defined in the compound of formula (IB) according to any one of the preceding claims but excluding provisos (1) and (2) of claim 1.

69. The method of any one of claims 64 to 68, the lipid containing conjugation partners are in stoichiometric excess to the amino acid-comprising conjugation partner.

70. The method of any one of claims 64 to 69, wherein the conditions effective to conjugate the lipid-containing conjugation partner to the amino acid-comprising conjugation partner comprises the generation of one or more free radicals initiated by the thermal degradation of a thermal initiator or the photochemical degradation of a photochemical initiator.

71. The method of claim 70, wherein the thermal initiator is AIBN or the photoinitiator is DMPA.

72. The method of claim 70 or 71, wherein photochemical degradation of the free radical initiator comprises irradiation with ultraviolet light, preferably having a frequency compatible with the side chains of naturally occurring amino acids, preferably about 365 nm.

73. The method of any one of claims 64 to 72, wherein the reaction is carried out in a liquid medium comprising a solvent, wherein the solvent comprises NMP, DMF, DMSO, or a mixture thereof.

74. The method of claim 73, wherein the solvent comprises NMP.

75. The method of any one of claims 64 to 74, wherein the reaction is carried out in the presence of one or more additives that inhibit the formation of by-products and/or that improve the yield of or conversion to the desired conjugate.

76. The method of claim 74, wherein the one or more additive is an extraneous thiol, an acid, an organosilane, or a combination of any two or more thereof.

77. The method of claim 75, wherein the extraneous thiol is a sterically hindered thiol, for example tert-butyl mercaptan.

78. The method of claim 75 or 76, wherein the acid is a strong organic acid, for example TFA.

79. The method of any one of claims 75 to 77, wherein the organosilane is a trialkylsilane, for example TIPS.

80. The method of any one of claims 75 to 78, wherein the amino acid conjugate or peptide conjugate is separated from the reaction medium after the reaction and optionally purified.

81. A method of making a peptide conjugate of the formula (IF) or a pharmaceutically acceptable salt or solvate thereof as defined in any one of the preceding claims, the method comprising:
(A) reacting
an epoxide of the formula (XVI): 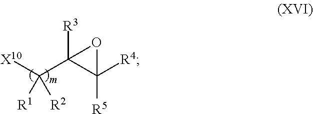
and
an amino acid-comprising conjugation partner comprising a thiol of the formula (III): 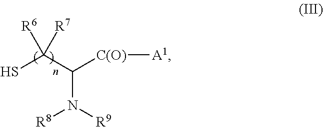
under conditions effective to conjugate the epoxide and amino acid-comprising conjugation partner and provide a compound of formula (XV): 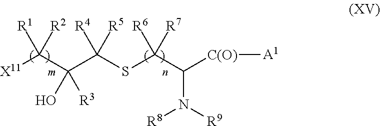
wherein
X10 is L1-Z1-, —OH, —SH, —NHR, HNRC(O)O—, P10-O—, P11-S—, P12-NR—, or P12-NRC(O)O—;
X11 is X10 or —OH, —SH, —NHR, or HNRC(O)O— when X10 is P10-O—, P11-S—, P12-NR—, or P12-NRC(O)O— and said conditions are effective to remove P10, P11, or P12;
P10, P11, and P12 are each independently a protecting group;
m, n, L1, Z1, R, R1, R2, R3, R4, R5, R6, R7, R8, R9, and A1 are as defined in the compound of formula (IF) according to any one the preceding claims (including provisos (1) and/or (2) of claim 1); and
converting the compound of formula (XV) to the peptide-conjugate of the formula (IF) according to in any one of the preceding compound claims (including provisos (1) and/or (2) of claim 1) or a pharmaceutically acceptable salt or solvate thereof by one or more additional synthetic steps: 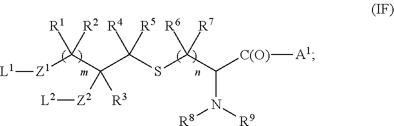
or
(B) reacting
an epoxide of the formula (XVI): 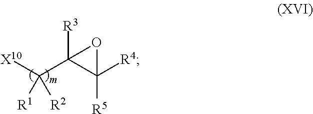
and
an amino acid-comprising conjugation partner comprising a thiol of the formula (III): 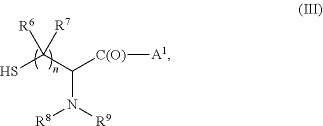
under conditions effective to conjugate the epoxide and amino acid-comprising conjugation partner and provide a compound of formula (XV): 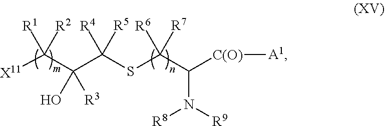
wherein
X10 is L1-Z1-, —OH, —SH, —NHR, HNRC(O)O—, P10-O—, P11-S—, P12-NR—, or P12-NRC(O)O—;
X11 is X10 or —OH, —SH, —NHR, or HNRC(O)O— when X10 is P10-O—, P11-S—, P12-NR—, or P12-NRC(O)O— and said conditions are effective to remove P10, P11, or P12;
P10, P11, and P12 are each independently a protecting group;
m, n, L1, Z1, R, R1, R2, R3, R4, R5, R6, R7, R8, R9, and A1 are as defined in the compound of formula (IF) according to any one the preceding claims but excluding provisos (1) and (2) of claim 1; and
converting the compound of formula (XV) to an amino acid- or peptide-conjugate of the formula (IF) according to any one of the preceding claims but excluding provisos (1) and (2) of claim 1 or a salt or solvate thereof by one or more additional synthetic steps: 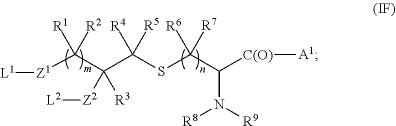
and
coupling the amino acid of the amino acid conjugate or an amino acid of the peptide conjugate to an amino acid or an amino acid of a peptide to provide the peptide-conjugate of formula (IF) according to any one of the preceding compound claims (including provisos (1) and/or (2) of claim 1) or pharmaceutically acceptable salt or solvate thereof.

82. The method of claim 81, wherein X10 is L1-C(O)O—, OH, or P10-O—; and X11 is L1-C(O)O—, P10-O—, or OH.

83. The method of claim 81 or 82, wherein the method comprises reacting the epoxide and amino acid-comprising conjugation partner in the presence of an acid.

84. The method of any one of claims 81 to 83, wherein the method comprises providing the epoxide by reacting an alkene of the formula (XVII): 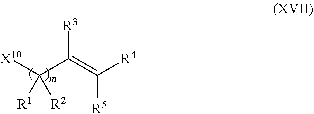
and an oxidant under conditions effective to epoxidise the alkene.

85. The method of any one of claims 81 to 83, wherein the method comprises providing the epoxide by reacting an compound of the formula (XVII-A), wherein LG is a leaving group: 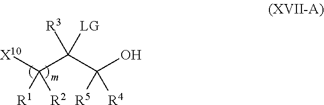
and a base under conditions effective for epoxidation.

86. The method of any one of claims 81 to 85, wherein X11 is P10-O— or OH; and the one or more synthetic steps comprise acylating the compound of formula (XV) so as to replace P10 or the hydrogen atom of the hydroxyl group of X11 with L1-C(O)—; and/or acylating the compound of formula (XV) so as to replace the hydrogen atom of the hydroxyl group bound to the carbon to which R3 is attached with L2-C(O)—.

87. A method of making a peptide-conjugate of the formula (I) or a pharmaceutically acceptable salt or solvate thereof as defined in any one of the preceding claims, the method comprising:
(A) reacting
a compound of the formula (XXI): 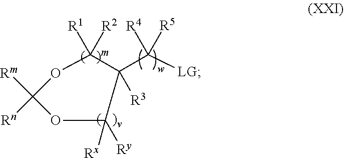
and
an amino acid-comprising conjugation partner comprising a thiol of the formula (III): 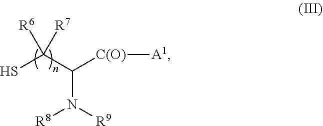
under conditions effective to conjugate the compound of formula (XXI) and amino acid-comprising conjugation partner and provide a compound of formula (XX): 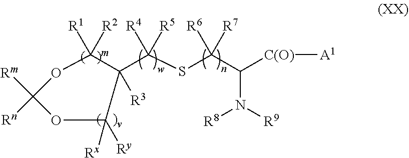
wherein
Rm and Rn are each independently hydrogen, C1-6alkyl, aryl, or heteroaryl;
LG is a leaving group; and
m, w, v, n, Rx, Ry, R1, R2, R3, R4, R5, R6, R7, R8, R9, and A1 are as defined in the compound of formula (I) according to any one of the preceding claims (including provisos (1) and/or (2) of claim 1); and
converting the compound of formula (XX) to a peptide conjugate of the formula (I) according to any one of the preceding claims (including provisos (1) and/or (2) of claim 1) or a pharmaceutically acceptable salt or solvate thereof by one or more additional synthetic steps: 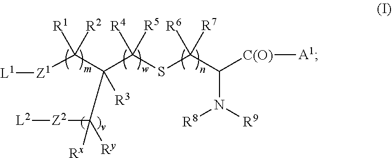
or
(B) reacting
a compound of the formula (XXI): 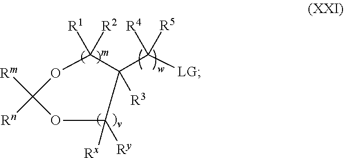
and
an amino acid-comprising conjugation partner comprising a thiol of the formula (III): 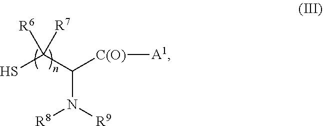
under conditions effective to conjugate the compound of formula (XXI) and amino acid-comprising conjugation partner and provide a compound of formula (XX): 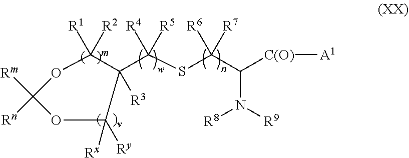
wherein
Rm and Rn are each independently hydrogen, C1-6alkyl, aryl, or heteroaryl;
LG is a leaving group; and
m, w, v, n, Rx, Ry, R1, R2, R3, R4, R5, R6, R7, R8, R9, and A1 are as defined in the compound of formula (I) according to any one of the preceding claims but excluding provisos (1) and (2) of claim 1; and
converting the compound of formula (XX) to an amino acid- or peptide conjugate of the formula (I) according to any one of the preceding claims but excluding provisos (1) and (2) of claim 1 or a salt or solvate thereof by one or more additional synthetic steps: 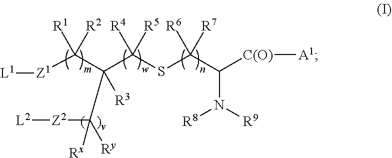
and
coupling the amino acid of the amino acid conjugate or an amino acid of the peptide conjugate to an amino acid or an amino acid of a peptide to provide the peptide-conjugate of formula (I) according to any one of the preceding compound claims (including provisos (1) and/or (2) of claim 1) or pharmaceutically acceptable salt or solvate thereof.

88. The method of claim 87, wherein Rm and Rn are each independently selected from hydrogen, C1-6alkyl, or aryl.

89. The method of claim 87 or 88, wherein Rm is hydrogen, C1-6alkyl, or aryl; and Rn is C1-6alkyl or aryl.

90. The method of any one of claims 87 to 89, wherein m and v are such that the compound of formula (XXI) comprises a 5-7-membered cyclic acetal.

91. The method of claim 90, wherein the cyclic acetal is a 6-membered cyclic acetal.

92. The method of any one of claims 87 to 91, wherein the method comprises reacting the compound of formula (XXI) and the amino acid-comprising conjugation partner of formula (III) in the presence of a base.

93. The method of any one of claims 87 to 92, wherein the one or more synthetic steps comprises removing the acetal in the compound of formula (XX) to provide a compound of the formula (XXIII-1):

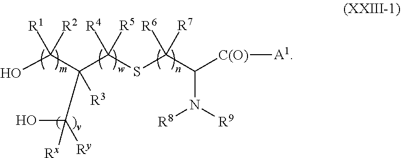

94. The method of any one of claims 87 to 92, wherein Rm is optionally substituted aryl, for example phenyl or methoxy substituted phenyl, and the method comprises removing the acetal in the compound of formula (XX) to provide a compound of the formula (XXIII-2) or (XXIII-3):

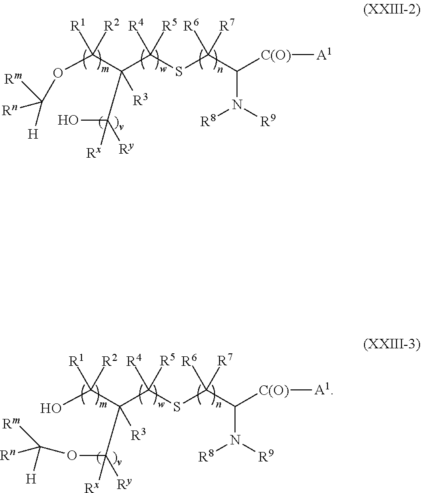

95. The method of claim 93, wherein the one or more synthetic steps comprise converting the hydroxyl group bound to the carbon to which R1 and R2 are attached in the compound of formula (XXIII-1) to L1-Z1-, and/or converting the hydroxyl group bound to the carbon to which Rx and Ry are attached to L2-Z2.

96. The method of claim 94, wherein the one or more synthetic steps comprise
converting the hydroxyl group bound to the carbon atom to which Rx and Ry are attached in the compound of formula (XXIII-2) to L2-Z2-, removing the RmRnCH- group to provide a hydroxyl group, and converting the hydroxyl group to L1-Z1; or
converting the hydroxyl group bound to the carbon to which Rx and Ry are attached in the compound of formula (XXIII-2) to L1-Z1-, removing the RmRnCH-group to provide a hydroxyl group, and converting the hydroxyl group to L2-Z2-.

97. The method of claim 95 or 96, wherein converting said hydroxyl group to L1-Z1- or L2-Z2- comprises acylating so as to replace the hydrogen atom of the hydroxyl group with L1-C(O)— or L2-C(O)—.

98. The method of any one of claims 64 to 97, wherein the amino acid-comprising conjugation partner is a peptide-containing conjugation partner.

99. The method of claim 98, wherein the peptide-containing conjugation partner comprises an epitope.

100. The method of any one of claims 64 to 99, wherein the amino acid-comprising conjugation partner consists of a peptide.

101. The method of any one of claims 64 to 100, wherein the amino acid-comprising conjugation partner is a peptide-containing conjugation partner comprising 15 or less, 14 or less, 13 or less, 12 or less, 11 or less, 10 or less, 9 or less, 8 or less, 7 or less, 6 or less, 5 or less, 4 or less, or 3 or less amino acid residues.

102. The method of any one of claims 64 to 97, wherein the method is (B) and the amino acid-comprising conjugation partner consists of an amino acid.

103. The method of any one of claims 64 to 102, the C-terminus of the amino acid comprising conjugation partner is protected with a protecting group and/or the Nα-amino group of the amino acid comprising conjugation partner is protected with a protecting group.

104. The method of any one of claims 64 to 101 and 103, wherein the amino acid residue comprising the thiol is an N-terminal amino acid residue.

105. The method of any one of claims 64 to 104, wherein the thiol is the thiol of a cysteine residue.

106. The method of any one of claims 64 to 105, wherein R9 in the amino acid comprising conjugation partner comprising the thiol is L3-C(O)—, for example Me-C(O)—.

107. The method of any one of claims 64 to 106, wherein the method is (B).

108. A method of making a peptide conjugate, the method comprising
providing an amino acid- or peptide conjugate of the formula (I) according to any one of claims 1 to 63 but excluding provisos (1) and (2) of claim 1 or a salt or solvate thereof, and
coupling the amino acid of the amino acid conjugate or an amino acid of the peptide conjugate to an amino acid or an amino acid of a peptide to provide a peptide conjugate of the formula (I) according to any one of claims 1 to 63 (including provisos (1) and/or (2) of claim 1) or a salt or solvate thereof.

109. The method of claim 107 or 108, wherein the method comprises coupling the amino acid of the amino acid conjugate to an amino acid or an amino acid of a peptide to provide the peptide conjugate.

110. The method of any one of claims 107 to 109, wherein the method comprises coupling the amino acid of the amino acid conjugate or an amino acid of the peptide conjugate to an amino acid or a peptide so as to provide a peptide conjugate comprising a peptide epitope.

111. The method of any one of claims 107 to 110, wherein the method comprises coupling an epitope to the amino acid of the amino acid conjugate or an amino acid of the peptide conjugate.

112. The method of claim 108 or 109, wherein the peptide comprises an epitope.

113. The method of any one of claims 99, 111, and 112, wherein the epitope is a peptide epitope.

114. The method of claim 113, wherein the epitope is coupled or bound via a linker group.

115. The method of any one of claims 107 to 114, wherein the amino acid of the peptide conjugate to which the lipid moieties are conjugated is an N-terminal amino acid residue.

116. The method of any one of claims 64 to 115, wherein the method further comprises acylating the Nα-amino group of the amino acid of the amino acid conjugate or the amino acid residue of the peptide conjugate to which the lipid moieties are conjugated.

117. The method of claim 116, wherein the amino group is acylated with a C2-20 fatty acid, such as acetyl.

118. The method of any one of claims 64 to 117, wherein the peptide conjugate or amino acid-comprising conjugation partner comprises one or more solubilising groups.

119. The method of claim 118, wherein the solubilising group is an amino acid sequence comprising a sequence of two or more consecutive hydrophilic amino acid residues in the peptide chain.

120. The method of any one of claims 64 to 118, wherein the peptide conjugate or amino acid-comprising conjugation partner comprises a serine residue adjacent to the amino acid residue to which the lipid moieties are conjugated.

121. An amino acid or peptide conjugate of the formula (I) of any one of claims 1 to 63 or a salt or solvate thereof made by a method of any one of claims 64 to 120.

122. A pharmaceutical composition comprising an effective amount of a peptide conjugate compound of any one of claims 1 to 63 and 121 or a pharmaceutically acceptable salt or solvate thereof, and a pharmaceutically acceptable carrier.

123. The pharmaceutical composition of claim 122 comprising an effective amount of two or more peptide conjugate compounds of any one of claims 1 to 63 and 121.

124. A method of vaccinating or eliciting an immune response in a subject comprising administering to the subject an effective amount of one or more peptide conjugate compounds of any one of claims 1 to 63 and 121 or a pharmaceutically acceptable salt or solvate thereof, or an effective amount of a pharmaceutical composition of claim 122 or 123.

125. Use of one or more peptide conjugate compounds of any one of claims 1 to 63 and 121 or a pharmaceutically acceptable salt or solvate thereof or a pharmaceutical composition of claim 122 or 123 in the manufacture of a medicament for vaccinating or eliciting an immune response in a subject.

126. One or more peptide conjugate compounds of any one of claims 1 to 63 and 121 or a pharmaceutically acceptable salt or solvate thereof or a pharmaceutical composition of claim 122 or 123 for vaccinating or eliciting an immune response in a subject.

127. A method of activating TLR2 in a subject, the method comprising administering to the subject an effective amount of one or more peptide conjugate of any one of claims 1 to 63 or a pharmaceutically acceptable salt or solvate thereof, or an effective amount of a pharmaceutical composition of claim 122 or 123.

128. Use of one or more peptide conjugate compounds of any one of claims 1 to 63 and 121 or a pharmaceutically acceptable salt or solvate thereof or a pharmaceutical composition of claim 122 or 123 in the manufacture of a medicament for activating TLR2 in a subject.

129. One or more peptide conjugate compounds of any one of claims 1 to 63 and 121 or a pharmaceutically acceptable salt or solvate thereof or a pharmaceutical composition of claim 122 or 123 for activating TLR2 in a subject.

130. The compound of any one of claims 1 to 63 and 121, method of any one of claims 64 to 120, pharmaceutical composition of claim 122 or 123, method of claim 124 or 127, use of claim 125 or 128, or peptide conjugate of claim 126 or 129, wherein the peptide conjugate compound of formula (I) is as defined in any one of claims 1 to 63 and 121 and has an EC₅₀ for TLR2 agonism (preferably hTLR2) of less than about about 500 nM as determined using a HEK-Blue™ cell assay.

131. The compound of any one of claims 1 to 63 and 121, method of any one of claims 64 to 120, pharmaceutical composition of claim 122 or 123, method of claim 124 or 127, use of claim 125 or 128, or peptide conjugate of claim 126 or 129, wherein the peptide conjugate compound of formula (I) is as defined in any one of claims 1 to 63 and 121 and comprises a peptide comprising, consists of, or consists essentially of an amino acid sequence selected from the group consisting of:
(a) 8 or more contiguous amino acid residues from the sequence
[SEQ ID NO: 7]
SKKKKSLLMWITQXaa₂₂,
(b) the sequence of SEQ ID NO: 7,
(c) 8 or more contiguous amino acid residues from the sequence SLLMWITQXaa₂₂ [SEQ ID NO:8],
(d) the sequence of SEQ ID NO: 8,
(e) or any combination of two or more of (a) to (d) above.

132. The compound of any one of claims 1 to 63 and 121, method of any one of claims 64 to 120, pharmaceutical composition of claim 122 or 123, method of claim 124 or 127, use of claim 125 or 128, or peptide conjugate of claim 126 or 129, wherein the peptide conjugate compound of formula (I) is as defined in any one of claims 1 to 63 and 121 and is a compound selected from the group consisting of compounds 910, 911, 912, 913, 930, 931, and 932 of the Examples herein.

133. The compound of any one of claims 1 to 63 and 121, method of any one of claims 64 to 120, pharmaceutical composition of claim 122 or 123, method of claim 124 or 127, use of claim 125 or 128, or peptide conjugate of claim 126 or 129, wherein the peptide conjugate compound of formula (I) is as defined in any one of claims 1 to 63 and 121 and comprises a peptide comprising, consisting essentially of, or consisting of an amino acid sequence selected from the group consisting of 8 or more contiguous amino acids from the amino acid sequence of any one of SEQ ID NOs 1-139.

134. A compound of the formula (XV): 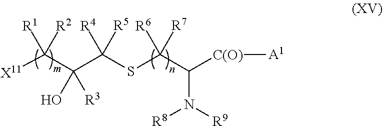
wherein
X11 is L1-Z1-, —OH, —SH, —NHR, HNRC(O)O—, P10-O—, P11-S—, P12-NR—, or P12-NRC(O)O—;
P10, P11, and P12 are each independently a protecting group;
m is an integer from 2 to 6; and
n, L1, Z1, R, R1, R2, R3, R4, R5, R6, R7, R8, R9, and A1 are as defined in the compound of formula (I) as defined in any one of the preceding claims (including provisos (1) and/or (2) of claim 1); or a salt or solvate thereof.

135. A compound of the formula (XX): 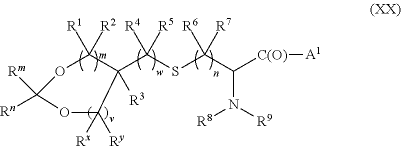
wherein:
Rm and Rn are each independently hydrogen, C1-6alkyl, aryl, or heteroaryl;
m and w are each independently an integer from 0 to 7 and v is an integer from 0 to 5,
provided that:
the sum of m, v, and w is at least 3; and
the sum of m and w is from 0 to 7; and
n, Rx, Ry, R1, R2, R3, R4, R5, R6, R7, R8, R9, and A1 are as defined in the compound of formula (I) as defined in any one of the preceding claims (including provisos (1) and/or (2) of claim 1); or a salt or solvate thereof.


SEQUENCE LISTING (139 sequences)

SEQ ID NO: 1 - protein, 32 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
  MISC_FEATURE (17)..(17): Xaa22 is any naturally occurring amino acid except C
XXXXLQQLSLLMWITQXFLPVFLAQPPSGQRR

SEQ ID NO: 2 - protein, 31 aa, Artificial Sequence
  Synthetic peptide sequence
  MISC_FEATURE (1)..(1): Xaa1 is absent or is S
  MISC_FEATURE (2)..(2): Xaa2 is absent or is a hydrophilic amino acid
  MISC_FEATURE (3)..(3): Xaa3 is absent or is from one to ten hydrophilic amino acids
  MISC_FEATURE (16)..(16): Xaa22 is any naturally occurring amino acid except C
XXXLQQLSLLMWITQXFLPVFLAQPPSGQRR

SEQ ID NO: 3 - protein, 30 aa, Artificial Sequence
  Synthetic peptide sequence
  MISC_FEATURE (1)..(1): Xaa1 is absent or is S
  MISC_FEATURE (2)..(2): Xaa2 is absent or is from one to four hydrophilic amino acids
  MISC_FEATURE (15)..(15): Xaa22 is any naturally occurring amino acid except C
XXLQQLSLLMWITQXFLPVFLAQPPSGQRR

SEQ ID NO: 4 - protein, 33 aa, Artificial Sequence
  Synthetic peptide sequence
  MISC_FEATURE (18)..(18): Xaa22 is any naturally occurring amino acid except C
SKKKKLQQLSLLMWITQXFLPVFLAQPPSGQRR

SEQ ID NO: 5 - protein, 28 aa, Artificial Sequence
  Synthetic peptide sequence
  MISC_FEATURE (13)..(13): Xaa22 is any naturally occurring amino acid except C
LQQLSLLMWITQXFLPVFLAQPPSGQRR

SEQ ID NO: 6 - protein, 14 aa, Artificial Sequence
  Synthetic peptide sequence
  MISC_FEATURE (9)..(9): Xaa22 is any naturally occurring amino acid except C
SLLMWITQXFLPVF

SEQ ID NO: 7 - protein, 14 aa, Artificial Sequence
  Synthetic peptide sequence
  MISC_FEATURE (14)..(14): Xaa22 is any naturally occurring amino acid except C
SKKKKSLLMWITQX

SEQ ID NO: 8 - protein, 9 aa, Artificial Sequence
  Synthetic peptide sequence
  MISC_FEATURE (9)..(9): Xaa22 is any naturally occurring amino acid except C
SLLMWITQX

SEQ ID NO: 9 - protein, 32 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXDRHSDYQPLGTQDQSLYLGLQHDGNDGL

SEQ ID NO: 10 - protein, 28 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
DRHSDYQPLGTQDQSLYLGLQHDGNDGL

SEQ ID NO: 11 - protein, 30 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXDRHSDYQPLGTQDQSLYLGLQHDGNDGL

SEQ ID NO: 12 - protein, 33 aa, Artificial Sequence
  Synthetic peptide sequence
SKKKKDRHSDYQPLGTQDQSLYLGLQHDGNDGL

SEQ ID NO: 13 - protein, 28 aa, Artificial Sequence
  Synthetic peptide sequence
DRHSDYQPLGTQDQSLYLGLQHDGNDGL

SEQ ID NO: 14 - protein, 37 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXSLYLGLQHDGNDGLPPPPYSPRDDSSQHIYEEA

SEQ ID NO: 15 - protein, 36 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXSLYLGLQHDGNDGLPPPPYSPRDDSSQHIYEEA

SEQ ID NO: 16 - protein, 35 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXSLYLGLQHDGNDGLPPPPYSPRDDSSQHIYEEA

SEQ ID NO: 17 - protein, 38 aa, Artificial Sequence
  Synthetic peptide sequence
SKKKKSLYLGLQHDGNDGLPPPPYSPRDDSSQHIYEEA

SEQ ID NO: 18 - protein, 33 aa, Artificial Sequence
  Synthetic peptide sequence
SLYLGLQHDGNDGLPPPPYSPRDDSSQHIYEEA

SEQ ID NO: 19 - protein, 29 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXSDYQPLGTQDQSLYLGLQHDGNDGL

SEQ ID NO: 20 - protein, 28 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXSDYQPLGTQDQSLYLGLQHDGNDGL

SEQ ID NO: 21 - protein, 27 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
XXSDYQPLGTQDQSLYLGLQHDGNDGL

SEQ ID NO: 22 - protein, 30 aa, Artificial Sequence
  Synthetic peptide sequence
SKKKKSDYQPLGTQDQSLYLGLQHDGNDGL

SEQ ID NO: 23 - protein, 25 aa, Artificial Sequence
  Synthetic peptide sequence
SDYQPLGTQDQSLYLGLQHDGNDGL

SEQ ID NO: 24 - protein, 51 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXDRHSDYQPLGTQDQSLYLGLQHDGNDGLPPPPYSPRDDSSQHIYEEA

SEQ ID NO: 25 - protein, 50 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXDRHSDYQPLGTQDQSLYLGLQHDGNDGLPPPPYSPRDDSSQHIYEEA

SEQ ID NO: 26 - protein, 49 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXDRHSDYQPLGTQDQSLYLGLQHDGNDGLPPPPYSPRDDSSQHIYEEA

SEQ ID NO: 27 - protein, 52 aa, Artificial Sequence
  Synthetic peptide sequence
SKKKKDRHSDYQPLGTQDQSLYLGLQHDGNDGLPPPPYSPRDDSSQHIYEEA

SEQ ID NO: 28 - protein, 47 aa, Artificial Sequence
  Synthetic peptide sequence
DRHSDYQPLGTQDQSLYLGLQHDGNDGLPPPPYSPRDDSSQHIYEEA

SEQ ID NO: 29 - protein, 40 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLLWTLVVLLICSSCSSCPLSKILLARLFLYALALLL

SEQ ID NO: 30 - protein, 39 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLLWTLVVLLICSSCSSCPLSKILLARLFLYALALLL

SEQ ID NO: 31 - protein, 38 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLLWTLVVLLICSSCSSCPLSKILLARLFLYALALLL

SEQ ID NO: 32 - protein, 41 aa, Artificial Sequence
  Synthetic peptide sequence
SKKKKLLWTLVVLLICSSCSSCPLSKILLARLFLYALALLL

SEQ ID NO: 33 - protein, 36 aa, Artificial Sequence
  Synthetic peptide sequence
LLWTLVVLLICSSCSSCPLSKILLARLFLYALALLL

SEQ ID NO: 34 - protein, 44 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLMLLWTLVVLLICSSCSSCPLSKILLARLFLYALALLLLA

SEQ ID NO: 35 - protein, 43 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLMLLWTLVVLLICSSCSSCPLSKILLARLFLYALALLLLA

SEQ ID NO: 36 - protein, 42 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLMLLWTLVVLLICSSCSSCPLSKILLARLFLYALALLLLA

SEQ ID NO: 37 - protein, 45 aa, Artificial Sequence
  Synthetic peptide sequence
SKKKKLMLLWTLVVLLICSSCSSCPLSKILLARLFLYALALLLLA

SEQ ID NO: 38 - protein, 40 aa, Artificial Sequence
  Synthetic peptide sequence
LMLLWTLVVLLICSSCSSCPLSKILLARLFLYALALLLLA

SEQ ID NO: 39 - protein, 30 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLMLLWTLVVLLICSSCSSCPLSKILL

SEQ ID NO: 40 - protein, 29 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLMLLWTLVVLLICSSCSSCPLSKILL

SEQ ID NO: 41 - protein, 28 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLMLLWTLVVLLICSSCSSCPLSKILL

SEQ ID NO: 42 - protein, 31 aa, Artificial Sequence
  Synthetic peptide sequence
SKKKKLMLLWTLVVLLICSSCSSCPLSKILL

SEQ ID NO: 43 - protein, 26 aa, Artificial Sequence
  Synthetic peptide sequence
LMLLWTLVVLLICSSCSSCPLSKILL

SEQ ID NO: 44 - protein, 35 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLLICSSCSSCPLSKILLARLFLYALALLLLA

SEQ ID NO: 45 - protein, 34 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLLICSSCSSCPLSKILLARLFLYALALLLLA

SEQ ID NO: 46 - protein, 33 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLLICSSCSSCPLSKILLARLFLYALALLLLA

SEQ ID NO: 47 - protein, 36 aa, Artificial Sequence
  Synthetic peptide sequence
SKKKKLLICSSCSSCPLSKILLARLFLYALALLLLA

SEQ ID NO: 48 - protein, 31 aa, Artificial Sequence
  Synthetic peptide sequence
LLICSSCSSCPLSKILLARLFLYALALLLLA

SEQ ID NO: 49 - protein, 57 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLNLTTMFLLMLLWTLVVLLICSSCSSCPLSKILLARLFLYALALLLLASALIA

SEQ ID NO: 50 - protein, 56 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLNLTTMFLLMLLWTLVVLLICSSCSSCPLSKILLARLFLYALALLLLASALIA

SEQ ID NO: 51 - protein, 55 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLNLTTMFLLMLLWTLVVLLICSSCSSCPLSKILLARLFLYALALLLLASALIA

SEQ ID NO: 52 - protein, 62 aa, Artificial Sequence
  Synthetic peptide sequence
SKKKKLNLTTMFLLMLLWTLVVLLICSSCSSCPLSKILLARLFLYALALLLLASALIAGGSI

SEQ ID NO: 53 - protein, 57 aa, Artificial Sequence
  Synthetic peptide sequence
LNLTTMFLLMLLWTLVVLLICSSCSSCPLSKILLARLFLYALALLLLASALIAGGSI

SEQ ID NO: 54 - protein, 48 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXFLLMLLWTLVVLLICSSCSSCPLSKILLARLFLYALALLLLASA

SEQ ID NO: 55 - protein, 47 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXFLLMLLWTLVVLLICSSCSSCPLSKILLARLFLYALALLLLASA

SEQ ID NO: 56 - protein, 46 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXFLLMLLWTLVVLLICSSCSSCPLSKILLARLFLYALALLLLASA

SEQ ID NO: 57 - protein, 49 aa, Artificial Sequence
  Synthetic peptide sequence
SKKKKFLLMLLWTLVVLLICSSCSSCPLSKILLARLFLYALALLLLASA

SEQ ID NO: 58 - protein, 44 aa, Artificial Sequence
  Synthetic peptide sequence
FLLMLLWTLVVLLICSSCSSCPLSKILLARLFLYALALLLLASA

SEQ ID NO: 59 - protein, 55 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLQGIYVLVMLVLLILAYRRRWRRLTVCGGIMFLACVLVLIVDAVLQLSPLL

SEQ ID NO: 60 - protein, 54 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLQGIYVLVMLVLLILAYRRRWRRLTVCGGIMFLACVLVLIVDAVLQLSPLL

SEQ ID NO: 61 - protein, 53 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLQGIYVLVMLVLLILAYRRRWRRLTVCGGIMFLACVLVLIVDAVLQLSPLL

SEQ ID NO: 62 - protein, 56 aa, Artificial Sequence
  Synthetic peptide sequence
SKKKKLQGIYVLVMLVLLILAYRRRWRRLTVCGGIMFLACVLVLIVDAVLQLSPLL

SEQ ID NO: 63 - protein, 51 aa, Artificial Sequence
  Synthetic peptide sequence
LQGIYVLVMLVLLILAYRRRWRRLTVCGGIMFLACVLVLIVDAVLQLSPLL

SEQ ID NO: 64 - protein, 56 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXSGNRTYGPVFMCSLGGLLTMVAGAVWLTVMSNTLLSAWILTAGFLIFLIGFA

SEQ ID NO: 65 - protein, 55 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXSGNRTYGPVFMCSLGGLLTMVAGAVWLTVMSNTLLSAWILTAGFLIFLIGFA

SEQ ID NO: 66 - protein, 54 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXSGNRTYGPVFMCSLGGLLTMVAGAVWLTVMSNTLLSAWILTAGFLIFLIGFA

SEQ ID NO: 67 - protein, 57 aa, Artificial Sequence
  Synthetic peptide sequence
SKKKKSGNRTYGPVFMCSLGGLLTMVAGAVWLTVMSNTLLSAWILTAGFLIFLIGFA

SEQ ID NO: 68 - protein, 52 aa, Artificial Sequence
  Synthetic peptide sequence
SGNRTYGPVFMCSLGGLLTMVAGAVWLTVMSNTLLSAWILTAGFLIFLIGFA

SEQ ID NO: 69 - protein, 51 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXSNEEPPPPYEDPYWGNGDRHSDYQPLGTQDQSLYLGLQHDGNDGLPP

SEQ ID NO: 70 - protein, 50 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXSNEEPPPPYEDPYWGNGDRHSDYQPLGTQDQSLYLGLQHDGNDGLPP

SEQ ID NO: 71 - protein, 49 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXSNEEPPPPYEDPYWGNGDRHSDYQPLGTQDQSLYLGLQHDGNDGLPP

SEQ ID NO: 72 - protein, 52 aa, Artificial Sequence
  Synthetic peptide sequence
SKKKKSNEEPPPPYEDPYWGNGDRHSDYQPLGTQDQSLYLGLQHDGNDGLPP

SEQ ID NO: 73 - protein, 47 aa, Artificial Sequence
  Synthetic peptide sequence
SNEEPPPPYEDPYWGNGDRHSDYQPLGTQDQSLYLGLQHDGNDGLPP

SEQ ID NO: 74 - protein, 55 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXGNDGLPPPPYSPRDDSSQHIYEEAGRGSMNPVCLPVIVAPYLFWLAAIAAS

SEQ ID NO: 75 - protein, 54 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXGNDGLPPPPYSPRDDSSQHIYEEAGRGSMNPVCLPVIVAPYLFWLAAIAAS

SEQ ID NO: 76 - protein, 53 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXGNDGLPPPPYSPRDDSSQHIYEEAGRGSMNPVCLPVIVAPYLFWLAAIAAS

SEQ ID NO: 77 - protein, 56 aa, Artificial Sequence
  Synthetic peptide sequence
SKKKKGNDGLPPPPYSPRDDSSQHIYEEAGRGSMNPVCLPVIVAPYLFWLAAIAAS

SEQ ID NO: 78 - protein, 51 aa, Artificial Sequence
  Synthetic peptide sequence
GNDGLPPPPYSPRDDSSQHIYEEAGRGSMNPVCLPVIVAPYLFWLAAIAAS

SEQ ID NO: 79 - protein, 54 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXAAIAASCFTASVSTVVTATGLALSLLLLAAVASSYAAAQRKLLTPVTVLT

SEQ ID NO: 80 - protein, 53 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXAAIAASCFTASVSTVVTATGLALSLLLLAAVASSYAAAQRKLLTPVTVLT

SEQ ID NO: 81 - protein, 52 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXAAIAASCFTASVSTVVTATGLALSLLLLAAVASSYAAAQRKLLTPVTVLT

SEQ ID NO: 82 - protein, 55 aa, Artificial Sequence
  Synthetic peptide sequence
SKKKKAAIAASCFTASVSTVVTATGLALSLLLLAAVASSYAAAQRKLLTPVTVLT

SEQ ID NO: 83 - protein, 50 aa, Artificial Sequence
  Synthetic peptide sequence
AAIAASCFTASVSTVVTATGLALSLLLLAAVASSYAAAQRKLLTPVTVLT

SEQ ID NO: 84 - protein, 10 aa, Artificial Sequence
  Synthetic peptide sequence
ESNEEPPPPY

SEQ ID NO: 85 - protein, 9 aa, Artificial Sequence
  Synthetic peptide sequence
SNEEPPPPY

SEQ ID NO: 86 - protein, 9 aa, Artificial Sequence
  Synthetic peptide sequence
HSDYQPLGT

SEQ ID NO: 87 - protein, 9 aa, Artificial Sequence
  Synthetic peptide sequence
PLGTQDQSL

SEQ ID NO: 88 - protein, 10 aa, Artificial Sequence
  Synthetic peptide sequence
PLGTQDQSLY

SEQ ID NO: 89 - protein, 9 aa, Artificial Sequence
  Synthetic peptide sequence
LGTQDQSLY

SEQ ID NO: 90 - protein, 9 aa, Artificial Sequence
  Synthetic peptide sequence
GTQDQSLYL

SEQ ID NO: 91 - protein, 9 aa, Artificial Sequence
  Synthetic peptide sequence
GTQDQSLYL

SEQ ID NO: 92 - protein, 10 aa, Artificial Sequence
  Synthetic peptide sequence
GTQDQSLYLG

SEQ ID NO: 93 - protein, 9 aa, Artificial Sequence
  Synthetic peptide sequence
QSLYLGLQH

SEQ ID NO: 94 - protein, 9 aa, Artificial Sequence
  Synthetic peptide sequence
SLYLGLQHD

SEQ ID NO: 95 - protein, 10 aa, Artificial Sequence
  Synthetic peptide sequence
GLQHDGNDGL

SEQ ID NO: 96 - protein, 10 aa, Artificial Sequence
  Synthetic peptide sequence
GNDGLPPPPY

SEQ ID NO: 97 - protein, 9 aa, Artificial Sequence
  Synthetic peptide sequence
GLPPPPYSP

SEQ ID NO: 98 - protein, 10 aa, Artificial Sequence
  Synthetic peptide sequence
GLPPPPYSPR

SEQ ID NO: 99 - protein, 10 aa, Artificial Sequence
  Synthetic peptide sequence
PRDDSSQHIY

SEQ ID NO: 100 - protein, 9 aa, Artificial Sequence
  Synthetic peptide sequence
RDDSSQHIY

SEQ ID NO: 101 - protein, 9 aa, Artificial Sequence
  Synthetic peptide sequence
HIYEEAGRG

SEQ ID NO: 102 - protein, 9 aa, Artificial Sequence
  Synthetic peptide sequence
ILLARLFLY

SEQ ID NO: 103 - protein, 11 aa, Artificial Sequence
  Synthetic peptide sequence
SSCSSCPLSKI

SEQ ID NO: 104 - protein, 9 aa, Artificial Sequence
  Synthetic peptide sequence
LLWTLVVLL

SEQ ID NO: 105 - protein, 9 aa, Artificial Sequence
  Synthetic peptide sequence
FLYALALLL

SEQ ID NO: 106 - protein, 9 aa, Artificial Sequence
  Synthetic peptide sequence
CLGGLLTMV

SEQ ID NO: 107 - protein, 9 aa, Artificial Sequence
  Synthetic peptide sequence
LIVDAVLQL

SEQ ID NO: 108 - protein, 9 aa, Artificial Sequence
  Synthetic peptide sequence
LTAGFLIFL

SEQ ID NO: 109 - protein, 9 aa, Artificial Sequence
  Synthetic peptide sequence
TVCGGIMFL

SEQ ID NO: 110 - protein, 42 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXGARGPESRLLEFYLAMPFATPMEAELARRSLAQDAPPL

SEQ ID NO: 111 - protein, 41 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXGARGPESRLLEFYLAMPFATPMEAELARRSLAQDAPPL

SEQ ID NO: 112 - protein, 40 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXGARGPESRLLEFYLAMPFATPMEAELARRSLAQDAPPL

SEQ ID NO: 113 - protein, 43 aa, Artificial Sequence
  Synthetic peptide sequence
SKKKKGARGPESRLLEFYLAMPFATPMEAELARRSLAQDAPPL

SEQ ID NO: 114 - protein, 38 aa, Artificial Sequence
  Synthetic peptide sequence
GARGPESRLLEFYLAMPFATPMEAELARRSLAQDAPPL

SEQ ID NO: 115 - protein, 9 aa, Artificial Sequence
  Synthetic peptide sequence
LAMPFATPM

SEQ ID NO: 116 - protein, 9 aa, Artificial Sequence
  Synthetic peptide sequence
FATPMEAEL

SEQ ID NO: 117 - protein, 30 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXVPGVLLKEFTVSGNILTIRLTAADHR

SEQ ID NO: 118 - protein, 29 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXVPGVLLKEFTVSGNILTIRLTAADHR

SEQ ID NO: 119 - protein, 28 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXVPGVLLKEFTVSGNILTIRLTAADHR

SEQ ID NO: 120 - protein, 31 aa, Artificial Sequence
  Synthetic peptide sequence
SKKKKVPGVLLKEFTVSGNILTIRLTAADHR

SEQ ID NO: 121 - protein, 26 aa, Artificial Sequence
  Synthetic peptide sequence
VPGVLLKEFTVSGNILTIRLTAADHR

SEQ ID NO: 122 - protein, 9 aa, Artificial Sequence
  Synthetic peptide sequence
EFTVSGNIL

SEQ ID NO: 123 - protein, 32 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXLQQLSLLMWITQCFLPVFLAQPPSGQRR

SEQ ID NO: 124 - protein, 31 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXLQQLSLLMWITQCFLPVFLAQPPSGQRR

SEQ ID NO: 125 - protein, 30 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXLQQLSLLMWITQCFLPVFLAQPPSGQRR

SEQ ID NO: 126 - protein, 33 aa, Artificial Sequence
  Synthetic peptide sequence
SKKKKLQQLSLLMWITQCFLPVFLAQPPSGQRR

SEQ ID NO: 127 - protein, 28 aa, Artificial Sequence
  Synthetic peptide sequence
LQQLSLLMWITQCFLPVFLAQPPSGQRR

SEQ ID NO: 128 - protein, 14 aa, Artificial Sequence
  Synthetic peptide sequence
SLLMWITQCFLPVF

SEQ ID NO: 129 - protein, 9 aa, Artificial Sequence
  Synthetic peptide sequence
SLLMWITQC

SEQ ID NO: 130 - protein, 35 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is a hydrophilic amino acid
  MISC_FEATURE (4)..(4): Xaa4 is absent or is one or more hydrophilic amino acids
XXXXKISQAVHAAHAEINEAGRESIINFEKLTEWT

SEQ ID NO: 131 - protein, 34 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is a hydrophilic amino acid
  MISC_FEATURE (3)..(3): Xaa3 is absent or is from one to ten hydrophilic amino acids
XXXKISQAVHAAHAEINEAGRESIINFEKLTEWT

SEQ ID NO: 132 - protein, 33 aa, Artificial Sequence
  Synthetic peptide sequence
  MISC_FEATURE (1)..(1): Xaa1 is absent or is S or a hydrophilic amino acid
  MISC_FEATURE (2)..(2): Xaa2 is absent or is from one to four hydrophilic amino acids
XXKISQAVHAAHAEINEAGRESIINFEKLTEWT

SEQ ID NO: 133 - protein, 36 aa, Artificial Sequence
  Synthetic peptide sequence
SKKKKKISQAVHAAHAEINEAGRESIINFEKLTEWT

SEQ ID NO: 134 - protein, 31 aa, Artificial Sequence
  Synthetic peptide sequence
KISQAVHAAHAEINEAGRESIINFEKLTEWT

SEQ ID NO: 135 - protein, 8 aa, Artificial Sequence
  Synthetic peptide sequence
SIINFEKL

SEQ ID NO: 136 - protein, 17 aa, Artificial Sequence
  Synthetic peptide sequence
ISQAVHAAHAEINEAGR

SEQ ID NO: 137 - protein, 9 aa, Artificial Sequence
  Synthetic peptide sequence
NLVPMVATV

SEQ ID NO: 138 - protein, 15 aa, Artificial Sequence
  Synthetic peptide sequence
CSKKKKNLVPMVATV

SEQ ID NO: 139 - protein, 14 aa, Artificial Sequence
  Synthetic peptide sequence
SKKKKSLLMWITQV